(19) United States
(12) Patent Application Publication
(10) Pub. No.: US 2025/0041461 A1
(43) Pub. Date: Feb. 6, 2025

(54) [177LU] LUTETIUM-PSMA I&T COMPOSITION AND DOSIMETRY, KIT, METHOD OF MAKING, AND METHOD OF USING THEREOF

(71) Applicant: Curium US LLC, St. Louis, MO (US)

(72) Inventors: Jarno Jalomäki, Helsinki (FI); Salla Seppänen, Helsinki (FI); Amanda Donovan, St. Louis, MO (US); Jessica Salem, St. Louis, MO (US); Allan Casciola, St. Louis, MO (US); Anthony Vaughn, St. Louis, MO (US)

(21) Appl. No.: 18/791,288

(22) Filed: Jul. 31, 2024

Related U.S. Application Data
(60) Provisional application No. 63/529,986, filed on Jul. 31, 2023.
(60) Provisional application No. 63/620,262, filed on Jan. 12, 2024.
(60) Provisional application No. 63/626,839, filed on Jan. 30, 2024.
(60) Provisional application No. 63/671,633, filed on Jul. 15, 2024.
(60) Provisional application No. 63/671,625, filed on Jul. 15, 2024.
(60) Provisional application No. 63/677,137, filed on Jul. 30, 2024.
(60) Provisional application No. 63/677,276, filed on Jul. 30, 2024.

(51) Int. Cl. A61K 51/04; A61K 47/18; A61K 47/22

(52) U.S. Cl. CPC A61K 51/0402; A61K 47/183; A61K 47/22; A61K 2121/00; A61K 2123/00

(57) ABSTRACT

The present disclosure relates to a radiopharmaceutical compositions, methods, and kits comprising ¹⁷⁷Lu-PSMA I&T. The compositions, methods, and kits may have a molar ratio of the PSMA I&T to ¹⁷⁷Lu from 4.4:1.0 to 7.6:1.0. The composition may be formulated as a solution for injection and the solution is suitable for administration at least 72 hours after formulation. The composition(s) have a radiochemical purity (RCP) of 95% or greater, 95.5% or greater, 96% or greater, 96.5% or greater, 97% or greater, 97.5% or greater, 98.0% or greater, 98.5% or greater, 99% or greater, or 99.5% or greater at administration.

### CROSS-REFERENCE TO RELATED APPLICATIONS

The present invention claims priority to the following U.S. Provisional Applications: Nos. 63/529,986, 63/620,262, 63/626,839, 63/671,633, 63/671,625, 63/677,137 and 63/677,276 filed Jul. 31, 2023, Jan. 12, 2024, Jan. 30, 2024, Jul. 15, 2024, Jul. 15, 2024, Jul. 30, 2024, and Jul. 30, 2024, respectively, which are all hereby incorporated in their entirety, including all tables, figures, and claims.

### FIELD

The present disclosure relates to a composition of a [¹⁷⁷Lu]Lutetium-PSMA I&T ([¹⁷⁷Lu]Lu-PSMA I&T or ¹⁷⁷Lu-PSMA I&T) solution for injection or intravenous infusion, as well as a kit comprising ¹⁷⁷Lu-PSMA I&T. The ¹⁷⁷Lu-PSMA I&T solution and/or kit thereof may be used for prostate cancer radioligand therapy (PRLT).

### BACKGROUND

Prostate cancer (PC) is the most frequent non-cutaneous cancer and the second most frequent cause of cancer deaths for adult men. Overall, increasing the survival rate for patients with metastatic castration-resistant prostate cancer (mCRPC) is challenging and there exists a clinical need for an effective treatment method for mCRPC patients.

Prostate-specific membrane antigen (PSMA) is highly expressed on prostate epithelial cells and strongly up-regulated in prostate cancer, which makes PSMA a promising molecular target for diagnosis and therapy of PC including, mCRPC. [¹⁷⁷Lu]Lu-PSMA-617 and [¹⁷⁷Lu]Lu-PSMA I&T are small molecule inhibitors of PSMA that are extremely desirable for targeted radionuclide therapy due to their low toxicity. However, the use of these small molecule inhibitors targeting PSMA with ¹⁷⁷Lu to treat prostate cancer can also result in undesirable absorbed doses of radiation to healthy organs.

There continues to be a need for an improved formulation containing [¹⁷⁷Lu]Lu-PSMA I&T that can be administered to patients which minimizes undesired cumulative absorbed doses of radiation to the patients' healthy organ tissue that is not being targeted for the treatment of cancer. Provided herein are solutions to overcome these and other problems in the art by providing an improved [¹⁷⁷Lu]Lu-PSMA I&T composition for PC and mCRPC treatment. Further, provided herein is an improved [¹⁷⁷Lu]Lu-PSMA I&T composition that provides lower cumulative absorbed dose of radiation, on a per administration basis.

### BRIEF SUMMARY

Provided herein, inter alia, are compositions directed to a radiopharmaceutical composition comprising ¹⁷⁷Lu-PSMA I&T for administration to a human patient in need thereof. The compositions are formulated as solutions for injection and the solutions are suitable for administration to a human patient in need thereof more than 48 hours, more than 72 hours, more than 96 hours, or more than 100 hours after formulation. The compositions are formulated as solutions for injection and the solutions radiochemical purity of ≥95% at least 48 hours after formulation, at least 72 hours after formulation, at least 96 hours after formulation, at least 100 hours after formulation, or at least 120 hours after formulation.

The compositions, methods, and kits described herein comprise ¹⁷⁷Lu-PSMA I&T suitable for administration to a human patient wherein the healthy organs exhibit reduced cumulative absorbed dose post administration.

The compositions, methods, and kits described herein comprise ¹⁷⁷Lu-PSMA I&T suitable for administration to a human patient at radiochemical purity of ≥95% and having a molar ratio of the PSMA I&T to ¹⁷⁷Lu that is from 3.0:1.0 to 8.0:1.0. and/or from 4.4:1.0 to 7.6:1.0. This is highly surprising and unexpected because our own initial testing suggested that this embodiment would not be feasible, and that a molar ratio of the PSMA I&T to ¹⁷⁷Lu of at least 11.0:1.0 or more would be required to maintain a radiochemical purity ≥95% for 72 hours or more. Indeed, according to our own initial expectations, anything below a ratio of 11.0:1.0 would likely have an unacceptable radiochemical purity (e.g., below 95%) at formation, and would continue to deteriorate such that it would be further unacceptable for a human patient at 24 hours after formation, 48 hours at formation, 72 hours after formation, or 96 hours after formation. See below PSMA:Lu-177 (mol/mol) graph showing unsuitable projected radiochemical formation at formulation below 11.0:1.0.

Yet, utilizing the unique parameters described herein, the compositions, methods, and kits described herein comprising ¹⁷⁷Lu-PSMA I&T suitable for administration to a human patient at radiochemical purity of ≥95% and having a molar ratio of the PSMA I&T to ¹⁷⁷Lu that is 3.0:1.0 to 8.0:1.0 and/or from 4.4:1.0 to 7.6:1.0, wherein the composition is stable for 72 hours or more, are provided herein.

In another embodiment, the compositions, methods, and kits described herein comprise ¹⁷⁷Lu-PSMA I&T suitable for administration to a human patient at radiochemical purity of ≥95% and having a PSMA I&T to [177Lu]Lu3+ ratio (in μg:mCi) from about 0.20 to about 0.60. In another embodiment, the compositions, methods, and kits described herein comprise ¹⁷⁷Lu-PSMA I&T suitable for administration to a human patient at radiochemical purity of ≥95% and having a PSMA I&T to [177Lu]Lu3+ ratio (in μg:mCi)≤0.60. This is also a highly surprising and unexpected because our own initial testing suggested that this embodiment would not be feasible, and having a PSMA I&T to [177Lu]Lu3+ ratio (in μg:mCi) of at least 0.70 or greater would be required to maintain a radiochemical purity ≥95% for 72 hours or more. See below PSMA:Lu-177 (mol/mol) graph showing unsuitable projected radiochemical formation at formulation at 0.60 and below.

Yet, utilizing the unique parameters described herein, the compositions, methods, and kits described herein comprising ¹⁷⁷Lu-PSMA I&T suitable for administration to a human patient at radiochemical purity of ≥95% and having a PSMA I&T to [177Lu]Lu3+ ratio (in μg:mCi) of from about 0.20 to about 0.64, from about 0.20 to about 0.63, from about 0.20 to about 0.62, from about 0.20 to about 0.61, or from about 0.20 to about 0.60, wherein the composition is stable for 72 hours or more, are provided herein.

The compositions, methods, and kits described herein comprise ¹⁷⁷Lu-PSMA I&T suitable for administration to a human patient at radiochemical purity of ≥95% and having a molar ratio of the PSMA I&T to ¹⁷⁷Lu that is from 3.0:1.0 to 8.0:1.0. and/or from 4.4:1.0 to 7.6:1.0. This is highly surprising and unexpected because our own initial testing suggested that this embodiment would not be feasible, and that a molar ratio of the PSMA I&T to ¹⁷⁷Lu of at least 11.0:1.0 or more would be required to maintain a radiochemical purity ≥95% for 72 hours or more. Indeed, according to our own initial expectations, anything below a ratio of 11.0:1.0 would likely have an unacceptable radiochemical purity (e.g., below 95%) at formation, and would continue to deteriorate such that it would be further unacceptable for a human patient at 24 hours after formation, 48 hours at formation, 72 hours after formation, or 96 hours after formation. See below PSMA:Lu-177 (mol/mol) graph showing unsuitable projected radiochemical formation at formulation below 11.0:1.0. Yet, utilizing the unique parameters described herein, the compositions, methods, and kits described herein comprising ¹⁷⁷Lu-PSMA I&T suitable for administration to a human patient at radiochemical purity of ≥95% and having a molar ratio of the PSMA I&T to ¹⁷⁷Lu that is 3.0:1.0 to 8.0:1.0 and/or from 4.4:1.0 to 7.6:1.0, wherein the composition is stable for 72 hours or more, are provided herein.

In another embodiment, the compositions, methods, and kits described herein comprise ¹⁷⁷Lu-PSMA I&T suitable for administration to a human patient at radiochemical purity of ≥95% and having a PSMA I&T to [177Lu]Lu3+ ratio (in μg:mCi) from about 0.20 to about 0.60. In another embodiment, the compositions, methods, and kits described herein comprise ¹⁷⁷Lu-PSMA I&T suitable for administration to a human patient at radiochemical purity of ≥95% and having a PSMA I&T to [177Lu]Lu3+ ratio (in μg:mCi)≤0.60. This is also a highly surprising and unexpected because our own initial testing suggested that this embodiment would not be feasible, and having a PSMA I&T to [177Lu]Lu3+ ratio (in μg:mCi) of at least 0.70 or greater would be required to maintain a radiochemical purity ≥95% for 72 hours or more. Yet, utilizing the unique parameters described herein, the compositions, methods, and kits described herein comprising ¹⁷⁷Lu-PSMA I&T suitable for administration to a human patient at radiochemical purity of ≥95% and having a PSMA I&T to [177Lu]Lu3+ ratio (in μg:mCi) of from about 0.20 to about 0.64, from about 0.20 to about 0.63, from about 0.20 to about 0.62, from about 0.20 to about 0.61, or from about 0.20 to about 0.60, wherein the composition is stable for 72 hours or more, are provided herein.

The present disclosure is further directed to a radiopharmaceutical composition comprising [¹⁷⁷Lu]Lu-PSMA I&T and ascorbic acid at a pH of 3.0 to 6.0 in solution. In some examples, the composition is suitable for administration to a human patient in need thereof at least 90 hours after formulation, and the composition has a radiochemical purity of 95.0% or greater at administration. In some aspects, the radiopharmaceutical composition may comprise a metal scavenger or chelating agent. The metal scavenger or chelating agent may comprise DTPA, EDTA, EDDS, DFOA, or a combination thereof. In other embodiments, the metal scavenger or chelating agent may comprise DTPA and an absence of EDTA. In some aspects, the radiopharmaceutical composition may comprise an absence of gentisic acid and/or gentisate (i.e., no gentisic acid or gentisate).

Another aspect of the present disclosure is a reaction composition comprising [¹⁷⁷Lu]LuCl₃, about 463 to 500 μg/mL PSMA I&T precursor in an ascorbic acid and acetate buffer. Another aspect of the present disclosure is a reaction composition comprising [¹⁷⁷Lu]LuCl₃, about 463 to 500 μg/mL PSMA I&T precursor, about 4 ml 0.4 M sodium acetate, about 1.6 mL 0.05 M hydrochloric acid, and about 150 μl 20% L-ascorbic acid in a total of 6 to 8 mL of solution. In yet another aspect of the present disclosure is a reaction composition comprising ¹⁷⁷Lu, about 463 to 1000 μg/mL PSMA I&T precursor in an ascorbic acid and acetate buffer. In some examples, the reaction composition has a ¹⁷⁷Lu radioactivity ≤61 GBq.

Another aspect of the present disclosure is a reaction composition comprising [¹⁷⁷Lu]Lu-, PSMA I&T precursor, ascorbic acid, acetate buffer, hydrochloric acid, and L-ascorbic acid in a solution. In some examples, the reaction composition has a ¹⁷⁷Lu radioactivity ≤296 GBq (i.e., ≤8,000 mCi).

The present disclosure is further directed to a radiopharmaceutical composition comprising [¹⁷⁷Lu]Lu-PSMA I&T solution for injection comprising [¹⁷⁷Lu]Lu-PSMA I&T and related substances (i.e., unlabeled PSMA I&T and PSMA I&T labeled with other metals) in an amount from about 3 μg/ml to about 16 μg/ml, ascorbic acid in a concentration from about 25 mg/ml to about 40 mg/ml, and a chelating agent in an amount from about 0.075 mg/ml to 0.15 mg/ml, wherein pH of the solution is between about 3 and about 5, wherein the total volume of the composition is about 15 ml and wherein upon administration of the composition to a subject, the subject maintains low levels of hematotoxic and nephrotoxic toxicity. The present disclosure is also further directed to a radiopharmaceutical composition comprising [¹⁷⁷Lu]Lu-PSMA I&T solution for injection comprising [¹⁷⁷Lu]Lu-PSMA I&T and related substances (i.e., unlabeled PSMA I&T and PSMA I&T labeled with other metals) in an amount from about 1 μg/ml to about 8 μg/ml, ascorbic acid in a concentration from about 25 mg/ml to about 40 mg/ml, and a chelating agent in an amount from about 0.075 mg/ml to 0.15 mg/ml, wherein pH of the solution is between about 3 and about 5, wherein the total volume of the composition is about 15 ml and wherein upon administration of the composition to a subject, the subject maintains low levels of hematotoxic and nephrotoxic toxicity. The present disclosure is also further directed to a radiopharmaceutical composition comprising [¹⁷⁷Lu]Lu-PSMA I&T solution for injection comprising [¹⁷⁷Lu]Lu-PSMA I&T and related substances (i.e., unlabeled PSMA I&T and PSMA I&T labeled with other metals) in an amount from about 3 μg/ml to about 10 μg/ml, ascorbic acid in a concentration from about 25 mg/ml to about 40 mg/ml, and a chelating agent in an amount from about 0.075 mg/ml to 0.15 mg/ml, wherein pH of the solution is between about 3 and about 5, wherein the total volume of the composition is about 15 ml and wherein upon administration of the composition to a subject, the subject maintains low levels of hematotoxic and nephrotoxic toxicity.

The present disclosure is further directed to a radiopharmaceutical composition comprising [¹⁷⁷Lu]Lu-PSMA I&T solution for injection comprising [¹⁷⁷Lu]Lu-PSMA I&T, ascorbic acid, and ethanol. Wherein the [¹⁷⁷Lu]Lu-PSMA I&T is in sufficient amounts of radioactivity for intended use, wherein the total amount of ascorbic acid in the solution is about 210-700 mg and the total amount of ethanol in the solution is about 274-706 mg; wherein pH of the solution is about 5 or below, wherein upon administration of the composition to a subject, the subject maintains low levels of hematotoxic and nephrotoxic toxicity, and wherein the prostate-specific antigen decline is more than about 50%.

The present disclosure is further directed to a radiopharmaceutical composition comprising [¹⁷⁷Lu]Lu-PSMA I&T solution for injection comprising [¹⁷⁷Lu]Lu-PSMA-I&T in an amount from about 3 μg/ml to about 15 μg/ml, ascorbic acid in a concentration from about 10 mg/ml to about 50 mg/ml, and ethanol in a concentration of about 0% (v/v), about 1% (v/v) to about 10% (v/v), about 2.5% (v/v %) to about 8.5% (v/v %), about 3.0% (v/v %) to about 8.0% (v/v %), about 3.8% (v/v %) to about 7.5% (v/v %), or about 4.5% (v/v %) to about 7.0% (v/v %) ethanol, wherein pH of the solution is between about 3 and about 5, and wherein upon administration of the composition to a subject, the subject maintains low levels of hematotoxic and nephrotoxic toxicity.

The present disclosure is further directed to a radiopharmaceutical composition comprising [¹⁷⁷Lu]Lu-PSMA I&T solution for injection comprising [¹⁷⁷Lu]Lu-PSMA I&T in an amount from about 3 μg/ml to about 15 μg/ml, or about 3 μg/ml to about 12 μg/ml; ascorbic acid in a concentration from about 10 mg/ml to about 50 mg/ml, ethanol in a concentration of about 0% (v/v) to about 10% (v/v), and a chelating agent in an amount from about 0.01 mg/ml to about 0.15 mg/ml or about, wherein pH of the solution is between about 3 and about 5, and wherein upon administration of the composition to a subject, the subject maintains low levels of hematotoxic and nephrotoxic toxicity.

The present disclosure is further directed to a radiopharmaceutical composition comprising [¹⁷⁷Lu]Lu-PSMA I&T solution for injection comprising [¹⁷⁷Lu]Lu-PSMA I&T and related substances (i.e., unlabeled PSMA I&T and PSMA I&T labeled with other metals) in an amount from about 4 μg/ml to about 15 μg/ml, ascorbic acid in a concentration from about 25 mg/ml to about 40 mg/ml, and a chelating agent in an amount from about 0.075 mg/ml to 0.15 mg/ml, wherein pH of the solution is between about 3 and about 5, wherein the total volume of the composition is about 15 ml and wherein upon administration of the composition to a subject, the subject maintains low levels of hematotoxic and nephrotoxic toxicity.

The present disclosure is further related to a radiopharmaceutical kit, comprising a vial containing at least a single dose of a [¹⁷⁷Lu]Lu-PSMA I&T solution for injection to a human patient in need thereof, wherein the injection comprises a dose of [¹⁷⁷Lu]Lu-PSMA-I&T, and wherein [¹⁷⁷Lu]Lu-PSMA I&T treatment with 1, 2, 3, 4, 5, 6, 7, 8 or more cycles at the dose is possible without the risk of kidney toxicities and/or wherein [¹⁷⁷Lu]Lu-PSMA I&T treatment with 1, 2, 3, 4, 5, 6, 7, 8 or more cycles provides a mean projected dose that is below a cumulative absorbed dose to the kidneys of 23 Gy.

The present disclosure is further related to a radiopharmaceutical kit, comprising a vial containing at least a single dose of a [¹⁷⁷Lu]Lu-PSMA I&T solution for injection to a human patient in need thereof, wherein the injection comprises a dose of [¹⁷⁷Lu]Lu-PSMA-I&T, and wherein [¹⁷⁷Lu]Lu-PSMA I&T treatment with 1, 2, 3, 4, 5, 6, 7, or 8 or more cycles at the dose that is below a cumulative absorbed dose to the kidneys of 23 Gy and/or wherein [¹⁷⁷Lu]Lu-PSMA I&T treatment with 1, 2, 3, 4, 5, 6, 7, or 8 or more cycles provides a mean projected cumulative absorbed dose to the kidneys of ≤23.0 Gy, ≤22.9 Gy, ≤22.8 Gy, ≤22.7 Gy, ≤22.6 Gy, ≤22.5 Gy, ≤22.4 Gy, ≤22.3 Gy, ≤22.2 Gy, ≤22.1 Gy, ≤22.0 Gy, ≤21.9 Gy, ≤21.8 Gy, ≤21.7 Gy, ≤21.6 Gy, ≤21.5 Gy, ≤21.4 Gy, ≤21.3 Gy, ≤21.2 Gy, ≤21.1 Gy, ≤21.0 Gy, ≤20.9 Gy, ≤20.8 Gy, ≤20.7 Gy, ≤20.6 Gy, ≤20.5 Gy, ≤20.4 Gy, ≤20.3 Gy, ≤20.2 Gy, ≤20.1 Gy, ≤20.0 Gy, ≤19.9 Gy, ≤19.8 Gy, ≤19.7 Gy, ≤19.6 Gy, ≤19.5 Gy, ≤19.4 Gy, ≤19.3 Gy, ≤19.2 Gy, or ≤19.1 Gy.

The present disclosure is further related to a radiopharmaceutical kit, comprising a vial containing at least a single dose of a [¹⁷⁷Lu]Lu-PSMA I&T solution for injection to a human patient in need thereof, wherein the injection comprises a dose of [¹⁷⁷Lu]Lu-PSMA-I&T, and wherein [¹⁷⁷Lu}Lu-PSMA I&T treatment with 1, 2, 3, 4, 5, 6, 7, or 8 or more cycles at the dose is possible without the risk of kidney toxicities and/or wherein [¹⁷⁷Lu]Lu-PSMA I&T treatment with 1, 2, 3, 4, 5, 6, 7, or 8 or more cycles provides a mean projected cumulative absorbed dose to the kidneys of ≤19.2 Gy, ≤19.1 Gy, ≤19.0 Gy, ≤18.9 Gy, ≤18.8 Gy, ≤18.7 Gy, ≤18.6 Gy, ≤18.5 Gy, ≤18.4 Gy, ≤18.3 Gy, ≤18.2 Gy, ≤18.1 Gy, ≤18.0 Gy, ≤17.9 Gy, ≤17.8 Gy, ≤17.7 Gy, ≤17.6 Gy, ≤17.5 Gy, ≤17.4 Gy, ≤17.3 Gy, ≤17.2 Gy, ≤17.1 Gy, ≤17.0 Gy, ≤16.8 Gy, ≤16.7 Gy, ≤16.6 Gy, ≤16.5 Gy, ≤16.4 Gy, ≤16.3 Gy, ≤16.2 Gy, ≤16.1 Gy, ≤16.0 Gy, or ≤15.9 Gy.

The present disclosure is further related to a radiopharmaceutical kit, comprising a vial containing at least a single dose of a [¹⁷⁷Lu]Lu-PSMA I&T solution for injection to a human patient in need thereof, wherein the injection comprises a dose of [177Lu-PSMA-I&T, and wherein [¹⁷⁷Lu]Lu-PSMA I&T treatment with 1, 2, 3, 4, 5, 6, 7, or 8 or more cycles at the dose is possible without the risk of kidney toxicities and/or wherein [¹⁷⁷Lu]Lu-PSMA I&T treatment with 1, 2, 3, 4, 5, 6, 7, or 8 or more cycles provides a mean projected dose that has a cumulative absorbed dose to the kidneys of ≤15.8 Gy, ≤15.7 Gy, ≤15.6 Gy, ≤15.5 Gy, ≤15.4 Gy, ≤15.3 Gy, ≤15.2 Gy, ≤15.1 Gy, ≤15.0 Gy, ≤14.9 Gy, ≤14.8 Gy, ≤14.7 Gy, ≤14.6 Gy, ≤14.3 Gy, ≤14.2 Gy, ≤14.1 Gy, ≤14.0 Gy, ≤13.9 Gy, ≤13.8 Gy, ≤13.7 Gy, ≤13.6 Gy, ≤13.5 Gy, ≤13.4 Gy, ≤13.3 Gy, ≤13.2 Gy, ≤13.1 Gy, ≤13.0 Gy, ≤12.9 Gy, ≤12.8 Gy, or ≤12.7 Gy.

The present disclosure is further related to a radiopharmaceutical kit, comprising a vial containing at least a single dose of a [¹⁷⁷Lu]Lu-PSMA I&T solution for injection to a human patient in need thereof, wherein the injection comprises a dose of [¹⁷⁷Lu]Lu-PSMA-I&T, and wherein [¹⁷⁷Lu]Lu-PSMA I&T treatment with 1, 2, 3, 4, 5, 6, 7, or 8 or more cycles at the dose is possible without the risk of kidney toxicities and/or wherein [¹⁷⁷Lu]Lu-PSMA I&T treatment with 1, 2, 3, 4, 5, 6, 7, or 8 or more cycles provides a mean projected dose that has a cumulative absorbed dose to the kidneys of ≤12.6 Gy, ≤12.5 Gy, ≤12.4 Gy, ≤12.3 Gy, ≤12.2 Gy, ≤12.1 Gy, ≤12.0 Gy, ≤11.9 Gy, ≤11.8 Gy, ≤11.7 Gy, ≤11.6 Gy, ≤11.5 Gy, ≤11.4 Gy, ≤11.3 Gy, ≤11.2 Gy, ≤11.1 Gy, ≤11.0 Gy, ≤10.9 Gy, ≤10.8 Gy, ≤10.7 Gy, ≤10.6 Gy, ≤10.5 Gy, ≤10.4 Gy, ≤10.3 Gy, ≤10.2 Gy, ≤10.1 Gy, ≤10.0 Gy, ≤9.9 Gy, ≤9.8 Gy, ≤9.7 Gy, ≤9.6 Gy, or ≤9.5 Gy.

The present disclosure is further related to a radiopharmaceutical kit, comprising a vial containing at least a single dose of a [¹⁷⁷Lu]Lu-PSMA I&T solution for injection to a human patient in need thereof, wherein the injection comprises a dose of [¹⁷⁷Lu]Lu-PSMA-I&T, and wherein [¹⁷⁷Lu]Lu-PSMA I&T treatment with 1, 2, 3, 4, 5, 6, 7, or 8 or more cycles at the dose is possible without the risk of kidney toxicities and/or wherein [¹⁷⁷Lu]Lu-PSMA I&T treatment with 1, 2, 3, 4, 5, 6, 7, or 8 or more cycles provides a mean projected dose that has a cumulative absorbed dose to the kidneys of ≤9.4 Gy, ≤9.3 Gy, ≤9.2 Gy, ≤9.1 Gy, ≤9.0 Gy, ≤8.9 Gy, ≤8.8 Gy, ≤8.7 Gy, ≤8.6 Gy, ≤8.5 Gy, ≤8.4 Gy, ≤8.3 Gy, ≤8.2 Gy, ≤8.1 Gy, ≤8.0 Gy, ≤7.9 Gy, ≤7.8 Gy, ≤7.7 Gy, ≤7.6 Gy, ≤7.5 Gy, ≤7.3 Gy, ≤7.2 Gy, ≤7.1 Gy, ≤7.0 Gy, ≤6.9 Gy, ≤6.8 Gy, ≤6.7 Gy, ≤6.6 Gy, ≤6.5 Gy, or ≤6.4 Gy.

The present disclosure is further related to a radiopharmaceutical kit, comprising a vial containing at least a single dose of a [¹⁷⁷Lu]-PSMA I&T solution for injection to a human patient in need thereof, wherein the injection comprises a dose of [¹⁷⁷Lu]Lu-PSMA-I&T, and wherein [¹⁷⁷Lu]Lu-PSMA I&T treatment with 1, 2, 3, 4, 5, 6, 7, or 8 or more cycles at the dose is possible without the risk of kidney toxicities and/or wherein [¹⁷⁷Lu]Lu-PSMA I&T treatment with 1, 2, 3, 4, 5, 6, 7, or 8 or more cycles provides a mean projected dose that has a cumulative absorbed dose to the kidneys of ≤6.3 Gy, ≤6.2 Gy, ≤6.1 Gy, ≤6.0 Gy, ≤5.9 Gy, ≤5.8 Gy, ≤5.7 Gy, ≤5.6 Gy, ≤5.5 Gy, ≤5.4 Gy, ≤5.3 Gy, ≤5.2 Gy, ≤5.1 Gy, ≤5.0 Gy, ≤4.9 Gy, ≤4.8 Gy, ≤4.7 Gy, ≤4.6 Gy, ≤4.5 Gy, ≤4.3 Gy, ≤4.2 Gy, ≤4.1 Gy, ≤4.0 Gy, ≤3.9 Gy, ≤3.8 Gy, ≤3.7 Gy, ≤3.6 Gy, ≤3.5 Gy, ≤3.4 Gy, ≤3.3 Gy, or ≤3.2 Gy.

The present disclosure is also related to a radiopharmaceutical kit, comprising a vial containing at least a single dose of a [¹⁷⁷Lu]Lu-PSMA I&T solution for injection to a human patient in need thereof, wherein the injection comprises a dose of [¹⁷⁷Lu]Lu-PSMA-I&T, and the projected or actual cumulative absorbed dose to the kidneys at 1, 2, 3, 4, 5, 6, 7, or 8 or more cycles is less than 23 Gy and no renal toxicities are observed.

The present disclosure is further related to a radiopharmaceutical kit, comprising a vial containing at least a single dose of a [¹⁷⁷Lu]Lu-PSMA I&T solution for injection to a human patient in need thereof, wherein the injection comprises less than 10 mg/ml gentisate, gentisic acid, alternative antioxidant, or a combination thereof. In some embodiments, the injection comprises ≤9.5 mg/ml, ≤9 mg/ml, ≤8.5 mg/ml, ≤8 mg/ml, ≤7.5 mg/ml, ≤7 mg/ml, ≤6.5 mg/ml, ≤6 mg/ml, ≤5.5 mg/ml, 5 mg/ml, ≤4.5 mg/ml, ≤4 mg/ml, ≤3.5 mg/ml, ≤3 mg/ml, ≤2.5 mg/ml, ≤2 mg/ml, ≤1.5 mg/ml, ≤1 mg/ml, or ≤0.5 mg/ml gentisate, gentisic acid, alternative antioxidant, or a combination thereof. In one embodiment, the present disclosure is related to a radiopharmaceutical kit comprising a vial containing at least a single dose of a [¹⁷⁷Lu]Lu-PSMA I&T solution for injection to a human patient in need thereof, wherein the injection comprises no gentisate, gentisic acid, alternative antioxidant, or a combination thereof.

The present disclosure is further related to a radiopharmaceutical kit, comprising a vial containing at least a single dose of a ¹⁷⁷Lu-PSMA I&T solution for injection to a human patient in need thereof, wherein the injection comprises less than 10 mg/ml ascorbate, ascorbic acid, alternative stabilizer or radioprotectant, or a combination thereof. In some embodiments, the injection comprises ≤40 mg/ml, ≤35 mg/ml, ≤33 mg/ml, ≤31 mg/ml, ≤30 mg/ml, ≤25 mg/ml, ≤20 mg/ml, ≤15 mg/ml, ≤10 mg/ml, ≤9.5 mg/ml, ≤9 mg/ml, ≤8.5 mg/ml, ≤8 mg/ml, ≤7.5 mg/ml, ≤7 mg/ml, ≤6.5 mg/ml, ≤6 mg/ml, ≤5.5 mg/ml, 5 mg/ml, ≤4.5 mg/ml, ≤4 mg/ml, ≤3.5 mg/ml, ≤3 mg/ml, ≤2.5 mg/ml, ≤2 mg/ml, ≤1.5 mg/ml, ≤1 mg/ml, or ≤0.5 mg/ml ascorbate, ascorbic acid, alternative stabilizer, or a combination thereof.

The present disclosure is further related to a radiopharmaceutical kit, comprising a vial containing at least a single dose of a [¹⁷⁷Lu]Lu-PSMA I&T solution for injection to a human patient in need thereof, wherein the injection comprises: (1) less than 10 mg/ml gentisate and or gentisic acid; and (2) less than 10 mg/ml ascorbate and/or ascorbic acid. In some embodiments, the injection comprises: (1) ≤9.5 mg/ml, ≤9 mg/ml, ≤8.5 mg/ml, ≤8 mg/ml, ≤7.5 mg/ml, ≤7 mg/ml, ≤6.5 mg/ml, ≤6 mg/ml, ≤5.5 mg/ml, 5 mg/ml, ≤4.5 mg/ml, ≤4 mg/ml, ≤3.5 mg/ml, ≤3 mg/ml, ≤2.5 mg/ml, ≤2 mg/ml, ≤1.5 mg/ml, ≤1 mg/ml, or ≤0.5 mg/ml gentisate and/or gentisic acid; and, (2) ≤9.5 mg/ml, ≤9 mg/ml, ≤8.5 mg/ml, ≤8 mg/ml, ≤7.5 mg/ml, ≤7 mg/ml, ≤6.5 mg/ml, ≤6 mg/ml, ≤5.5 mg/ml, 5 mg/ml, ≤4.5 mg/ml, ≤4 mg/ml, ≤3.5 mg/ml, ≤3 mg/ml, ≤2.5 mg/ml, ≤2 mg/ml, ≤1.5 mg/ml, ≤1 mg/ml, or ≤0.5 mg/ml ascorbate and/or ascorbic acid.

The present disclosure is further related to a radiopharmaceutical kit, comprising a vial containing at least a single dose of a [¹⁷⁷Lu]Lu-PSMA I&T solution for injection to a human patient in need thereof, wherein the injection comprises: (1) less than 10 mg/ml antioxidant; and, (2) less than 40 mg/ml stabilizer. In some embodiments, the injection comprises: (1) ≤9.5 mg/ml, ≤9 mg/ml, ≤8.5 mg/ml, ≤8 mg/ml, ≤7.5 mg/ml, ≤7 mg/ml, ≤6.5 mg/ml, ≤6 mg/ml, ≤5.5 mg/ml, 5 mg/ml, ≤4.5 mg/ml, ≤4 mg/ml, ≤3.5 mg/ml, ≤3 mg/ml, ≤2.5 mg/ml, ≤2 mg/ml, ≤1.5 mg/ml, ≤1 mg/ml, or ≤0.5 mg/ml antioxidant; and, (2) ≤40 mg/ml, ≤35 mg/ml, ≤33 mg/ml, ≤31 mg/ml, ≤30 mg/ml, ≤25 mg/ml, ≤20 mg/ml, ≤15 mg/ml, ≤10 mg/ml, ≤9.5 mg/ml, ≤9 mg/ml, ≤8.5 mg/ml, ≤8 mg/ml, ≤7.5 mg/ml, ≤7 mg/ml, ≤6.5 mg/ml, ≤6 mg/ml, ≤5.5 mg/ml, 5 mg/ml, ≤4.5 mg/ml, ≤4 mg/ml, ≤3.5 mg/ml, ≤3 mg/ml, ≤2.5 mg/ml, ≤2 mg/ml, ≤1.5 mg/ml, ≤1 mg/ml, or ≤0.5 mg/ml stabilizer.

The present disclosure is further related to a radiopharmaceutical kit, comprising a vial containing at least a single dose of a [¹⁷⁷Lu]Lu-PSMA I&T solution for injection to a human patient in need thereof, wherein the injection comprises a 6.8 GBq±0.10 GBq dose, 6.8 GBq±0.15 GBq dose, 6.8 GBq±0.20 GBq dose, 6.8 GBq±0.25 GBq dose, or 6.8 GBq±0.30 GBq dose, and wherein [¹⁷⁷Lu]Lu-PSMA I&T treatment with 1, 2, 3, 4, 5, 6, 7, or 8 cycles at the dose is possible without the risk of kidney toxicities and/or wherein [¹⁷⁷Lu]Lu-PSMA I&T treatment with 1, 2, 3, 4, 5, 6, 7, or 8 cycles provides a mean projected dose that is below a cumulative absorbed dose to the kidneys of 23 Gy and/or the projected or actual cumulative absorbed dose to the kidneys at 1, 2, 3, 4, 5, 6, 7, or 8 cycles is less than 23 Gy and no renal toxicities are observed.

The present disclosure is further related to a radiopharmaceutical kit, comprising a vial containing at least a single dose of a [¹⁷⁷Lu]Lu-PSMA I&T solution for injection to a human patient in need thereof, wherein the injection comprises a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, and wherein [¹⁷⁷Lu]Lu-PSMA I&T treatment with 1, 2, 3, 4, 5, 6, 7, or 8 cycles at the dose is possible without the risk of kidney toxicities and/or wherein [¹⁷⁷Lu]Lu-PSMA I&T treatment with 1, 2, 3, 4, 5, 6, 7, or 8 cycles provides a mean projected dose that is below a cumulative absorbed dose to the kidneys of 23 Gy and/or the projected or actual cumulative absorbed dose to the kidneys at 1, 2, 3, 4, 5, 6, 7, or 8 cycles is less than 23 Gy and no renal toxicities are observed.

The present disclosure is further related to a radiopharmaceutical kit, comprising a vial containing at least a single dose of a ¹⁷⁷Lu-PSMA I&T solution for injection to a human patient in need thereof, wherein the injection comprises a 7.4 GBq (mean 7.52±0.16 GBq) dose of ¹⁷⁷Lu-PSMA-I&T, and wherein ¹⁷⁷Lu-PSMA I&T treatment with 1, 2, 3, 4, 5, 6, 7, or 8 cycles at the dose is possible without the risk of kidney toxicities and/or wherein ¹⁷⁷Lu-PSMA I&T treatment with 1, 2, 3, 4, 5, 6, 7, or 8 cycles provides a mean projected dose that is below a cumulative absorbed dose to the kidneys of 23 Gy and/or the projected or actual cumulative absorbed dose to the kidneys at 1, 2, 3, 4, 5, 6, 7, or 8 cycles is less than 23 Gy and no renal toxicities are observed. The present disclosure is also further related to a radiopharmaceutical kit, comprising a vial containing at least a single dose of a ¹⁷⁷Lu-PSMA I&T solution for injection to a human patient in need thereof, wherein the injection comprises a 7.4 GBq (mean 7.52±0.16 GBq) dose of ¹⁷⁷Lu-PSMA-I&T, wherein ¹⁷⁷Lu-PSMA I&T treatment with 1, 2, 3, 4, 5, 6, 7, or 8 cycles provides a mean projected dose that is below a cumulative absorbed dose to the kidneys of 23 Gy and/or the projected or actual cumulative absorbed dose to the kidneys at 1, 2, 3, 4, 5, 6, 7, or 8 cycles is less than 23 Gy and no renal toxicities are observed.

The present disclosure is also further related to a radiopharmaceutical kit, comprising a vial containing at least a single dose of a ¹⁷⁷Lu-PSMA I&T solution for injection to a human patient in need thereof, wherein the injection comprises a 6.8 GBq±10% GBq dose, 6.8 GBq±5% GBq dose, or 6.8 GBq±3% GBq dose of ¹⁷⁷Lu-PSMA-I&T, and wherein ¹⁷⁷Lu-PSMA I&T treatment with 1, 2, 3, 4, 5, 6, 7, or 8 cycles at the dose is possible without the risk of kidney toxicities and/or wherein ¹⁷⁷Lu-PSMA I&T treatment with 1, 2, 3, 4, 5, 6, 7, or 8 cycles provides a mean projected dose that is below a cumulative absorbed dose to the kidneys of 23 Gy and/or the projected or actual cumulative absorbed dose to the kidneys at 1, 2, 3, 4, 5, 6, 7, or 8 cycles is less than 23 Gy and no renal toxicities are observed.

The present disclosure is also further related to a radiopharmaceutical kit, comprising a vial containing at least a single dose of a ¹⁷⁷Lu-PSMA I&T solution for injection to a human patient in need thereof, wherein the injection comprises a 7.4 GBq±10% GBq dose, 7.4 GBq±5% GBq dose, or 7.4 GBq±3% GBq dose of ¹⁷⁷Lu-PSMA-I&T, and wherein ¹⁷⁷Lu-PSMA I&T treatment with 1, 2, 3, 4, 5, 6, 7, or 8 cycles at the dose is possible without the risk of kidney toxicities and/or wherein ¹⁷⁷Lu-PSMA I&T treatment with 1, 2, 3, 4, 5, 6, 7, or 8 cycles provides a mean projected dose that is below a cumulative absorbed dose to the kidneys of 23 Gy and/or the projected or actual cumulative absorbed dose to the kidneys at 1, 2, 3, 4, 5, 6, 7, or 8 cycles is less than 23 Gy and no renal toxicities are observed.

The present disclosure is also further related to a radiopharmaceutical kit, comprising a vial containing at least a single dose of a ¹⁷⁷Lu-PSMA I&T solution for injection to a human patient in need thereof, wherein the injection comprises a 7.4 (±10%) GBq dose of ¹⁷⁷Lu-PSMA-I&T, and wherein ¹⁷⁷Lu-PSMA I&T treatment with 6 cycles at the dose is possible without the risk of kidney toxicities and/or wherein ¹⁷⁷Lu-PSMA I&T treatment with 6 cycles provides a mean projected dose that is below a cumulative absorbed dose to the kidneys of 23 Gy and/or the projected or actual cumulative absorbed dose to the kidneys at 6 cycles is less than 23 Gy and no renal toxicities are observed.

The present disclosure is further related to a radiopharmaceutical kit, comprising a vial containing at least a single dose of a ¹⁷⁷Lu-PSMA I&T solution for injection to a human patient in need thereof, wherein the injection comprises a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, and wherein the projected cumulative absorbed dose to the kidneys at 6 cycles will be 20.4±10.2 Gy. The present disclosure is further related to a radiopharmaceutical kit, comprising a vial containing at least a single dose of a ¹⁷⁷Lu-PSMA I&T solution for injection to a human patient in need thereof, wherein the injection comprises a 7.5 GBq±0.10 GBq dose, 7.5 GBq±0.15 GBq dose, 7.5 GBq±0.20 GBq dose, 7.5 GBq±0.25 GBq dose, or 7.5 GBq±0.30 GBq dose, and wherein the projected cumulative absorbed dose to the kidneys at 6 cycles will be 20.4±10.2 Gy.

The present disclosure is further related to a radiopharmaceutical kit, comprising a vial containing at least a single dose of a ¹⁷⁷Lu-PSMA I&T solution for injection to a human patient in need thereof, wherein the injection comprises a 7.1 GBq±0.10 GBq dose, 7.1 GBq±0.15 GBq dose, 7.1 GBq±0.20 GBq dose, 7.1 GBq±0.25 GBq dose, or 7.1 GBq±0.30 GBq dose, and wherein the projected cumulative absorbed dose to the kidneys at 6, 7 or 8 cycles will be 20.4±10.2 Gy.

The present disclosure is further related to a radiopharmaceutical kit, comprising a vial containing at least a single dose of a ¹⁷⁷Lu-PSMA I&T solution for injection to a human patient in need thereof, wherein the injection comprises a 6.8 GBq±0.10 GBq dose, 6.8 GBq±0.15 GBq dose, 6.8 GBq±0.20 GBq dose, 6.8 GBq±0.25 GBq dose, or 6.8 GBq±0.30 GBq dose, and wherein the projected cumulative absorbed dose to the kidneys at 6, 7 or 8 cycles will be 20.4±10.2 Gy.

The present disclosure is further related to a radiopharmaceutical kit, comprising a vial containing at least a single dose of a ¹⁷⁷Lu-PSMA I&T solution for injection to a human patient in need thereof, wherein the injection comprises a 6.5 GBq±0.10 GBq dose, 6.5 GBq±0.15 GBq dose, 6.5 GBq±0.20 GBq dose, 6.5 GBq±0.25 GBq dose, or 6.5 GBq±0.30 GBq dose, and wherein the projected cumulative absorbed dose to the kidneys at 6 7 or 8 cycles will be 20.4±10.2 Gy.

The present disclosure is further related to a radiopharmaceutical kit, comprising a vial containing at least a single dose of a ¹⁷⁷Lu-PSMA I&T solution for injection to a human patient in need thereof, wherein the injection comprises a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, and wherein the projected cumulative absorbed dose to the kidneys at 6 or 7 cycles will be ≤23.00 Gy, ≤22.50 Gy, ≤22.00 Gy, ≤21.50 Gy, ≤21.00 Gy, ≤20.50 Gy, or ≤20.40 Gy. The present disclosure is further related to a radiopharmaceutical kit, comprising a vial containing at least a single dose of a ¹⁷⁷Lu-PSMA I&T solution for injection to a human patient in need thereof, wherein the injection comprises a 7.5 GBq±0.10 GBq dose, 7.5 GBq±0.15 GBq dose, 7.5 GBq±0.20 GBq dose, 7.5 GBq±0.25 GBq dose, or 7.5 GBq±0.30 GBq dose, and wherein the projected cumulative absorbed dose to the kidneys at 6 or 7 cycles will be ≤23.00 Gy, ≤22.50 Gy, ≤22.00 Gy, ≤21.50 Gy, ≤21.00 Gy, ≤20.50 Gy, or ≤20.40 Gy.

The present disclosure is further related to a radiopharmaceutical kit, comprising a vial containing at least a single dose of a ¹⁷⁷Lu-PSMA I&T solution for injection to a human patient in need thereof, wherein the injection comprises a 6.8 GBq±0.10 GBq dose, 6.8 GBq±0.15 GBq dose, 6.8 GBq±0.20 GBq dose, 6.8 GBq±0.25 GBq dose, or 6.8 GBq±0.30 GBq dose, and wherein the projected cumulative absorbed dose to the kidneys at 6, 7 or 8 cycles will be ≤23.00 Gy, ≤22.50 Gy, ≤22.00 Gy, ≤21.50 Gy, ≤21.00 Gy, ≤20.50 Gy, or ≤20.40 Gy.

The present disclosure is further related to a radiopharmaceutical kit, comprising a vial containing at least a single dose of a ¹⁷⁷Lu-PSMA I&T solution for injection to a human patient in need thereof, wherein the injection comprises a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, and wherein a mean absorbed dose of the radiopharmaceutical is about 0.1 Gy/MBq to 1.0 Gy/MBq in the kidney of the human patient. The present disclosure is further related to a radiopharmaceutical kit, comprising a vial containing at least a single dose of a ¹⁷⁷Lu-PSMA I&T solution for injection to a human patient in need thereof, wherein the injection comprises a 7.5 GBq±0.10 GBq dose, 7.5 GBq±0.15 GBq dose, 7.5 GBq±0.20 GBq dose, 7.5 GBq±0.25 GBq dose, or 7.5 GBq±0.30 GBq dose, and wherein a mean absorbed dose of the radiopharmaceutical is about 0.1 Gy/MBq to 1.0 Gy/MBq in the kidney of the human patient.

The present disclosure is further related to a radiopharmaceutical kit, comprising a vial containing at least a single dose of a ¹⁷⁷Lu-PSMA I&T solution for injection to a human patient in need thereof, wherein the injection comprises a 6.8 GBq±0.10 GBq dose, 6.8 GBq±0.15 GBq dose, 6.8 GBq±0.20 GBq dose, 6.8 GBq±0.25 GBq dose, or 6.8 GBq±0.30 GBq dose, and wherein a mean absorbed dose of the radiopharmaceutical is about 0.1 Gy/MBq to 1.0 Gy/MBq in the kidney of the human patient.

The present disclosure is further related to a radiopharmaceutical kit, comprising a vial containing at least a single dose of a ¹⁷⁷Lu-PSMA I&T solution for injection to a human patient in need thereof, wherein the injection comprises a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, and wherein a mean whole body effective dose of the radiopharmaceutical composition is about 0.001 mSv/MBq to 0.1 mSv/MBq. The present disclosure is further related to a radiopharmaceutical kit, comprising a vial containing at least a single dose of a ¹⁷⁷Lu-PSMA I&T solution for injection to a human patient in need thereof, wherein the injection comprises a 7.5 GBq±0.10 GBq dose, 7.5 GBq±0.15 GBq dose, 7.5 GBq±0.20 GBq dose, 7.5 GBq±0.25 GBq dose, or 7.5 GBq±0.30 GBq dose, and wherein a mean whole body effective dose of the radiopharmaceutical composition is about 0.001 mSv/MBq to 0.1 mSv/MBq.

The present disclosure is further related to a radiopharmaceutical kit, comprising a vial containing at least a single dose of a ¹⁷⁷Lu-PSMA I&T solution for injection to a human patient in need thereof, wherein the injection comprises a 6.8 GBq±0.10 GBq dose, 6.8 GBq±0.15 GBq dose, 6.8 GBq±0.20 GBq dose, 6.8 GBq±0.25 GBq dose, or 6.8 GBq±0.30 GBq dose, and wherein a mean whole body effective dose of the radiopharmaceutical composition is about 0.001 mSv/MBq to 0.1 mSv/MBq.

The present disclosure is further related to a method of administering a radiopharmaceutical composition, the method comprising administering the radiopharmaceutical composition into a human patient in need thereof, optionally administering more than 48 hours after formulation, the radiopharmaceutical composition comprising a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose of ¹⁷⁷Lu-PSMA I&T in a solution comprising a pH of 3.5 to 5.0, the solution optionally comprising ascorbic acid and/or ethanol, and the solution optionally comprising a radiochemical purity of more than 95%, more than 96% more than 97%, more than 98%, more than 99%, or more than 99.5% when administered, and wherein ¹⁷⁷Lu-PSMA I&T treatment with 1, 2, 3, 4, 5, 6, and/or 7 cycles at the dose is possible without the risk of kidney toxicities and/or wherein ¹⁷⁷Lu-PSMA I&T treatment with 1, 2, 3, 4, 5, 6, and/or 7 cycles provides a mean projected dose that is below a cumulative absorbed dose to the kidney of 23 Gy and/or the projected or actual cumulative absorbed dose to the kidneys at 1, 2, 3, 4, 5, 6, and/or 7 cycles is less than 23 Gy and no renal toxicities are observed. In another aspect, the radiopharmaceutical composition may comprise radionuclidic identification (gamma spectrometry) of the composition showing gamma ray energy peaks at 113±2 keV and 208±4 keV, and no other significant peaks with gamma energy >100 keV are detected.

The present disclosure is further related to a method of administering a radiopharmaceutical composition, the method comprising administering the radiopharmaceutical composition into a human patient in need thereof, optionally administering more than 48 hours after formulation, the radiopharmaceutical composition comprising a 6.8 GBq±0.10 GBq dose, 6.8 GBq±0.15 GBq dose, 6.8 GBq±0.20 GBq dose, 6.8 GBq±0.25 GBq dose, or 6.8 GBq±0.30 GBq dose of [¹⁷⁷Lu]Lu-PSMA I&T in a solution comprising a pH of 3.5 to 4.5, the solution optionally comprising ascorbic acid and/or ethanol, and the solution optionally comprising a radiochemical purity of more than 95%, more than 96% more than 97%, more than 98%, more than 99%, or more than 99.5% when administered, and wherein [¹⁷⁷Lu]Lu-PSMA I&T treatment with 1, 2, 3, 4, 5, 6, 7 and/or 8 cycles at the dose is possible without the risk of kidney toxicities and/or wherein ¹⁷⁷Lu-PSMA I&T treatment with 1, 2, 3, 4, 5, 6, 7 and/or 8 cycles provides a mean projected dose that is below a cumulative absorbed dose to the kidney of 23 Gy and/or the projected or actual cumulative absorbed dose to the kidneys at 1, 2, 3, 4, 5, 6, 7 and/or 8 cycles is less than 23 Gy and no renal toxicities are observed. In another aspect, the radiopharmaceutical composition may comprise radionuclidic identification (gamma spectrometry) of the composition showing gamma ray energy peaks at 113±2 keV and 208±4 keV, and no other significant peaks with gamma energy >100 keV are detected.

The present disclosure is further related to a method of administering a radiopharmaceutical composition, the method comprising administering the radiopharmaceutical composition into a human patient in need thereof, optionally administering more than 48 hours after formulation, the radiopharmaceutical composition comprising a 6.5 GBq±0.10 GBq dose, 6.5 GBq±0.15 GBq dose, 6.5 GBq±0.20 GBq dose, 6.5 GBq±0.25 GBq dose, or 6.5 GBq±0.30 GBq dose of ¹⁷⁷Lu-PSMA I&T in a solution comprising a pH of 3.5 to 5.0, the solution optionally comprising ascorbic acid and/or ethanol, and the solution optionally comprising a radiochemical purity of more than 95%, more than 96% more than 97%, more than 98%, more than 99%, or more than 99.5% when administered, and wherein ¹⁷⁷Lu-PSMA I&T treatment with 1, 2, 3, 4, 5, 6, and/or 7 cycles at the dose is possible without the risk of kidney toxicities and/or wherein ¹⁷⁷Lu-PSMA I&T treatment with 1, 2, 3, 4, 5, 6, 7 and/or 8 cycles provides a mean projected dose that is below a cumulative absorbed dose to the kidney of 23 Gy and/or the projected or actual cumulative absorbed dose to the kidneys at 1, 2, 3, 4, 5, 6, 7 and/or 8 cycles is less than 23 Gy and no renal toxicities are observed. In another aspect, the radiopharmaceutical composition may comprise radionuclidic identification (gamma spectrometry) of the composition showing gamma ray energy peaks at 113±2 keV and 208±4 keV, and no other significant peaks with gamma energy >100 keV are detected.

The present disclosure is further related to a radiopharmaceutical kit comprising a vial containing at least a single dose of a ¹⁷⁷Lu-PSMA I&T solution for injection to a human patient in need thereof, wherein the injection comprises a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, and wherein the radionuclidic identification (gamma spectrometry) of the solution shows gamma ray energy peaks at 113±2 keV and 208±4 keV, and no other significant peaks with gamma energy >100 keV are detected.

The present disclosure is also related to a method comprising administering to a human patient in need thereof a radiopharmaceutical composition comprising ¹⁷⁷Lu-PSMA I&T that provides a mean absorbed dose to the patient's kidneys of 0.41±0.18 Gy/GBq, 0.41±0.17 Gy/GBq, 0.41±0.16 Gy/GBq, 0.41±0.15 Gy/GBq, 0.41±0.14 Gy/GBq, 0.41±0.13 Gy/GBq, 0.41±0.12 Gy/GBq, 0.41±0.11 Gy/GBq, 0.41±0.10 Gy/GBq, 0.41±0.09 Gy/GBq, 0.41±0.08 Gy/GBq, 0.41±0.07 Gy/GBq, 0.41±0.06 Gy/GBq, 0.41±0.05 Gy/GBq, 0.41±0.04 Gy/GBq, 0.41±0.03 Gy/GBq, 0.41±0.02 Gy/GBq, or 0.41±0.01 Gy/GBq, and wherein the radionuclidic identification (gamma spectrometry) of the solution shows gamma ray energy peaks at 113±2 keV and 208±4 keV, and no other significant peaks with gamma energy >100 keV are detected. The disclosure also relates to a method comprising administering to a human patient in need thereof a radiopharmaceutical composition comprising ¹⁷⁷Lu-PSMA I&T that provides a mean absorbed dose to the patient's kidneys that is ≤0.70 Gy/GBq, ≤0.65 Gy/GBq, ≤0.60 Gy/GBq, ≤0.55 Gy/GBq, ≤0.50 Gy/GBq, ≤0.45 Gy/GBq, ≤0.40 Gy/GBq, ≤0.35 Gy/GBq, ≤0.30 Gy/GBq, or ≤0.25 Gy/GBq.

The present disclosure also relates to a method comprising administering to a human patient in need thereof a radiopharmaceutical composition comprising ¹⁷⁷Lu-PSMA I&T that provides a mean absorbed dose to the patient's salivary glands that is 0.19±0.18 Gy/GB, 0.19±0.17 Gy/GBq, 0.19±0.16 Gy/GBq, 0.19±0.15 Gy/GBq, 0.19±0.14 Gy/GBq, 0.19±0.13 Gy/GBq, 0.19±0.12 Gy/GBq, 0.19±0.11 Gy/GBq, or 0.19±0.10 Gy/GBq. The present disclosure is also related to a method comprising administering to a human patient in need thereof a radiopharmaceutical composition comprising ¹⁷⁷Lu-PSMA I&T that provides a mean absorbed dose to the patient's salivary glands that is ≤0.30 Gy/GBq, ≤0.25 Gy/GBq, ≤0.25 Gy/GBq, ≤0.20 Gy/GBq, ≤0.15 Gy/GBq, ≤0.10 Gy/GBq, or ≤0.05 Gy/GBq.

The present disclosure also relates to a method comprising administering to a human patient in need thereof a radiopharmaceutical composition comprising ¹⁷⁷Lu-PSMA I&T that provides a mean absorbed dose to the patient's lacrimal glands that is 0.40±0.40 Gy/GBq, 0.40±0.39 Gy/GBq, 0.40±0.38 Gy/GBq, 0.40±0.37 Gy/GBq, 0.40±0.36 Gy/GBq, 0.40±0.35 Gy/GBq, 0.40±0.34 Gy/GBq, 0.40±0.33 Gy/GBq, 0.40±0.32 Gy/GBq, 0.40±0.31 Gy/GBq, 0.40±0.30 Gy/GBq, 0.40±0.29 Gy/GBq, 0.40±0.28 Gy/GBq, 0.40±0.27 Gy/GBq, 0.40±0.26 Gy/GBq, 0.40±0.25 Gy/GBq, 0.40±0.24 Gy/GBq, 0.40±0.23 Gy/GBq, 0.40±0.22 Gy/GBq, 0.40±0.21 Gy/GBq, 0.40±0.20 Gy/GBq, 0.40±0.19 Gy/GBq, 0.40±0.18 Gy/GBq, 0.40±0.17 Gy/GBq, 0.40±0.16 Gy/GBq, 0.40±0.15 Gy/GBq, 0.40±0.14 Gy/GBq, 0.40±0.13 Gy/GBq, 0.40±0.12 Gy/GBq, 0.40±0.11 Gy/GBq, 0.40±0.10 Gy/GBq, 0.40±0.09 Gy/GBq, 0.40±0.08 Gy/GBq, 0.40±0.07 Gy/GBq, 0.40±0.06 Gy/GBq, 0.40±0.05 Gy/GBq, 0.40±0.04 Gy/GBq, 0.40±0.03 Gy/GBq, 0.40±0.02 Gy/GBq, or 0.40±0.01 Gy/GBq. The present disclosure also relates to a method comprising administering to a human patient in need thereof a radiopharmaceutical composition comprising ¹⁷⁷Lu-PSMA I&T that provides a mean absorbed dose to the patient's lacrimal glands that is ≤1.10 Gy/GBq, ≤1.05 Gy/GBq, ≤1.00 Gy/GBq, ≤0.95 Gy/GBq, ≤0.90 Gy/GBq, ≤0.85 Gy/GBq, ≤0.80 Gy/GBq, ≤0.75 Gy/GBq, ≤0.70 Gy/GBq, ≤0.65 Gy/GBq, ≤0.60 Gy/GBq, ≤0.55 Gy/GBq, ≤0.50 Gy/GBq, ≤0.45 Gy/GBq, ≤0.40 Gy/GBq, or ≤0.35 Gy/GBq.

The present disclosure also relates to a method comprising administering to a human patient in need thereof a radiopharmaceutical composition comprising ¹⁷⁷Lu-PSMA I&T that provides a mean absorbed dose to the patient's liver that is 0.04±0.10 Gy/GBq, 0.04±0.09 Gy/GBq, 0.04±0.08 Gy/GBq, 0.04±0.07 Gy/GBq, 0.04±0.06 Gy/GBq, 0.04±0.05 Gy/GBq, 0.04±0.04 Gy/GBq, 0.04±0.03 Gy/GBq, 0.04±0.02 Gy/GBq, or 0.04±0.01 Gy/GBq. The present disclosure also relates to a method comprising administering to a human patient in need thereof a radiopharmaceutical composition comprising ¹⁷⁷Lu-PSMA I&T that provides a mean absorbed dose to the patient's liver that is ≤0.10 Gy/GBq, ≤0.09 Gy/GBq, ≤0.08 Gy/GBq, ≤0.07 Gy/GBq, ≤0.06 Gy/GBq, ≤0.05 Gy/GBq, ≤0.04 Gy/GBq, ≤0.03 Gy/GBq, ≤0.02 Gy/GBq, or ≤0.01 Gy/GBq.

The present disclosure is also related to a method comprising administering to a human patient in need thereof a radiopharmaceutical composition comprising ¹⁷⁷Lu-PSMA I&T that provides a mean absorbed dose to the patient's kidneys that is ≤0.70 Gy/GBq, a mean absorbed dose to the patient's lacrimal glands that is ≤1.10 Gy/GBq, and a mean absorbed dose to the patient's salivary glands that is ≤0.30 Gy/GBq. In another aspect, the present disclosure related to a method comprising administering to a human patient in need thereof a radiopharmaceutical composition comprising ¹⁷⁷Lu-PSMA I&T that provides a mean absorbed dose to the patient's kidneys that is ≤0.60 Gy/GBq, a mean absorbed dose to the patient's lacrimal glands that is ≤1.00 Gy/GBq, and a mean absorbed dose to the patient's salivary glands that is ≤0.25 Gy/GBq. In addition, the present disclosure relates to a method comprising administering to a human patient in need thereof a radiopharmaceutical composition comprising ¹⁷⁷Lu-PSMA I&T that provides a mean absorbed dose to the patient's kidneys that is ≤0.50 Gy/GBq, a mean absorbed dose to the patient's lacrimal glands that is ≤0.90 Gy/GBq, and a mean absorbed dose to the patient's salivary glands that is ≤0.20 Gy/GBq.

The present disclosure is further related to a method of diagnosing or treating a tumor of a patient in need thereof, the method comprising administering by injection a radiopharmaceutical composition comprising ¹⁷⁷Lu-PSMA I&T and ascorbic acid at a pH of 3.5 to 5.0 in solution, wherein 20 hours after injection the radiopharmaceutical composition has an activity of at least 20% IA to 30% IA in the whole body.

The present disclosure is further related to a method of diagnosing a tumor of a patient in need thereof, the method comprising administering to the patient by injection a radiopharmaceutical composition comprising ¹⁷⁷Lu-PSMA I&T and ascorbic acid at a pH of 3.5 to 4.5 in solution, wherein less than 20 hours after injection the radiopharmaceutical composition has an activity of at least 8% IA to 10% IA in the kidneys.

The present disclosure is further related to a method of diagnosing a tumor in a patient in need thereof, the method comprising administering to the patient by injection a radiopharmaceutical composition comprising ¹⁷⁷Lu-PSMA I&T and ascorbic acid at a pH of 3.5 to 4.5 in solution, wherein less than 20 hours after injection the radiopharmaceutical composition has an activity of at least 0.7% IA to 1% IA in the parotid glands.

The present disclosure is further related to a method of diagnosing a tumor in a patient in need thereof, the method comprising administering to the patient by injection a radiopharmaceutical composition comprising ¹⁷⁷Lu-PSMA I&T and ascorbic acid at a pH of 3.5 to 4.5 in solution, wherein less than 20 hours after injection the radiopharmaceutical composition has an activity of at least 0.2% IA to 0.5% IA in lymph node lesions of the patient.

The present disclosure is further related to a method of diagnosing a tumor in a patient in need thereof, the method comprising administering to the patient by injection a radiopharmaceutical composition comprising ¹⁷⁷Lu-PSMA I&T and ascorbic acid at a pH of 3.5 to 4.5 in solution, wherein less than 20 hours after injection the radiopharmaceutical composition has an activity of at least 0.1% IA to 0.4% IA in bone lesions of the patient.

The present disclosure is further related to a method of diagnosing a tumor in a patient in need thereof, the method comprising administering to the patient by injection a radiopharmaceutical composition comprising ¹⁷⁷Lu-PSMA I&T and ascorbic acid at a pH of 3.5 to 4.5 in solution, wherein the radiopharmaceutical composition has an effective half-life of about 30 hours to 40 hours in the whole body of the patient.

Various refinements exist of the features noted above in relation to the various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. Again, the brief summary presented above is intended only to familiarize the reader with certain aspects and contexts of the present disclosure without limitation to the claimed subject matter.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Various features, aspects, and advantages of the present disclosure will become better understood when the following detailed description is read with reference to the accompanying figures in which like characters represent like parts throughout the figures, wherein:

FIG. 1A presents the structural formula of the precursor PSMA I&T.

FIG. 1B presents the structural formula for the R isomer of ¹⁷⁷Lu-PSMA I&T. However, the PSMA I&T doesn't have a specification for enantiomeric purity of R and S isomers.

FIG. 2 is a flowchart representation of an example method of preparing the disclosed radiopharmaceutical composition.

FIG. 3A is a flowchart of the synthetic procedure of the ¹⁷⁷Lu-PSMA I&T in one embodiment.

FIG. 3B is a flowchart of the synthetic procedure of the ¹⁷⁷Lu-PSMA I&T in one embodiment.

FIG. 4 depicts a drawing of an example product vial. The drug product is delivered in a sterile pyrogen free glass vial of Type 1 glass with a fluorocoated bromobutyl rubber septum. The septum is sealed with a crimped aluminum capsule. During transportation, the glass vial containing the radiopharmaceutical is kept in a lead shielded container. The transport container including lead shield and outer packaging complies to type A requirements (IAEA standards).

FIG. 5 shows [¹⁷⁷Lu]Lu-PSMA I&T radiochemical purity measured by HPLC at different time points.

FIGS. 6A and 6B show HPLC radio-chromatograms of high radioactivity concentration formulation containing 42.5 mg/ml ascorbic acid at pH of 7±0.1, 0 hours and 71 hours post EOS as detailed in example 3.

FIGS. 7A and 7B show HPLC radio-chromatograms of high radioactivity concentration formulation containing 42.4 mg/ml ascorbic acid at pH of 4.5±0.1, 0 hours and 71 hours post end of synthesis (“EOS”) as detailed in example 3.

FIGS. 8A and 8B show HPLC radio-chromatograms of high radioactivity concentration formulation containing 42.5 mg/ml ascorbic acid at pH of 3.5±0.1, 0 hours and 90 hours post EOS as detailed in example 3.

FIGS. 9A and 9B show HPLC radio-chromatograms of low radioactivity concentration formulation containing 21 mg/ml ascorbic acid at pH of 4.5±0.1, 0 hours and 92 hours post EOS as detailed in example 3.

FIGS. 10A and 10B show HPLC radio-chromatograms of low radioactivity concentration formulation containing 31 mg/ml ascorbic acid at pH of 5±0.1, 0 hours and 71 hours post EOS as detailed in example 3.

FIGS. 11A and 11B show HPLC radio-chromatograms of low radioactivity concentration formulation containing 31 mg/ml ascorbic acid at pH of 4.5±0.1, 0 hours and 93 hours post EOS as detailed in example 3.

FIG. 13 presents the baseline and on-treatment disease status evaluation and treatment decisions.

FIG. 14 presents time activity curves for all region of interest (ROI) delineated organs in one embodiment. Data (dots) represent averages over the 12 subjects analyzed.

FIG. 15 presents the mean time-integrated activity coefficients (TIAC) and standard deviations (SD) for all delineated region for the first 6 patients in one embodiment.

FIG. 16 presents maximum intensity projection of the SPECT/CT images acquired for one subject at 4 h, 24 h, 48 h, and 168 h post injection in one embodiment.

FIG. 17 presents mean TIAC and SD for all delineated regions for the first 12 patients in one embodiment.

FIG. 18 presents the SPLASH study design in one embodiment.

FIG. 19 presents RCP as a Function of Radiolabeling pH.

FIG. 20 presents RCP as a Function of Sodium Ascorbate Concentration (Reaction Buffer).

FIG. 21 presents Impact of Reaction Time and Temp on RCP.

FIG. 22 presents RCP as a function of ascorbate content (T0+3d).

FIG. 23 presents CP as a function of ascorbate content (T0+7d).

FIG. 24 presents a RadioHPLC Chromatogram of ¹⁷⁷Lu-PSMA I&T composition at formation/production (i.e., T0), wherein PSMA content is about 120 μg.

FIG. 25 presents RCP as a function of Reaction Buffer Age.

FIG. 26 presents an embodiment of the invention.

FIG. 27 presents organ delineation on a representative patient.

FIG. 28 presents maximum intensity projection of the Cycle 1 SPECT/CT images.

FIG. 29 presents whole Body time-activity curve expressed as fraction of injected activity for each patient imaged at cycle 1.

FIG. 30 presents kidney time-activity curve expressed as fraction of injected activity for each patient imaged at cycle 1.

FIG. 31 presents red marrow time-activity curve expressed as fraction of injected activity for each patient imaged at cycle 1.

FIG. 32 presents salivary glands time-activity curve expressed as fraction of injected activity for each patient imaged at cycle 1.

FIG. 33 presents GI tract time-activity curve expressed as fraction of injected activity for each patient imaged at cycle 10.

FIG. 34 presents liver time-activity curve expressed as fraction of injected activity for each patient imaged at cycle 1.

FIG. 35 presents spleen time-activity curve expressed as fraction of injected activity for each patient imaged at cycle 1.

FIG. 36 presents lacrimal glands time-activity curve expressed as fraction of injected activity for each patient imaged at cycle 1.

FIG. 37 presents Cycle 1 Averaged Time Activity Curves for all source organs, in semi-logarithmic scale.

FIG. 38 presents plasma time-activity curve in fraction of injected activity per Liter for patients at cycle 1 (n=27) in semi-logarithmic scale.

FIG. 39 presents plasma time-activity curve in fraction of injected activity per Liter for patients at cycle 3 (n=15).

FIG. 40 presents pH of the eight formulations prepared in this report as a function of time.

FIG. 41 presents the concentration of PSMA I&T and related substances at T0, T0+72 h, and T0+168 h (when measured) for the eight formulations prepared at various pH values.

FIG. 42 presents the HPLC-UV chromatograms for the formulation at a starting pH of 11.4 at (A) release (T0 h) and (B) expiry (T0+72 h).

FIG. 43 presents RCP at expiry (T0+72 h) of 7 of the formulations plotted against the pH of the formulations measured at release (T0 h).

FIG. 44 presents RCP values at T0, T0+72 h, and T0+168 h for the four formulations held for 7 days (T0+168 h).

FIG. 45 presents HPLC-UV chromatograms for the formulation at a starting pH of 13.1 at (A) release (T0 h) and (B) expiry (T0+72 h) and (C) for the formulation at a starting pH of 11.4 at expiry (T0+72 h). D) HPLC-Radiometric chromatogram for the formulation at a starting pH of 13.1 at expiry (T0+72 h).

FIG. 46 presents HPLC-UV chromatograms at T0+168 h for the two formulations at starting pH values of (A) 3.6, (B) 4.1, (C) 4.5, and (D) 4.9.

FIG. 47 presents effect of mole ratio on RCP.

FIG. 48 presents RCP of the four formulations prepared in this report plotted against the target DTPA concentration.

FIG. 49 presents A) DTPA content and B) percent free [177Lu]Lu3+ for the formulations prepared in this report at release (TO) and expiry (T0+72 h). The red dashed line in panel B denotes the 1% limit specification on the presence of free [177Lu]Lu3+.

FIG. 50 presents HPLC-UV chromatograms at T0+72 h for formulations at target DTPA concentrations of 0, 10, 75, 150 ppm. Relative peak areas are calculated by dividing the area of the specified impurity peak by the total sum of the area for all integrated peaks.

FIG. 51 presents HPLC-UV chromatograms at release (T0 h) for the formulation at a target DTPA concentrations of 150 ppm.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Disclosed herein is a small molecular inhibitor of PSMA that has the desirable attributes of large monoclonal antibodies with reduced negative aspects, e.g., poor permeability and toxicity. The radiopharmaceutical composition disclosed herein comprises ¹⁷⁷Lu-PSMA I&T. ¹⁷⁷Lu-PSMA I&T is a short-lived radiolabeled substance from which the product is formulated immediately after finished synthesis.

Headings included herein are simply for ease of reference and are not intended to limit the disclosure in any way.

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims or can be learned by the practice of the principles set forth herein. All publications, patents and patent applications cited herein, whether supra or infra, are hereby incorporated by reference in their entirety.

### I. Definitions

Several definitions that apply throughout the above disclosure will now be presented. As used herein, the terms “comprising,” “having,” and “including” are used interchangeably in their open, non-limiting sense. The terms “a,” “an,” and “the” are understood to encompass the plural as well as the singular. Thus, the term “a mixture thereof” also relates to “mixtures thereof”

Generally, the ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

As used herein, “about” refers to numeric values, including whole numbers, fractions, per-centages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

As used herein, “PSMA” refers to prostate-specific membrane antigen, also known as folate hydrolase I or glutamate carboxypeptidase II, is a type II transmembrane protein, which is anchored in the cell membrane of prostate epithelial cells. PSMA is highly expressed on prostate epithelial cells and strongly up-regulated in prostate cancer. The PSMA expression levels are directly correlated to androgen independence, metastasis, and prostate cancer progression. Thus, PSMA is a promising molecular target for diagnosis and therapy of metastatic prostate cancer at present.

As used herein, “Lutetium-177” and “¹⁷⁷Lu” are used interchangeably. ¹⁷⁷Lu is a β- and 7-emitting radionuclide with a physical half-life of 6.7 days. It has a maximum and mean β-particle energy of 0.498 MeV and 0.133 MeV, respectively. The maximum and mean soft-tissue penetration depth of ¹⁷⁷Lu is 1.7 mm and 0.23 mm, respectively. It has two main gamma emission lines: 113 keV (6% relative abundance) and 208 keV (11% relative abundance).

As used herein, “¹⁷⁷Lu-PSMA-617,” refers to a DOTA derivative of the Glu-urea-Lys motif that has been developed in the German Cancer Research Center (DKFZ) Heidelberg, Germany, for the treatment of patients with metastatic prostate cancer.

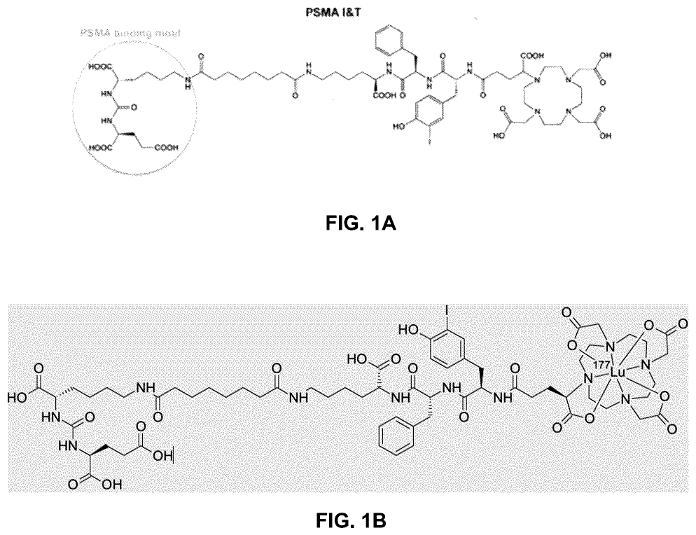

As used herein, “[¹⁷⁷Lu]Lu-PSMA I&T” and “¹⁷⁷Lu-PSMA I&T” are interchangeably used and refer to ¹⁷⁷Lu-PSMA for imaging and therapy (I&T), a third-generation derivative of ¹⁷⁷Lu-PSMA-compounds which has been used here. The chemical name of ¹⁷⁷Lu-PSMA I&T is (3S,7S,26R,29R,32R,37R)-29-benzyl-32-(4-hydroxy-3-iodobenzyl)-5,13,20,28,31,34-hexaoxo-37-(4,7,10-tris(carboxymethyl)-1,4,7,10-tetraazacyclododecan-1-yl)-4,6,12,21,27,30,33-heptaazaheptatriacontane-1,3,7,26,37-pentacarboxylic acid; lutetium-177 (III). The chemical structure of ¹⁷⁷Lu-PSMA I&T is provided in FIG. 1B.

The term “half-life” as used herein refers to the biological half-life, for example, the time required for a drug's blood or plasma concentration to decrease by one half. This decrease in drug concentration is a reflection of its excretion or elimination after absorption is complete and distribution has reached an equilibrium or quasi equilibrium state. The half-life of a drug in the blood may be determined graphically off of a pharmacokinetic plot of a drug's blood-concentration time plot, typically after intravenous administration to a sample population. The half-life can also be determined using mathematical calculations that are well known in the art. Further, as used herein the term “half-life” also includes the “apparent half-life” of a drug. The apparent half-life may be a composite number that accounts for contributions from other processes besides elimination, such as absorption, reuptake, or enterohepatic recycling.

As used herein, “PRLT” refers to prostate radioligand therapy and “RLT” refers to radioligand therapy. PRLT in this context involves the systemic intravenous administration of a specific radiopharmaceutical composed of a β-emitting radionuclide chelated to a small molecule for the purpose of delivering cytotoxic radiation to cancer cells. All compositions and methods described herein may be used for PRLT and/or treating cancer.

The term “CRPC,” as used herein, refers to castrate resistant prostate cancer. In an example, a patient with CRPC may have castrate serum testosterone <50 μg/l or 1.7 nmol/1 plus one of the following types of progression: biochemical progression or radiologic progression, as defined below. All compositions and methods described herein may be used for CRPC and/or treating cancer.

The term “biochemical progression,” as used herein, refers to three consecutive rises in PSA one week apart, resulting in two 50% increases over the nadir, and PSA >2 μg/l.

The term “RAC” as used herein, refers to radioactivity concentration.

The term “radiologic progression,” as used herein, refers to the appearance of new lesions; either two or more new bone lesions on bone scan or a soft tissue lesion using the Response Evaluation Criteria in Solid Tumors (RECIST).

As used herein, the terms “end of synthesis,” “after formulation,” and “end of formulation” are used interchangeably to mean when the process of preparing the composition has completed. This may also include the time after quality control and release of the drug product by a Qualified Person.

The term “active agent” or “drug,” as used herein, refers to any chemical that elicits a biochemical response when administered to a human or an animal. The drug may act as a substrate or product of a biochemical reaction, or the drug may interact with a cell receptor and elicit a physiological response, or the drug may bind with and block a receptor from eliciting a physiological response.

The term “adverse event” (AE) is any untoward medical occurrence in a subject administered an investigational drug, which does not necessarily have a causal relationship with the treatment. An AE can be any unfavorable or unintended sign (e.g., an abnormal laboratory finding), symptom, or disease temporally associated with the use of a drug, whether or not it is considered to be drug related. This includes any newly occurring event or previous condition that has increased in severity or frequency since the administration of the drug.

The terms “subject” or “patient” are used interchangeably herein and refer to a vertebrate, preferably a mammal. Mammals include, but are not limited to, humans.

As used herein, “composition” refers to radiopharmaceutical composition and vice-versa. Accordingly, “composition” and “radiopharmaceutical composition” can be used interchangeably.

The term “effective amount” or “effective dose” refers to the amount of a therapy (e.g., radiation provided herein, or another active agent described herein such as an anti-cancer treatment described herein) which is sufficient to accomplish a stated purpose or otherwise achieve the effect for which it is administered. An effective dose can be sufficient to reduce and/or ameliorate the progression, development, recurrence, severity and/or duration of a given disease, disorder or condition and/or a symptom related thereto. An effective dose can be a “therapeutically effective dose” which refers to an amount sufficient to provide a therapeutic benefit such as, for example, the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. A therapeutically effective amount of a composition described herein can also enhance the therapeutic efficacy of another therapeutic agent.

The terms “therapies,” “therapy” and/or “treatment” refer to any protocol(s), method(s), and/or agent(s) that can be used in the prevention, treatment, management, and/or amelioration of a disease, disorder, or condition or one or more symptoms thereof. In certain instances the term refers to radioligand therapy (RLT) described herein. The terms “therapy” can refer to anti-viral therapy, anti-bacterial therapy, anti-fungal therapy, anti-cancer therapy, biological therapy, supportive therapy, and/or other therapies useful in treatment, management, prevention, or amelioration of a disease, disorder, or condition or one or more symptoms thereof known to one skilled in the art, for example, a medical professional such as a physician.

The term “cancer” refers to any physiological condition in mammals characterized by unregulated cell growth. Cancers described herein include solid tumors and hematological (blood) cancers, including but not limited to mCRPC. A “hematological cancer” refers to any blood home cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being. In the context of treating a cancer or tumor, treating may comprise slowing growth of a tumor, ceasing growth of a tumor, shrinking or decreasing the size of a tumor, preventing a change in shape or morphology of a tumor, preventing the spread of a tumor (e.g., preventing metastases), increasing survivability, and/or decreasing mortality.

The term “enhance” refers to an increase or improvement in the function or activity of a protein or cell or improve the overall wellbeing of a patient after administration of the “treatment” or “therapy” described herein compared to the protein or cell prior to such administration or contact.

The term “administering” refers to the act of delivering a pharmaceutical composition or a radiopharmaceutical composition described herein into a subject by parenteral routes including intravenous, intramuscular, intra-arterial, intradermal, subcutaneous, intraperitoneal, intraventricular, and intracranial administration. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition). For the intravenous route of administration as used herein, the term “injection” and “intravenous infusion” can be used interchangeably.

### II. Introduction

The present disclosure is directed to a radiopharmaceutical composition comprising ¹⁷⁷Lu-PSMA I&T. In some embodiments, the composition may be formulated as a radiopharmaceutical solution for injection or intravenous infusion. The present disclosure further relates to a high-energy, high purity, and/or low toxicity radiopharmaceutical composition comprising ¹⁷⁷Lu-PSMA I&T that performs as an anti-tumor agent for targeted radionuclide therapy.

The present disclosure is also directed to methods of making the radiopharmaceutical composition. Provided herein are methods of increasing the shelf life of a radiopharmaceutical product.

The present disclosure further relates to the properties of the radiopharmaceutical composition and methods of use of the radiopharmaceutical composition.

¹⁷⁷Lu-PSMA I&T is also known by its synonyms as follows: [¹⁷⁷Lu]Lutetium-PSMA I&T, ¹⁷⁷Lu-ITG-PSMA-1, PSMA-TUM3, ¹⁷⁷Lu-DOTAGA-(I-y)fk(Sub-KuE) or ¹⁷⁷Lu-(3S, 7S)-29-benzyl-32(3-iodo, 4-hydroxy)-benzyl-5, 12, 20, 28, 31, 34-hexaoxo-37-(4,7,10-tris(carboxymethyl)-1, 4, 7, 10-tetraazacyclododecan-1-yl)-4, 6, 12, 21, 27, 30, 33-heptaazaheptatriacontane-1, 3, 7, 26, 37-pentacarboxylic acid. The molecular formula of the unlabeled precursor is C₆₃H₉₂IN₁₁O₂₃·4TFA·3 H₂O with a relative molecular mass of 1498 g/mol.

The labelled substance ¹⁷⁷Lu-PSMA I&T may be labelled with non-carrier-added Lutetium-177 (T_{1/2}=6.6d) solution. ¹⁷⁷Lu-PSMA I&T is a short-lived radiolabeled substance from which the product is formulated immediately after finished synthesis. Therefore, there are no specifications or batch analysis results for the labelled substance. Controls are performed on the labelled drug product.

The synthesized ¹⁷⁷Lu-PSMA I&T solution may be formulated in an injections grade water solution containing stabilizing agents such as ascorbic acid. The solution may be sterilized by aseptic filtration through a 0.22 μm filter prior to dispensing into vials. Administration of the formulated solution may be within 72 h of the end of the synthesis after quality control and release of the drug product. Administration of the formulated solution may be via injection or intravenous infusion to a human patient in need thereof within 72 h of the end of the synthesis after quality control and release of the drug product.

Ascorbic acid may be employed to minimize radiolysis of radiolabeled preparations. In addition to ascorbic acid, maintaining the pH of the drug product at or below 5 may stabilize the labelled product against radiolytic decomposition and enhancing its shelf life. Thus, in another aspect, the present disclosure further provides a dose formulation containing ascorbic acid at pH of 5 or below that improves stability of the radiopharmaceutical composition against radiolytic decomposition, thus improving the shelf life of the composition.

The stability enhancing conditions may be applied as early as possible in the manufacturing process. For example, ascorbic acid solutions at pH 5 or below (e.g., pH of 5.0, 4.9, 4.8, 4.7, 4.6, 4.5, 4.4, 4.3, 4.2, 4.1, 4.0, 3.9, 3.8, 3.7, 3.6, 3.5, 3.3, 3.2, 3.1, or 3.0) may be used instead of water in the purification steps of the labelled ¹⁷⁷Lu-PSMA I&T to minimize radiolytic damage.

The composition, when administered to a subject, may result in low hepatotoxicity and nephrotoxicity profiles, providing better effects and fewer adverse effects than monoclonal antibody treatments and other comparable third-line treatments.

The composition is an improved composition in that it has a shelf life of 72 hours or more after formulation. Additionally, the improved composition has a radiochemical purity of greater than 95% at administration. That is, the improved formulation maintains a high level of radiochemical purity for 72 hours or more after formulation. Therefore, the improved formulation is suitable for administration up to 24, up to 72 hours, or longer than other compositions comprising ¹⁷⁷Lu-PSMA I&T.

### III. Compositions

Disclosed herein inter alia is a composition that includes ¹⁷⁷Lu, PSMA I&T and one or more optional agents including buffering agents and/or solvents. In one embodiment, the composition is suitable for administration to a human patient in need thereof.

In one embodiment, the composition has a radiochemical purity (RCP) of 95% or greater at administration. In another embodiment, the composition has a radiochemical purity (RCP) of 97% or greater at administration. In another embodiment, the composition has a radiochemical purity (RCP) of 97.5% or greater at administration. In another embodiment, the composition has a radiochemical purity (RCP) of 98.0% or greater, 98.5% or greater, 99.0% or greater, or 99.5% or greater at administration.

In one embodiment, the composition has a radiochemical purity (RCP) of 95% or greater at 72 hours after production. In another embodiment, the composition has a radiochemical purity (RCP) of 97% or greater at 72 hours after production. In another embodiment, the composition has a radiochemical purity (RCP) of 97.5% or greater at 72 hours after production. In another embodiment, the composition has a radiochemical purity (RCP) of 98.0% or greater, 98.5% or greater, 99.0% or greater, or 99.5% or greater at 72 hours after production. In another embodiment, the composition has a radiochemical purity (RCP) of 90% or greater, 91% or greater, 92% or greater, 93% or greater, 94% or greater, 95% or greater at 7 days after production.

In one embodiment, the composition has a radiochemical purity (RCP) of 96.0% or greater, 96.5% or greater, 97.0% or greater, 97.5% or greater, 98.0% or greater, 98.5% or greater, 99.0% or greater, or 99.5% or greater at 7 days after production.

In one embodiment, the composition is produced as part of a 4 Ci to 10 Ci batch scale. In another embodiment, the composition is produced as part of a 4 Ci to 15 Ci batch scale.

Further disclosed herein is a radiopharmaceutical composition or formulation that includes a dose of ¹⁷⁷Lu-PSMA I&T and at least one of a stabilizing agent, an antioxidant, a pH adjuster, a metal ion chelator, water, or a combination thereof.

In one specific embodiment, the stabilizing agent is ascorbic acid. In another embodiment the antioxidant may be ethanol, ascorbic acid, gentisic acid, or a combination thereof. In another embodiment, the pH adjuster includes but is not limited to sodium hydroxide, sodium bicarbonate, hydrochloric acid, or combinations thereof. In yet another embodiment, the chelator may be EDTA or DTPA. In another specific embodiment, the stabilizing agent comprises no ethanol (i.e., 0% ethanol, less than 0.5% ethanol, or less than 1.0% ethanol (w/w) in the composition).

In one embodiment, the medicinal product or radiopharmaceutical composition (or formulation) may be a sterile filtered radiopharmaceutical solution containing a dose of ¹⁷⁷Lu-PSMA I&T in an aqueous ascorbic acid solution containing ethanol. For example, the total amount of ascorbic acid in the solution may be about 25 to about 65 mg/mL and the total amount of ethanol in the solution may be about 3.8% (v/v) to about 7.5% (v/v). In some embodiments, the total amount of ascorbic acid in the solution is from about 21 mg/mL to about 42.5 mg/mL. The ¹⁷⁷Lu-PSMA I&T is present in sufficient amounts of radioactivity for intended use. Experiments performed with various dose formulations suggests that ¹⁷⁷Lu-PSMA I&T formulation composition containing about 31 mg/ml of ascorbic acid at pH of about 4.5 and radioactivity concentration of about 640 MBq/ml or below may provide adequate radiochemical stability of four days. The adequate radiochemical stability referred herein is radiopharmaceutical composition where the radiochemical purity of the ¹⁷⁷Lu-PSMA I&T is at least 95%, 95.5% or greater, 96.5% or greater, 97.0% or greater, 97.5% or greater, 98.0% or greater, 98.5% or greater, 99.0% or greater, or 99.5% or greater at administration.

In one embodiment, the radiopharmaceutical composition is a sterile filtered radiopharmaceutical solution containing a small amount of ¹⁷⁷Lu-PSMA I&T in an aqueous ascorbic acid and ethanol solution. In another embodiment, the radiopharmaceutical composition is a sterile filtered radiopharmaceutical solution containing a small amount of ¹⁷⁷Lu-PSMA I&T in an aqueous ascorbic acid without ethanol solution. For example, the radiopharmaceutical composition may be a sterile filtered radiopharmaceutical solution containing a micro dose of ¹⁷⁷Lu-PSMA I&T in an aqueous ascorbic acid and DTPA or EDTA (e.g., comprising ethanol or in the complete absence of ethanol). The product is diluted to a standard radioactivity concentration and therefore the final volume of the bulk product varies depending on the starting radioactivity of ¹⁷⁷Lu introduced.

One aspect of the disclosure provides for a radiopharmaceutical composition with a pH from about 3 to about 9, from about 4 to about 9, from about 5 to about 9, from about 3 to about 8, from about 4 to about 8, from about 3 to about 5, or from about 5 to about 8. The pH of the radiopharmaceutical composition may be about 3.0, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, or about 9.

Having a pH of 5 or below may stabilize the radiopharmaceutical composition against radiolytic decomposition and may enhance its shelf life.

In one embodiment, the pH of the radiopharmaceutical composition is from about 3 to about 5. This pH range may stabilize the radiopharmaceutical composition against radiolytic decomposition and may enhance its shelf life. In yet another embodiment, a radiopharmaceutical composition comprising ascorbic acid and having a pH from about 3.5 to about 5 has improved stability and extended shelf life compared to known radiopharmaceutical compositions of ¹⁷⁷Lu-PSMA I&T which have higher pH values and may comprise gentisic acid. In other embodiments, the radiopharmaceutical composition may comprise an absence of gentisic acid (i.e., no gentisic acid).). In yet another embodiment, a radiopharmaceutical composition comprising ascorbic acid and having a pH from about 4.0 to about 4.5 has improved stability and extended shelf life

The pH of the radiopharmaceutical composition may range from 3.0 to 5.0, 3.0 to 3.5, 3.0 to 3.05, 3.05 to 3.1, 3.0 to 3.1, 3.1 to 3.15, 3.1 to 3.2, 3.15 to 3.2, 3.2 to 3.25, 3.0 to 3.25, 3.2 to 3.3, 3.25 to 3.3, 3.3 to 3.35, 3.3 to 3.4, 3.35 to 3.4, 3.4 to 3.45, 3.4 to 3.5, 3.45 to 3.5, 3.25 to 3.5, 3.5 to 3.55, 3.5 to 3.6, 3.55 to 3.6, 3.6 to 3.65, 3.6 to 3.7, 3.65 to 3.7, 3.7 to 3.75, 3.5 to 3.75, 3.7 to 3.8, 3.75 to 3.8, 3.8 to 3.85, 3.8 to 3.9, 3.85 to 3.9, 3.9 to 3.95, 3.9 to 4.0, 3.95 to 4.0, 3.5 to 4.0, 3.75 to 4.0, 4.0 to 4.05, 4.0 to 4.1, 4.05 to 4.1, 4.1 to 4.15, 4.1 to 4.2, 4.15 to 4.2, 3.5 to 4.2, 4.2 to 4.25, 4.0 to 4.25, 4.2 to 4.3, 4.25 to 4.3, 4.3 to 4.35, 4.3 to 4.4, 4.35 to 4.4, 4.4 to 4.45, 4.4 to 4.5, 4.45 to 4.5, 4.25 to 4.5, 4.0 to 4.5, 4.5 to 4.55, 4.5 to 4.6, 4.55 to 4.6, 4.6 to 4.65, 4.6 to 4.7, 4.65 to 4.7, 4.7 to 4.75, 4.7 to 4.8, 4.75 to 4.8, 4.8 to 4.85, 4.8 to 4.9, 4.85 to 4.9, 4.9 to 4.95, 4.9 to 5.0, 4.95 to 5.0, 4.5 to 5.0, or 4.75 to 5.0. In some examples, the pH of the radiopharmaceutical composition may be adjusted to a final pH of 3.0, 3.5, 4.0, 4.5, or 5.0. In some embodiments, including the above listed pH numbers and ranges, the pH values are inclusive of ±0.05, ±0.10,±0.15,±0.20, or ±0.25.

In another embodiment, the radiopharmaceutical composition or formulation has a purity of at least about 90%, at least about 95%, at least about 97%, or at least about 99%. In another embodiment, the radiopharmaceutical composition or formulation has a purity of about 90%, about 91%, about 92%, about 93%, about 94%, about 95%, about 96%, about 97%, about 98%, about 99%, or about 99.5%.

In another embodiment, radiopharmaceutical composition or formulation has a purity of at least about 90%, at least about 95%, at least about 97%, or at least about 99% as measured by HPLC, TLC, or liquid chromatography. In another embodiment, the radiopharmaceutical composition or formulation has a purity of at least about 90.0%, at least about 91.0%, at least about 92.0%, at least about 93.0%, at least about 94.0%, at least about 95.0%, at least about 96.0%, at least about 97.0%, at least about 98.0%, at least about 99.0%, or at least about 99.5% as measured by HPLC or TLC. In some examples, the radiopharmaceutical composition may have a radiochemical purity of 95.0% or greater, 95.5% or greater, 96.0% or greater, 96.5% or greater, 97.0% or greater, 97.5% or greater, 98.0% or greater, 98.5% or greater, 99.0% or greater, or 99.5% or greater at administration. FIG. 5 shows the radiochemical purity of formulations prepared at different pH values.

In another embodiment, the purity of the radiopharmaceutical composition or formulation is measured by HPLC or TLC, at any time post end of synthesis (EOS). In one embodiment, the purity of the radiopharmaceutical composition or formulation is measured by HPLC or TLC, at about 0 hour, about 10 hours, about 20 hours, about 30 hours, about 40 hours, about 50 hours, about 50 hours, about 60 hours, about 70 hours, about 80 hours, about 90 hours, about 100 hours, about 120 hours, and about 168 hours post EOS.

In one specific embodiment, the radiopharmaceutical composition or formulation has a purity of at least about 99% as measured by HPLC or TLC 0 hours post EOS. In another specific embodiment, the radiopharmaceutical composition or formulation has a purity of at least about 96.5% as measured by HPLC or TLC 24 hours post EOS, at least about 93% as measured by HPLC, or TLC 46 hours post EOS, at least about 95% as measured by HPLC or TLC 67 hours post EOS, at least about 96% as measured by HPLC or TLC 92 hours post EOS.

In another embodiment, the radioactivity is measured in a dose calibrator. The radioactive amount of ¹⁷⁷Lu-PSMA I&T is determined when the dose is dispensed prior to patient administration.

In yet another embodiment, the radiochemical purity of ¹⁷⁷Lu-PSMA I&T is determined by liquid chromatography with radioactivity detection and thin layer chromatography.

In one embodiment, bacterial endotoxin content is determined for each batch before release using a PTS-tester (Ph Eur method D or USP<85>) and sterility is determined according to Ph Eur and USP<71>.

In one embodiment, the radiopharmaceutical composition or formulation is stored at a temperature from about +5° C. to +55° C., +5° C. to +40° C., about +10° C. to +35° C. or about +20° C. to +30° C. In one specific embodiment, the radiopharmaceutical composition or formulation is stored at a temperature at about +10° C., about +15° C., about +22° C., about +22.5° C., about +25° C., or at room temperature.

Another aspect of the disclosure provides for a radioactive content of about 70% to 130%. The radioactive content of the radiopharmaceutical composition may be about 90% to 125%, 90% to 120%, 90% to 115%, or 90% to 110%.

In one specific embodiment, the radioactive content of the formulation is about 70%, about 75%, about 80%, about 85%, about 90%, about 95%, about 100%, about 105%, about 110%, about 115%, about 120%, about 125%, or about 130%.

Another aspect of the disclosure provides for a radiopharmaceutical composition with a mean whole-body effective dose of about 23±20 Gy (3.3 Gy/GBq), with a mean absorbed organ doses of about 26±20 Gy (3.4 Gy/GBq), 24±16 Gy (3.2 Gy/GBq), 8.5±4.7 Gy (1.28 Gy/GBq), and 13±7.4 Gy (1.7 Gy/GBq) for the bone, lymph node, liver, and lung metastases, respectively.

In some embodiments, the radiopharmaceutical composition may have a low radioactivity concentration (“low RAC”) of about 563 MBq/ml to about 734 MBq/ml. For example, the radiopharmaceutical composition may have a low radioactivity that may be about 11,580 MBq (313 mCi), about 11,770 MBq (318 mCi), or about 12,520 MBq (338 mCi) in a 20 ml volume of the solution. In other embodiments the radiopharmaceutical composition may have a low radioactivity concentration of at least about 550 MBq/ml, at least about 560 MBq/ml, at least about 570 MBq/ml, at least about 580 MBq/ml, at least about 590 MBq/ml, at least about 600 MBq/ml, at least about 610 MBq/ml, at least about 620 MBq/ml, at least about 630 MBq/ml, at least about 640 MBq/ml, at least about 650 MBq/ml, at least about 660 MBq/mL, at least about 670 MBq/mL, at least about 680 MBq/mL, at least about 690 MBq/mL, at least about 700 MBq/mL, at least about 710 MBq/mL, at least about 720 MBq/mL, at least about 730 MBq/mL, at least about 740 MBq/mL, or at least about 750 MBq/mL. In still other embodiments the radiopharmaceutical composition may have a low radioactivity concentration from about 550 MBq/ml to about 575 MBq/ml, from about 575 MBq/ml to about 600 MBq/ml, from about 600 MBq/ml to about 625 MBq/ml, from about 625 MBq/ml to about 650 MBq/ml, from about 650 MBq/ml to about 675 MBq/ml, from about 675 MBq/ml to about 700 MBq/ml, from about 700 MBq/ml to about 725 MBq/ml, from about 725 MBq/ml to about 750 MBq/ml or from about 625 MBq/ml to about 650.

In additional embodiments, the radiopharmaceutical composition may have a high radioactivity concentration (“High RAC”) of about 1,270 MBq/ml to about 1,311 MBq/ml. For example, the radiopharmaceutical composition may have a high radioactivity that may be about 12,780 MBq (345 mCi), about 12,810 MBq (346 mCi), or about 13,110 MBq (354 mCi) in a 10 ml volume of the solution. In other embodiments the radiopharmaceutical composition may have a high radioactivity concentration of at least about 1,100 MBq/ml, at least about 1,110 MBq/ml, at least about 1,120 MBq/ml, at least about 1,130 MBq/ml, at least about 1,140 MBq/ml, at least about 1,150 MBq/ml, at least about 1,160 MBq/ml, at least about 1,170 MBq/ml, at least about 1,180 MBq/ml, at least about 1,190 MBq/ml, at least about 1,200 MBq/ml, 1,200 MBq/ml, at least about 1,210 MBq/ml, at least about 1,220 MBq/ml, at least about 1,230 MBq/ml, at least about 1,240 MBq/ml, at least about 1,250 MBq/ml, at least about 1,260 MBq/ml, at least about 1,270 MBq/ml, at least about 1,280 MBq/ml, at least about 1,290 MBq/ml, at least about 1,300 MBq/ml, at least about 1,310 MBq/ml, at least about 1,320 MBq/ml, at least about 1,330 MBq/ml, at least about 1,340 MBq/ml, or at least about 1,350 MBq/ml. In still other embodiments the radiopharmaceutical composition may have a high radioactivity concentration from about 1,000 MBq/ml to about 1,400 MBq/ml, from about 1,050 MBq/ml to about 1,350 MBq/ml, from about 1,100 MBq/ml to about 1,300 MBq/ml, from about 1,150 MBq/ml to about 1,250 MBq/ml, from about 1,200 MBq/ml to about 1,300 MBq/ml, from about 1,250 MBq/ml to about 1,350 MBq/ml, or from about 1,250 MBq/ml to about 1,300 MBq/ml.

### (i) ¹⁷⁷Lu-PSMA I&T

The total amount of ¹⁷⁷Lu-PSMA I&T present in the radiopharmaceutical composition can and will vary. FIGS. 1A and 1B show the chemical structure of precursor PSMA I&T and ¹⁷⁷Lu-PSMA I&T respectively.

In one embodiment, the mass of radioactive pharmaceutical ingredient (¹⁷⁷Lu-PSMA I&T) in the drug product is less than about 40 μg, less than about 35 μg, less than about 30 μg, less than about 25 μg, less than about 20 μg, less than about 15 μg, or less than about 10 μg per vial. In yet another embodiment, the mass of radioactive pharmaceutical ingredient (¹⁷⁷Lu-PSMA I&T) in the drug product is about 1 μg, about 2 μg, about 3 μg, about 4 μg, about 5 μg, about 6 μg, about 7 μg, about 8 μg, about 9 μg, or about 10 μg, about 11 μg, about 12 μg about, about 13 μg, about 14 μg, about 15 μg, about 16 μg, about 17 μg, about 18 μg, about 17.2 μg, about 18 μgm about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, or about 40 μg of ¹⁷⁷Lu-PSMA I&T per vial.

In one embodiment, the total amount of ¹⁷⁷Lu-PSMA I&T present in the pharmaceutical composition can and will vary. In the labeling process, the PSMA I&T ligand may be labeled with trace metals that are present to form a chelated trace metal-PSMA I&T complex (i.e., “M-PSMA I&T”). Excess PSMA I&T present in the labeling process may remain unlabeled with either ¹⁷⁷Lu or trace metals. The amount of M-PSMA I&T and unlabeled PSMA I&T in the composition is hereinafter referred to as “related substances” or “RS”. The composition may then contain both ¹⁷⁷Lu-PSMA I&T, M-PSMA I&T as well as unlabeled PSMA I&T. In an embodiment, the PSMA content comprising PSMA I&T and related substances (RS) is 250 μg/dose±15%, ±10%, or ±5%. In another embodiment, the PSMA I&T content is 120 μg/dose±15%, ±10%, or ±5% to about 250 μg/dose±15%, ±10%, or ±5%. In another embodiment, the PSMA I&T content is about 100 μg/dose±15%, ±10%, or ±5% to about 120 μg/dose±15%, ±10%, or ±5%. In another embodiment, the PSMA I&T content is 120 μg/dose±15%, ±10%, or ±5% to about 200 μg/dose±15%, ±10%, or ±5%. In yet another embodiment, the PSMA I&T content is about 40 μg/dose±15%, ±10%, or ±5% to about 100 μg/dose±15%, ±10%, or ±5%, 50 μg/dose±15%, ±10%, or ±5% to about 100 μg/dose±15%, ±10%, or ±5%, 60 μg/dose±15%, ±10%, or ±5% to about 100 μg/dose±15%, ±10%, or ±5%, or 70 μg/dose±15%, ±10%, or ±5% to about 100 μg/dose±15%, ±10%, or ±5%. In yet another embodiment, the PSMA I&T content is about 40 μg/dose±15%, ±10%, or ±5% to about 90 μg/dose±15%, ±10%, or ±5%, 50 μg/dose±15%, ±10%, or ±5% to about 90 μg/dose±15%, ±10%, or ±5%, 60 μg/dose±15%, ±10%, or ±5% to about 90 μg/dose±15%, ±10%, or ±5%, or 70 μg/dose±15%, ±10%, or ±5% to about 90 μg/dose±15%, ±10%, or ±5%. In yet another embodiment, the PSMA I&T content is about 40 μg/dose±15%, ±10%, or ±5% to about 80 μg/dose±15%, ±10%, or ±5%, 50 μg/dose±15%, ±10%, or ±5% to about 80 μg/dose±15%, ±10%, or ±5%, 60 μg/dose±15%, ±10%, or ±5% to about 80 μg/dose±15%, ±10%, or ±5%, or 70 μg/dose±15%, ±10%, or ±5% to about 80 μg/dose±15%, ±10%, or ±5%.

In another embodiment, the compositions comprising ¹⁷⁷Lu-PSMA I&T described herein may comprise a PSMA I&T content that is about 120 μg/dose to about 250 μg/dose, about 130 μg/dose to about 250 μg/dose, about 140 μg/dose to about 250 μg/dose, about 150 μg/dose to about 250 μg/dose, about 160 μg/dose to about 250 μg/dose, about 170 μg/dose to about 250 μg/dose, about 180 μg/dose to about 250 μg/dose, about 190 μg/dose to about 250 μg/dose, about 200 μg/dose to about 250 μg/dose, about 210 μg/dose to about 250 μg/dose, about 220 μg/dose to about 250 μg/dose, about 230 μg/dose to about 250 μg/dose, or about 240 μg/dose to about 250 μg/dose. In another embodiment, the compositions comprising ¹⁷⁷Lu-PSMA I&T described herein may comprise a PSMA I&T content that is about 100 μg/dose to about 120 μg/dose, about 105 μg/dose to about 120 μg/dose, about 110 μg/dose to about 120 μg/dose, or about 115 μg/dose to about 120 μg/dose. In another embodiment, the compositions comprising ¹⁷⁷Lu-PSMA I&T described herein may comprise a PSMA I&T content that is about 40 μg/dose to about 100 μg/dose, about 45 μg/dose to about 100 μg/dose, about 50 μg/dose to about 100 μg/dose, about 55 μg/dose to about 100 μg/dose, about 60 μg/dose to about 100 μg/dose, about 65 μg/dose to about 100 μg/dose, about 70 μg/dose to about 100 μg/dose, about 75 μg/dose to about 100 μg/dose, about 80 μg/dose to about 100 μg/dose, about 85 μg/dose to about 100 μg/dose, about 90 μg/dose to about 100 μg/dose, or about 95 μg/dose to about 100 μg/dose. In another embodiment, the compositions comprising ¹⁷⁷Lu-PSMA I&T described herein may comprise a PSMA I&T content that is about 45 μg/dose to about 95 μg/dose, 50 μg/dose to about 100 μg/dose, 55 μg/dose to about 95 μg/dose, 60 μg/dose to about 95 μg/dose, 65 μg/dose to about 95 μg/dose, 70 μg/dose to about 95 μg/dose, 75 μg/dose to about 95 μg/dose, 80 μg/dose to about 95 μg/dose, 85 μg/dose to about 95 μg/dose, or about 90 μg/dose to about 95 μg/dose. In another embodiment, the compositions comprising ¹⁷⁷Lu-PSMA I&T described herein may comprise a PSMA I&T content that is about 40 μg/dose to about 90 μg/dose, about 45 μg/dose to about 90 μg/dose, about 50 μg/dose to about 90 μg/dose, about 55 μg/dose to about 90 μg/dose, about 60 μg/dose to about 90 μg/dose, about 65 μg/dose to about 90 μg/dose, about 70 μg/dose to about 90 μg/dose, about 75 μg/dose to about 90 μg/dose, about 80 μg/dose to about 90 μg/dose, or about 85 μg/dose to about 90 μg/dose. In another embodiment, the compositions comprising ¹⁷⁷Lu-PSMA I&T described herein may comprise a PSMA I&T content that is about 40 μg/dose to about 85 μg/dose, about 45 μg/dose to about 85 μg/dose, about 50 μg/dose to about 85 μg/dose, about 55 μg/dose to about 85 μg/dose, about 60 μg/dose to about 85 μg/dose, about 65 μg/dose to about 85 μg/dose, about 70 μg/dose to about 85 μg/dose, about 75 μg/dose to about 85 μg/dose, about 80 μg/dose to about 85 μg/dose. In another embodiment, the compositions comprising ¹⁷⁷Lu-PSMA I&T described herein may comprise a PSMA I&T content that is about 40 μg/dose to about 80 μg/dose, about 45 μg/dose to about 80 μg/dose, about 50 μg/dose to about 80 μg/dose, about 55 μg/dose to about 80 μg/dose, about 60 μg/dose to about 80 μg/dose, about 65 μg/dose to about 80 μg/dose, about 70 μg/dose to about 80 μg/dose, or about 75 μg/dose to about 80 μg/dose. In another embodiment, the compositions comprising ¹⁷⁷Lu-PSMA I&T described herein may comprise a PSMA I&T content that is about 40 μg/dose to about 75 μg/dose, about 45 μg/dose to about 75 μg/dose, about 50 μg/dose to about 75 μg/dose, about 55 μg/dose to about 75 μg/dose, about 60 μg/dose to about 75 μg/dose, about 65 μg/dose to about 75 μg/dose, about 70 μg/dose to about 75 μg/dose.

In one embodiment, the PSMA I&T amount present in the pharmaceutical composition is about 30 μg to 120 μg per dose, 35 μg to 120 μg per dose, 40 μg to 120 μg per dose, 45 μg to 120 μg per dose, 50 μg to 120 μg per dose, 55 μg to 120 μg per dose, 60 μg to 120 μg per dose, 65 μg to 120 μg per dose, 70 μg to 120 μg per dose, 75 μg to 120 μg per dose, 85 μg to 120 μg per dose, 90 μg to 120 μg per dose, 95 μg to 120 μg per dose, 100 μg to 120 μg per dose, 105 μg to 120 μg per dose, 110 μg to 120 μg per dose, or 115 μg to 120 μg per dose. In one embodiment, the PSMA I&T amount present in the pharmaceutical composition is about 30 μg to 100 μg per dose, 35 μg to 100 μg per dose, 40 μg to 100 μg per dose, 45 μg to 100 μg per dose, 50 μg to 100 μg per dose, 55 μg to 100 μg per dose, 60 μg to 100 μg per dose, 65 μg to 100 μg per dose, 70 μg to 100 μg per dose, 75 μg to 100 μg per dose, 85 μg to 100 μg per dose, or 90 μg to 100 μg per dose. In another embodiment, the PSMA I&T amount present in the pharmaceutical composition is about 30 μg to 90 μg per dose, 35 μg to 90 μg per dose, 40 μg to 90 μg per dose, 45 μg to 90 μg per dose, 50 μg to 90 μg per dose, 55 μg to 90 μg per dose, 60 μg to 90 μg per dose, 65 μg to 90 μg per dose, 70 μg to 90 μg per dose, 75 μg to 90 μg per dose, or 85 μg to 90 μg per dose. In another embodiment, the PSMA I&T amount present in the pharmaceutical composition is about 30 μg to 80 μg per dose, 35 μg to 80 μg per dose, 40 μg to 80 μg per dose, 45 μg to 80 μg per dose, 50 μg to 80 μg per dose, 55 μg to 80 μg per dose, 60 μg to 80 μg per dose, 65 μg to 80 μg per dose, 70 μg to 80 μg per dose, or 75 μg to 80 μg per dose. In yet another embodiment, the PSMA I&T amount present in the pharmaceutical composition is about 30 μg to 70 μg per dose, 35 μg to 70 μg per dose, 40 μg to 70 μg per dose, 45 μg to 70 μg per dose, 50 μg to 70 μg per dose, 55 μg to 70 μg per dose, 60 μg to 70 μg per dose, or 65 μg to 70 μg per dose.

In one embodiment, the PSMA I&T and related substances (RS) amount present in the pharmaceutical composition is about 30 μg to 120 μg per dose, 35 μg to 120 μg per dose, 40 μg to 120 μg per dose, 45 μg to 120 μg per dose, 50 μg to 120 μg per dose, 55 μg to 120 μg per dose, 60 μg to 120 μg per dose, 65 μg to 120 μg per dose, 70 μg to 120 μg per dose, 75 μg to 120 μg per dose, 85 μg to 120 μg per dose, 90 μg to 120 μg per dose, 95 μg to 120 μg per dose, 100 μg to 120 μg per dose, 105 μg to 120 μg per dose, 110 μg to 120 μg per dose, or 115 μg to 120 μg per dose. In one embodiment, the PSMA I&T and related substances (RS) amount present in the pharmaceutical composition is about 30 μg to 100 μg per dose, 35 μg to 100 μg per dose, 40 μg to 100 μg per dose, 45 μg to 100 μg per dose, 50 μg to 100 μg per dose, 55 μg to 100 μg per dose, 60 μg to 100 μg per dose, 65 μg to 100 μg per dose, 70 μg to 100 μg per dose, 75 μg to 100 μg per dose, 85 μg to 100 μg per dose, or 90 μg to 100 μg per dose. In another embodiment the PSMA I&T and related substances (RS) amount present in the pharmaceutical composition is about 30 μg to 90 μg per dose, 35 μg to 90 μg per dose, 40 μg to 90 μg per dose, 45 μg to 90 μg per dose, 50 μg to 90 μg per dose, 55 μg to 90 μg per dose, 60 μg to 90 μg per dose, 65 μg to 90 μg per dose, 70 μg to 90 μg per dose, 75 μg to 90 μg per dose, or 85 μg to 90 μg per dose. In another embodiment, the PSMA I&T and related substances (RS) amount present in the pharmaceutical composition is about 30 μg to 80 μg per dose, 35 μg to 80 μg per dose, 40 μg to 80 μg per dose, 45 μg to 80 μg per dose, 50 μg to 80 μg per dose, 55 μg to 80 μg per dose, 60 μg to 80 μg per dose, 65 μg to 80 μg per dose, 70 μg to 80 μg per dose, or 75 μg to 80 μg per dose. In yet another embodiment, the PSMA I&T and related substances (RS) amount present in the pharmaceutical composition is about 30 μg to 70 μg per dose, 35 μg to 70 μg per dose, 40 μg to 70 μg per dose, 45 μg to 70 μg per dose, 50 μg to 70 μg per dose, 55 μg to 70 μg per dose, 60 μg to 70 μg per dose, or 65 μg to 70 μg per dose.

In another embodiment, the PSMA I&T and related substances (RS) amount present in the pharmaceutical composition is about 55 μg to 110 μg per dose. In another embodiment, the PSMA I&T and related substances (RS) amount present in the pharmaceutical composition is about 75 μg to 100 μg per dose.

In one embodiment, the PSMA I&T and related substances (RS) amount present in the pharmaceutical composition is about 80 μg to 110 μg per dose. In another embodiment, the PSMA I&T and related substances (RS) amount present in the pharmaceutical composition is about 70 μg to 85 μg per dose. In another embodiment, the PSMA I&T and related substances (RS) amount present in the pharmaceutical composition is about 73 μg to 85 μg per dose. In another embodiment, the PSMA I&T and related substances (RS) amount present in the pharmaceutical composition is about 90 μg to 115 μg per dose. In another embodiment, the PSMA I&T and related substances (RS) amount present in the pharmaceutical composition is about 100 μg to 80 μg per dose.

In one embodiment, the PSMA I&T and related substances (RS) amount present in the pharmaceutical composition is about 110 μg to 80 μg per dose. In another embodiment, the PSMA I&T and related substances (RS) amount present in the pharmaceutical composition is about 115 μg to 125 μg per dose. In another embodiment, the PSMA I&T and related substances (RS) amount present in the pharmaceutical composition is about 115 μg to 130 μg per dose

In one embodiment, the PSMA I&T and related substances (RS) amount present in the pharmaceutical composition is about 45 μg 50 μg, 57 μg, 60 μg, 70 μg, 75 μg, 80 μg, 85 μg, 99 μg, 100 μg, 115 μg, 80 μg, 125 μg, 130 μg, per vial. In another embodiment, the PSMA I&T and related substances (RS) amount present in the pharmaceutical composition is about 80 μg.

In one embodiment, the PSMA I&T and related substances (RS) amount present in the pharmaceutical composition is about 57 μg. In one embodiment, the PSMA I&T and related substances (RS) amount present in the pharmaceutical composition is about 99 μg.

In one embodiment, the total volume of the vial comprising PSMA I&T and related substances (RS) is about 5 to 30 mL. In another embodiment, the total volume of the vial comprising PSMA I&T and related substances (RS) is about 10 to 20 mL. In another embodiment, the total volume of the vial comprising PSMA I&T and related substances (RS) is about 15 to 20 mL. In another embodiment, the total volume of the vial comprising PSMA I&T and related substances (RS) is about 15 to 17 mL. In another embodiment, the total volume of the vial comprising PSMA I&T and related substances (RS) is about 15 mL.

In one embodiment, the PSMA I&T and related substances (RS) concentration per vial is 3 to 8 μg/mL. In another embodiment, the PSMA I&T and related substances (RS) concentration per vial is 4 to 7 μg/mL. In another embodiment, the PSMA I&T and related substances (RS) concentration per vial is 4.5 to 6.5 μg/mL. In another embodiment, the PSMA I&T and related substances (RS) concentration per vial is 4.8 to 6 μg/mL.

In one embodiment, the PSMA I&T and related substances (RS) concentration per vial is 4 μg/mL. In another embodiment, the PSMA I&T and related substances (RS) concentration per vial is 5 μg/mL. In another embodiment, the PSMA I&T and related substances (RS) concentration per vial is 6 μg/mL. In another embodiment, the PSMA I&T and related substances (RS) concentration per vial is 7 μg/mL. In another embodiment, the PSMA I&T and related substances (RS) concentration per vial is 8 μg/mL.

In one embodiment, the molar ratio of the PSMA I&T to ¹⁷⁷Lu in the composition is from 5.0:1.0 to 12.0:1.0. In another embodiment, the molar ratio of the PSMA I&T to ¹⁷⁷Lu in the composition is from 5.0:1.0 to 12.0:1.0, 5.0:1.0 to 11.5:1.0, 5.0:1.0 to 11.0:1.0, 5.0:1.0 to 10.5:1.0, 5.0:1.0 to 10.0:1.0, 5.0:1.0 to 9.5:1.0, 5.0:1.0 to 9.0:1.0, 5.0:1.0 to 8.5:1.0, 5.0:1.0 to 8.0:1.0, 5.0:1.0 to 7.5:1.0, 5.0:1.0 to 7.0:1.0, 5.0:1.0 to 6.5:1.0, or 5.0:1.0 to 6.0:1.0. In another embodiment, the molar ratio of the PSMA I&T to ¹⁷⁷Lu in the composition is from 4.0:1.0 to 11.0:1.0, 4.0:1.0 to 10.5:1.0, 4.0:1.0 to 10.0:1.0, 4.0:1.0 to 9.5:1.0, 4.0:1.0 to 9.0:1.0, 4.0:1.0 to 8.5:1.0, 4.0:1.0 to 8.0:1.0, 4.0:1.0 to 7.9:1.0, 4.0:1.0 to 7.8:1.0, 4.0:1.0 to 7.7:1.0, 4.0:1.0 to 7.6:1.0, 4.0:1.0 to 7.5:1.0, 4.0:1.0 to 7.4:1.0, 4.0:1.0 to 7.3:1.0, 4.0:1.0 to 7.2:1.0, 4.0:1.0 to 7.1:1.0, 4.0:1.0 to 7.0:1.0, 4.0:1.0 to 6.5:1.0, or 4.0:1.0 to 6.0:1.0.

In one embodiment, at the time of administration, the molar ratio of the PSMA I&T to ¹⁷⁷Lu in the composition is from 3.0:1.0 to 11.0:1.0. In another embodiment, the molar ratio of the PSMA I&T to ¹⁷⁷Lu in the composition is from 3.0:1.0 to 11.0:1.0, 3.0:1.0 to 10.5:1.0, 3.0:1.0 to 10.0:1.0, 3.0:1.0 to 9.5:1.0, 30:1.0 to 9.0:1.0, 3.0:1.0 to 8.5:1.0, 3.0:1.0 to 8.0:1.0, 3.0:1.0 to 7.9:1.0, 3.0:1.0 to 7.8:1.0, 3.0:1.0 to 7.7:1.0, 3.0:1.0 to 7.6:1.0, 3.0:1.0 to 7.5:1.0, 3.0:1.0 to 7.4:1.0, 3.0:1.0 to 7.3:1.0, 3.0:1.0 to 7.2:1.0, 3.0:1.0 to 7.1:1.0, 3.0:1.0 to 7.0:1.0, 3.0:1.0 to 6.5:1.0, or 3.0:1.0 to 6.0:1.0.

In one embodiment, at the time of administration, the molar ratio of the PSMA I&T to ¹⁷⁷Lu in the composition is from 11.0:1.0 to 12.0:1.0, 11.1:1.0 to 11.9:1.0, 11.2:1.0 to 11.8:1.0, 11.3:1.0 to 11.7:1.0, or 11.4:1.0 to 11.6:1.0. In another embodiment, the molar ratio of the PSMA I&T to ¹⁷⁷Lu in the composition is from 10.0:1.0 to 11.0:1.0, 10.1:1.0 to 10.9:1.0, 10.2:1.0 to 10.8:1.0, 10.3:1.0 to 10.7:1.0, or 10.4:1.0 to 10.6:1.0.

In one embodiment, the molar ratio of the PSMA I&T to ¹⁷⁷Lu in the composition is from 9.0:1.0 to 10.0:1.0, 9.1:1.0 to 9.9:1.0, 9.2:1.0 to 9.8:1.0, 9.3:1.0 to 9.7:1.0, or 9.4:1.0 to 9.6:1.0. In another embodiment, the molar ratio of the PSMA I&T to ¹⁷⁷Lu in the composition is from 8.0:1.0 to 9.0:1.0, 8.1:1.0 to 8.9:1.0, 8.2:1.0 to 8.8:1.0, 8.3:1.0 to 8.7:1.0, or 8.4:1.0 to 8.6:1.0. In another embodiment, the molar ratio of the PSMA I&T to ¹⁷⁷Lu in the composition is from 7.0:1.0 to 8.0:1.0, 7.1:1.0 to 7.9:1.0, 7.2:1.0 to 7.8:1.0, 7.3:1.0 to 7.7:1.0, or 7.4:1.0 to 7.6:1.0. In another embodiment, the molar ratio of the PSMA I&T to ¹⁷⁷Lu in the composition is from 6.0:1.0 to 7.0:1.0, 6.1:1.0 to 6.9:1.0, 6.2:1.0 to 7.8:1.0, 7.3:1.0 to 7.7:1.0, or 7.4:1.0 to 6.6:1.0. In another embodiment, the molar ratio of the PSMA I&T to ¹⁷⁷Lu in the composition is from 5.0:1.0 to 6.0:1.0, 5.1:1.0 to 5.9:1.0, 5.2:1.0 to 5.8:1.0, 5.3:1.0 to 5.7.0:1.0, or 5.4:1.0 to 5.6:1.0. In another embodiment, the molar ratio of the PSMA I&T to ¹⁷⁷Lu in the composition is from 3.0:1.0 to 5.0:1.0, 3.1:1.0 to 4.9:1.0, 3.2:1.0 to 4.8:1.0, 3.3:1.0 to 4.7:1.0, or 3.4:1.0 to 4.6:1.0.

In another embodiment, the molar ratio of the PSMA I&T to ¹⁷⁷Lu in the composition is from 4.0:1.0 to 5.0:1.0, about 4.0:1.0 to about 4.5:1.0, about 4.5:1.0 to about 5.0:1.0, 4.1:1.0 to 4.9:1.0, 4.2:1.0 to 4.8:1.0, 4.3:1.0 to 4.7.0:1.0, or 4.4:1.0 to 4.6:1.0. In an embodiment, the molar ratio of the PSMA I&T to ¹⁷⁷Lu in the composition is about 5.0:1.0 to about 5.5:1.0, about 5.5:1.0 to about 6.0:1.0, about 6.0:1.0 to about 6.5:1.0, about 6.5:1.0 to about 7.0:1.0, about 7.0:1.0 to about 7.5:1.0, about 7.5:1.0 to about 8.0:1.0, about 8.0:1.0 to about 8.5:1.0, about 8.5:1.0 to about 9.0:1.0, about 9.0:1.0 to about 9.5:1.0, about 9.5:1.0 to about 10.0:1.0, about 10.0:1.0 to about 10.5:1.0, about 10.5:1.0 to about 11.0:1.0, about 11.0:1.0 to about 11.5:1.0, or about 11.5:1.0 to about 12.0:1.0.

In some embodiments, the total amount of ¹⁷⁷Lu-PSMA I&T present in the radiopharmaceutical composition may range from about 1.0 μg/ml to about 3 μg/ml, from about 1 μg/ml to about 2 μg/ml, from about 1 μg/ml to about 2.5 μg/ml, from about 1.1 μg/ml to about 2 μg/ml, from about 1.1 μg/ml to about 1.9 μg/ml, from about 1.1 μg/ml to about 1.9 μg/ml, from about 1.1 μg/ml to about 1.7 μg/ml, from about 1.1 μg/ml to about 1.6 μg/ml, from about 1.1 μg/ml to about 1.5 μg/ml, from about 1.1 μg/ml to about 1.4 μg/ml, or from about 1.1 μg/ml to about 1.3 μg/ml. In another embodiment, the total amount of ¹⁷⁷Lu-PSMA I&T in the radiopharmaceutical composition may range from about 0.5 μg/ml to about 1.5 μg/ml. In various embodiments, the total amount of ¹⁷⁷Lu-PSMA I&T present in the radiopharmaceutical composition may be about 0.5 μg/ml, about 0.6 μg/ml, about 0.7 μg/ml, about 0.8 μg/ml, about 0.9 μg/ml, about 1.0 μg/ml, about 1.1 μg/ml, about 1.2 μg/ml, about 1.3 μg/ml, about 1.4 μg/ml, about 1.5 μg/ml, about 1.6 μg/ml, about 1.7 μg/ml, about 1.8 μg/ml, about 1.9 μg/ml, about 2.0 μg/ml, or about 2.1 μg/ml.

In some embodiments, the total amount of ¹⁷⁷Lu-PSMA I&T present in the radiopharmaceutical composition may range from about 3.0 μg/ml to about 9.0 μg/ml, from about 3.5 μg/ml to about 8.5 μg/ml, from about 4.0 μg/ml to about 8.0 μg/ml, from about 4.5 μg/ml to about 7.5 μg/ml, from about 5.0 μg/ml to about 7.0 μg/ml, or from about 5.5 μg/ml to about 6.5 μg/ml. In another embodiment, the total amount of ¹⁷⁷Lu-PSMA I&T in the radiopharmaceutical composition may range from about 0.5 μg/ml to about 1.5 μg/ml.

In some embodiments, the total amount of ¹⁷⁷Lu-PSMA I&T present in the radiopharmaceutical composition may be less than 2.0 μg/ml. In other embodiments, the total amount of ¹⁷⁷Lu-PSMA I&T present in the radiopharmaceutical composition may be less than 4.0 μg/ml. In other embodiments, the total amount of ¹⁷⁷Lu-PSMA I&T present in the radiopharmaceutical composition may be less than 5.0 μg/ml. In other embodiments, the total amount of ¹⁷⁷Lu-PSMA I&T present in the radiopharmaceutical composition may be less than 6.0 μg/ml. In other embodiments, the total amount of ¹⁷⁷Lu-PSMA I&T present in the radiopharmaceutical composition may be less than 3.0 μg/ml.

In some embodiments, the total amount of ¹⁷⁷Lu-PSMA I&T present in the radiopharmaceutical composition may range from about 9 μg/ml to 20 μg/ml, 10 μg/ml to 20 μg/ml, 11 μg/ml to 20 μg/ml, 11 μg/ml to 15 μg/ml, 11 μg/ml to 14 μg/ml, or 11 μg/ml to 13 μg/ml. In another embodiment, the total amount of ¹⁷⁷Lu-PSMA I&T in the radiopharmaceutical composition may range from about 5 μg/ml to about 15 μg/ml. In various embodiments, the total amount of ¹⁷⁷Lu-PSMA I&T present in the radiopharmaceutical composition may be about 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, or 18 μg/ml. The composition may have less than 12 μg/ml or less than 6 μg/ml of Lu-PSMA I&T.

The radioactivity/volume of ¹⁷⁷Lu-PSMA I&T in the composition may be adjusted according to dose strength. In an embodiment, the composition may include 0.5 GBq (13.5 mCi) of ¹⁷⁷Lu-PSMA I&T in a 1 ml solution. In other words, the composition may include 10 GBq (270 mCi) of ¹⁷⁷Lu-PSMA I&T in a 20 ml solution. In another embodiment, the composition may include 1 GBq (27 mCi) of ¹⁷⁷Lu-PSMA I&T in a 1 ml solution. In other words, the composition may include 10 GBq (270 mCi) of ¹⁷⁷Lu-PSMA I&T in a 10 ml solution at time of synthesis.

In one embodiment, the radioactivity concentration of the ¹⁷⁷Lu-PSMA I&T in the radiopharmaceutical composition is less than about 50 mCi/ml, less than about 45 mCi/ml, less than about 40 mCi/ml, less than about 35 mCi/ml, less than about 30 mCi/ml, less than about 25 mCi/ml, less than about 20 mCi/ml, or less than about 15 mCi/ml. In another embodiment, the radioactivity concentration of the ¹⁷⁷Lu-PSMA I&T in the radiopharmaceutical composition is from about 5 mCi/ml to about 30 mCi/ml, from about 10 mCi/ml to about 20 mCi/ml, from about 10 mCi/ml to about 15 mCi/ml, from about 10 mCi/ml to about 20 mCi/ml, or from about 13 mCi/ml to about 30 mCi/ml. In one specific embodiment, the radioactivity concentration of the ¹⁷⁷Lu-PSMA I&T in the radiopharmaceutical composition is about 5 mCi/ml, about 10 mCi/ml, about 13.5 mCi/ml, about 15 mCi/ml, about 20 mCi/ml, about 27 mCi/ml, about 30 mCi/ml, about 30 mCi/ml, about 35 mCi/ml or about 40 mCi/ml. In another embodiment, the radioactivity concentration of the ¹⁷⁷Lu-PSMA I&T in the radiopharmaceutical composition is from about 10 mCi/ml to about 15 mCi/ml, from about 10 mCi/ml to about 20 mCi/ml.

In one embodiment, the radioactivity of the ¹⁷⁷Lu-PSMA I&T in the radiopharmaceutical composition is less than about 500 mCi, less than about 450 mCi, less than about 400 mCi, less than about 350 mCi, less than about 300 mCi, less than about 250 mCi, or less than about 200 mCi per vial. In another embodiment, the radioactivity of the ¹⁷⁷Lu-PSMA I&T in the radiopharmaceutical composition is from about 10 mCi to about 400 mCi per vial. In one specific embodiment, the radioactivity of the ¹⁷⁷Lu-PSMA I&T in the radiopharmaceutical composition is about 27 mCi, 150 mCi, about 160 mCi, about 170 mCi, about 180 mCi, about 190 mCi, about 200 mCi, about 250 mCi, about 270 mCi, about 300 mCi, about 313 mCi, about 318 mCi, about 338 mCi, about 345 mCi, about 346 mCi, about 354 mCi, about 360 mCi, about 370 mCi, about 380 mCi, about 390 mCi per vial.

In yet another embodiment, the ¹⁷⁷Lu-PSMA I&T drug product has a standard radioactivity concentration of about 12 mCi/ml or about 32 mCi/ml at the end of production. In one embodiment, the ¹⁷⁷Lu-PSMA I&T drug product has standard radioactivity concentration of about 13.5 mCi/ml or about 27 mCi/ml at the end of production.

### (ii) Antioxidant

The antioxidant may act as a buffer and/or stabilizing agent. The total amount of antioxidant in the radiopharmaceutical composition can and will vary. Examples of suitable antioxidants include but are not limited to ascorbic acid or gentisic acid. The amount of antioxidant in the composition may range from about 10 mg/ml to 90 mg/ml, about 15 mg/ml to 85 mg/ml, about 20 mg/ml to 80 mg/ml, about 25 mg/ml to 75 mg/ml, about 30 mg/ml to 70 mg/ml, about 35 mg/ml to 65 mg/ml, about 40 mg/ml to 60 mg/ml, or about 45 mg/ml to 55 mg/ml. Said in another way, the amount of antioxidant in the composition may range from about 10 mg to 90 mg, about 15 mg to 85 mg, about 20 mg to 80 mg, about 25 mg to 75 mg, about 30 mg to 70 mg, about 35 mg to 65 mg, about 40 mg to 60 mg, or about 45 mg to 55 mg per ml.

In an embodiment, there may be ≤10 mg/ml, ≤9.5 mg/ml, ≤9 mg/ml, ≤8.5 mg/ml, ≤8 mg/ml, ≤7.5 mg/ml, ≤7 mg/ml, ≤6.5 mg/ml, ≤6 mg/ml, ≤5.5 mg/ml, 5 mg/ml, ≤4.5 mg/ml, ≤4 mg/ml, ≤3.5 mg/ml, ≤3 mg/ml, ≤2.5 mg/ml, ≤2 mg/ml, ≤1.5 mg/ml, ≤1 mg/ml, or ≤0.5 mg/ml antioxidant.

In an embodiment, the antioxidant may be ascorbic acid and/or ascorbate. Ascorbic acid and/or ascorbate may minimize or reduce radiolysis of radiolabeled compositions.

In some embodiments, ascorbic acid present in the radiopharmaceutical composition may range from about 10 to about 50 mg, from about 20 to about 50 mg, from about 30 to about 50 mg or from about 35 to about 45 mg per ml. In another embodiment, ascorbic acid in the radiopharmaceutical composition may range from about 5 mg to about 50 mg per ml. In another embodiment, there may be ≤10 mg/ml, ≤9.5 mg/ml, ≤9 mg/ml, ≤8.5 mg/ml, ≤8 mg/ml, ≤7.5 mg/ml, ≤7 mg/ml, ≤6.5 mg/ml, ≤6 mg/ml, ≤5.5 mg/ml, 5 mg/ml, ≤4.5 mg/ml, ≤4 mg/ml, ≤3.5 mg/ml, ≤3 mg/ml, ≤2.5 mg/ml, ≤2 mg/ml, ≤1.5 mg/ml, ≤1 mg/ml, or ≤0.5 mg/ml ascorbic acid or ascorbate.

In various embodiments, ascorbic acid present in the radiopharmaceutical composition may be about 5 mg, about 10 mg, about 15 mg, about 20 mg, about 25 mg, about 30 mg, about 31 mg, about 35 mg, about 36 mg, about 37 mg, about 38 mg, about 39 mg, about 40 mg, about 40.5 mg, about 41 mg, about 41.5 mg, about 42 mg, about 42.5 mg, about 43 mg, about 43.5 mg, about 44 mg, about 44.5 mg, about 45 mg, about 46 mg, about 47 mg, about 48 mg, about 49 mg, about 50 mg, about 55 mg, about 60 mg, about 65 mg, about 70 mg, about 75 mg, about 80 mg, about 85 mg, or about 90 mg per ml. For example, the amount of ascorbic acid in 1 ml of the composition may be about 25 mg to 30 mg, about 30 mg to 35 mg, about 35 mg to 40 mg, or about 40 mg to 45 mg per ml.

In yet another embodiment, the concentration of ascorbic acid in the radiopharmaceutical composition may be from about 10 mg/ml to about 80 mg/ml, from about 10 mg/ml to about 75 mg/ml, from about 10 mg/ml to about 70 mg/ml, from about 15 mg/ml to about 80 mg/ml, from about 15 mg/ml to about 75 mg/ml, from about 15 mg/ml to about 70 mg/ml, from about 20 mg/ml to about 80 mg/ml, from about 20 mg/ml to about 75 mg/ml, from about 25 mg/ml to about 40 mg/ml, from about 20 mg/ml to about 80 mg/ml, from about 20 mg/ml to about 75 mg/ml, or from about 20 mg/ml to about 70 mg/ml.

In one specific embodiment, the concentration of ascorbic acid in the radiopharmaceutical composition is about 10 mg/ml, about 15 mg/ml, about 20 mg/ml, about 25 mg/ml, about 21 mg/ml, about 25 mg/ml, about 30 mg/ml, about 31 mg/ml, about 35 mg/ml, about 40 mg/ml, about 42.5 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, or about 100 mg/ml.

In at least one embodiment, the total amount of ascorbic acid in the radiopharmaceutical composition may be about 31 mg/ml. In a further embodiment, the total amount of ascorbic acid in the radiopharmaceutical composition may be about 15 mg/ml, about 21 mg/ml, about 25 mg/ml, about 31 mg/ml, 33 mg/ml, 35 mg/ml, or about 42.5 mg/ml.

(iii) Stabilizing Agent

The stabilizing agent may be separate from the antioxidant. The total amount of stabilizing agent present in the radiopharmaceutical composition can and will vary. The stabilizing agent may further be used to limit or reduce radiolysis. The stabilizing agent may also function as a vehicle for the composition.

The stabilizing agent includes but is not limited to ethanol, para-aminebenzoic acid (PABA), dihydroxybenzoic acid (gentisate compounds), gentisic acid, cysteine, selenomethionine, ascorbic acid/sodium ascorbate, methionine, and combinations thereof. In an embodiment, the stabilizing agent includes ascorbic acid and sodium ascorbate.

In some embodiments, the stabilizing agent is ethanol. Ethanol may be present in the pharmaceutical composition at about 0.01% (v/v) to about 10% (v/v), 0.01% (v/v) to 3% (v/v), about 0.5% (v/v) to 1% (v/v), about 1% (v/v) to 2% (v/v), about 2% (v/v) to about 3% (v/v), about 3% (v/v) to 4% (v/v), about 3.5% to 4.5% (v/v), about 4% to 5% (v/v), about 4.5% (v/v) to 5.5% (v/v), about 5% (v/v) to 6% (v/v), about 5.5% (v/v) to 6.5% (v/v), about 6% (v/v) to 7% (v/v), about 6.5% (v/v) to 7.5% (v/v), or about 7% (v/v) to 8% (v/v). In some embodiments, the pharmaceutical composition comprises zero (0.00% v/v) ethanol (i.e., ethanol may be absent from the pharmaceutical composition).

In one embodiment, the total amount of ethanol present in the radiopharmaceutical composition is from about 3% (v/v) to about 8% (v/v), or from 2% (v/v) to about 4% (v/v), or from about 7% (v/v) to about 8% (v/v). In various embodiments, the total amount of ethanol present in the radiopharmaceutical composition may be about 1% (v/v), about 2% (v/v), about 3% (v/v), about 3.5% (v/v), about 3.8% (v/v), about 4% (v/v), about 4.5% (v/v), about 5% (v/v), about 5.5% (v/v), about 6% (v/v), about 6.5% (v/v), about 7% (v/v), about 7.5% (v/v), about 8% (v/v), about 8.5% (v/v), about 9% (v/v), about 9.5% (v/v), or about 10% (v/v).

In another embodiment, there may be ≤10 mg/ml, ≤9.5 mg/ml, ≤9 mg/ml, ≤8.5 mg/ml, ≤8 mg/ml, ≤7.5 mg/ml, ≤7 mg/ml, ≤6.5 mg/ml, ≤6 mg/ml, ≤5.5 mg/ml, 5 mg/ml, ≤4.5 mg/ml, ≤4 mg/ml, ≤3.5 mg/ml, ≤3 mg/ml, ≤2.5 mg/ml, ≤2 mg/ml, ≤1.5 mg/ml, ≤1 mg/ml, or ≤0.5 mg/ml gentisic acid or gentisate.

In at least one example, the radiopharmaceutical composition includes 3.8% (v/v) ethanol. In another example, the radiopharmaceutical composition includes 7.5% (v/v).

Stated alternatively, the total amount of ethanol present in the radiopharmaceutical composition may be about 5 mg, about 10 mg, about 15 mg, about 20 mg, about 25 mg, about 30 mg, about 35 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 95 mg, about 100 mg, about 105 mg, about 110 mg, about 115 mg, or about 80 mg per ml.

In some embodiments, the total amount of ethanol present in the radiopharmaceutical composition may range from about 20 mg to about 35 mg per ml. In another embodiment, the total amount of ethanol in the radiopharmaceutical composition may range from about 43 mg to about 63 mg per ml.

In some embodiments, the total amount of ethanol present in the radiopharmaceutical composition may range from about 25 mg to 80 mg, about 30 to 40 mg, about 40 to 50 mg, about 50 to 60 mg, about 60 to 70 mg, or about 70 to 80 mg per. In another embodiment, the total amount of ethanol in the radiopharmaceutical composition may range from about 30 mg to about 60 mg per ml.

In a further embodiment, the ratio of ethanol in the radiopharmaceutical composition may be about 300 mg per 10 ml or about 30 mg/ml. In another embodiment, the ratio of ethanol in the radiopharmaceutical composition may be about 200 mg per 10 ml. In still another embodiment, the ratio of ethanol in the radiopharmaceutical composition may be about 350 mg per 10 ml.

Stated alternatively, the amount of ethanol in the composition may range from about 35 μl/ml to about 75 μl/ml. For example, the amount of ethanol in 1 ml of the composition may be about 35 μl to 40 μl, about 40 μl to 45 μl, about 45 μl to 50 μl, about 50 μl to 55 μl, about 55 μl to 60 μl, about 60 μl to 65 μl, about 65 μl to 70 μl, or about 70 μl to 75 μl. In at least one example, 1 ml of the composition includes 37.5 μl (29.5 mg) of ethanol. In another example, 1 ml of the composition includes 75 μl (58.9 mg) of ethanol.

### (iv) Metal Ion Chelator (Chelating Agent)

In some embodiments, the disclosure provides for a radiopharmaceutical composition with a micro dose of ¹⁷⁷Lu-PSMA I&T solution and at least one metal ion chelator. A suitable chelating agent may include ethylenediamine tetracetic acid (EDTA) and its salts, N-(hydroxy-ethyl)ethylenediaminetriacetic acid, nitrilotriacetic acid (NTA), ethylene-bis(oxyethylene-nitrilo)tetraacetic acid, 1,4,7,10-tetraazacyclodo-decane-N,N′,N″,N′″-tetraacetic acid, 1,4,7,10-tetraaza-cyclododecane-N,N′,N″-triacetic acid, 1,4,7-tris(carboxymethyl)-10-(2′-hydroxypropyl)-1,4,7,10-tetraazocyclodecane, 1,4,7-triazacyclonane-N,N′,N″-triacetic acid, 1,4,8,11-tetraazacyclotetra-decane-N,N′,N″,N′″-tetraacetic acid; diethylenetriamine-pentaacetic acid (DTPA), ethylenedicysteine, bis(aminoethanethiol)carboxylic acid, triethylenetetramine-hexaacetic acid,1,2-diaminocyclohexane-N,N,N′,N′-tetraacetic acid, or a combination thereof. In one embodiment, the chelating agent may be the sodium salt of EDTA. In one embodiment, the chelating agent may comprise DTPA and an absence of EDTA.

In some embodiments, the metal ion chelator may be ethylenediamine tetracetic acid (EDTA) and its salts, N-(hydroxy-ethyl)ethylenediaminetriacetic acid, nitrilotriacetic acid (NTA), ethylene-bis(oxyethylene-nitrilo)tetraacetic acid, 1,4,7,10-tetraazacyclodo-decane-N,N′,N″,N′″-tetraacetic acid, 1,4,7,10-tetraaza-cyclododecane-N,N′,N″-triacetic acid, 1,4,7-tris(carboxymethyl)-10-(2′-hydroxypropyl)-1,4,7,10-tetraazocyclodecane, 1,4,7-triazacyclonane-N,N′,N″-triacetic acid, 1,4,8,11-tetraazacyclotetra-decane-N,N′,N″,N′″-tetraacetic acid; diethylenetriamine-pentaacetic acid (DTPA), ethylenedicysteine, bis(aminoethanethiol)carboxylic acid, triethylenetetramine-hexaacetic acid, and 1,2-diaminocyclohexane-N,N,N′,N′-tetraacetic acid. In one embodiment, the metal ion chelator may be disodium EDTA. In one embodiment, the metal ion chelator may be DPTA.

In one embodiment, the amount of chelating agent present in the radiopharmaceutical composition may range from about 5 μg to 500 μg. In some embodiments, the amount of metal ion chelator present in the radiopharmaceutical composition may range from about 5 μg to 50 μg.

In some embodiments, the amount of chelating agent present may be about 5 μg, about 6 μg, about 7 μg, about 8 μg, about 9 μg, about 10.5 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 45 μg, about 50 μg, about 60 μg, about 70 μg, about 80 μg, about 90 μg, about 100 μg, about 110 μg, about 80 μg, about 130 μg, about 140 μg, about 150 μg, about 160 μg, about 170 μg, about 180 μg, about 190 μg, about 200 μg, about 210 μg, about 220 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg, about 410 μg, about 420 μg, about 430 μg, about 440 μg, about 450 μg, about 460 μg, about 470 μg, about 480 μg, about 490 μg, or about 500 μg.

The concentration of metal ion chelator in the composition may range from about 5 μg/ml to about 500 μg/ml. In another embodiment, the concentration of chelating agent present in the radiopharmaceutical composition may range from about 5 μg/ml to about 200 μg/ml. In another embodiment, the concentration of chelating agent present in the radiopharmaceutical composition may range from about 5 μg/ml to 75 μg/ml, 10 μg/ml to about 25 μg/ml, about 25 μg/ml to about 50 μg/ml, about 50 μg/ml to about 75 μg/ml, or about 75 μg/ml to about 100 μg/ml, about 100 μg/ml to about 125 μg/ml, about 125 μg/ml to about 150 μg/ml, or about 150 μg/ml to about 200 μg/ml.

In some embodiments, the concentration of chelating agent present may be about 5 μg/ml, about 6 μg/ml, about 7 μg/ml, about 8 μg/ml, about 9 μg/ml, about 10.5 μg/ml, about 11 μg/ml, about 12 μg/ml, about 13 μg/ml, about 14 μg/ml, about 15 μg/ml, about 16 μg/ml, about 17 μg/ml, about 18 μg/ml, about 19 μg/ml, about 20 μg/ml, about 21 μg/ml, about 22 μg/ml, about 23 μg/ml, about 24 μg/ml, about 25 μg/ml, about 26 μg/ml, about 27 μg/ml, about 28 μg/ml, about 29 μg/ml, about 30 μg/ml, about 31 μg/ml, about 32 μg/ml, about 33 μg/ml, about 34 μg/ml, about 35 μg/ml, about 36 μg/ml, about 37 μg/ml, about 38 μg/ml, about 39 μg/ml, about 40 μg/ml, about 45 μg/ml, or about 50 μg/ml. In another embodiment, the concentration of chelating agent present in the radiopharmaceutical composition may range from about 100 μg/ml to about 125 μg/ml, about 125 μg/ml to about 150 μg/ml, or about 150 μg/ml to about 200 μg/ml.

In other embodiments, the concentration of chelating agent present may be about 80 μg/ml, about 90 μg/ml, about 91 μg/ml, about 92 μg/ml, about 93 μg/ml, about 94 μg/ml, about 95 μg/ml, about 96 μg/ml, about 97 μg/ml, about 98 μg/ml, about 99 μg/ml, about 100 μg/ml, about 101 μg/ml, about 102 μg/ml, about 103 μg/ml, about 104 μg/ml, about 105 μg/ml, about 106 μg/ml, about 107 μg/ml, about 108 μg/ml, about 109 μg/ml, about 110 μg/ml, about 115 μg/ml, about 120 μg/ml, about 125 μg/ml, about 130 μg/ml, about 135 μg/ml, about 140 μg/ml, about 145 μg/ml, about 150 μg/ml, about 155 μg/ml, about 160 μg/ml, about 170 μg/ml, about 180 μg/ml, about 190 μg/ml, or about 200 μg/ml.

In another embodiment, the amount of metal ion chelator in the radiopharmaceutical composition may be from about 0.001% to about 0.20% (w/w), about 0.20% to about 0.40% (w/w), about 0.40% to about 0.60% (w/w), about 0.60% to about 0.80% (w/w), or about 0.80% to about 1.00% (w/w) of such radiopharmaceutical composition. In some embodiments, the amount of metal ion chelator present in a radiopharmaceutical composition may be about 0.001%, 0.002%, 0.003%, 0.004%, 0.005%, 0.01%, 0.02%, 0.03%, 0.04%, 0.05%, 0.06%, 0.07%, 0.08%, 0.09%, 0.10%, 0.11%, 0.12%, 0.13%, 0.14%, or 0.15% (w/w) of the total weight of the radiopharmaceutical composition.

For example, the amount of disodium EDTA, diethylenetriamine-pentaacetic acid (DTPA), or a combination thereof in 1 ml of the composition may be about 10 μg to 15 μg, about 13 μg to 18 μg, about 15 μg to 20 μg, about 20 μg to 25 μg, about 25 μg to 50 μg, about 50 μg to 75 μg, or about 75 μg to 150 μg. In some embodiments, the amount of disodium EDTA present may be about 5 μg, about 6 μg, about 7 μg, about 8 μg, about 9 μg, about 10.5 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 45 μg, or about 50 μg.

In at least one example, 1 ml of the composition includes 15.5 μg of disodium EDTA. In another example, 1 ml of the composition includes 21 μg of disodium EDTA.

In one embodiment, trace metal content in the composition is undetectable. In another embodiment, the Fe metal content in the composition is ≤0.05 μg/GBq, ≤0.03 μg/GBq, ≤0.01 μg/GBq, or below the detectable limit. In another embodiment, the Cu metal content in the composition is ≤0.05 μg/GBq, ≤0.03 μg/GBq, ≤0.01 μg/GBq, or below the detectable limit. In another embodiment, the Zn metal content in the composition is ≤0.05 μg/GBq, ≤0.03 μg/GBq, ≤0.01 μg/GBq, or below the detectable limit. In another embodiment, the Pb metal content in the composition is ≤0.05 μg/GBq, ≤0.03 μg/GBq, ≤0.01 μg/GBq, or below the detectable limit. In another embodiment, the Co metal content in the composition is ≤0.05 μg/GBq, ≤0.03 μg/GBq, ≤0.01 μg/GBq, or below the detectable limit. In another embodiment, the Ni metal content in the composition is ≤0.05 μg/GBq, ≤0.03 μg/GBq, ≤0.01 μg/GBq, or below the detectable limit. In another embodiment, the Zn metal content in the composition is ≤0.05 μg/GBq, ≤0.03 μg/GBq, ≤0.01 μg/GBq, or below the detectable limit. In another embodiment, the Cr metal content in the composition is ≤0.05 μg/GBq, ≤0.03 μg/GBq, ≤0.01 μg/GBq, or below the detectable limit. In another embodiment, the Yb metal content in the composition is ≤0.05 μg/GBq, ≤0.03 μg/GBq, ≤0.01 μg/GBq, or below the detectable limit.

In another embodiment, the compositions described herein comprise M-PSMA I&T wherein M=Cu, Pb, Co, Fe, Ni, Zn, Cr, and/or Yb, and the total combined concentration of M-PSMA I&T is less than 500 ppm, less than 400 ppm, less than 300 ppm, less than 200 ppm, less than 100 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm. In another embodiment, the compositions described herein comprise less than 500 ppm, less than 400 ppm, less than 300 ppm, less than 200 ppm, less than 100 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm of Cu-PSMA I&T. In another embodiment, the compositions described herein comprise less than 500 ppm, less than 400 ppm, less than 300 ppm, less than 200 ppm, less than 100 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm of Pb-PSMA I&T. In another embodiment, the compositions described herein comprise less than 500 ppm, less than 400 ppm, less than 300 ppm, less than 200 ppm, less than 100 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm of Co-PSMA I&T. In another embodiment, the compositions described herein less than 500 ppm, less than 400 ppm, less than 300 ppm, less than 200 ppm, less than 100 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm of Fe-PSMA I&T. In another embodiment, the compositions described herein comprise less than 500 ppm, less than 400 ppm, less than 300 ppm, less than 200 ppm, less than 100 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm Ni-PSMA I&T. In another embodiment, the compositions described herein comprise less than 500 ppm, less than 400 ppm, less than 300 ppm, less than 200 ppm, less than 100 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm Zn-PSMA I&T. In another embodiment, the compositions described herein comprise less than 500 ppm, less than 400 ppm, less than 300 ppm, less than 200 ppm, less than 100 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm Cr-PSMA I&T. In another embodiment, the compositions described herein comprise less than 500 ppm, less than 400 ppm, less than 300 ppm, less than 200 ppm, less than 100 ppm, less than 50 ppm, less than 40 ppm, less than 30 ppm, less than 20 ppm, less than 10 ppm, less than 9 ppm, less than 8 ppm, less than 7 ppm, less than 6 ppm, less than 5 ppm, less than 4 ppm, less than 3 ppm, less than 2 ppm, or less than 1 ppm Yb-PSMA I&T.

### (v) pH Adjuster

Suitable pH adjusters include but are not limited to any one of hydrochloric acid, sodium hydroxide, sodium bicarbonate, or combinations thereof.

In some embodiments, hydrochloric acid may be used to adjust the pH of the radiopharmaceutical composition. In an embodiment, the amount of hydrochloric acid in the composition may range from 0 mg/ml to about 2 mg/ml. In some embodiments, the amount of HCl may range from 1.6 ml of 0.05 M HCl to 2 ml of 0.04 M HCl in a production batch. In one embodiment, the batch has an activity of about 10 to 20 Ci. In another embodiment the batch has an activity of about 16 Ci. The amount of HCl in the composition may vary, to adjust the final formulation pH. In various embodiments, the final formulation pH ranges from pH 3.0 to 5.0. In at least one example, HCl is added to the composition until a final pH of 3.5±0.1 to 4.5±0.1 is reached.

In an embodiment, the amount of sodium bicarbonate in the composition may be a sufficient quantity control the pH of the composition to 5.5 to 7.0 prior to the addition of HCl.

In an embodiment, the amount of NaOH in the composition may be a sufficient quantity control the pH of the composition to 5.5 to 7.0 prior to the addition of HCl.

### (vi) Water

The composition may further include a sufficient amount of water to make the desired final volume for the solution for injection. For example, water may be added to make a final volume of 1 ml, 10 ml, 15 ml, or 20 ml.

### IV. Process of Making the Radiopharmaceutical Composition

The whole manufacturing process is a one-step radiolabelling process using a PSMA I&T precursor. The success of the labelling is dependent on temperature, time and pH. The reaction takes place in a reactor vial at elevated temperatures. For example, the reactor maybe heated at setpoint of 110° C. and the maximum temperature reached in the reaction solution is about 95° C. The radiolabeled product is isolated on a C18 cartridge and formulated to the final composition after elution to the bulk vial. The final product is dispensed in a grade A controlled environment.

In another embodiment, the radiolabelling process uses a PSMA I&T precursor and wherein the composition is radiolabeled for 5 to 15 minutes at a temperature of about 65 to about 75 degrees C. In yet another embodiment, the composition is radiolabeled for 5 to 15 minutes at a temperature of about 65 to about 80 degrees C. In another embodiment, the composition is radiolabeled for 5 to 15 minutes at a temperature of about 65, about 70, about 75, or about 80 degrees C. In another embodiment, the composition is radiolabeled for 5 to 15 minutes at a temperature of about 70 to about 80 degrees C. In yet another embodiment, the composition is radiolabeled for 5 to 15 minutes at a temperature of about 75 to 80 degrees C.

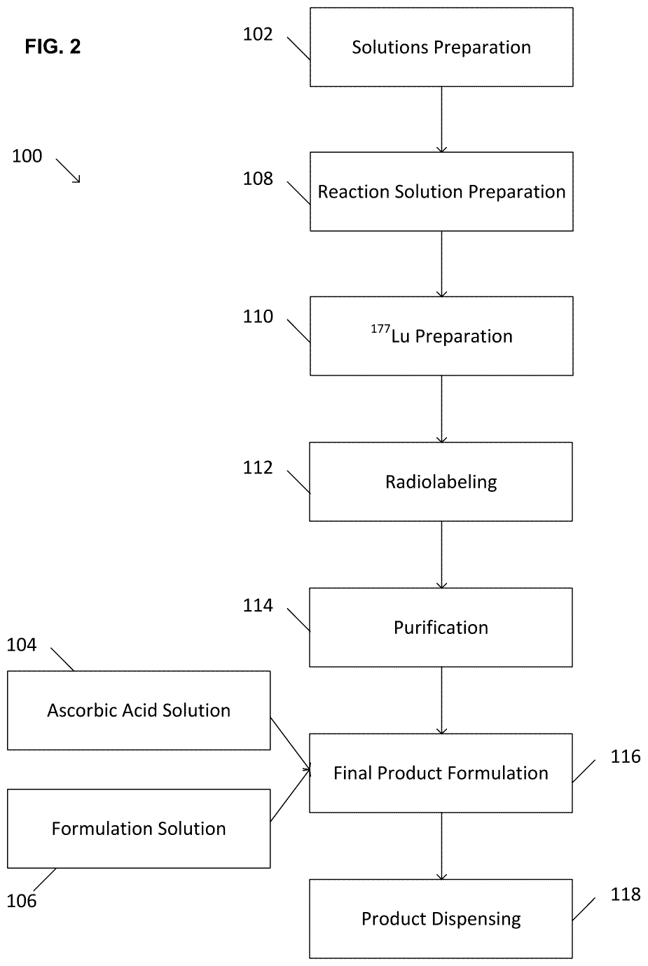

The ¹⁷⁷Lu-PSMA I&T composition solution may be prepared using the following method 100, for example as shown in FIG. 2. The order of the steps may vary, such as the order of preparing the various solutions.

In an embodiment, step 102 may include preparing four solutions for the synthesis. The four solutions may include 0.04 M hydrochloric acid, 0.4 M sodium acetate, 20% (w/w) L-ascorbic acid, and PSMA I&T in water at about 460 to about 500 μg/ml. The PSMA I&T precursor may be dissolved in sterile water for injection. For example, 80 μg to 600 μg of precursor may be used in the reaction depending on the number of doses produced. In at least one example, 463 μl/ml of PSMA I&T precursor may be used to produce the composition.

In an embodiment, step 104 may include preparing an ascorbic acid solution (dilution buffer). In some examples, the ascorbic acid solution may be a 50 mg/ml ascorbic acid solution. The solution pH may be adjusted to 4.5±0.25, 4.5±0.30, 4.5±0.35, 4.5±0.40, 4.5±0.45, or 4.5±0.50. For example, 50 mg/ml ascorbic acid solution is prepared, and the pH of the solution is adjusted to 4.5 using 30% hydrochloric acid. In another example, the ascorbic acid solution may include 33 mg/ml ascorbic acid/sodium ascorbate at pH 4.25±0.25 and 0.1 mg/ml DTPA.

In an embodiment, optional step 106 may include preparing a formulation solution/buffer. The formulation solution is prepared from an injections grade solution containing ascorbic acid, absolute ethanol and injections grade water. In an example, the formulation solution is prepared by adding sufficient amounts of following solutions into the bulk vial: about 50 mg/ml ascorbic acid pH 4.5 solution (prepared in step 104), 30% ethanol solution, and water. The formulation solution may include 31 mg/ml to 42.5 mg/ml ascorbic acid and 3.8% to 7.5% ethanol (v/v %). In some embodiments, the formulation buffer may be adjusted to enable the final composition to have an extended shelf life. In at least one example, the formulation solution comprises 31 mg/ml ascorbic acid, 3.8% (v/v) ethanol, and pH 4.5. The formulation buffer is prepared ex tempore as part of the synthesis preparation and predetermined amount is added to the bulk vial as part of the synthesis preparations.

In an embodiment, step 108 may include preparing a reaction solution. A reaction solution may include sodium acetate, HCl, and L-ascorbic acid. Alternatively, a reaction solution may include sodium ascorbate. The reaction solution may be prepared in the reactor using solutions prepared in step 102. In an example, the reaction solution may include 4 ml 0.4 M sodium acetate, a volume of about 463 μg/ml PSMA I&T solution, and 150 μl 20% (w/w) L-ascorbic acid. In another example, the reaction solution may include 0.33M sodium ascorbate (reaction buffer) and PSMA I&T in the reaction buffer. In some examples, the reaction solution may include 1.6 ml 0.05 M HCl or 2 ml 0.04 M HCl (0.08 mmol HCl). Ascorbic acid concentration in the reaction solution may range from 3.75 mg/ml to 5.00 mg/ml.

In an embodiment, step 110 may include preparing ¹⁷⁷Lu. In some embodiments, ¹⁷⁷Lu may be provided in HCl. [¹⁷⁷Lu]LuCl₃ may be provided in 0.04 M or 0.05 M HCl. For example, 40-44 GBq/ml of ¹⁷⁷Lu may be provided in 0.04 M HCl. In another example, less than 61 GBq of ¹⁷⁷Lu may be provided in 0.05 M HCl. The [¹⁷⁷Lu]LuCl₃ in 0.04 M or 0.05 M hydrochloric acid may be transferred into the reactor and the [¹⁷⁷Lu]LuCl₃ vial may be rinsed with an additional required volume of 0.04 M hydrochloric acid (prepared in step 102) that is also then transferred into the reactor.

The reaction volume may range from 6 ml to 8 ml. The volume may be dependent on the amount of precursor used.

In an embodiment, step 112 may include radiolabeling the PSMA-I&T with ¹⁷⁷Lu. The reaction mixture may be heated up to about 75° C., up to about 80° C., up to about 85° C., up to about 90° C., or up to about 95° C. In an example, the setpoint for heating is 110° C. and the actual maximum temperature reached is about 95° C. The reaction volume may be heated for up to 5 minutes, up to 10 minutes, up to 15 minutes, or up to 20 minutes. In at least one example, the reaction mixture is heated at setpoint of 110° C. for 15 minutes. In at least one additional example, the reaction mixture is heated at a setpoint of 75° C. for 10 minutes.

In an embodiment, optional step 114 may include purifying the reaction mixture. For example, the solution may be run through a cassette/cartridge containing a hydrophobic, reverse-phase, silica-based bonded phase (e.g., C18 Sep-Pak). In at least one example, the reaction mixture may be passed through a C18 Sep Pak cartridge and the cartridge is rinsed with water. ¹⁷⁷Lu-PSMA I&T product is retained in the cartridge. In some embodiments, the reaction mixture may not be purified.

In an embodiment, step 116 may include eluting or diluting the final product. In an example, the ¹⁷⁷Lu-PSMA I&T is diluted with dilution buffer prepared in step 104 to the desired radioactivity concentration. The composition may be eluted using 1.5 ml of ethanol-water in a 1:1 ratio. The cassette may then be flushed using 8.5 ml ascorbic acid 50 mg/ml. A formulation solution may then be added to form the final composition. In at least one example, ¹⁷⁷Lu-PSMA I&T is eluted from the C18 cartridge with 1.5 ml of 50% (v/v) ethanol followed by 8.5 ml of 50 mg/ml pH 4.5 ascorbic acid solution (prepared in step 104) into the bulk vial where it is diluted with formulation solution/buffer (prepared in step 106, already in the bulk vial). The resulting solution may have a pH of 3.5 to 4.5. In some embodiments, the pH may be adjusted. In an example, the 50 mg/ml ascorbic acid solution pH is adjusted to 3.5 to 4.5. In other examples the pH is adjusted to 5.0 or less.

Presumably the stability enhancing conditions, such as ascorbic acid solution at pH of about 5 or below, should preferably be applied as early as possible in the process. For example, ascorbic acid solutions at pH 5 or below may be used instead of water at step 114 to minimize radiolytic damage.

The final composition may be formulated as a solution suitable for injection. The product is diluted to a standard radioactivity concentration, and therefore, the final volume of the bulk composition varies depending on the starting radioactivity of ¹⁷⁷Lu introduced. The solution meets the requirements for sterility and bacterial endotoxins according to the European and United States pharmacopoeia, confirming an acceptable manufacturing process from a microbial point of view.

In some embodiments, at step 118 the final composition may be sterile filtered. The sterile filter may be a 0.22 μm sterile filter. The final product may be dispensed through a 0.22 μm sterile filter into single dose vials containing suitable volume and radioactivity referenced to the prescribed calibration time. For example, the final composition may be dispensed through a 0.22 μm sterile filter under a Class A environment into doses containing suitable volume and radioactivity at a calibration time.

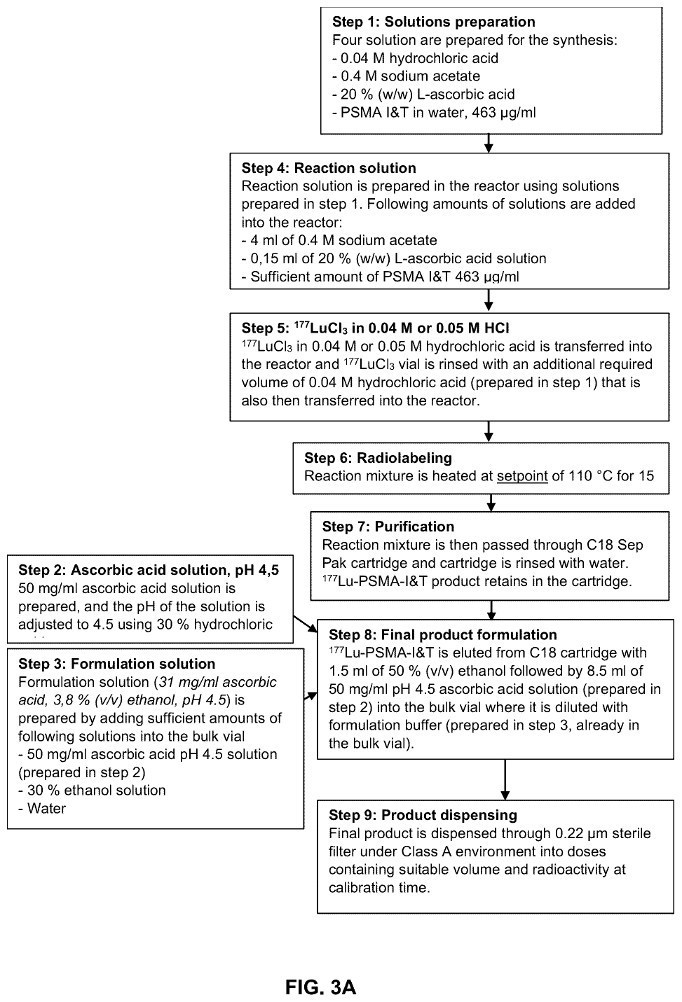

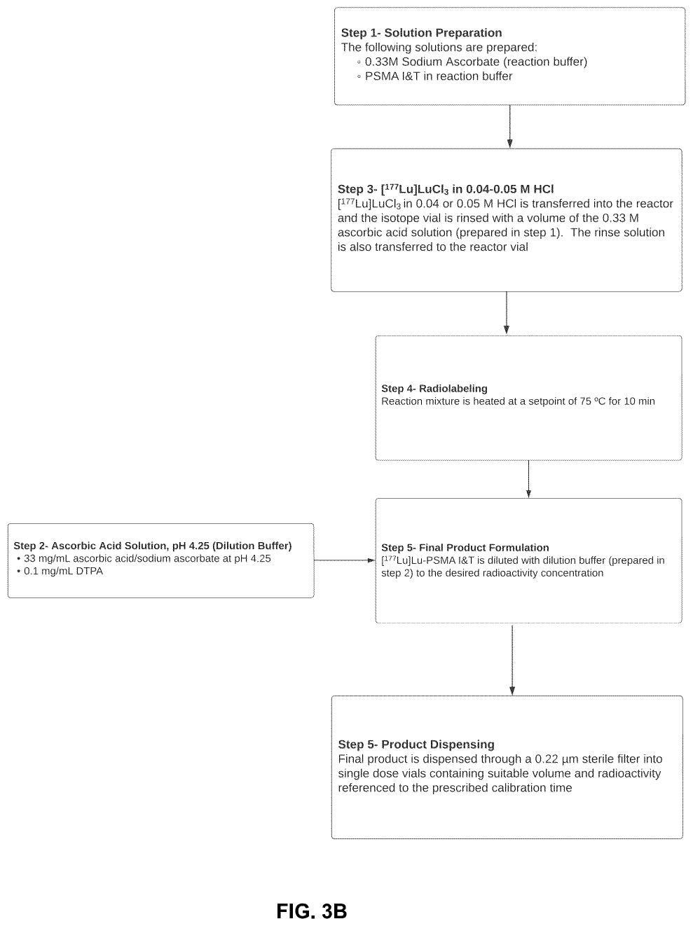

FIG. 3A provides one example of the process of making a radiopharmaceutical composition with purification of the reaction mixture and the formulation solution with ethanol. FIG. 3B provides one example of the process of making a radiopharmaceutical composition without purification and without ethanol.

Provided herein are methods for increasing the shelf life of a radiopharmaceutical product comprising ¹⁷⁷Lu-PSMA I&T. The methods may include adjusting the pH of the composition to 3.5, 3.75, 4.0, 4.25, or 4.5, adjusting the amount of ascorbic acid in the composition, and/or adjusting the radioactivity to increase the shelf life of the composition by 0.25, 0.5, 0.75, 1, 1.25, 1.5, 2, 2.25, 2.5, 2.75, or 3 days. For example, the radiopharmaceutical composition may have a shelf life of 1, 1.5, 2, 2.25, 2.5, 2.75, 3, 3.25, 3.5, 3.75, 4, 4.25, 4.5, 4.75, or 5 days. In an embodiment, adjusting the pH, radioactivity, and/or ascorbic acid may increase the radiochemical purity of the composition to at least 99%, at least 98.5%, at least 98%, at least 97.5%, at least 97%, at least 96.5%, at least 96%, at least 95.5%, or at least 95% for up to 1, 1.5, 2, 2.25, 2.5, 2.75, 3, 3.25, 3.5, 3.75, 4, 4.25, 4.5, 4.75, or 5 days.

Target pharmaceutical formulations in accordance with the present disclosure are as provided in Table 1A.

TABLE 1A
Target pharmaceutical formulations

| | Target Pharmaceutical Formulation | | | | | |
|---|---|---|---|---|---|---|
| Component | Compositions 1 and 2 | | Composition 3 | | Composition 4 | |
| Radioactivity concentration | ~1.0 GBq/mL or | | 0.5-0.6 GBq/mL or | | 0.5 GBq/mL or | |
| | 27 mCi/mL | | 13.5-16.2 mCi/mL | | 13.3 mCi/mL | |
| Ascorbic acid | 42.5 | mg/mL | 31 | mg/mL | 33 | mg/mL |
| Disodium EDTA | 21 | μg/mL | 15.5 | μg/mL | N/A | |
| DTPA | N/A | | N/A | | 100 | μg/mL |
| Ethanol | 7.5% | (v/v %) | 3.8% | (v/v %) | N/A | |
| pH | 5.5 | | 4.5 | | 4.25 | |
| Shelf-life (tested up to 40° C.) | 48 | h | 72 | h | 72 | h |

In another embodiment in making the Composition 4 disclosed in Table 1A above may be performed using a one-step radiolabelling process performed in the following steps.

The success of the labelling is dependent on temperature, time and pH. The reaction takes place in a reactor vial at elevated temperatures. For example, the reactor may be pre-heated at setpoint of 100° C. for 5 minutes and then reduced to 85° C. to achieve a reaction temperature of about 75° C. for 10 minutes. The radiolabeled product is formulated to the final composition, sterile filtered, and dispensed in a grade A controlled environment.

The ¹⁷⁷Lu-PSMA I&T composition solutions may be prepared as shown in FIG. 2b (201, 202, 203). The order of the steps may vary, such as the order of preparing the various solutions.

In an embodiment, step 201 may include preparing reaction buffer for the synthesis. The solution may include 82 mg/mL sodium ascorbate in water with pH >5. The PSMA I&T precursor may be dissolved in reaction buffer as shown in step 202. For example, 1000 μg to 5000 μg of precursor may be used in the reaction depending on the batch size.

In an embodiment, the amount of [¹⁷⁷Lu]LuCl₃ used in radiolabeling, step 204, can range from 50 mCi up to 15,200 mCi. The corresponding amount of PSMA I&T used during radiolabeling can range from 0.1 to 0.9 μg/mCi. For example, 15,000 mCi of [¹⁷⁷Lu]LuCl₃ and 4200 μg of PSMA I&T are added to the reactor during radiolabeling.

In an embodiment, step 203 may include preparing an ascorbic acid solution (dilution buffer). In some examples, the ascorbic acid solution may be a 33 mg/ml ascorbic acid solution. The solution pH may be adjusted to 4.25±0.05, 4.25±0.10, 4.25±0.15, 4.25±0.20, or 4.25±0.25. For example, the ascorbic acid solution may include 33 mg/ml ascorbic acid/sodium ascorbate at pH 4.25±0.25 and 0.1 mg/ml DTPA.

In an embodiment, step 204 may include preparing ¹⁷⁷Lu. In some embodiments, ¹⁷⁷Lu may be provided in HCl. [¹⁷⁷Lu]LuCl₃ may be provided in 0.05 M HCl. For example, 2 Ci/ml of ¹⁷⁷Lu may be provided in 0.05 M HCl. [¹⁷⁷Lu]LuCl₃ in 0.05 M hydrochloric acid may be transferred into the reactor and the [¹⁷⁷Lu]LuCl₃ vial may be rinsed with an additional required volume of 82 mg/mL sodium ascorbate (prepared in step 201) that is also then transferred into the reactor.

The reaction volume may range from 8 ml to 15 ml. The volume may be dependent on the amount of [¹⁷⁷Lu]LuCl₃ used in the radiolabeling reaction.

In an embodiment, step 204 may include radiolabeling the PSMA-I&T with ¹⁷⁷Lu. The reaction mixture may be heated up to about 70° C., up to about 75° C., up to about 80° C., up to about 85° C., up to about 90° C., or up to about 95° C. In an example, the setpoint for heating is 85° C. and the actual maximum temperature reached is about 75° C. The reaction volume may be heated for up to 5 minutes, up to 10 minutes, up to 15 minutes, up to 20 minutes, up to 25 minutes, up to 30 minutes, up to 35 minutes, up to 40 minutes, or up to 45 minutes. In at least one example, the reaction mixture is heated at setpoint of 80° C. for 10 minutes. In at least one additional example, the reaction mixture is heated at a setpoint of 70° C. for 10 minutes.

Presumably the stability enhancing conditions, such as ascorbic acid solution at pH of about 5 or below, should preferably be applied as early as possible in the process. For example, ascorbic acid solutions at pH 5 or below may be used instead of water at step 205 to minimize radiolytic damage.

The final composition may be formulated as a solution suitable for injection. The product is diluted to a standard radioactivity concentration, and therefore, the final volume of the bulk composition varies depending on the starting radioactivity of ¹⁷⁷Lu introduced.

In some embodiments, at step 206 the final composition may be sterile filtered. The sterile filter may be a 0.22 μm sterile filter. The final product may be dispensed through a 0.22 μm sterile filter into single dose vials containing suitable volume and radioactivity referenced to the prescribed calibration time. For example, the final composition may be dispensed through a 0.22 μm sterile filter under a Class A environment into doses containing suitable volume and radioactivity at a calibration time.

FIG. 3B provides one example of the process of making a radiopharmaceutical composition without purification and without ethanol.

Provided herein are methods for increasing the shelf life of a radiopharmaceutical product comprising ¹⁷⁷Lu-PSMA I&T. The methods may include adjusting the pH of the composition to 4.0, 4.25, 4.5, or 4.75 by adjusting the amount of ascorbic acid in the composition, and/or adjusting the radioactivity to increase the shelf life of the composition by 0.25, 0.5, 0.75, 1, 1.25, 1.5, 2, 2.25, 2.5, 2.75, or 3 days. For example, the radiopharmaceutical composition may have a shelf life of 1, 1.5, 2, 2.25, 2.5, 2.75, 3, 3.25, 3.5, 3.75, or 4 days. In an embodiment, adjusting the pH, radioactivity, and/or ascorbic acid may increase the radiochemical purity of the composition to at least 99%, at least 98.5%, at least 98%, at least 97.5%, at least 97%, at least 96.5%, at least 96%, at least 95.5%, or at least 95% for up to 1, 1.5, 2, 2.25, 2.5, 2.75, 3, 3.25, 3.5, 3.75, 4 days.

### V. Stability

A stable non-radioactive labelled standard may be used for identifying the product peak in the HPLC analysis. The formulation may be prepared from an injection grade solution containing ascorbic acid, chelating agent (EDTA, DTPA, or combination thereof), optionally dehydrated ethanol, and injections grade water. The formulation matrix may be prepared ex tempore as part of the synthesis preparation and a predetermined amount is added to the bulk vial as part of the synthesis preparations.

Without being limited to any one theory, the radioactivity, the amount of ascorbic acid, and/or the pH of the solution may have an impact on the shelf life of the composition. Surprisingly, a lower concentration of ascorbic acid (e.g. 31 mg/ml vs. 42.5 mg/ml) in the composition, a pH 4.5 or lower, a low RAC, and/or the combinations thereof may result in a higher stability profile and a longer shelf life for the composition as compared to compositions with a pH 5 or higher, a high RAC, and/or combinations thereof. For example, this may be seen in FIG. 5. The shelf life may generally be determined based on the radiochemical purity of the composition after formulation or at the time of expiration of the composition. The radiochemical purity may be confirmed by HPLC.

In one or more embodiments, ¹⁷⁷Lu-PSMA I&T formulation compositions with 31 mg/ml of ascorbic acid and pH of about 4.5 in a dose formulation at radioactivity concentration of 640 MBq/ml or below, can provide adequate stability of four days.

In an embodiment the composition has a low radioactivity concentration (e.g., 588.5 MBq/ml), a pH of 4.5, and 31 mg/ml ascorbic acid has 99.1% radiochemical purity 0 hours post EOS, 98.7% radiochemical purity 20 hours post EOS, 98.0% radiochemical purity 44 hours post EOS, 97.4% radiochemical purity 69 hours post EOS, and 97.0% radiochemical purity 93 hours post EOS. A composition having a low radioactivity concentration (e.g., 626 MBq/ml), a pH of 5.0, and 31 mg/ml ascorbic acid has 99.2% radiochemical purity 0 ours post EOS, 98.4% radiochemical purity 25 hours post EOS, 97.3% radiochemical purity 47 hours post EOS, and 96.5% radiochemical purity 71 hours post EOS. A composition having a low radioactivity concentration (e.g. 579 MBq/ml), a pH of 4.5, and 21 mg/ml ascorbic acid has 99.4% radiochemical purity 0 ours post EOS, 98.3% radiochemical purity 19 hours post EOS, 97.5% radiochemical purity 46 hours post EOS, 96.8% radiochemical purity 71 hours post EOS, and 96.0% radiochemical purity 92 hours post EOS. A composition having a high radioactivity concentration (e.g., 1,278 MBq/ml), a pH of 4.5, and 42.5 mg/ml ascorbic acid has 99.4% radiochemical purity 0 hours post EOS, 98.0% radiochemical purity 24 hours post EOS, 96.7% radiochemical purity 46 hours post EOS, 95.3% radiochemical purity 67 hours post EOS, and 95.2% radiochemical purity 71 hours post EOS.

The radiopharmaceutical composition may be stored at temperatures ranging from 2° C. to 40° C., about 2° C. to 5° C., about 5° C. to 10° C., about 10° C. to 15° C., about 15° C. to 20° C., about 20° C. to 25° C., about 25° C. to 30° C., about 30° C. to 35° C., or about 35° C. to 40° C.

In an embodiment, the radiopharmaceutical composition is stored at a temperature from about 5° C. to 40° C., about 10° C. to 35° C. or about 20° C. to 30° C. In one specific embodiment, the radiopharmaceutical composition is stored at a temperature at about 10° C., about 15° C., about 22° C., about 22.5° C., about 25° C., or at room temperature.

In one embodiment, the radiopharmaceutical composition is stored at about 22.5° C. In another embodiment, the radiopharmaceutical composition is stored at room temperature.

### VI. Specific Radiopharmaceutical Composition

In some embodiments, the medicinal product is a sterile filtered radiopharmaceutical solution containing a micro dose of [¹⁷⁷Lu]Lu-PSMA I&T solution in a 42.5 mg/ml aqueous ascorbic acid solution containing 7.5% (v/v) or 59 mg/ml ethanol. The product is diluted to a standard radioactivity concentration and therefore the final volume of the bulk product varies depending on the starting radioactivity introduced. The composition of the final product described in Table 1B (¹⁷⁷Lu-PSMA I&T composition 1):

TABLE 1B
Composition of Final Product^{a} (¹⁷⁷Lu-PSMA I&T composition 1)

| Component | Quantity | | Function |
|---|---|---|---|
| ¹⁷⁷Lu-PSMA I&T | q.s.^{b} | | API |
| PSMA I&T | 5-12 | μg/ml | Precursor |
| Ethanol | 7.5% | (v/v) | Stabilizing agent |
| | | | (radiolysis) |
| Ascorbic acid | 42.5 | mg | Stabilizing agent |
| | | | (radiolysis) |
| Disodium EDTA | 21 | μg | Metal scavenger |

| | | |
|---|---|---|
| Sodium bicarbonate* | q.s. | pH adjuster |
| Sodium hydroxide* | q.s. | pH adjuster |
| WFI (injections grade water) | ad 1 ml | Vehicle |

^{a}Max volume per vial is 10 ml
^{b}sufficient amount of radioactivity for intended use

In yet another embodiment, the medicinal product is a sterile filtered radiopharmaceutical solution containing a micro dose of ¹⁷⁷Lu-PSMA I&T solution in 31 mg/ml aqueous ascorbic acid solution containing 3.8% (v/v) or 30 mg/ml ethanol, at pH of about 4.5. The product is diluted to a standard radioactivity concentration, and therefore, the final volume of the bulk product varies depending on the starting radioactivity introduced. The composition is described below in Table 1C (¹⁷⁷Lu-PSMA I&T composition 2):

TABLE 1C
Composition of Final Product*
([¹⁷⁷Lu]Lu-PSMA I&T Composition 2)

| Component | Quantity | | Function |
|---|---|---|---|
| ¹⁷⁷Lu-PSMA I&T | q.s.** | | API |
| PSMA I&T | 45-120 | μg | |
| Ethanol | 3.8% | (v/v) | Stabilizing agent |
| | | | (radiolysis) |
| Ascorbic acid | 31 | mg/ml | Stabilizing agent |
| | | | (radiolysis) |
| Disodium EDTA | 15.5 | μg | Metal scavenger |

| | | |
|---|---|---|
| Sodium bicarbonate | q.s | pH adjuster |
| Sodium hydroxide | q.s | pH adjuster |
| Hydrochloric acid | q.s | pH adjuster |
| WFI (injections grade water) | ad 1 ml | Vehicle |

*Max volume per vial is 20 ml
**sufficient amount of radioactivity for intended use

In yet another embodiment, the medicinal product is a radiopharmaceutical solution containing a small amount of [¹⁷⁷Lu]Lu-PSMA I&T solution in 28-38 mg/ml aqueous ascorbic acid solution, at pH of about 4.25. The product is diluted to a standard radioactivity concentration, and therefore, the final volume of the bulk product varies depending on the starting radioactivity introduced. The composition is described below in Table 1D (¹⁷⁷Lu-PSMA I&T composition 3):

TABLE 1D
Composition of Final Product* (¹⁷⁷Lu-PSMA I&T composition 3)

| Component | Quantity | | Function |
|---|---|---|---|
| ¹⁷⁷Lu-PSMA I&T | q.s.** | | API |
| PSMA I&T | 45-120 | μg | |
| Ascorbic acid | 420-570 | mg | Stabilizing agent |
| | | | (radiolysis) |
| DTPA | 0.075-0.15 | mg/ml | Metal ion chelator |

| | | |
|---|---|---|
| WFI (injections grade water) | ad 1 ml | Vehicle |

*Max volume per vial is 15 ml
**sufficient amount of radioactivity for intended use

### VII. Formulating the Drug Product

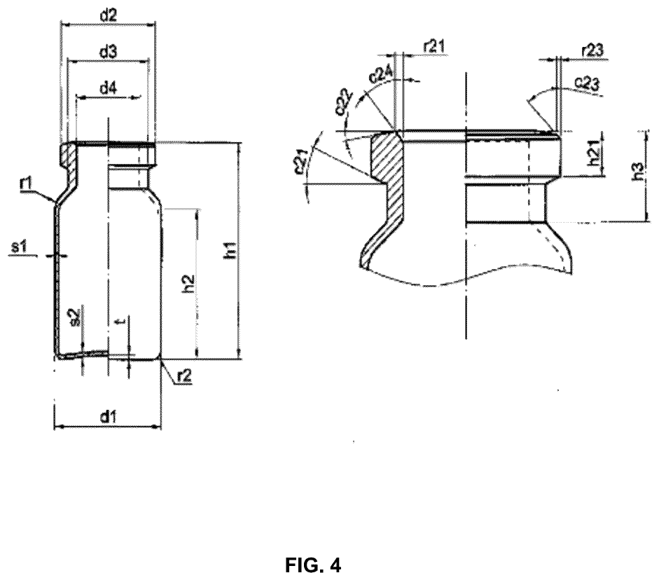

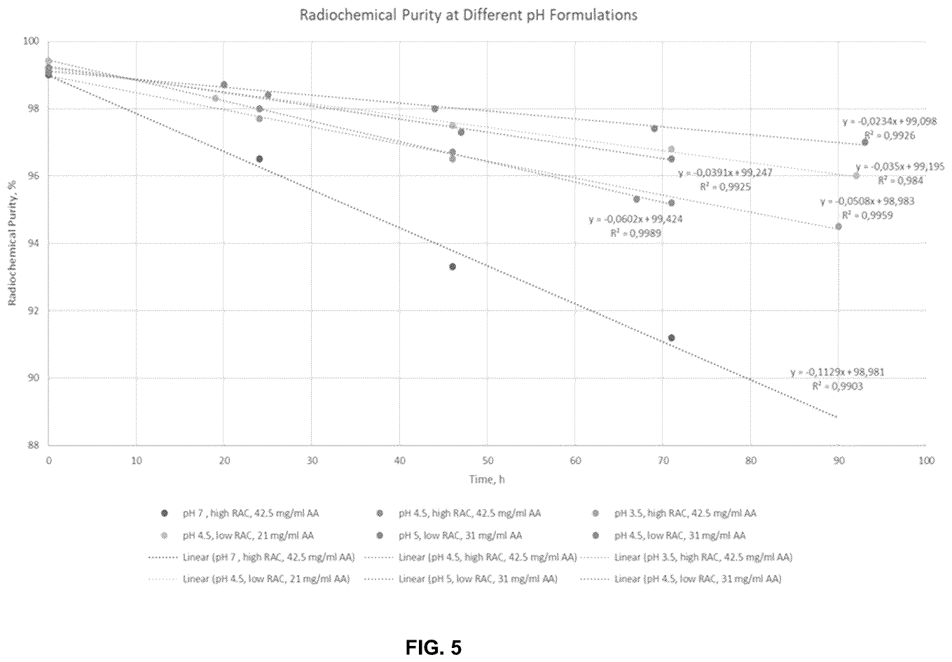

The drug product may be delivered in a sterile pyrogen free glass vial of Type 1 glass with a fluorocoated bromobutyl rubber septum. The septum is sealed with a crimped aluminum seal. During transportation, the glass vial containing the radiopharmaceutical is kept in a lead shielded container. The transport container including lead shield and outer packaging complies to type A requirements (IAEA standards). FIG. 4 depicts a drawing of a product vial that can be used in this embodiment.

In one embodiment, the volume of the solution comprising the formulation or radiopharmaceutical composition is from about 10 ml to about 20 ml, from about 20 ml to about 30 ml, from about 30 ml to about 40 ml, from about 40 ml to about 50 ml, from about 50 ml to about 60 ml, from about 60 ml to about 70 ml, from about 70 ml to about 80 ml, from about 80 ml to about 90 ml, or from about 90 ml to about 100 ml. In one specific embodiment, the volume of the solution comprising the formulation or radiopharmaceutical composition is about 1 ml, about 5 ml, about 7 ml, about 8 ml, about 9 ml, about 10 ml, about 15 ml, about 20 ml, about 25 ml, or about 30 ml. In one embodiment, the volume of the solution comprising the formulation or radiopharmaceutical composition is from about 11 ml, about 12 ml, about 13 ml, about 14 ml, about 15 ml, about 16 ml, about 17 ml, about 18 ml, about 19 ml, about 20 ml, about 25 ml, or about 30 ml.

In one embodiment, the volume of the solution comprising the formulation or radiopharmaceutical composition is from about 100 ml to about 200 ml, from about 200 ml to about 300 ml, from about 300 ml to about 400 ml, from about 400 ml to about 500 ml, from about 500 ml to about 600 ml, from about 600 ml to about 700 ml, from about 700 ml to about 800 ml, from about 800 ml to about 900 ml, or from about 900 ml to about 1000 ml. In one specific embodiment, the volume of the solution comprising the formulation or radiopharmaceutical composition is about 200 ml, about 225 ml, about 250 ml, about 275 ml, about 300 ml, about 325 ml, about 350 ml, about 375 ml, about 400 ml, about 425 ml, about 450 ml, about 475 ml, about 500 ml, about 525 ml, about 550 ml, about 575 ml, about 600 ml, about 625 ml, about 650 ml, about 675 ml, about 700 ml, about 725 ml or about 750 ml.

In one specific embodiment, the final volume in the dose vial is adjusted to between 7 ml and 10 ml, between 10 ml and 15 ml, or between 15 ml and 20 ml in order to provide the required amount of radioactivity at the date and time of infusion.

In another embodiment, ¹⁷⁷Lu-PSMA I&T injection is supplied as a single-dose vial or multi-dose vial. For example, provided herein is a radiopharmaceutical kit including a vial comprising a single dose of the ¹⁷⁷Lu-PSMA I&T injection product composition. In one embodiment, the strength of the ¹⁷⁷Lu-PSMA I&T injection product composition is about 0.1 GBq/ml, about 0.2 GBq/ml, about 0.3 GBq/ml, about 0.4 GBq/ml, about 0.5 GBq/ml, about 0.6 GBq/ml, about 0.7 GBq/ml, about 0.8 GBq/ml, about 0.9 GBq/ml, about 1.0 GBq/ml, about 1.1 GBq/ml, about 1.2 GBq/ml, about 1.3 GBq/ml, about 1.4 GBq/ml, about 1.5 GBq/ml, about 1.6 GBq/ml, about 1.7 GBq/ml, about 1.8 GBq/ml, about 1.9 GBq/ml, or about 2.0 GBq/ml. In another embodiment, the strength of the ¹⁷⁷Lu-PSMA I&T injection product composition is less than about 2.0 GBq/ml, less than about 1.5 GBq/ml, less than about 1.0 GBq/ml, or less than about 0.5 GBq/ml.

In yet another embodiment, the shelf life of the [¹⁷⁷Lu]Lu-PSMA I&T injection product composition is from about 30 hours to about 90 hours, from about 40 hours to about 80 hours, or from about 48 hours to about 72 hours. In one specific embodiment, the shelf life of the [¹⁷⁷Lu]Lu-PSMA I&T injection product composition is about 30 hours, about 35 hours, about 40 hours, about 45 hours, about 48 hours, about 50 hours, about 55 hours, about 60 hours, about 65 hours, about 70 hours, about 72 hours, about 75 hours, about 80 hours, about 85 hours, or about 90 hours.

In some embodiments, the radiopharmaceutical composition should have a radiochemical purity of ≥95% for [¹⁷⁷Lu]Lu-PSMA I&T to be sufficient for administration to a patient. The combined radiochemical impurities in the composition may be ≤5%. In various embodiments, the radiopharmaceutical composition may have a chemical purity such that Lu-PSMA I&T is present in the composition in a concentration of less than about 12 μg/ml, less than about 11 μg/ml, less than about 10 μg/ml, less than about 9 μg/ml, less than about 8 μg/ml, less than about 7 μg/ml, less than about 6 μg/ml, less than about 5 μg/ml, less than about 4 μg/ml, less than about 3 μg/ml, less than about 2 μg/ml, less than about 1.75 μg/ml, less than about 1.5 μg/ml, or less than about 1 μg/ml.

In some embodiments, the radiopharmaceutical composition may have an amount of colloidal ¹⁷⁷Lu of less than about 5% of radioactivity, less than about 4.5% of radioactivity, less than about 4% of radioactivity, less than about 3.5% of radioactivity, less than about 3% of radioactivity, less than about 2.5% of radioactivity, less than about 2% of radioactivity, less than about 1.5% of radioactivity, less than about 1% of radioactivity, less than about 0.5% of radioactivity, less than about 0.3% of radioactivity, less than about 0.2% of radioactivity, or less than about 0.1% of radioactivity In one embodiment, the radiopharmaceutical composition administered to the human patient in need thereof comprises less than 0.5%, less than 0.4%, less than 0.3%, less than 0.2%, or less than 0.1% colloidal ¹⁷⁷Lu.

In some embodiments, the radiopharmaceutical composition may have less than about 17.5 EU/ml, less than about 17 EU/ml, less than about 16.5 EU/ml, less than about 16 EU/ml, less than about 15.5 EU/ml, less than about 15 EU/ml, less than about 14.5 EU/ml, less than about 14 EU/ml less than about 13.5 EU/ml, less than about 13 EU/ml, less than about 12.5 EU/ml, less than about 12 EU/ml, less than about 11.5 EU/ml, less than about 11 EU/ml, less than about 10.5 EU/ml, less than about 10 EU/ml, less than about 9.5 EU/ml, less than about 9 EU/ml, less than about 8.5 EU/ml, less than about 8 EU/ml, less than about 7.5 EU/ml, less than about 7 EU/ml, less than about 6.5 EU/ml, less than about 6 EU/ml, less than about 5.5 EU/ml, less than about 5 EU/ml, less than about 4.5 EU/ml, less than about 4 EU/ml, less than about 3.5 EU/ml, less than about 3 EU/ml, less than about 2.5 EU/ml, less than about 2 EU/ml, less than about 1.5 EU/ml, less than about 1 EU/ml, less than about 0.5 EU/ml, or no bacterial endotoxins.

In an embodiment, the radiochemical purity of the composition is ≥95% at 1 day, up to 2 days, up to 3 days, up to 4 days, or up to 5 days after formulation. In additional embodiments, the radiochemical purity of the composition is ≥95% at 24 hours, up to 36 hours, up to 48 hours, up to 72 hours, or up to 96 hours after formulation. In further embodiments, the radiochemical purity of the composition is suitable for injection and suitable for administration to a patient in need thereof more than 72 hours after formulation, more than 96 hours after formulation, or more than 100 hours after formulation. The radiopharmaceutical composition may have a radiochemical purity of at least 95%, at least 95.5%, at least 96%, at least 96.5%, at least 97%, at least 97.5%, at least 98%, at least 98.5%, or at least 99% at 24 hours, 48 hours, 72 hours, and/or 96 hours after formulation. In some examples, the radiopharmaceutical composition may have a radiochemical purity of 95.0% or greater, 95.5% or greater, 96.0% or greater, 96.5% or greater, 97.0% or greater, 97.5% or greater, 98.0% or greater, 98.5% or greater, 99.0% or greater, or 99.5% or greater at administration. For example, the radiopharmaceutical composition may have a radiochemical purity of more than 95% at 46 to 48 hours after formulation, a radiochemical purity of more than 96% at 46 to 48 hours after formulation, a radiochemical purity of more than 97% at 46 to 48 hours after formulation, a radiochemical purity of more than 95% at 69 to 72 hours after formulation, a radiochemical purity of more than 96% at 69 to 72 hours after formulation, a radiochemical purity of more than 97% at 69 to 72 hours after formulation, a radiochemical purity of more than 95% at 90 to 93 hours after formulation, a radiochemical purity of more than 96% at 90 to 93 hours after formulation, and/or a radiochemical purity of more than 97% at 90 to 93 hours after formulation.

In some examples, the radiochemical purity of the composition may range from about 99.0% to about 99.4% 0 hours post EOS. In various embodiments, the radiochemical purity of the composition may range from about 96.5% to about 98.7% 19-25 hours post EOS. In other examples, the radiochemical purity of the composition may range from about 93.3% to about 98.0% 44-47 hours post EOS. In additional examples, the radiochemical purity of the composition may range from about 91.2% to about 97.4% 69-71 hours post EOS. In some examples, the radiochemical purity of the composition may range from about 95% to about 97.0% 90-93 hours post EOS.

In another embodiment, [¹⁷⁷Lu]Lu-PSMA I&T injection is supplied as a single-dose vial or multi-dose vial.

In yet another embodiment, a patient in need of radioligand therapy during a treatment receives a single intravenous radioactive dose at the beginning of a treatment cycle. The treatment cycle is from 1 to 10 weeks. In one embodiment, the treatment includes 1 to 6 treatment cycles. In another embodiment, a dose reduction or a dose increase is introduced during treatment.

In one embodiment, the volume of the patient dose is calculated depending on the radioactive dose to be administered.

In another embodiment, [¹⁷⁷Lu]Lu-PSMA I&T is infused by intravenous (IV) route slowly, over about 10 minutes, and followed by infusion of 500-1000 mL of Ringer's or normal saline solution. An extra 7 mL injection when the total blood volume is over 5,000 mL is of no consequence. The dose is administered every 6 weeks for 4 cycles.

In yet another embodiment, a patient in need of radioligand therapy during a treatment receives a single intravenous radioactive dose at the beginning of a treatment cycle. The treatment cycle is from 1 to 10 weeks. In one embodiment, the treatment includes 1-6 treatment cycles. In another embodiment, a dose reduction or a dose increase is introduced during treatment.

In one embodiment, the volume of the patient dose is calculated depending on the radioactive dose to be administered.

### VIII. Method of Diagnosing or Treatment of Prostate Cancer

Provided herein are methods of diagnosing or treating a tumor of a patient in need thereof. The method may include administering by injection a radiopharmaceutical composition comprising ¹⁷⁷Lu-PSMA I&T and ascorbic acid at a pH of 3.5 to 4.5 in solution.

In some embodiments, the mean absorbed dose of the radiopharmaceutical composition may be about 0.1 mGy/MBq to 0.5 mGy/MBq, about 0.5 mGy/MBq to 1.0 mGy/MBq, 0.5 mGy/MBq to 0.6 mGy/MBq, 0.6 mGy/MBq to 0.7 mGy/MBq, 0.7 mGy/MBq to 0.8 mGy/MBq, 0.8 mGy/MBq to 0.9 mGy/MBq, 0.9 mGy/MBq to 1.0 mGy/MBq, 1 mGy/MBq to 1.5 mGy/MBq, 1.0 mGy/MBq to 1.1 mGy/MBq, 1.1 mGy/MBq to 1.2 mGy/MBq, 1.2 mGy/MBq to 1.3 mGy/MBq, 1.3 mGy/MBq to 1.4 mGy/MBq, or 1.4 mGy/MBq to 1.5 mGy/MBq, 1.5 mGy/MBq to 2.5 mGy/MBq, 2.5 mGy/MBq to 3.5 mGy/MBq, 2.5 mGy/MBq to 2.7 mGy/MBq, 2.7 mGy/MBq to 2.9 mGy/MBq, 2.9 mGy/MBq to 3.1 mGy/MBq, 3.1 mGy/MBq to 3.3 mGy/MBq, 3.3 mGy/MBq to 3.5 mGy/MBq, or 3.5 mGy/MBq to 4.5 mGy/MBq in the kidneys of the patient. In some embodiments, the mean absorbed dose of the radiopharmaceutical composition may be about 1 mGy/MBq to 1.5 mGy/MBq, 1.0 mGy/MBq to 1.1 mGy/MBq, 1.1 mGy/MBq to 1.2 mGy/MBq, 1.2 mGy/MBq to 1.3 mGy/MBq, 1.3 mGy/MBq to 1.4 mGy/MBq, or 1.4 mGy/MBq to 1.5 mGy/MBq in the parotid glands of the patient. In some embodiments, the mean absorbed dose of the radiopharmaceutical composition may be about 2.5 mGy/MBq to 3.5 mGy/MBq, 2.5 mGy/MBq to 2.7 mGy/MBq, 2.7 mGy/MBq to 2.9 mGy/MBq, 2.9 mGy/MBq to 3.1 mGy/MBq, 3.1 mGy/MBq to 3.3 mGy/MBq, or 3.3 mGy/MBq to 3.5 mGy/MBq in bone lesions of the patient. In some embodiments, the mean absorbed dose of the radiopharmaceutical composition may be about 3.5 mGy/MBq to 4.5 mGy/MBq, 3.5 mGy/MBq to 3.7 mGy/MBq, 3.7 mGy/MBq to 3.9 mGy/MBq, 3.9 mGy/MBq to 4.1 mGy/MBq, 4.1 mGy/MBq to 4.3 mGy/MBq, or 4.3 mGy/MBq to 4.5 mGy/MBq in lymph node lesions of the patient.

Treatment aimed at eradicating the primary tumor, typically with surgery or radiation, is unsuccessful in ˜30% of men, who develop recurrent disease that usually manifests first as a rise in plasma prostate-specific antigen (PSA) followed by metastasis to distant sites (Stephenson et al. J Clin Oncol, 2005; 23:8253-61). Given that prostate cancer cells depend on androgen receptors (AR) for their proliferation and survival, the standard treatment for patients with recurrent disease is androgen deprivation therapy (ADT) with a gonadotropin releasing hormone analog (GnRHa)) with or without an anti-androgen.

Treatment results with ADT are generally predictable: a decline in PSA followed by tumor regression, a period of stability in which the tumor does not proliferate and stable PSA, followed by rising PSA and regrowth defined as a castration-resistant disease. Nearly all men with progressive prostate cancer eventually develop castration-resistant disease. Prostate cancer progression despite castrate levels of testosterone represents a transition to a lethal disease stage. Docetaxel with prednisone, cabazitaxel with prednisone, enzalutamide, and abiraterone with prednisone have become the Standard of Care based on the National Comprehensive Cancer Network (NCCN) guidelines (Mohler et al., NCCN Clinical practice guidelines in oncology. Prostate Cancer, (Version 2.2019). JNCCN.org; 17(5),479-505) in men with metastatic castration-resistant prostate cancer (mCRPC).

Abiraterone, enzalutamide, and docetaxel with prednisone are all indicated for patients with mCRPC as first line of treatment, while cabazitaxel with prednisone is indicated only for mCRPC patients who progressed on docetaxel. George et al. reported the sequencing of treatments for mCRPC patients in a real-world clinical setting in the United States (George et al. 2020). A higher proportion of patients in the US receive androgen receptor axis-targeted therapy (ARAT, namely abiraterone and enzalutamide) as a first line treatment versus docetaxel; similarly, a higher proportion of mCRPC patients receive the alternative ARAT as second line of treatment (enzalutamide after abiraterone, or vice versa).

Targeted radionuclide therapy is an emerging therapy option for many different cancers, including lymphoma, melanoma, and neuroendocrine tumors (Kraeber-Bodéré et al., Semin Oncol, 2014, 41, 613-22; Mier et al., J Nucl Med, 2014, 55, 9-14; Bodei et al., Eur J Nucl Med Mol Imaging, 2015, 42, 5-19). Prostate-specific membrane antigen (PSMA) is a key target for radionuclide diagnosis and therapy for PC. PSMA is normally expressed in prostate cells as well as some extra prostatic tissue but its overexpression in prostate cancer cells makes it an attractive target for therapeutic agents with the potential to limit systemic toxicity (Silver et al, Clin Cancer Res. 1997 January; 3(1):81-5. PMID: 9815541). Initial clinical experience with PSMA-based radionuclide treatment of PC using ¹³¹I-labeled PSMA showed promising results with a PSA decrease >50% in 60% of all treated prostate cancer patients and with mild hematotoxicity (Zechmann et al., Eur J Nucl Med Mol Imaging, 2014, 41, 1280-92).

### IX. Administration

Further provided herein are methods of administering the radiopharmaceutical composition. The radiopharmaceutical composition may be administered by injection or infusion to a human patient in need thereof.

There can be about six main aspects of administration.

First, cooling the salivary glands, the patients receive ice packs over the parotid and submandibular glands from 30 min prior to and up to 4 hours after administration of ¹⁷⁷Lu-PSMA I&T to reduce the risk of salivary gland radiation injuries. There is no scientific evidence of whether cooling the salivary glands is an effective therapy for saving these glands from radiation; however, it is tolerable and not harmful for the patients.

Second, using a urinary catheter in incontinent patients in the first 48 hours to avoid any contamination.

Third, activity of 6.5-7.5 GBq (range: 6.0-8.0 GBq)¹⁷⁷Lu-PSMA I&T. The amount of activity can be reduced to 4.0-5.0 GBq in the case of impaired renal function (e.g., Creatinine within 1.0-1.5 UNL). According to the preliminary results, an activity of 7.4 GBq can be administered safely; however, more data are required to increase the amount of activity.

Fourth, infusion of the activity intravenously as a slow bolus (over about 1-15 minutes) followed by 500-1000 ml Ringer or NaCl solution. The patients should be encouraged to void as frequently as possible and drink about 2 liters of water daily. In patients with dilated non-obstructive renal disease an administration of diuretics may be meaningful.

Fifth, in average 3-5 cycles of the RLT every 5-8 weeks, experience up to 11 cycles has been reported. In the case of continuously increasing PSA, after the first two cycles accompanied by worsening of the general condition, the indication of further RLTs should be re-evaluated. In case of a decreasing PSA to <1.0 μg/l during the therapy cycles, a PSMA imaging could evaluate existence of small PSMA-positive metastases after completion of RLT when post injection SPECT study is not enough informative. In case of a significant decline of platelets or leukocytes, the time interval between the 2 cycles can be prolonged.

Sixth, at least one whole body scan 24-48 hours post injection (preferably with SPECT(/CT). In patients with diffuse bone and bone marrow metastases as well as in patients with brain metastases a concomitant corticosteroid therapy (e.g., prednisolone 20 mg/daily) in the first two weeks after administration is advisable.

In some embodiments, the method may include injecting the radiopharmaceutical composition into a patient in need thereof more than 48 hours after formulation. In some examples, the radiopharmaceutical composition may include ¹⁷⁷Lu-PSMA I&T and ascorbic acid in a solution having a pH of 3.5 to 4.5, and the solution may have a radiochemical purity of more than 96% when administered. In an embodiment, the pH of the solution is about 3.5 to 4.2. The composition may include <6 μg/ml of Lu-PSMA I&T, about 7 μg/ml to about 18 μg/ml disodium EDTA, about 25 μl/ml to about 45 μl/ml ethanol, and/or about 15 to about 35 mg/ml ascorbic acid. The composition may have a radioactivity of about 0.5 GBq/ml or about 13.5 mCi/ml and have a radiochemical purity of at least 98% at 44 hours after formulation, at least 97% at 69 hours after formulation, and/or at least 97% at 93 hours after formulation.

The pharmaceutical composition may be administered as 2-11 cycles/treatments every 5-8 weeks. In some embodiments, the patient may be administered up to 1, 2, 3, 4, 5, 6, 7, 8, 9, 10, or 11 treatments and the treatments may be administered every 4, 5, 6, 7, or 8 weeks. In an example, the patient may be administered up to 4 treatments, with each treatment administered every 6 weeks.

In various embodiments, the patient may be administered ¹⁷⁷Lu-PSMA I&T at a dose of 0.5 GBq to 10 GBq per dose cycle. For example, the radiopharmaceutical composition may contain a standard radioactivity of about 200 mCi at the time of administration with a standard radioactivity concentration of about 27 mCi/mL at end of production; therefore, the final volume of the dose vial may be adjusted to between 7 and 15 mL in order to provide the required amount of radioactivity at the date and time of infusion. In some embodiments, the dose vial contains between 10 to 20 mL. In at least one example, the patient may be administered a dose of about 200 mCi (7.4 GBq±0.1 GBq) for each treatment. In one aspect, the patient may be administered a dose of about 200 mCi (7.4 GBq±0.1 GBq) for each treatment for four, five, six, seven, or more treatments. In another aspect, the patient may be administered a dose of about 200 mCi (7.4 GBq±0.1 GBq) for each treatment for four or more treatments, five or more, six or more, seven or more, or eight or more treatments. In yet another aspect, the patient may be administered a dose of about 200 mCi (≥7.1 GBq) for each treatment for four, five, six, seven, eight, or more treatments. In another example, the patient may be administered a dose of about 6.8 GBq±0.3 GBq for each treatment. In one aspect, the patient may be administered a dose of about 6.8 GBq±0.3 GBq for each treatment for four, five, six, seven, eight or more treatments.

In various embodiments, the patient may be administered ¹⁷⁷Lu-PSMA I&T at a dose of about 0.5 GBq to about 10 GBq, about 1.0 GBq to about 9.0 GBq, about 1.5 GBq to about 8.5 GBq, about 2.0 GBq to about 8.0 GBq, about 2.5 GBq to about 7.5 GBq, or about 3.0 GBq to about 7.0 GBq, wherein the total cumulative dose to the patient's kidneys per each administration is ≤3.9 Gy, ≤3.8 Gy, ≤3.7 Gy, ≤3.6 Gy, ≤3.5 Gy, ≤3.4 Gy, ≤3.3 Gy, ≤3.2 Gy, ≤3.1 Gy, ≤3.0 Gy, ≤2.9 Gy, ≤2.8 Gy, ≤2.7 Gy, ≤2.6 Gy, ≤2.5 Gy, or ≤2.4 Gy, and wherein the patient may be administered a dose for each treatment for one, two, three, four, five, six, seven, eight, or more treatments (i.e., cycles of treatment). for four, five, six, seven, eight, or more treatments (i.e., cycles of treatment). In various embodiments, the patient may be administered ¹⁷⁷Lu-PSMA I&T at a dose of about 0.5 GBq to about 10.0 GBq, about 0.5 GBq to about 9.5 GBq, about 0.5 GBq to about 9.0 GBq, about 0.5 GBq to about 8.5 GBq, about 0.5 GBq to about 8.0 GBq, about 0.5 GBq to about 7.5 GBq, about 1.0 GBq to about 10.0 GBq, about 1.0 GBq to about 9.5 GBq, about 1.0 GBq to about 9.0 GBq, about 1.0 GBq to about 8.5 GBq, about 1.0 GBq to about 1.0 GBq, about 1.0 GBq to about 7.5 GBq, about 1.5 GBq to about 10.0 GBq, about 1.5 GBq to about 9.5 GBq, about 1.5 GBq to about 9.0 GBq, about 1.5 GBq to about 8.5 GBq, about 1.5 GBq to about 8.0 GBq, about 1.5 GBq to about 7.5 GBq, about 2.0 GBq to about 10.0 GBq, about 2.0 GBq to about 9.5 GBq, about 2.0 GBq to about 9.0 GBq, about 2.0 GBq to about 8.5 GBq, about 2.0 GBq to about 8.0 GBq, about 2.5 GBq to about 10.0 GBq, about 2.5 GBq to about 2.5 GBq, about 2.5 GBq to about 9.0 GBq, about 2.5 GBq to about 8.5 GBq, about 2.5 GBq to about 8.0 GBq, about 2.5 GBq to about 7.5 GBq, about 3.0 GBq to about 10.0 GBq, about 3.0 GBq to about 9.5 GBq, about 3.0 GBq to about 9.0 GBq, about 3.0 GBq to about 8.5 GBq, about 3.0 GBq to about 8.0 GBq, about 3.0 GBq to about 7.5 GBq, about 3.5 GBq to about 10.0 GBq, about 3.5 GBq to about 9.5 GBq, about 3.5 GBq to about 9.0 GBq, about 3.5 GBq to about 8.5 GBq, about 3.5 GBq to about 8.0 GBq, about 3.5 GBq to about 7.5 GBq, about 0.5 GBq to about 7.5 GBq, about 0.5 GBq to about 7.4 GBq, about 1.0 GBq to about 7.4 GBq, about 1.5 GBq to about 7.4 GBq, about 2.0 GBq to about 7.4 GBq, about 2.5 GBq to about 7.4 GBq, about 3.0 GBq to about 7.4 GBq, about 3.5 GBq to about 7.4 GBq, about 4.0 GBq to about 7.4 GBq, about 4.5 GBq to about 7.4 GBq, about 5.0 GBq to about 7.4 GBq, about 5.5 GBq to about 7.4 GBq, about 6.0 GBq to about 7.4 GBq, about 6.5 GBq to about 7.4 GBq, about 6.6 GBq to about 7.4 GBq, about 6.7 GBq to about 7.4 GBq, about 6.8 GBq to about 7.4 GBq, about 6.9 GBq to about 7.4 GBq, about 7.0 GBq to about 7.4 GBq, about 7.1 GBq to about 7.4 GBq, about 7.2 GBq to about 7.4 GBq, or about 7.3 GBq to about 7.4 GBq, wherein the total cumulative dose to the patient's kidneys per each administration is ≤3.9 Gy, ≤3.8 Gy, ≤3.7 Gy, ≤3.6 Gy, ≤3.5 Gy, ≤3.4 Gy, ≤3.3 Gy, ≤3.2 Gy, ≤3.1 Gy, ≤3.0 Gy, ≤2.9 Gy, ≤2.8 Gy, ≤2.7 Gy, ≤2.6 Gy, ≤2.5 Gy, or ≤2.4 Gy, and wherein the patient may be administered a dose for each treatment for one, two, three, four, five, six, seven, eight, or more treatments (i.e., cycles of treatment). In other various embodiments, the patient may be administered ¹⁷⁷Lu-PSMA I&T at a dose of about 0.5 GBq to about 6.8 GBq, about 1.0 GBq to about 6.8 GBq, about 1.5 GBq to about 6.8 GBq, about 2.0 GBq to about 6.8 GBq, about 2.5 GBq to about 6.8 GBq, about 3.0 GBq to about 6.8 GBq, about 3.5 GBq to about 6.8 GBq, about 4.0 GBq to about 6.8 GBq, about 4.5 GBq to about 6.8 GBq, about 5.0 GBq to about 6.8 GBq, about 5.5 GBq to about 6.8 GBq, about 6.0 GBq to about 6.8 GBq, about 6.1 GBq to about 6.8 GBq, about 6.2 GBq to about 6.8 GBq, about 6.3 GBq to about 6.8 GBq, about 6.4 GBq to about 6.8 GBq, about 6.5 GBq to about 6.8 GBq, about 6.6 GBq to about 6.8 GBq, or about 6.7 GBq to about 7.4 GBq, wherein the total cumulative dose to the patient's kidneys per each administration is ≤3.9 Gy, ≤3.8 Gy, ≤3.7 Gy, ≤3.6 Gy, ≤3.5 Gy, ≤3.4 Gy, ≤3.3 Gy, ≤3.2 Gy, ≤3.1 Gy, ≤3.0 Gy, ≤2.9 Gy, ≤2.8 Gy, ≤2.7 Gy, ≤2.6 Gy, ≤2.5 Gy, or ≤2.4 Gy, and wherein the patient may be administered a dose for each treatment for one, two, three, four, five, six, seven, eight, or more treatments (i.e., cycles of treatment).

In various embodiments, the patient may be administered greater than 10 treatments, and the treatments may be administered every 4, 5, 6, 7, or 8 weeks, so long as the total cumulative dose to the patient's kidneys after all treatments remains below 23 grays (Gy). For example, the patient may be administered greater than 10, greater than 15, greater than 20, greater than 25, greater than 30, greater than 35, greater than 40, greater than 45, greater than 50, greater than 55, greater than 60, greater than 65, greater than 70, or greater than 75 treatments, and the treatments may be administered every 4, 5, 6, 7, or 8 weeks, so long as the total cumulative dose to the patient's kidneys after all treatments remains below 23 grays (Gy).

In some embodiments, the administration of the [¹⁷⁷Lu]Lu-PSMA I&T results in an absorbed dose to the patient's kidneys of 0.46 Gy/GBq±0.23 Gy/GBq (i.e., 0.46 Gy per GBq of the administered ¹⁷⁷Lu-PSMA I&T). In some additional embodiments, the administration of the ¹⁷⁷Lu-PSMA I&T results in an absorbed dose to the patient's kidneys of less than or equal to 0.46 Gy/GBq.

In various embodiments, the administration of the [¹⁷⁷Lu]Lu-PSMA I&T results in an absorbed dose to the patient's kidneys of 0.43 Gy/GBq±0.05 Gy/GBq (i.e., 0.43 Gy per GBq of the administered [¹⁷⁷Lu]Lu-PSMA I&T). In some additional embodiments, the administration of the [¹⁷⁷Lu]Lu-PSMA I&T results in an absorbed dose to the patient's kidneys of less than or equal to 0.43 Gy/GBq.

In other embodiments, the administration of the [¹⁷⁷Lu]Lu-PSMA I&T results in an absorbed dose to the patient's kidneys of 0.41 Gy/GBq±0.15 Gy/GBq (i.e., 0.41 Gy per GBq of the administered [¹⁷⁷Lu]Lu-PSMA I&T). In some additional embodiments, the administration of the [¹⁷⁷Lu]Lu-PSMA I&T results in an absorbed dose to the patient's kidneys of less than or equal to 0.41 Gy/GBq.

In various embodiments, the administration of the [¹⁷⁷Lu]Lu-PSMA I&T results in an absorbed dose to the patient's lacrimal glands of 0.67 Gy/GBq±0.33 Gy/GBq (i.e., 0.67 Gy per GBq of the administered [¹⁷⁷Lu]Lu-PSMA I&T). In some additional embodiments, the administration of the [¹⁷⁷Lu]Lu-PSMA I&T results in an absorbed dose to the patient's lacrimal glands of less than or equal to 0.67 Gy/GBq.

In other embodiments, the administration of the [¹⁷⁷Lu]Lu-PSMA I&T results in an absorbed dose to the patient's lacrimal glands of 0.40 Gy/GBq±0.37 Gy/GBq (i.e., 0.40 Gy per GBq of the administered [¹⁷⁷Lu]Lu-PSMA I&T). In some additional embodiments, the administration of the [¹⁷⁷Lu]Lu-PSMA I&T results in an absorbed dose to the patient's lacrimal glands of less than or equal to 0.40 Gy/GBq.

In other embodiments, the administration of the [¹⁷⁷Lu]Lu-PSMA I&T results in an absorbed dose to the patient's salivary glands of 0.10 Gy/GBq±0.06 Gy/GBq (i.e., 0.10 Gy per GBq of the administered [¹⁷⁷Lu]Lu-PSMA I&T). In some additional embodiments, the administration of the ¹⁷⁷Lu-PSMA I&T results in an absorbed dose to the patient's salivary glands of less than or equal to 0.10 Gy/GBq.

In various embodiments, the administration of the [¹⁷⁷Lu]Lu-PSMA I&T results in an absorbed dose to the patient's salivary glands of 0.13 Gy/GBq±0.08 Gy/GBq (i.e., 0.13 Gy per GBq of the administered [¹⁷⁷Lu]Lu-PSMA I&T). In some additional embodiments, the administration of the [¹⁷⁷Lu]Lu-PSMA I&T results in an absorbed dose to the patient's salivary glands of less than or equal to 0.13 Gy/GBq.

In other embodiments, the administration of the [¹⁷⁷Lu]Lu-PSMA I&T results in an absorbed dose to the patient's salivary glands of 0.18 Gy/GBq±0.16 Gy/GBq (i.e., 0.18 Gy per GBq of the administered [¹⁷⁷Lu]Lu-PSMA I&T). In some additional embodiments, the administration of the [¹⁷⁷Lu]Lu-PSMA I&T results in an absorbed dose to the patient's salivary glands of less than or equal to 0.18 Gy/GBq.

In various embodiments, the administration of the [¹⁷⁷Lu]Lu-PSMA I&T results in an absorbed dose to the patient's liver of 0.03 Gy/GBq±0.02 Gy/GBq (i.e., 0.03 Gy per GBq of the administered [¹⁷⁷Lu]Lu-PSMA I&T). In some additional embodiments, the administration of the [¹⁷⁷Lu]Lu-PSMA I&T results in an absorbed dose to the patient's liver of less than or equal to 0.03 Gy/GBq.

In other embodiments, the administration of the [¹⁷⁷Lu]Lu-PSMA I&T results in an absorbed dose to the patient's liver of 0.04 Gy/GBq±0.02 Gy/GBq (i.e., 0.04 Gy per GBq of the administered ¹⁷⁷Lu-PSMA I&T). In some additional embodiments, the administration of the ¹⁷⁷Lu-PSMA I&T results in an absorbed dose to the patient's liver of less than or equal to 0.04 Gy/GBq.

The administration of ¹⁷⁷Lu-PSMA I&T may be described as a mathematical formula to ensure that the total cumulative dose to the patient's kidneys after all treatments remains below 23 Gy. An example formula is shown below to determine the number of cycles allowable.

$$X = \frac{23}{Y * Z}$$

where X is the total number of cycles allowable at a given activity of ¹⁷⁷Lu-PSMA I&T, Y is the activity of each dose of the ¹⁷⁷Lu-PSMA I&T, Y is the absorbed dose of radiation in Gy per MBq of the administered ¹⁷⁷Lu-PSMA I&T, and Z is the activity of the of the administered ¹⁷⁷Lu-PSMA I&T in MBq.

In various embodiments, the patient may be administered 1 GBq of ¹⁷⁷Lu-PSMA I&T for 53 treatments, 2 GBq of ¹⁷⁷Lu-PSMA I&T for 26 treatments, 3 GBq of ¹⁷⁷Lu-PSMA I&T for 17 treatments, 4 GBq of ¹⁷⁷Lu-PSMA I&T for 13 treatments, 5 GBq of ¹⁷⁷Lu-PSMA I&T for 10 treatments, 6 GBq of ¹⁷⁷Lu-PSMA I&T for 8 treatments, 7 GBq of ¹⁷⁷Lu-PSMA I&T for 7 treatments, 8 GBq of ¹⁷⁷Lu-PSMA I&T for 6 treatments, 9 GBq of ¹⁷⁷Lu-PSMA I&T for 5 treatments, 10 GBq of ¹⁷⁷Lu-PSMA I&T for 5 treatments, etc.

In various embodiments, the present disclosure is further related to a radiopharmaceutical kit, comprising a vial containing at least a single dose of a ¹⁷⁷Lu-PSMA I&T solution for injection to a human patient in need thereof, wherein the injection comprises a 6.5 GBq±0.10 GBq dose, 6.5 GBq±0.15 GBq dose, 6.5 GBq±0.20 GBq dose, 6.5 GBq±0.25 GBq dose, or 6.5 GBq±0.30 GBq dose, and wherein ¹⁷⁷Lu-PSMA I&T treatment with 1, 2, 3, 4, 5, 6, or 7 cycles at the dose is possible without the risk of kidney toxicities and/or wherein ¹⁷⁷Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

In various embodiments, the present disclosure is further related to a radiopharmaceutical kit, comprising a vial containing at least a single dose of a ¹⁷⁷Lu-PSMA I&T solution for injection to a human patient in need thereof, wherein the injection comprises a 6.6 GBq±0.10 GBq dose, 6.6 GBq±0.15 GBq dose, 6.6 GBq±0.20 GBq dose, 6.6 GBq±0.25 GBq dose, or 6.6 GBq±0.30 GBq dose, and wherein ¹⁷⁷Lu-PSMA I&T treatment with 1, 2, 3, 4, 5, 6, or 7 cycles at the dose is possible without the risk of kidney toxicities and/or wherein ¹⁷⁷Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

In various embodiments, the present disclosure is further related to a radiopharmaceutical kit, comprising a vial containing at least a single dose of a ¹⁷⁷Lu-PSMA I&T solution for injection to a human patient in need thereof, wherein the injection comprises a 6.7 GBq±0.10 GBq dose, 6.7 GBq±0.15 GBq dose, 6.7 GBq±0.20 GBq dose, 6.7 GBq±0.25 GBq dose, or 6.7 GBq±0.30 GBq dose, and wherein ¹⁷⁷Lu-PSMA I&T treatment with 1, 2, 3, 4, 5, 6, or 7 cycles at the dose is possible without the risk of kidney toxicities and/or wherein ¹⁷⁷Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

In various embodiments, the present disclosure is further related to a radiopharmaceutical kit, comprising a vial containing at least a single dose of a ¹⁷⁷Lu-PSMA I&T solution for injection to a human patient in need thereof, wherein the injection comprises a 6.8 GBq±0.10 GBq dose, 6.8 GBq±0.15 GBq dose, 6.8 GBq±0.20 GBq dose, 6.8 GBq±0.25 GBq dose, or 6.8 GBq±0.30 GBq dose, and wherein ¹⁷⁷Lu-PSMA I&T treatment with 1, 2, 3, 4, 5, 6, or 7 cycles at the dose is possible without the risk of kidney toxicities and/or wherein ¹⁷⁷Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

In various embodiments, the present disclosure is further related to a radiopharmaceutical kit, comprising a vial containing at least a single dose of a ¹⁷⁷Lu-PSMA I&T solution for injection to a human patient in need thereof, wherein the injection comprises a 6.9 GBq±0.10 GBq dose, 6.9 GBq±0.15 GBq dose, 6.9 GBq±0.20 GBq dose, 6.9 GBq±0.25 GBq dose, or 6.9 GBq±0.30 GBq dose, and wherein ¹⁷⁷Lu-PSMA I&T treatment with 1, 2, 3, 4, 5, 6, or 7 cycles at the dose is possible without the risk of kidney toxicities and/or wherein ¹⁷⁷Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

In various embodiments, the present disclosure is further related to a radiopharmaceutical kit, comprising a vial containing at least a single dose of a ¹⁷⁷Lu-PSMA I&T solution for injection to a human patient in need thereof, wherein the injection comprises a 7.0 GBq±0.10 GBq dose, 7.0 GBq±0.15 GBq dose, 7.0 GBq±0.20 GBq dose, 7.0 GBq±0.25 GBq dose, or 7.0 GBq±0.30 GBq dose, and wherein ¹⁷⁷Lu-PSMA I&T treatment with 1, 2, 3, 4, 5, 6, or 7 cycles at the dose is possible without the risk of kidney toxicities and/or wherein ¹⁷⁷Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

In various embodiments, the present disclosure is further related to a radiopharmaceutical kit, comprising a vial containing at least a single dose of a ¹⁷⁷Lu-PSMA I&T solution for injection to a human patient in need thereof, wherein the injection comprises a 7.1 GBq±0.10 GBq dose, 7.1 GBq±0.15 GBq dose, 7.1 GBq±0.20 GBq dose, 7.1 GBq±0.25 GBq dose, or 7.1 GBq±0.30 GBq dose, and wherein ¹⁷⁷Lu-PSMA I&T treatment with 1, 2, 3, 4, 5, 6, or 7 cycles at the dose is possible without the risk of kidney toxicities and/or wherein ¹⁷⁷Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

In various embodiments, the present disclosure is further related to a radiopharmaceutical kit, comprising a vial containing at least a single dose of a ¹⁷⁷Lu-PSMA I&T solution for injection to a human patient in need thereof, wherein the injection comprises a 7.2 GBq±0.10 GBq dose, 7.2 GBq±0.15 GBq dose, 7.2 GBq±0.20 GBq dose, 7.2 GBq±0.25 GBq dose, or 7.2 GBq±0.30 GBq dose, and wherein ¹⁷⁷Lu-PSMA I&T treatment with 1, 2, 3, 4, 5, 6, or 7 cycles at the dose is possible without the risk of kidney toxicities and/or wherein ¹⁷⁷Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

In various embodiments, the present disclosure is further related to a radiopharmaceutical kit, comprising a vial containing at least a single dose of a ¹⁷⁷Lu-PSMA I&T solution for injection to a human patient in need thereof, wherein the injection comprises a 7.3 GBq±0.10 GBq dose, 7.3 GBq±0.15 GBq dose, 7.3 GBq±0.20 GBq dose, 7.3 GBq±0.25 GBq dose, or 7.3 GBq±0.30 GBq dose, and wherein ¹⁷⁷Lu-PSMA I&T treatment with 1, 2, 3, 4, 5, 6, or 7 cycles at the dose is possible without the risk of kidney toxicities and/or wherein ¹⁷⁷Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

In various embodiments, the present disclosure is further related to a radiopharmaceutical kit, comprising a vial containing at least a single dose of a ¹⁷⁷Lu-PSMA I&T solution for injection to a human patient in need thereof, wherein the injection comprises a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, and wherein ¹⁷⁷Lu-PSMA I&T treatment with 6 cycles at the dose is possible without the risk of kidney toxicities and/or wherein ¹⁷⁷Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

In various embodiments, the present disclosure is further related to a radiopharmaceutical kit, comprising a vial containing at least a single dose of a [¹⁷⁷Lu]Lu-PSMA I&T solution for injection to a human patient in need thereof, wherein the injection comprises a 7.5 GBq±0.10 GBq dose, 7.5 GBq±0.15 GBq dose, 7.5 GBq±0.20 GBq dose, 7.5 GBq±0.25 GBq dose, or 7.5 GBq±0.30 GBq dose, and wherein [¹⁷⁷Lu]Lu-PSMA I&T treatment with 1, 2, 3, 4, 5, 6, or 7 cycles at the dose is possible without the risk of kidney toxicities and/or wherein [¹⁷⁷Lu]Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 6 cycles is less than 23 Gy and no renal toxicities are observed.

In various embodiments, the present disclosure is further related to a radiopharmaceutical kit, comprising a vial containing at least a single dose of a [¹⁷⁷Lu]Lu-PSMA I&T solution for injection to a human patient in need thereof, wherein the injection comprises a 7.4 GBq (mean 7.52±0.16 GBq) dose of [¹⁷⁷Lu]Lu-PSMA-I&T, and wherein [¹⁷⁷Lu]Lu-PSMA I&T treatment with 6 cycles at the dose is possible without the risk of kidney toxicities and/or wherein [¹⁷⁷Lu]Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed. The present disclosure is also further related to a radiopharmaceutical kit, comprising a vial containing at least a single dose of a [¹⁷⁷Lu]Lu-PSMA I&T solution for injection to a human patient in need thereof, wherein the injection comprises a 7.4 GBq (mean 7.52±0.16 GBq) dose of [¹⁷⁷Lu]Lu-PSMA-I&T, wherein [¹⁷⁷Lu]Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

The present disclosure is also further related to a radiopharmaceutical kit, comprising a vial containing at least a single dose of a [¹⁷⁷Lu]Lu-PSMA I&T solution for injection to a human patient in need thereof, wherein the injection comprises a 6.5 GBq+/−10% GBq dose, 6.5 GBq+/−5% GBq dose, or 6.5 GBq+/−3% GBq dose of ¹⁷⁷Lu-PSMA-I&T, and wherein [¹⁷⁷Lu]Lu-PSMA I&T treatment with 1, 2, 3, 4, 5, 6, or 7 cycles at the dose is possible without the risk of kidney toxicities and/or wherein [¹⁷⁷Lu]Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

The present disclosure is also further related to a radiopharmaceutical kit, comprising a vial containing at least a single dose of a [¹⁷⁷Lu]Lu-PSMA I&T solution for injection to a human patient in need thereof, wherein the injection comprises a 6.8 GBq+/−10% GBq dose, 6.8 GBq+/−5% GBq dose, or 6.8 GBq+/−3% GBq dose of [¹⁷⁷Lu]Lu-PSMA-I&T, and wherein [¹⁷⁷Lu]Lu-PSMA I&T treatment with 1, 2, 3, 4, 5, 6, or 7 cycles at the dose is possible without the risk of kidney toxicities and/or wherein [¹⁷⁷Lu]Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

The present disclosure is also further related to a radiopharmaceutical kit, comprising a vial containing at least a single dose of a [¹⁷⁷Lu]Lu-PSMA I&T solution for injection to a human patient in need thereof, wherein the injection comprises a 7.4 GBq+/−10% GBq dose, 7.4 GBq+/−5% GBq dose, or 7.4 GBq+/−3% GBq dose of ¹⁷⁷Lu-PSMA-I&T, and wherein ¹⁷⁷Lu-PSMA I&T treatment with 1, 2, 3, 4, 5, 6, or 7 cycles at the dose is possible without the risk of kidney toxicities and/or wherein [¹⁷⁷Lu]Lu-PSMA I&T treatment with 1, 2, 3, 4, 5, 6, or 7 cycles provides a mean projected dose that is below a cumulative absorbed dose to the kidney of 23 Gy and/or the projected or actual cumulative absorbed dose to the kidneys at 1, 2, 3, 4, 5, 6, or 7 cycles is less than 23 Gy and no renal toxicities are observed.

The present disclosure is also further related to a radiopharmaceutical kit, comprising a vial containing at least a single dose of a [¹⁷⁷Lu]Lu-PSMA I&T solution for injection to a human patient in need thereof, wherein the injection comprises a 7.4 (+/−10%) GBq dose of [¹⁷⁷Lu]Lu-PSMA-I&T, and wherein [¹⁷⁷Lu]Lu-PSMA I&T treatment with 6 cycles at the dose is possible without the risk of kidney toxicities and/or wherein [¹⁷⁷Lu]Lu-PSMA I&T treatment with 6 cycles provides a mean projected dose that is below a cumulative absorbed dose to the kidney of 23 Gy and/or the projected or actual cumulative absorbed dose to the kidneys at 6 cycles is less than 23 Gy and no renal toxicities are observed.

The present disclosure is further related to a radiopharmaceutical kit, comprising a vial containing at least a single dose of a [¹⁷⁷Lu]Lu-PSMA I&T solution for injection to a human patient in need thereof, wherein the injection comprises a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, and wherein the projected cumulative absorbed dose to the kidneys at 6 cycles will be 20.4±10.2 Gy. The present disclosure is further related to a radiopharmaceutical kit, comprising a vial containing at least a single dose of a [¹⁷⁷Lu]Lu-PSMA I&T solution for injection to a human patient in need thereof, wherein the injection comprises a 7.5 GBq±0.10 GBq dose, 7.5 GBq±0.15 GBq dose, 7.5 GBq±0.20 GBq dose, 7.5 GBq±0.25 GBq dose, or 7.5 GBq±0.30 GBq dose, and wherein the projected cumulative absorbed dose to the kidneys at 6 cycles will be 20.4±10.2 Gy.

In some embodiments, the present disclosure includes a kit that includes a predetermined amount of a composition that includes ¹⁷⁷Lu-PSMA I&T. In another embodiment the molar ratio of the PSMA I&T to ¹⁷⁷Lu is from 5.0:1.0 to 12.0:1.0. In another embodiment, the composition is suitable for administration to a human patient in need thereof.

Upon administration of the radiopharmaceutical composition to a patient, the patient may maintain low levels of hematotoxic and nephrotoxic toxicity. In some embodiments, the prostate-specific antigen (PSA) decline is more than about 40%, more than about 45%, more than about 50%, more than about 55%, more than about 60%, more than about 65%, more than about 70%, more than about 75%, or more than about 80%.

Further provided herein are methods of treating a patient with cancer and/or mCRPC by administering the radiopharmaceutical composition comprising ¹⁷⁷Lu-PSMA I&T. The method may further comprise imaging the patient using PSMA-PET to document and confirm the patient is mCRPC positive prior to administering the radiopharmaceutical composition. For example, the patient may have a PSMA-PET scan (e.g. [⁶⁸Ga]Ga-PSMA-11 or [¹⁸F]DCFPyL) positive as determined by central reader. In an embodiment provided herein is a method of imaging cancer in a human patient in thereof. In another embodiment, the method further includes administering to the human patient a composition that includes ¹⁷⁷Lu-PSMA I&T. In another embodiment the molar ratio of the PSMA I&T to ¹⁷⁷Lu is from 5.0:1.0 to 12.0:1.0.

### Indications and Contraindications

RLT with ¹⁷⁷Lu-PSMA I&T may be indicated for the treatment of patients with cancer and/or mCRPC, who do not have any other approved therapy option planned by a multidisciplinary team. In an embodiment, a method of treating cancer in a patient in need thereof is provided herein.

In another embodiment, the method includes administering to the human patient a composition that includes ¹⁷⁷Lu-PSMA I&T. In another embodiment the molar ratio of the PSMA I&T to ¹⁷⁷Lu is from 1.0:1.0 to 12.0:1.0, 3.0:1.0 to 12.0:1.0, 5.0:1.0 to 12.0:1.0. In another embodiment the molar ratio of the PSMA I&T to ¹⁷⁷Lu is from 1.0:1.0 to 8.0:1.0, 1.5:1.0 to 8.0:1.0, 2.0:1.0 to 8.0:1.0, 2.5:1.0 to 8.0:1.0, 3.0:1.0 to 8.0:1.0, 3.5:1.0 to 8.0:1.0, or 4.0:1.0 to 8.0:1.0. In another embodiment the molar ratio of the PSMA I&T to ¹⁷⁷Lu is from 4.1:1.0 to 7.9:1.0, 4.2:1.0 to 7.8:1.0, 4.3:1.0 to 7.7:1.0, 4.4:1.0 to 7.6:1.0, 4.5:1.0 to 7.5:1.0, 4.6:1.0 to 7.4:1.0, 4.7:1.0 to 7.3:1.0, 4.8:1.0 to 7.2:1.0, 4.9:1.0 to 7.1:1.0, or 5.0:1.0 to 7.0:1.0. In another embodiment the molar ratio of the PSMA I&T to ¹⁷⁷Lu is from 5.0:1.0 to 7.6:1.0, 5.1:1.0 to 7.5:1.0, 5.2:1.0 to 7.4:1.0, 5.3:1.0 to 7.3:1.0, or 5.4:1.0 to 7.2:1.0. In another embodiment the molar ratio of the PSMA I&T to ¹⁷⁷Lu is from 8.0:1.0 to 10.0:1.0, 8.1:1.0 to 10.0:1.0, 8.2:1.0 to 10.0:1.0, 8.3:1.0 to 10.0:1.0, 8.4:1.0 to 10.0:1.0, 8.5:1.0 to 10.0:1.0, 8.6:1.0 to 10.0:1.0, 8.7:1.0 to 10.0:1.0, 8.8:1.0 to 10.0:1.0, 8.9:1.0 to 10.0:1.0, 9.0:1.0 to 10.0:1.0, 9.1:1.0 to 10.0:1.0, 9.2:1.0 to 10.0:1.0, 9.3:1.0 to 10.0:1.0, 9.4:1.0 to 10.0:1.0, 9.5:1.0 to 10.0:1.0, 9.6:1.0 to 10.0:1.0, 9.7:1.0 to 10.0:1.0, 9.8:1.0 to 10.0:1.0, or 9.9:1.0 to 10.0:1.0. In another embodiment the molar ratio of the PSMA I&T to ¹⁷⁷Lu is from 8.0:1.0 to 11.0:1.0, 8.1:1.0 to 11.0:1.0, 8.2:1.0 to 11.0:1.0, 8.3:1.0 to 11.0:1.0, 8.4:1.0 to 11.0:1.0, 8.5:1.0 to 11.0:1.0, 8.6:1.0 to 11.0:1.0, 8.7:1.0 to 11.0:1.0, 8.8:1.0 to 11.0:1.0, 8.9:1.0 to 11.0:1.0, 9.0:1.0 to 11.0:1.0, 9.1:1.0 to 11.0:1.0, 9.2:1.0 to 11.0:1.0, 9.3:1.0 to 11.0:1.0, 9.4:1.0 to 11.0:1.0, 9.5:1.0 to 11.0:1.0, 9.6:1.0 to 11.0:1.0, 9.7:1.0 to 11.0:1.0, 9.8:1.0 to 11.0:1.0, 9.9:1.0 to 11.0:1.0, 10.0:1.0 to 11.0:1.0, 10.1:1.0 to 11.0:1.0, 10.2:1.0 to 11.0:1.0, 10.3:1.0 to 11.0:1.0, 10.4:1.0 to 11.0:1.0, 10.5:1.0 to 11.0:1.0, 10.6:1.0 to 11.0:1.0, 10.7:1.0 to 11.0:1.0, 10.8:1.0 to 11.0:1.0, or 10.9:1.0 to 11.0:1.0. The molar ratio of the PSMA I&T to ¹⁷⁷Lu may be from 3.0:1.0 to 12.0:1.0, 3.5:1.0 to 12.0:1.0, 4.0:1.0 to 12.0:1.0, 4.4:1.0 to 12.0:1.0, 4.4:1.0 to 11.5:1.0, 4.4:1.0 to 11.0:1.0, 4.4:1.0 to 10.5:1.0, 4.4:1.0 to 10.0:1.0, 4.4:1.0 to 9.5:1.0, 4.4:1.0 to 9.0:1.0, 4.4:1.0 to 8.5:1.0, 4.4:1.0 to 8.0:1.0, 4.4:1.0 to 7.5:1.0, 4.4:1.0 to 7.0:1.0, 4.4:1.0 to 6.5:1.0, 4.4:1.0 to 6.0:1.0, 4.5:1.0 to 5.9:1.0, 4.6:1.0 to 4.7:1.0, 4.8:1.0 to 5.7:1.0, or 4.9:1.0 to 5.6:1.0. The molar ratio of the PSMA I&T to ¹⁷⁷Lu may be from 5.0:1.0 to 12.0:1.0, 5.0:1.0 to 11.5:1.0, 5.0:1.0 to 11.0:1.0, 5.0:1.0 to 10.5:1.0, 5.0:1.0 to 10.0:1.0, 5.0:1.0 to 9.5:1.0, 5.0:1.0 to 9.0:1.0, 5.0:1.0 to 8.5:1.0, 5.0:1.0 to 8.0:1.0, 5.0:1.0 to 7.5:1.0, 5.0:1.0 to 7.0:1.0, 5.0:1.0 to 6.5:1.0, 5.0:1.0 to 6.0:1.0, 5.1:1.0 to 5.9:1.0, 5.2:1.0 to 5.8:1.0, 5.3:1.0 to 5.7:1.0, 5.4:1.0 to 5.6:1.0, or 5.45:1.0 to 5.55:1.0. The molar ratio of the PSMA I&T to ¹⁷⁷Lu may be from 8.0:1.0 to 12.0:1.0, 8.0:1.0 to 11.5:1.0, 8.0:1.0 to 11.0:1.0, 8.0:1.0 to 10.5:1.0, 8.0:1.0 to 10.4:1.0, 8.0:1.0 to 10.3:1.0, 8.0:1.0 to 10.2:1.0, 8.0:1.0 to 10.1:1.0, 8.0:1.0 to 10.0:1.0, 8.0:1.0 to 9.9:1.0, 8.0:1.0 to 9.8:1.0, 8.0:1.0 to 9.7:1.0, 8.0:1.0 to 9.6:1.0, 8.0:1.0 to 9.5:1.0, 8.0:1.0 to 9.4:1.0, 8.0:1.0 to 9.3:1.0, 8.0:1.0 to 9.2:1.0, 8.0:1.0 to 9.1:1.0, or 8.0:1.0 to 9.0:1.0. The molar ratio of the PSMA I&T to ¹⁷⁷Lu may be from 9.0:1.0 to 12.0:1.0, 9.0:1.0 to 11.5:1.0, 9.0:1.0 to 11.0:1.0, 9.0:1.0 to 10.5:1.0, 9.0:1.0 to 10.4:1.0, 9.0:1.0 to 10.3:1.0, 9.0:1.0 to 10.2:1.0, 9.0:1.0 to 10.1:1.0, 9.0:1.0 to 10.0:1.0, 9.0:1.0 to 9.9:1.0, 9.0:1.0 to 9.8:1.0, 9.0:1.0 to 9.7:1.0, 9.0:1.0 to 9.6:1.0, or 9.0:1.0 to 9.5:1.0.

The molar ratio of the PSMA I&T to ¹⁷⁷Lu may be from 11.0:1.0 to 12.0:1.0, 11.1:1.0 to 11.9:1.0, 11.2:1.0 to 11.8:1.0, 11.3:1.0 to 11.7:1.0, or 11.4:1.0 to 11.6:1.0. The molar ratio of the PSMA I&T to ¹⁷⁷Lu may be from 10.0:1.0 to 11.0:1.0, 10.1:1.0 to 10.9:1.0, 10.2:1.0 to 10.8:1.0, 10.3:1.0 to 10.7:1.0, or 10.4:1.0 to 10.6:1.0. The molar ratio of the PSMA I&T to ¹⁷⁷Lu may be from 9.0:1.0 to 10.0:1.0, 9.1:1.0 to 9.9:1.0, 9.2:1.0 to 9.8:1.0, 9.3:1.0 to 9.7:1.0, or 9.4:1.0 to 9.6:1.0. The molar ratio of the PSMA I&T to ¹⁷⁷Lu may be from 8.0:1.0 to 9.0:1.0, 8.1:1.0 to 8.9:1.0, 8.2:1.0 to 8.8:1.0, 8.3:1.0 to 8.7:1.0, or 8.4:1.0 to 8.6:1.0. The molar ratio of the PSMA I&T to ¹⁷⁷Lu may be from 7.0:1.0 to 8.0:1.0, 7.1:1.0 to 7.9:1.0, 7.2:1.0 to 7.8:1.0, 7.3:1.0 to 7.7:1.0, or 7.4:1.0 to 7.6:1.0. The molar ratio of the PSMA I&T to ¹⁷⁷Lu may be from 6.0:1.0 to 7.0:1.0, 6.1:1.0 to 6.9:1.0, 6.2:1.0 to 7.8:1.0, 7.3:1.0 to 7.7:1.0, or 7.4:1.0 to 6.6:1.0. The molar ratio of the PSMA I&T to ¹⁷⁷Lu may be from 5.0:1.0 to 6.0:1.0, 5.1:1.0 to 5.9:1.0, 5.2:1.0 to 5.8:1.0, 5.3:1.0 to 5.7.0:1.0, or 5.4:1.0 to 5.6:1.0. The molar ratio of the PSMA I&T to ¹⁷⁷Lu may be from 4.4:1.0 to 5:0:1.0, 4.5:1.0 to 5:0:1.0, 4.6:1.0 to 5:0:1.0, 4.7:1.0 to 5:0:1.0, 4.8:1. to 5:0:1.0, or 4.9:1. to 5:0:1.0.

The molar ratio of the PSMA I&T to ¹⁷⁷Lu may be about 5.0:1.0 to about 5.5:1.0, about 5.5:1.0 to about 6.0:1.0, about 6.0:1.0 to about 6.5:1.0, about 6.5:1.0 to about 7.0:1.0, about 7.0:1.0 to about 7.5:1.0, about 7.5:1.0 to about 8.0:1.0, about 8.0:1.0 to about 8.5:1.0, about 8.5:1.0 to about 9.0:1.0, about 9.0:1.0 to about 9.5:1.0, about 9.5:1.0 to about 10.0:1.0, about 10.0:1.0 to about 10.5:1.0, about 10.5:1.0 to about 11.0:1.0, about 11.0:1.0 to about 11.5:1.0, or about 11.5:1.0 to about 12.0:1.0.

In another embodiment, in the compositions, kits, and methods described herein, the PSMA I&T to [177Lu]Lu3+ ratio in μg:mCi may be ≤0.65, ≤0.64, ≤0.63, ≤0.62, ≤0.61, or ≤0.60. In another embodiment, in the compositions, kits, and methods described herein, the PSMA I&T to [177Lu]Lu3+ ratio in μg:mCi may be from about 0.20 to about 0.64, about 0.20 to about 0.63, about 0.20 to about 0.62, about 0.20 to about 0.61, or about 0.20 to about 0.60. In another embodiment, in the compositions, kits, and methods described herein, the PSMA I&T to [177Lu]Lu3+ ratio in μg:mCi may be from about 0.21 to about 0.59, about 0.22 to about 0.58, about 0.23 to about 0.57, about 0.24 to about 0.56, about 0.25 to about 0.55, about 0.26 to about 0.54, about 0.27 to about 0.53, about 0.28 to about 0.52, about 0.29 to about 0.51, about 0.30 to about 0.50, about 0.31 to about 0.49, about 0.32 to about 0.48, about 0.33 to about 0.47, about 0.34 to about 0.46, about 0.35 to about 0.45, about 0.36 to about 0.44, about 0.37 to about 0.43, about 0.38 to about 0.42, about 0.39 to about 0.41. In another embodiment, in the compositions, kits, and methods described herein, the PSMA I&T to [177Lu]Lu3+ ratio in μg:mCi may be from about 0.50 to about 0.64, about 0.50 to about 0.63, about 0.50 to about 0.62, about 0.50 to about 0.61, about 0.50 to about 0.60, about 0.50 to about 0.59, about 0.50 to about 0.58, about 0.50 to about 0.57, about 0.50 to about 0.56, about 0.50 to about 0.55, about 0.50 to about 0.54, about 0.50 to about 0.53, about 0.50 to about 0.52, or about 0.50 to about 0.51. In another embodiment, in the compositions, kits, and methods described herein, the PSMA I&T to [177Lu]Lu3+ ratio in μg:mCi may be from about 0.40 to about 0.64, about 0.40 to about 0.63, about 0.40 to about 0.62, about 0.40 to about 0.61, about 0.40 to about 0.60, about 0.40 to about 0.59, about 0.40 to about 0.58, about 0.40 to about 0.57, about 0.40 to about 0.56, about 0.40 to about 0.55, about 0.40 to about 0.54, about 0.40 to about 0.53, about 0.40 to about 0.52, about 0.40 to about 0.51, about 0.40 to about 0.50, about 0.40 to about 0.49, about 0.40 to about 0.48, about 0.40 to about 0.47, about 0.40 to about 0.46, about 0.40 to about 0.45, about 0.40 to about 0.44, about 0.40 to about 0.43, about 0.40 to about 0.42, about 0.40 to about 0.41. In another embodiment, in the compositions, kits, and methods described herein, the PSMA I&T to [177Lu]Lu3+ ratio in μg:mCi may be from about 0.35 to about 0.64, about 0.35 to about 0.63, about 0.35 to about 0.62, about 0.35 to about 0.61, about 0.35 to about 0.60, about 0.35 to about 0.59, about 0.35 to about 0.58, about 0.35 to about 0.57, about 0.35 to about 0.56, about 0.35 to about 0.55, about 0.35 to about 0.54, about 0.35 to about 0.53, about 0.35 to about 0.52, about 0.35 to about 0.51, about 0.35 to about 0.50, about 0.35 to about 0.49, about 0.35 to about 0.48, about 0.35 to about 0.47, about 0.35 to about 0.46, about 0.35 to about 0.45, about 0.35 to about 0.44, about 0.35 to about 0.43, about 0.35 to about 0.42, about 0.35 to about 0.41, about 0.35 to about 0.40, about 0.35 to about 0.39, about 0.35 to about 0.38, about 0.35 to about 0.37, or about 0.35 to about 0.36. In another embodiment, in the compositions, kits, and methods described herein, the PSMA I&T to [177Lu]Lu3+ ratio in μg:mCi may be from about 0.30 to about 0.64, about 0.30 to about 0.63, about 0.30 to about 0.62, about 0.30 to about 0.61, about 0.30 to about 0.60, about 0.30 to about 0.59, about 0.30 to about 0.58, about 0.30 to about 0.57, about 0.30 to about 0.56, about 0.30 to about 0.55, about 0.30 to about 0.54, about 0.30 to about 0.53, about 0.30 to about 0.52, about 0.30 to about 0.51, about 0.30 to about 0.50, about 0.30 to about 0.49, about 0.30 to about 0.48, about 0.30 to about 0.47, about 0.30 to about 0.46, about 0.30 to about 0.45, about 0.30 to about 0.44, about 0.30 to about 0.43, about 0.30 to about 0.42, about 0.30 to about 0.41, about 0.30 to about 0.40, about 0.30 to about 0.39, about 0.30 to about 0.38, about 0.30 to about 0.37, about 0.30 to about 0.36, about 0.30 to about 0.35, about 0.30 to about 0.34, about 0.30 to about 0.33, about 0.30 to about 0.32, or about 0.30 to about 0.31. In another embodiment, in the compositions, kits, and methods described herein, the PSMA I&T to [177Lu]Lu3+ ratio in μg:mCi may be from about 0.25 to about 0.64, about 025 to about 0.63, about 0.25 to about 0.62, about 0.25 to about 0.61, about 0.25 to about 0.60, about 0.25 to about 0.59, about 0.25 to about 0.58, about 0.25 to about 0.57, about 0.25 to about 0.56, about 0.25 to about 0.55, about 0.25 to about 0.54, about 0.25 to about 0.53, about 0.25 to about 0.52, about 0.25 to about 0.51, about 0.25 to about 0.50, about 0.25 to about 0.49, about 0.25 to about 0.48, about 0.25 to about 0.47, about 0.25 to about 0.46, about 0.25 to about 0.45, about 0.25 to about 0.44, about 0.25 to about 0.43, about 0.25 to about 0.42, about 0.25 to about 0.41, about 0.25 to about 0.40, about 0.25 to about 0.39, about 0.25 to about 0.38, about 0.25 to about 0.37, about 0.25 to about 0.36, about 0.25 to about 0.35, about 0.25 to about 0.34, about 0.25 to about 0.33, about 0.25 to about 0.32, about 0.25 to about 0.31, about 0.25 to about 0.30, about 0.25 to about 0.29, about 0.25 to about 0.28, about 0.25 to about 0.27, or about 0.25 to about 0.26. In another embodiment, in the compositions, kits, and methods described herein, the PSMA I&T to [177Lu]Lu3+ ratio in μg:mCi may be from about 0.20 to about 0.30.

In another embodiment, the patient is treatment naïve. In another embodiment, the patient is not treatment naïve.

In an embodiment, the pharmaceutical composition is administered to the cancer patient as a first line therapy. In another embodiment, the pharmaceutical composition is administered to the patient as a regimen. In another embodiment, the pharmaceutical composition being administered has a radiochemical purity of greater than 95% at administration.

In an embodiment, the method of treating cancer prolongs a disease progression time of said cancer in a patient in thereof. In another embodiment, the method of treating cancer prolongs a survival of the patient. In another embodiment, the method of treating cancer increases progression-free survival of said patient. In another embodiment, the cancer is metastatic castration-resistant prostate cancer (mCRPC).

In some embodiments, the patient also has histologically or pathologically confirmed prostate adenocarcinoma without predominant small cell component, has progressive disease by one or more of the following criteria: a) Serum/plasma PSA progression defined as 2 consecutive increases in PSA over a previous reference value measured at least 1 week apart with a minimum start value of >2 ng/mL; or b) Progression of measurable disease (RECIST 1.1) or presence of at least two new bone lesions (PCWG3 criteria), and/or has had previous treatment with a next-generation androgen receptor (AR)-directed therapy (e.g. abiraterone, enzalutamide, apalutamide, darolutamide). In additional embodiments, the patient may have effective castration with a serum testosterone level of <50 ng/dL and plan to continue with chronic medical or surgical castration. mCRPC patients should undergo hormone therapy and chemotherapy as well as bone targeted therapy, if indicated.

In at least one example, a patient in need of RLT using ¹⁷⁷Lu-PSMA I&T may fulfill the following criteria:
1) mCRPC with PSMA positive metastatic disease based upon PSMA-PET or SPECT imaging. There are no limitations regarding the number or site of metastases, i.e. bone or soft tissue metastases. Caution should be given to patients e.g. with diffuse bone marrow, perineural and brain metastases.
2) After initial hormone therapy (LH-RH agonists/antagonists). Progressive disease, i.e. biochemical and/or radiologic progression, despite newly developed hormone therapies (Abiraterone/Enzalutamide) or these medications may be avoided by the patient. Progressive disease despite chemotherapy (Docetaxel and Cabazitaxel) or the patient being unfit for chemotherapy or avoiding chemotherapy.
3) Not suitable for ¹⁵³Sm-EDTMP or [²²³Ra]RaCl₂ or other local available radiopharmaceuticals for bone-targeted therapies due to extra-osseous metastases or diffuse bone marrow metastases or avoided by the patient. In patients without adequate response to bone-targeted therapies for pain palliation or exacerbation of pain even by such therapy, an RLT with ¹⁷⁷Lu-PSMA I&T can be evaluated.
4) Life expectancy longer than 4-6 months.
5) Decision for salvage therapy at the institutional interdisciplinary tumor board.

In summary, mCRPC patients should undergo hormone therapy and chemotherapy as well as bone targeted therapy, if indicated. In the case of any contraindication for one of these therapies, it should be discussed and documented in an interdisciplinary tumor board.

Contraindications are as follows:
(1) WBC≤1×10⁹/L.
(2) Hb≤80 g/l. (In the case of symptomatic anemia, a red blood cell transfusion should precede the therapy. RLT with ¹⁷⁷Lu-PSMA I&T may have a positive effect on bone marrow depression, causing the need for less blood to be transfused due to tumor regression in bone marrow. It should be noted that pure anemia without thrombocytopenia and leucopenia is not a contraindication for RLT.)
(3) Platelets ≤70×10⁹/L.
(4) Creatinine >1.5 UNL; Kidney failure with creatinine clearance <30 mL/min
(5) Absolute obstruction in renal excretion.
(6) Previous chemotherapy or bone-targeted radionuclide therapy and extended external beam irradiation fields to the bone marrow (pelvis, spine), if performed during 4 weeks preceding the RLT
(7) ECOG performance status >2.
(8) Hypersensitivity to the active substance or to any of the excipients.

After a patient in need thereof has been identified, the activity of the radiopharmaceutical composition may be confirmed prior to administration. The radioactivity of the ¹⁷⁷Lu-PSMA I&T composition may be 6.5-7.5 GBq or within a range of 6.0-8.0 GBq. The radioactivity may be reduced to 4.0-5.0 GBq in the case of impaired renal function (e.g. Creatinine within 1.0-1.5 UNL).

The radiopharmaceutical composition solution may be infused intravenously as a slow bolus (over about 10-15 minutes) followed by 500-1000 ml Ringer or NaCl solution. The patient may be encouraged to void as frequently as possible and drink about 2 liters of water daily. Patients with dilated non-obstructive renal disease may be administered diuretics.

The pharmaceutical composition may be administered as 2-11 cycles of the RLT every 5-8 weeks. In the case of continuously increasing PSA, after the first two cycles accompanied by worsening of the general condition, the indication of further RLTs may be re-evaluated. In the case of a decreasing PSA to <1.0 μg/l during the therapy cycles, a PSMA imaging may evaluate existence of small PSMA-positive metastases after completion of RLT when a post injection SPECT study is not enough to be informative. In the case of a significant decline of platelets or leukocytes, the time interval between 2 cycles may be prolonged.

At least one whole body scan 24-48 hours post injection (preferably with SPECT/CT) may be performed. In patients with diffuse bone and bone marrow metastases as well as in patients with brain metastases, a concomitant corticosteroid therapy (e.g. prednisolone 20 mg/daily) may be administered in the first two weeks after administration of the radiopharmaceutical composition.

In some embodiments, after administration of the radiopharmaceutical composition, the patient may have improved radiographic progression free survival (rPFS). A patient administered the radiopharmaceutical composition may have an rPFS of about 6 to about 12 months after initiating administration of the radiopharmaceutical composition. In various embodiments, the patient administered the radiopharmaceutical composition may have an rPFS of at least 6 months, 7 months, 8 months, 9 months, 10 months, 11 months, or 12 months after initiating administration of the radiopharmaceutical composition. For example, treatment of patients with ¹⁷⁷Lu-PSMA I&T may increase the rPFS from 6 months using standard of care to up to 10 months using the radiopharmaceutical composition. Radiographic progression free survival may be defined as the time from randomization to radiographic progression (using PCWG3 and RECIST 1.1 criteria as assessed by blinded independent central review [BICR]) or death due to any cause.

In an embodiment, the patient may have improved overall survival (OS) after initiating administration of the radiopharmaceutical composition. A patient administered the radiopharmaceutical composition may have an overall survival of about 18 to about 26 months after initiating administration of the radiopharmaceutical composition. In various embodiments, the patient administered the radiopharmaceutical composition may have an OS of at least 18 months, 19 months, 20 months, 21 months, 22 months, 23 months, 24 months, 25 months, or 26 months after initiating administration of the radiopharmaceutical composition. For example, treatment of patients with ¹⁷⁷Lu-PSMA I&T may increase the OS from 18 months using standard of care to up to 25 months using the radiopharmaceutical composition.

In another embodiment, the patient may have improved second radiographic progression free survival (rPFS 2) after initiating administration of the radiopharmaceutical composition.

In some embodiments, the patient may have improved progression free survival after initiating administration of the radiopharmaceutical composition. In additional embodiments, the patient may have improved second progression-free survival after initiating administration of the radiopharmaceutical composition. The second progression-free survival may be the second occurrence of PCWG3 progression, clinical/symptomatic progression and/or pain progression, or death due to any cause.

In an embodiment, the patient may have an improved PSA₅₀ response rate after initiating administration of the radiopharmaceutical composition. The PSA₅₀ response rate may be the response rate of patients who achieve a reduction of ≥50% in PSA from the baseline PSA assessment.

In an embodiment, the patient may have an improved time to first symptomatic skeletal event (SSE) after initiating administration of the radiopharmaceutical composition. An SSE may be the occurrence of either bone-directed radiotherapy to relieve bone pain, new symptomatic pathologic fractures, spinal cord compression, or tumor-related orthopedic surgery.

In an embodiment, the patient may have an improved time to soft tissue progression (STP) after initiating administration of the radiopharmaceutical composition. STP may include the occurrence of radiographic progression in soft tissue. In another embodiment, the patient may have an improved time to chemotherapy (TTC) after initiating administration of the radiopharmaceutical composition.

In an embodiment, the patient may have improved results on a Quality of Life Questionnaire after initiating administration of the radiopharmaceutical composition. For example, the Quality of Life (QoL) may be assessed via European Organisation for Research and Treatment of Cancer Quality of Life Questionnaire C30 (EORTC QLQ-C30). The EORTC QLQ-C30 is a questionnaire of thirty quality of life (QoL) questions developed to assess the QoL of cancer patients. The EORTC QLQ-C30 comprises 30 items, 24 of which are aggregated into nine multi-item scales, which are scored from 0 to 100.

### EXAMPLES

The following non-limiting examples are provided for illustrative purposes only, and therefore should not be viewed in a limiting sense.

### Analytical Procedures

The product was identified by subsequent injection of reference solution of Lu-PSMA I&T and formulated solution to a liquid chromatography system. Radio nuclidic identity was determined by gamma ray energy detection.

pH was estimated by pH paper. Radioactivity was measured in a dose calibrator. Radiochemical purity was determined by liquid chromatography with radioactivity detection and thin layer chromatography.

The radioactivity of [¹⁷⁷Lu]Lu-PSMA I&T was determined by a dose calibrator when the dose is dispensed.

Bacterial endotoxin content was determined for each batch before release using a PTS-tester (Ph Eur method D, USP<85>). Sterility was determined according to Ph Eur and in alignment with USP<71>.

Qualities of analytical procedures used for the drug product—e.g., specificity, linearity, and reproducibility—were investigated by using a known reference standard for the unlabeled precursor. All analytical procedures were found suitable for their intended use.

An acceptance criterion for the radioactivity in the formulation is not set since this will vary depending on the individual clinical need assessed by the health care professional responsible for administering the formulation. The radioactive content must be within 90%-110% of the stated value at the date and time stated on the label.

### Example 1: METHOD OF MANUFACTURING ¹⁷⁷LU-PSMA I&T RADIOPHARMACEUTICAL FORMULATION

Multiple radiopharmaceutical compositions were produced using the process as outlined in Table 2A and 2B below. Radiolabeling was performed using non-carrier added [¹⁷⁷Lu]LuCl₃. Compositions 1 and 2 are essentially the same. In Composition 3, the pH of the ascorbic acid solution was adjusted to 4.5, the amount of ascorbic acid was lowered, the amount of ethanol was lowered, and the pH of the final radiopharmaceutical composition was adjusted to 4.5. This resulted in Composition 3 having an extended shelf life as compared to Compositions 1 and 2.

The synthesis of ¹⁷⁷Lu-PSMA I&T utilizes an automated synthesis module in a controlled environment. The labelling solution containing [¹⁷⁷Lu]Lutetium chloride ([¹⁷⁷Lu]LuCl₃) is connected to the synthesis cassette containing the other chemicals components required for the labeling process. ¹⁷⁷LuCl₃ solution is transferred into reactor for radiolabeling and may be rinsed with additional required amount of 0.04 M HCl solution. The labelling solutions varies in volume depending on the ¹⁷⁷Lu radioactivity.

The [¹⁷⁷Lu]LuCl₃ solution is mixed in the reaction chamber with a solution of 0.4 M Sodium acetate buffer containing the diluted PSMA I&T precursor. The solution is heated in the reactor. After heating, the produced ¹⁷⁷Lu-PSMA I&T is trapped on a C18 cartridge pre-conditioned with water. The cartridge is rinsed with sterile water and the final product is eluted from the C18 cartridge with 1.5 ml of 50% sterile ethanol-water mixture into a bulk vial. The drug substance is formed in situ and is directly formulated into the drug product.

For final volume adjustment a formulation matrix containing 50 mg/ml ascorbic acid and ethanol in water for injection is added to the bulk vial. The composition of final product is fixed, and the amount of formulation matrix added depends on the radioactivity of ¹⁷⁷Lu used in the batch.

The formulation matrix is prepared from a solution of ascorbic acid that is diluted to 50 mg/ml concentration using water for injection. The ethanol concentration is adjusted to 3.8%±1.0% (v/v) to match the concentration of the synthesis bulk product regardless of the extent of dilution required.

The synthesis is a one-step labelling process with C18 purification using injections grade ethanol and water as the only solvents, therefore no residual solvents are present.

Radiochemical impurities are quantified by chromatographical methods (HPLC and TLC). Radiochemical purity determined by HPLC must not be less than 95.0%

Depending on the total produced radioactivity the bulk product is diluted to a fixed radioactivity concentration of about 500 MBq/ml.

The solution is filtered through a 0.22 μm membrane filter into sterile product vials. In addition to patient doses, sample vials are also dispensed from each production batch (chemical QC sample, microbiological QC sample, and a reference sample for retention). The final product is dispensed in a Grade A controlled environment. The integrity of the filters is tested after filtration by performing a bubble point test prior to product release. The fill weight/volume and radioactivity is checked for dispensed patient vials. The solution is ready for use after pre-release quality control and QP release.

Radioactivity is monitored with a dose calibrator after the labelling process in order to ensure successful labelling and during dispensing to verify dispensed doses.

TABLE 2A
Radiolabeling Process

| | Process 1 | | Process 2 | | Process 3 | | Process 4 | |
|---|---|---|---|---|---|---|---|---|
| Lu-177 | 40 GBq/ml in | | 40 GBq/ml in | | ~40 GBq/ml in | | 5500-15200 | mCi |
| | 0.04M HCl | | 0.04M HCl | | 0.05M HCl | | | |
| PSMA-I&T precursor | 1 000 | μg/ml | 463 | μg/ml | 463 | μg/ml | 1000 | μg/mL |

| Reaction Buffer | 4 ml 0.4M | | 4 ml 0.4M | | 4 ml 0.4M | | 82 mg/mL |
|---|---|---|---|---|---|---|---|
| | Sodium Acetate | | Sodium Acetate | | Sodium Acetate | | Sodium Ascorbate |
| | 2 ml 0.04M HCl | | 2 ml 0.04M HCl | | 1.6 ml 0.05M HCl | | |
| | (0.08 mmol HCl) | | (0.08 mmol HCl) | | (0.08 mmol HCl) | | |
| | 150 μl 20% L- | | 150 μl 20% L- | | 150 μl 20% L- | | |
| | ascorbic acid | | ascorbic acid | | ascorbic acid | | |
| Reaction Volume | 6-8 | ml | 6-8 | ml | 6-8 | ml | Dependent on |
| | | | | | | | reaction scale; |
| | | | | | | | 16 mL maximum |

| | | | | |
|---|---|---|---|---|
| Heating | Setpoint 110° C., | Setpoint 110° C., | Setpoint 110° C., | 85° C., 10 min |
| | 15 min | 15 min | 15 min | |
| Purification | Sep Pak C18 | Sep Pak C18 | Sep Pak C18 | N/A |
| Elution | 1.5 ml Ethanol- | 1.5 ml Ethanol- | 1.5 ml Ethanol- | N/A |
| | Water 1:1 | Water 1:1 | Water 1:1 | |
| Cassette flush | 8.5 ml Ascorbic | 8.5 ml Ascorbic | 8.5 ml Ascorbic | N/A |
| | acid 50 mg/ml | acid 50 mg/ml | acid 50 mg/ml, | |
| | (pH 5.5-7.0) | (pH 5.5-7.0) | pH 4.5 | |
| Formulation buffer | 42.5 mg/ml ascorbic | 42.5 mg/ml ascorbic | 31 mg/ml ascorbic | 33.0 mg/mL sodium |
| | acid, 7.5% Ethanol | acid, 7.5% Ethanol | acid, 3.8% Ethanol | ascorbate; 0.1 mg/mL |
| | (v/v %), pH 5.5-7.0 | (v/v %), pH 5.5-7.0 | (v/v %), pH 4.5 | DTPA; pH 4.25 |

The resulting Compositions 1-3 from Processes 1-3 are provided below in Table 2B, which provides the compositions for both a 1 ml volume and a vial of 10 ml or 20 ml for each composition.

TABLE 2B
[¹⁷⁷Lu]Lu-PSMA I&T compositions 1-3.

| | Compositions 1 and 2 | | | | Composition 3 | | | | |
|---|---|---|---|---|---|---|---|---|---|
| | Quantity | | Quantity/ | | Quantity | | Quantity/ | | |
| Component | per mL | | 10 mL vial | | per mL | | 20 mL vial | | Function |
| ¹⁷⁷Lu-PSMA | 1 GBq | | 10 GBq | | 0.5 GBq | | 10 GBq | | Radioactive |
| I&T | (27 mCi) | | (270 mCi) | | (13.5 mCi) | | (270 mCi) | | pharmaceutical |
| | | | | | | | | | ingredient (exact |
| | | | | | | | | | activity/volume |
| | | | | | | | | | adjusted according |
| | | | | | | | | | to dose strength) |
| Lu-PSMA I&T | <12 | μg | <80 | μg | <6 | μg | <80 | μg | Mass of |
| and related | | | | | | | | | pharmaceutical |
| substances | | | | | | | | | ingredient |
| Ascorbic acid | 42.5 | mg | 425 | mg | 31 | mg | 620 | mg | Radioprotectant |
| Disodium EDTA | 21 | μg | 210 | μg | 15.5 | μg | 310 | μg | Metal scavenger |

| Sodium | A sufficient | A sufficient | A sufficient | | A sufficient | | Present in |
|---|---|---|---|---|---|---|---|
| Bicarbonate | quantity | quantity | quantity | | quantity | | formulated |
| NaOH | A sufficient | A sufficient | A sufficient | | A sufficient | | Ascorbic acid |
| | quantity | quantity | quantity | | quantity | | Injection (USP) |
| | | | | | | | in sufficient |
| | | | | | | | amounts to control |
| | | | | | | | pH to 5.5-7.0 |
| Hydrochloric | None | None | 1.7 | mg/ml * | 34 | mg/ml * | *Theoretical amount. |
| acid | | | | | | | Added to adjust final |
| | | | | | | | formulation pH to 4.5 |

| | | | | | |
|---|---|---|---|---|---|
| Ethanol | 75 μL | 750 μL | 37.5 μL | 750 μL | Stabilizing agent |
| | (58.9 mg) | (589 mg) | (29.5 mg) | (589 mg) | |
| Water for | Sufficient to | Sufficient to | Sufficient to | Sufficient to | Vehicle |
| Injection | make 1 mL | make 10 mL | make 1 mL | make 20 mL | |

### Example 2: STABILITY OF THE RADIOCHEMICAL COMPOSITIONS

Stability of ¹⁷⁷Lu-PSMA I&T composition 1 was tested and the radiochemical purity and chemical properties were shown to provide adequate stability of 48 hours from the end-of-synthesis time for samples stored at +5° C., +20° C. and +40° C., Table 3.

Stability studies show that [¹⁷⁷Lu]Lu-PSMA I&T composition 3 (Tables 4A-H) comprising ascorbic acid 31 mg/ml and ethanol 3.8% (v/v) at a pH of 4.5 had improved stability and extended shelf life compared to [¹⁷⁷Lu]Lu-PSMA I&T composition 1.

The radiochemical purity and chemical properties (pH, impurities, visual properties) of [¹⁷⁷Lu]Lu-PSMA I&T in the formulation composition 3 were tested on the seven batches over a time span of 70 to 72 hours from the end-of-synthesis time. Stability samples of typical therapeutic dose radioactivity and volume were stored in different conditions covering typical storage, shipment and usage of the product, including temperatures ranging from +5° C. to +40° C.

Final radioactivity concentration in the sample solutions varied from 497 MBq/ml to 642 MBq/ml at the end of dispensing.

All stability samples met the set acceptance criteria. Radiochemical purity was ≥95,7% after 70 hours or 72 hours after end of synthesis time in all samples analyzed.

Based on the results ¹⁷⁷Lu-PSMA I&T solution in formulation composition 3 was stable under the different storage conditions tested.

TABLE 3
Stability Data of ¹⁷⁷Lu-PSMA I&T Composition 1.

| | | Stability data at | | | | |
|---|---|---|---|---|---|---|
| | | 48 h after end of synthesis | | | | |
| | | | | Batch | Batch | Batch |
| Stability criteria | | Batch | Batch | #3, | #3, | #3, |
| Test | Specification | #1 | #2 | ±5° C. | ±20° C. | ±40° C. |
| Visual | Clear, | Pass | Pass | Pass | Pass | Pass |
| inspection | colorless | | | | | |
| | or slightly | | | | | |
| | yellow liquid, | | | | | |
| | no visible | | | | | |
| | particles | | | | | |
| pH | 5.0-8.0 | 6.0 | 5.5 | 6.0 | 6.0 | 5.5 |
| Radio- | ≥95.0% as | 97.4% | 96.2% | 95.6% | 96.1 | 95.1% |
| chemical | ¹⁷⁷Lu- | | | | | |
| Purity | PSMA I&T | | | | | |
| (HPLC), | | | | | | |
| % | | | | | | |
| Radio- | ¹⁷⁷Lu- | 0% | 0% | 0% | 0% | 0% |
| chemical | colloid | | | | | |
| Purity by | ≤5.0% of | | | | | |
| TLC, % | radioactivity | | | | | |
| Chemical | ≤12.0 μg/ml | 1.6 | 2.2 | 1.9 | 1.8 | 1.8 |
| purity | Lu-PSMA | μg/ml | μg/ml | μg/ml | μg/ml | μg/ml |
| Lu-PSMA | I&T- | | | | | |
| I&T | concentration | | | | | |

TABLE 4A
Chemical Quality in the Validation Batches of ¹⁷⁷Lu-PSMA I&T Composition 3.

| QC TESTING | | | | | | | |
|---|---|---|---|---|---|---|---|
| Quality Attribute | | VALIDATION BATCH # | | | | | |
| Test | Specification | 1 | | 2 | | 3 | |
| Visual inspection | Clear, colorless to | Pass | | Pass | | Pass | |
| | yellow solution, no | | | | | | |
| | visible particles | | | | | | |
| Radiochemical | Co-elutes with | Pass | | Pass | | Pass | |
| Identity | reference standard ^{†} | | | | | | |
| pH | 4.0-5.0 | 4.5 | | 4.5 | | 4.5 | |
| Radionuclidic | Gamma ray energy | 113 keV and | | 113 keV and | | 113 keV and | |
| Identification keV | 113 ± 2 keV | 209 keV | | 209 keV | | 209 keV | |
| | 208 ± 4 keV | no other | | no other | | no other | |
| | (HPGe-detector) | significant | | significant | | significant | |
| | No other significant | peaks | | peaks | | peaks | |
| | peaks with gamma | detected | | detected | | detected | |
| | energy >100 keV | | | | | | |
| | are detected | | | | | | |
| Radiochemical | ≥95.0% as | 98.2% | | 98.2% | | 97.9% | |
| Purity (HPLC), % | ¹⁷⁷Lu-PSMA I&T | | | | | | |
| Radiochemical | ¹⁷⁷Lu-colloid ≤5.0% | 0% | | 0% | | 0% | |
| Purity by TLC, % | of radioactivity | | | | | | |
| Ethanol content, % | 2.5-4.5% | 3.5% | | 3.5% | | 3.2% | |
| (v/v) (GC) | | | | | | | |
| Chemical purity | Lu-PSMA I&T- | 1.2 | μg/ml | 1.2 | μg/ml | 1.2 | μg/ml |
| Lu-PSMA I&T | concentration ≤6.0 | | | | | | |
| | μg/ml | | | | | | |
| ¹⁷⁷Lu-PSMA-I&T | Reported | 271 | GBq/μmol | 275 | GBq/μmol | 191 | GBq/μmol |
| Specific activity | | | | | | | |
| Ascorbic acid | 15-35 mg/ml | 25 | mg/ml | 25 | mg/ml | 25 | mg/ml |
| concentration mg/ml | | | | | | | |
| Radionuclidic purity | ≥99.9% of the | 100.0% | | 100.0% | | 100.0% | |
| | total activity | | | | | | |
| | of ¹⁷⁷Lu | | | | | | |
| Bacterial endotoxins | <175 EU/20 ml | <5.00 | EU/ml | <5.00 | EU/ml | <5.00 | EU/ml |
| EU/ml | (measured <8.75 | | | | | | |
| | (EU/ml)) | | | | | | |

| | | | | |
|---|---|---|---|---|
| Filter integrity test | ≥3.32 bar tested | Pass | Pass | Pass |
| | with water | | | |
| Sterility | Sterile | Pass | Pass | Pass |

^{†} The radiometric RT of the test sample = UV RT of Reference Standard ± 5%.

TABLE 4B
Stability Data of ¹⁷⁷Lu-PSMA I&T Composition 3.

| STABILITY BATCH #1 | | 70 h post End of Synthesis | | | | | | | |
|---|---|---|---|---|---|---|---|---|---|
| VIAL # | | 1 | | 2 | | 3 | | 4 | |
| Radioactivity | | | | 9430 | MBq | 9440 | MBq | 9440 | MBq |
| Fill Volume | | 18.04 | ml | 18.10 | ml | 18.07 | ml | 18.03 | ml |
| Storage Condition | | +22.5° | C. | +32.5° | C. | +22.5° C./ | | +22.5° | C. |
| | | | | | | Inverted | | | |
| QC TESTING | | | | | | | | | |
| Validation criteria | | | | | | | | | |
| Test | Specification | | | | | | | | |
| Visual inspection | Clear, colorless to | Pass | | Pass | | Pass | | Pass | |
| | yellow solution, no | | | | | | | | |
| | visible particles | | | | | | | | |
| Radiochemical | Co-elutes with | Pass | | Pass | | Pass | | Pass | |
| Identity | reference standard ^{†} | | | | | | | | |
| pH | 4.0-5.0 | 4.5 | | 4.5 | | 4.5 | | 4.5 | |
| Radionuclidic | Gamma ray energy | 113 keV and | | 113 keV and | | 113 keV and | | 113 keV and | |
| Identification keV | 113 ± 2 keV | 209 keV | | 209 keV | | 209 keV | | 209 keV | |
| | 208 ± 4 keV | no other | | no other | | no other | | no other | |
| | (HPGe-detector) | significant | | significant | | significant | | significant | |
| | No other significant | peaks | | peaks | | peaks | | peaks | |
| | peaks with gamma | detected | | detected | | detected | | detected | |
| | energy >100 keV | | | | | | | | |
| | are detected | | | | | | | | |
| Radiochemical | ≥95.0% as | 96.7% | | 96.2% | | 95.9% | | 96.0% | |
| Purity (HPLC), % | ¹⁷⁷Lu-PSMA I&T | | | | | | | | |
| Radiochemical | ¹⁷⁷Lu-colloid ≤5.0% | 0% | | 0% | | 0% | | 0% | |
| Purity by TLC, % | of radioactivity | | | | | | | | |
| Ethanol content, % | 2.5-4.5% | N/A | | N/A | | N/A | | N/A | |
| (v/v) (GC) | | | | | | | | | |
| Chemical purity | Lu-PSMA I&T - | 1.3 | μg/ml | 1.3 | μg/ml | 1.2 | μg/ml | 1.3 | μg/ml |
| Lu-PSMA I&T | concentration ≤6.0 | | | | | | | | |
| | μg/ml | | | | | | | | |
| Ascorbic acid | 15-35 mg/ml | 25 | mg/ml | 25 | mg/ml | 25 | mg/ml | 25 | mg/ml |
| concentration mg/ml | | | | | | | | | |

^{†} The radiometric RT of the test sample = UV RT of Reference Standard ± 5%.

TABLE 4C
Stability Data of ¹⁷⁷Lu-PSMA I&T Composition 3.

| STABILITY BATCH #2 | | 72 h post End of Synthesis | | | | | | | |
|---|---|---|---|---|---|---|---|---|---|
| VIAL # | | 1 | | 2 | | 3 | | 4 | |
| Radioactivity | | 9650 | MBq | 9550 | MBq | 9540 | MBq | 9540 | MBq |
| Fill Volume | | 15.02 | ml | 14.86 | ml | 14.86 | ml | 14.87 | ml |
| Storage Condition | | +22.5° | C. | +32.5° | C. | +22.5° C./ | | +22.5° | C. |
| | | | | | | Inverted | | | |
| QC TESTING | | | | | | | | | |
| Validation criteria | | | | | | | | | |
| Test | Specification | | | | | | | | |
| Visual inspection | Clear, colorless to | Pass | | Pass | | Pass | | Pass | |
| | yellow solution, no | | | | | | | | |
| | visible particles | | | | | | | | |
| Radiochemical | Co-elutes with | Pass | | Pass | | Pass | | Pass | |
| Identity | reference standard ^{†} | | | | | | | | |
| pH | 4.0-5.0 | 4.5 | | 4.5 | | 4.5 | | 4.5 | |
| Radionuclidic | Gamma ray energy | 113 keV and | | 113 keV and | | 113 keV and | | 113 keV and | |
| Identification keV | 113 ± 2 keV | 209 keV | | 209 keV | | 209 keV | | 209 keV | |
| | 208 ± 4 keV | no other | | no other | | no other | | no other | |
| | (HPGe-detector) | significant | | significant | | significant | | significant | |
| | No other significant | peaks | | peaks | | peaks | | peaks | |
| | peaks with gamma | detected | | detected | | detected | | detected | |
| | energy >100 keV | | | | | | | | |
| | are detected | | | | | | | | |
| Radiochemical | ≥95.0% as | 96.0% | | 95.7% | | 96.1% | | 96.1% | |
| Purity (HPLC), % | ¹⁷⁷Lu-PSMA I&T | | | | | | | | |
| Radiochemical | ¹⁷⁷Lu-colloid ≤5.0% | 0% | | 0% | | 0% | | 0% | |
| Purity by TLC, % | of radioactivity | | | | | | | | |
| Ethanol content, % | 2.5-4.5% | N/A | | N/A | | N/A | | N/A | |
| (v/v) (GC) | | | | | | | | | |
| Chemical purity | Lu-PSMA I&T - | 1.2 | μg/ml | 1.2 | μg/ml | 1.2 | μg/ml | 1.2 | μg/ml |
| Lu-PSMA I&T | concentration ≤6.0 | | | | | | | | |
| | μg/ml | | | | | | | | |
| Ascorbic acid | 15-35 mg/ml | 25 | mg/ml | 25 | mg/ml | 25 | mg/ml | 25 | mg/ml |
| concentration mg/ml | | | | | | | | | |

^{†} The radiometric RT of the test sample = UV RT of Reference Standard ± 5%.

TABLE 4D
Stability Data of ¹⁷⁷Lu-PSMA I&T composition 3.

| STABILITY BATCH #3 | | 72 h post End of Synthesis | | | | | | | |
|---|---|---|---|---|---|---|---|---|---|
| VIAL # | | 1 | | 2 | | 3 | | 4 | |
| Radioactivity | | 9840 | MBq | 9540 | MBq | 9530 | MBq | 9530 | MBq |
| Fill Volume | | 17.51 | ml | 17.01 | ml | 16.99 | ml | 16.98 | ml |
| Storage Condition | | +22.5° | C. | +32.5° | C. | ≥75% RH | | +5° | C. |
| | | | | | | (Ambient) | | | |
| QC TESTING | | | | | | | | | |
| Validation criteria | | | | | | | | | |
| Test | Specification | | | | | | | | |
| Visual inspection | Clear, colorless to | Pass | | Pass | | Pass | | Pass | |
| | yellow solution, no | | | | | | | | |
| | visible particles | | | | | | | | |
| Radiochemical | Co-elutes with | Pass | | Pass | | Pass | | Pass | |
| Identity | reference standard ^{†} | | | | | | | | |
| pH | 4.0-5.0 | 4.5 | | 4.5 | | 4.5 | | 4.5 | |
| Radionuclidic | Gamma ray energy | 113 keV and | | 113 keV and | | 113 keV and | | 113 keV and | |
| Identification keV | 113 ± 2 keV | 209 keV | | 209 keV | | 209 keV | | 209 keV | |
| | 208 ± 4 keV | no other | | no other | | no other | | no other | |
| | (HPGe-detector) | significant | | significant | | significant | | significant | |
| | No other significant | peaks | | peaks | | peaks | | peaks | |
| | peaks with gamma | detected | | detected | | detected | | detected | |
| | energy >100 keV | | | | | | | | |
| | are detected | | | | | | | | |
| Radiochemical | ≥95.0% as | 95.8% | | 96.0% | | 96.1% | | 96.0% | |
| Purity (HPLC), % | ¹⁷⁷Lu-PSMA I&T | | | | | | | | |
| Radiochemical | ¹⁷⁷Lu-colloid ≤5.0% | 0% | | 0% | | 0% | | 0% | |
| Purity by TLC, % | of radioactivity | | | | | | | | |
| Ethanol content, % | 2.5-4.5% | N/A | | N/A | | N/A | | N/A | |
| (v/v) (GC) | | | | | | | | | |
| Chemical purity | Lu-PSMA I&T - | 1.0 | μg/ml | 0.9 | μg/ml | 1.0 | μg/ml | 1.0 | μg/ml |
| Lu-PSMA I&T | concentration ≤6.0 | | | | | | | | |
| | μg/ml | | | | | | | | |
| Ascorbic acid | 15-35 mg/ml | 15 | mg/ml | 15 | mg/ml | 15 | mg/ml | 15 | mg/ml |
| concentration mg/ml | | | | | | | | | |

^{†} The radiometric RT of the test sample = UV RT of Reference Standard ± 5%.

TABLE 4E
Stability Data of ¹⁷⁷Lu-PSMA I&T composition 3.

| | | STABILITY BATCH #4 | | | | |
|---|---|---|---|---|---|---|
| | | 72 h post End of Synthesis | | | | |
| VIAL # | | 1 | 2 | 3 | 4 | 5 |
| Radioactivity | | 9490 | 9600 | 9600 | 9600 | 9590 |
| | | MBq | MBq | MBq | MBq | MBq |
| Fill Volume | | 17.19 ml | 17.42 ml | 17.45 ml | 17.44 ml | 17.42 ml |
| Storage Condition | | ±22.5° C. | ±32.5° C. | ±40° C. | ±5° C. | ±5° C. |
| QC TESTING | | | | | | |
| Validation | | | | | | |
| criteria | Specifi- | | | | | |
| Test | cation | | | | | |
| Visual | Clear, | Pass | Pass | Pass | Pass | Pass |
| inspection | colorless to | | | | | |
| | yellow | | | | | |
| | solution, | | | | | |
| | no visible | | | | | |
| | particles | | | | | |
| Radiochemical | Co-elutes | Pass | Pass | Pass | Pass | Pass |
| Identity | with | | | | | |
| | reference | | | | | |
| | standard ^{†} | | | | | |
| pH | 4.0-5.0 | 4.5 | 4.5 | 4.5 | 4.5 | 4.5 |
| Radionuclidic | Gamma ray | 113 keV | 113 keV | 113 keV | 113 keV | 113 keV |
| Identification | energy | and | and | and | and | and |
| keV | 113 ± 2 keV | 209 keV | 209 keV | 209 keV | 209 keV | 209 keV |
| | 208 ± 4 keV | no other | no other | no other | no other | no other |
| | (HPGe- | signifi- | signifi- | signifi- | signifi- | signifi- |
| | detector) | cant | cant | cant | cant | cant |
| | No other | peaks | peaks | peaks | peaks | peaks |
| | significant | detected | detected | detected | detected | detected |
| | peaks with | | | | | |
| | gamma | | | | | |
| | energy | | | | | |
| | >100 | | | | | |
| | keV are | | | | | |
| | detected | | | | | |
| Radiochemical | ≥95.0% as | 96.5% | 95.9% | 95.8% | 96.3% | 96.6% |
| Purity | ¹⁷⁷Lu- | | | | | |
| (HPLC), | PSMA | | | | | |
| % | I&T | | | | | |
| Radiochemical | ¹⁷⁷Lu- | 0% | 0% | 0% | 0% | 0% |
| Purity by | colloid | | | | | |
| TLC, % | ≤5.0% of | | | | | |
| | radio- | | | | | |
| | activity | | | | | |
| Ethanol | 2.5-4.5% | N/A | N/A | N/A | N/A | N/A |
| content, | | | | | | |
| % (v/v) | | | | | | |
| (GC) | | | | | | |
| Chemical | Lu-PSMA | 1.0 μg/ml | 1.0 μg/ml | 1.0 μg/ml | 1.0 μg/ml | 1.0 μg/ml |
| purity | I&T- | | | | | |
| Lu- | concen- | | | | | |
| PSMA | tration | | | | | |
| I&T | ≤6.0 μg/ml | | | | | |
| Ascorbic | 15-35 | 25 mg/ml | 25 mg/ml | 25 mg/ml | 25 mg/ml | 25 mg/ml |
| acid | mg/ml | | | | | |
| concentration | | | | | | |
| mg/ml | | | | | | |

^{†} The radiometric RT of the test sample = UV RT of Reference Standard ±5%.

TABLE 4F
Stability Data of ¹⁷⁷Lu-PSMA I&T Composition 3.

| STABILITY BATCH #5 | | 72 h post End of Synthesis | | | | | | | |
|---|---|---|---|---|---|---|---|---|---|
| VIAL # | | 1 | | 2 | | 3 | | 4 | |
| Radioactivity | | 9930 | MBq | 9800 | MBq | 9800 | MBq | 9810 | MBq |
| Fill Volume | | 19.91 | ml | 19.69 | ml | 19.68 | ml | 19.73 | ml |
| Storage Condition | | +22.5° | C. | +32.5° | C. | +5° | C. | +5° | C. |
| QC TESTING | | | | | | | | | |
| Validation criteria | | | | | | | | | |
| Test | Specification | | | | | | | | |
| Visual inspection | Clear, colorless to | Pass | | Pass | | Pass | | Pass | |
| | yellow solution, no | | | | | | | | |
| | visible particles | | | | | | | | |
| Radiochemical | Co-elutes with | Pass | | Pass | | Pass | | Pass | |
| Identity | reference standard ^{†} | | | | | | | | |
| pH | 4.0-5.0 | 4.5 | | 4.5 | | 4.5 | | 4.5 | |
| Radionuclidic | Gamma ray energy | 113 keV and | | 113 keV and | | 113 keV and | | 113 keV and | |
| Identification keV | 113 ± 2 keV | 209 keV | | 209 keV | | 209 keV | | 209 keV | |
| | 208 ± 4 keV | no other | | no other | | no other | | no other | |
| | (HPGe-detector) | significant | | significant | | significant | | significant | |
| | No other significant | peaks | | peaks | | peaks | | peaks | |
| | peaks with gamma | detected | | detected | | detected | | detected | |
| | energy >100 keV | | | | | | | | |
| | are detected | | | | | | | | |
| Radiochemical | ≥95.0% as | 97.0% | | 97.0% | | 96.8% | | 97.3% | |
| Purity (HPLC), % | ¹⁷⁷Lu-PSMA I&T | | | | | | | | |
| Radiochemical | ¹⁷⁷Lu-colloid ≤5.0% | 0% | | 0% | | 0% | | 0% | |
| Purity by TLC, % | of radioactivity | | | | | | | | |
| Ethanol content, % | 2.5-4.5% | N/A | | N/A | | N/A | | N/A | |
| (v/v) (GC) | | | | | | | | | |
| Chemical purity | Lu-PSMA I&T- | 0.9 | μg/ml | 0.9 | μg/ml | 0.9 | μg/ml | 0.9 | μg/ml |
| Lu-PSMA I&T | concentration ≤6.0 | | | | | | | | |
| | μg/ml | | | | | | | | |
| Ascorbic acid | 15-35 mg/ml | 25 | mg/ml | 25 | mg/ml | 25 | mg/ml | 25 | mg/ml |
| concentration mg/ml | | | | | | | | | |

^{†} The radiometric RT of the test sample = UV RT of Reference Standard ± 5%.

TABLE 4G
Stability Data of ¹⁷⁷Lu-PSMA I&T Composition 3.

| | STABILITY BATCH #6 | | | |
|---|---|---|---|---|
| | 72 h post End of Synthesis | | | |
| VIAL # | 1 | | 4 * | |
| Radioactivity | 9960 | MBq | 9800 | MBq |
| Fill Volume | 19.69 | ml | 19.40 | ml |
| Storage Condition | +22.5° | C. | +40° | C. |

| QC TESTING | | | | | | |
|---|---|---|---|---|---|---|
| Validation criteria | | | | | | |
| Test | Specification | | | | | |
| Visual inspection | Clear, colorless to | | Pass | | Pass | |
| | yellow solution, no | | | | | |
| | visible particles | | | | | |
| Radiochemical | Co-elutes with | | Pass | | Pass | |
| Identity | reference standard ^{†} | | | | | |
| pH | 4.0-5.0 | | 4.5 | | 4.5 | |
| Radionuclidic | Gamma ray energy | | 113 keV and | | 113 keV and | |
| Identification | 113 ± 2 | keV | 209 keV | | 209 keV | |
| keV | 208 ± 4 | keV | no other significant | | no other significant | |
| | (HPGe-detector) | | peaks detected | | peaks detected | |
| | No other significant | | | | | |
| | peaks with gamma | | | | | |
| | energy > 100 keV are | | | | | |
| | detected | | | | | |
| Radiochemical | ≥95.0% as ¹⁷⁷Lu- | | 96.7% | | 95.8% | |
| Purity (HPLC), % | PSMA I&T | | | | | |
| Radiochemical | ¹⁷⁷Lu-colloid ≤ 5.0% | | 0% | | 0% | |
| Purity by TLC, % | of radioactivity | | | | | |
| Ethanol content, % | 2.5-4.5% | | N/A | | N/A | |
| (v/v) (GC) | | | | | | |
| Chemical purity | Lu-PSMA I&T - | | 1.0 | μg/ml | 1.0 | μg/ml |
| Lu-PSMA I&T | concentration ≤ | | | | | |
| | 6.0 μg/ml | | | | | |
| Ascorbic acid | 15-35 | mg/ml | 25 | mg/ml | 25 | mg/ml |
| concentration | | | | | | |
| mg/ml | | | | | | |

^{†} The radiometric RT of the test sample = UV RT of Reference Standard ± 5%.

TABLE 4H
Stability Data of ¹⁷⁷Lu-PSMA I&T composition 3.

| | STABILITY BATCH #7 | | | | | |
|---|---|---|---|---|---|---|
| | 72 h post End of Synthesis | | | | | |
| VIAL # | 1 | | 2 | | 3 | |
| Radioactivity | 9870 | MBq | 9880 | MBq | 9910 | MBq |
| Fill Volume | 19.31 | ml | 19.34 | ml | 19.43 | ml |

| | | | |
|---|---|---|---|
| Storage Condition | +22.5° C./ | +32.5° C./ | +5° C./ |
| | Inverted | Inverted | Inverted |

| QC TESTING | | | | | | | | |
|---|---|---|---|---|---|---|---|---|
| Validation | | | | | | | | |
| criteria | | | | | | | | |
| Test | Specification | | | | | | | |
| Visual | Clear, colorless to | | Pass | | Pass | | Pass | |
| inspection | yellow solution, | | | | | | | |
| | no visible | | | | | | | |
| | particles | | | | | | | |
| Radiochemical | Co-elutes with | | Pass | | Pass | | Pass | |
| Identity | reference standard ^{†} | | | | | | | |
| pH | 4.0-5.0 | | 4.5 | | 4.5 | | 4.5 | |
| Radionuclidic | Gamma ray | | 113 keV and | | 113 keV and | | 113 keV and | |
| Identification | energy | | 209 keV | | 209 keV | | 209 keV | |
| keV | 113 ± 2 | keV | no other | | no other | | no other | |
| | 208 ± 4 | keV | significant | | significant | | significant | |
| | (HPGe-detector) | | peaks detected | | peaks detected | | peaks detected | |
| | No other | | | | | | | |
| | significant peaks | | | | | | | |
| | with gamma | | | | | | | |
| | energy > 100 keV | | | | | | | |
| | are detected | | | | | | | |
| Radiochemical | ≥95.0% as ¹⁷⁷Lu- | | 96.5% | | 96.4% | | 96.3% | |
| Purity (HPLC), | PSMA I&T | | | | | | | |
| % | | | | | | | | |
| Radiochemical | ¹⁷⁷Lu-colloid ≤ | | 0% | | 0% | | 0% | |
| Purity by TLC, | 5.0% of | | | | | | | |
| % | radioactivity | | | | | | | |
| Ethanol content, | 2.5-4.5% | | N/A | | N/A | | N/A | |
| % (v/v) (GC) | | | | | | | | |
| Chemical purity | Lu-PSMA I&T - | | 1.0 | μg/ml | 0.9 | μg/ml | 1.0 | μg/ml |
| Lu-PSMA I&T | concentration ≤ | | | | | | | |
| | 6.0 μg/ml | | | | | | | |
| Ascorbic acid | 15-35 | mg/ml | 25 | mg/ml | 25 | mg/ml | 25 | mg/ml |
| concentration | | | | | | | | |
| mg/ml | | | | | | | | |

^{†} The radiometric RT of the test sample = UV RT of Reference Standard ± 5%.

TABLE 4I
Release Data from Composition 4 Commercial Scale Development Batches

| | Specification | | | | |
|---|---|---|---|---|---|
| Characteristics | (Composition 4) | | 8017-PD15 | 8017-PD16 | 8017-PD17 |
| Appearance | Clear, colorless to | | Pass | Pass | Pass |
| | yellow solution | | | | |
| | without visible | | | | |
| | particles | | | | |
| pH | 4.0-4.5 | | 4.2 | 4.2 | 4.2 |
| Ascorbic Acid | 28-38 | mg/mL | 32 | 33 | 35 |
| Ethanol | N/A | | N/A | N/A | N/A |
| Radionuclidic | Gamma ray energy | | 113 keV and | 113 keV and | 113 keV and |
| identification | 113 ± 1 keV and | | 209 keV | 209 keV | 209 keV |
| | 208 ± 1 keV | | no other | no other | no other |
| | | | significant | significant | significant |
| | | | peaks | peaks | peaks |
| | | | detected | detected | detected |
| Radiochemical Identity | Relative retention | | Pass | Pass | Pass |
| | time of 0.70 to 0.75 | | | | |
| | compared to PSMA | | | | |
| | I&T in HPLC | | | | |
| | standard | | | | |
| Radiochemical purity | Not less than | | 96 | 97 | 97 |
| by HPLC method | (NLT, ≤) 95% | | | | |
| Radiochemical purity | N/A | | N/A | N/A | N/A |
| by TLC method | | | | | |
| Unbound | Not more than | | 0% | 0% | 0% |
| [¹⁷⁷Lu]lutetium | (NMT, ≥) 1% | | | | |
| PSMA I&T Content | 3.0-8.0 | μg/mL | 4.5 | 5.0 | 5.0 |
| Radionuclidic purity | Not less than | | 100% | 100% | 100% |
| | (NLT, ≥) 99.9% | | | | |
| | [¹⁷⁷Lu]Lu³⁺ | | | | |
| ¹⁷⁷Lu-PSMA-I&T | N/A | | N/A | N/A | N/A |
| Specific activity | | | | | |
| Apparent Molar | 2.0-7.0 | mCi/nmol | 4.9 | 4.4 | 4.5 |
| Activity | | | | | |
| Endotoxins | 11.7 | EU/mL | N/A | N/A | N/A |
| Radioactivity | 12.0-14.6 | mCi/mL | 13.2 | 13.3 | 13.4 |
| Concentration | | | | | |

| | | | | |
|---|---|---|---|---|
| Sterility | Sterile | N/A | N/A | N/A |

TABLE 4J
Stability data from composition 4

| | Specification | | | | |
|---|---|---|---|---|---|
| Characteristics | (Composition 4) | | 8017-PD15 | 8017-PD16 | 8017-PD17 |
| pH | 4.0-4.5 | | 4.3 | 4.2 | 4.2 |
| Ascorbic Acid | 28-38 | mg/mL | 32 | 33 | 34 |
| Radiochemical purity | Not less than | | 95 | 95 | 95 |
| by HPLC method | (NLT, ≤) 95% | | | | |
| Unbound | Not more than | | 0% | 0% | 0% |
| [¹⁷⁷Lu]lutetium | (NMT, ≥) 1% | | | | |
| PSMA I&T Content | 3.0-8.0 | μg/mL | 4.5 | 4.8 | 5.0 |
| Apparent Molar | 2.0-7.0 | mCi/nmol | 4.9 | 4.6 | 4.5 |
| Activity | | | | | |
| Stability Testing Time | >72 | hours | 72 | 72 | 96 |

Specifications for the [¹⁷⁷Lu]Lu-PSMA I&T solution are presented in the Table 5 below. The specifications listed were used as release parameters except for sterility testing. Sterility was tested on all batches post-release.

TABLE 5
Specifications for [¹⁷⁷Lu]Lu-PSMA I&T

| | Specification | | Specification | | Specification | |
|---|---|---|---|---|---|---|
| Characteristics | (Composition 1) | | (Composition 3) | | (Composition 4) | |
| Appearance | Clear, colorless or | | Clear, colorless or | | Clear, colorless to | |
| | slightly yellow | | slightly yellow | | yellow solution | |
| | liquid, no visible | | liquid, no visible | | without visible | |
| | particles | | particles | | particles | |
| pH | 5.0-8.0 | | 4.0-5.0 | | 4.0-4.5 | |
| Ascorbic Acid | 20-70 | mg/ml | 15-35 | mg/ml | 28-38 | mg/mL |
| Ethanol | 5.0% to 8.0% | | 2.5% to 4.5% | | N/A | |
| Radionuclidic | Gamma ray energy | | Gamma ray energy | | Gamma ray energy | |
| identification | 113 ± 10 keV and | | 113 ± 10 keV and | | 113 ± 1 keV and | |
| | 208 ± 12 keV | | 208 ± 12 keV | | 208 ± 1 keV | |
| Radiochemical Identity | The radiometric RT | | The radiometric RT | | Relative retention | |
| | of the test sample = | | of the test sample = | | time of 0.70 to | |
| | UV RT of | | UV RT of | | 0.75 compared to | |
| | Reference | | Reference | | PSMA I&T in | |
| | Standard ± 5%. | | Standard ± 5%. | | HPLC standard | |
| Radiochemical purity | ≥95.0% | | ≥95.0% | | Not less than | |
| by HPLC method | | | | | (NLT, ≤) 95% | |
| Radiochemical purity | ¹⁷⁷Lu-colloid ≤ 5.0% | | ¹⁷⁷Lu-colloid ≤ 5.0% | | N/A | |
| by TLC method | | | | | | |
| Unbound | N/A | | N/A | | Not more than | |
| [¹⁷⁷Lu]lutetium | | | | | (NMT, ≥) 1% | |
| PSMA I&T Content | Lu-PSMA I&T - | | Lu-PSMA I&T - | | 3.0-8.0 | μg/mL |
| | concentration [symbol] | | concentration [symbol] | | | |
| | 12.0 μg/mL | | 12.0 μg/mL | | | |
| Radionuclidic purity | ≥99.9% of the total | | ≥99.9% of the total | | Not less than | |
| | activity of ¹⁷⁷Lu at | | activity of ¹⁷⁷Lu at | | (NLT, ≥) 99.9% | |
| | the end of the shelf | | the end of the shelf | | [¹⁷⁷Lu]Lu³⁺ | |
| | life | | life | | | |
| ¹⁷⁷Lu-PSMA-I&T | Reported | | Reported | | N/A | |
| Specific activity | | | | | | |
| Apparent Molar | N/A | | N/A | | 2.0-7.0 | mCi/nmol |
| Activity | | | | | | |
| Endotoxins | <17.5 | IU/ml | <17.5 | IU/ml | 11.7 | EU/mL |

| | | |
|---|---|---|
| Radioactive content | 90%-110% at the | 90%-110% at the |
| | date and time stated | date and time stated |
| | on the label | on the label |

| | | | |
|---|---|---|---|
| Sterility | Sterile | Sterile | Sterile |

### Example 3: RADIOCHEMICAL PURITY OF ¹⁷⁷Lu-PSMA I&T IN DIFFERENT FORMULATIONS COMPOSITIONS

The example demonstrates radiochemical stability of ¹⁷⁷Lu-PSMA I&T in formulation compositions at different pH values. The shelf life of ¹⁷⁷Lu-PSMA I&T is restricted by high rate of radiolysis during preparation and storage resulting in decomposition of ¹⁷⁷Lu-PSMA I&T and formation of radiochemical impurities. This eventually results in radiochemical purity of ¹⁷⁷Lu-PSMA I&T solution to fall below acceptance limit of 95.0%.

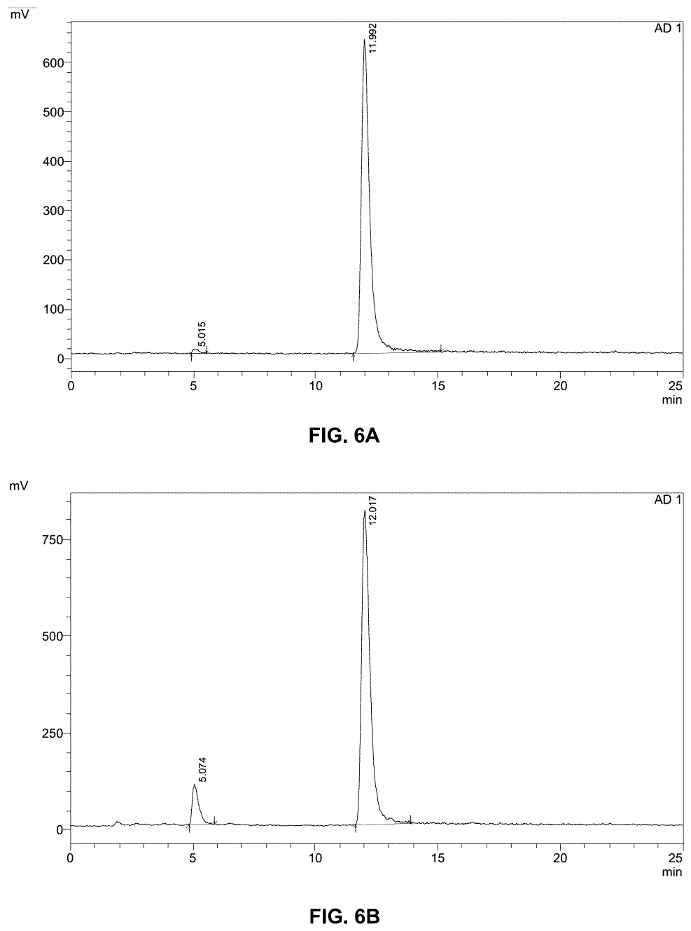

Formation of a particular radiochemical impurity of ¹⁷⁷Lu-PSMA I&T have been observed having retention time of about 5.2 minutes by HPLC with Phenomenex Luna C18 column (3 μm, 150 mm×4.6 mm) using 0.1% trifluoroacetic acid in water (Mobile phase A) and 0.1% trifluoroacetic acid in water:acetonitrile (10:90% v/v) (Mobile phase B) and isocratic method of 23% Mobile phase B at temperature of 40° C. The impurity referred herein having retention time of about 5.2 minutes is exemplified in the chromatograms in FIGS. 6A-11B.

In experiments conducted previously, lowering formulation radioactivity concentration was sufficient to reduce the formation of the impurity eluting at about 5.2 minutes and maintaining radiochemical stability of [¹⁷⁷Lu]Lu-PSMA I&T solutions above 95.0% for 72 hours.

In this example, six experiments were conducted where [¹⁷⁷Lu]Lu-PSMA I&T was prepared in different formulation compositions with varying ascorbic acid concentration, pH and radioactivity concentration. Product formulation details are described in Table 6.

TABLE 6
[¹⁷⁷Lu]Lu-PSMA I&T formulation compositions evaluated

| | | | | | Radio- | |
|---|---|---|---|---|---|---|
| Experi- | | | Sample | | activity | Storage |
| ment | Formulation | | Radio- | Sample | Concen- | Temper- |
| no | composition | pH | activity | Volume | tration | ature |
| 1 | high RAC, | 7 | 12 810 | 10 ml | 1281 | 22.5° C. |
| | 42.5 mg/ml | | MBq | | MBq/ml | |
| | Ascorbic acid | | | | | |
| 2 | high RAC, | 4.5 | 12 780 | 10 ml | 1278 | 22.5° C. |
| | 42.5 mg/ml | | MBq | | MBq/ml | |
| | Ascorbic acid | | | | | |
| 3 | high RAC, | 3.5 | 13 110 | 10 ml | 1311 | 22.5° C. |
| | 42.5 mg/ml | | MBq | | MBq/ml | |
| | Ascorbic acid | | | | | |
| 4 | low RAC, | 4.5 | 11 580 | 20 ml | 579 | 22.5° C. |
| | 31 mg/ml | | MBq | | MBq/ml | |
| | Ascorbic acid | | | | | |
| 5 | low RAC, | 5 | 12 520 | 20 ml | 626 | 22.5° C. |
| | 31 mg/ml | | MBq | | MBq/ml | |
| | Ascorbic acid | | | | | |
| 6 | low RAC, | 4.5 | 11 770 | 20 ml | 589 | 22.5° C. |
| | 31 mg/ml | | MBq | | MBq/ml | |
| | Ascorbic acid | | | | | |

High radioactivity concentration (high RAC) in sample solutions were 1278 MBq/ml, 1281 MBq/ml and 1311 MBq/ml measured at the end of production. Low radioactivity concentration (low RAC) in sample solutions were 579 MBq/ml, 589 MBq/ml and 626 MBq/ml measured at the end of production. Radiochemical purity of each solution was followed by HPLC up to 71-93 hours post-radiolabeling. All solutions were stored at 22.5° C.

FIG. 5 shows results of radiochemical purity analyses at different time points determined by HPLC.

FIG. 6A and FIG. 6B show HPLC radio-chromatograms of experiment 1 at 0 and 71 hours post EOS, respectively.

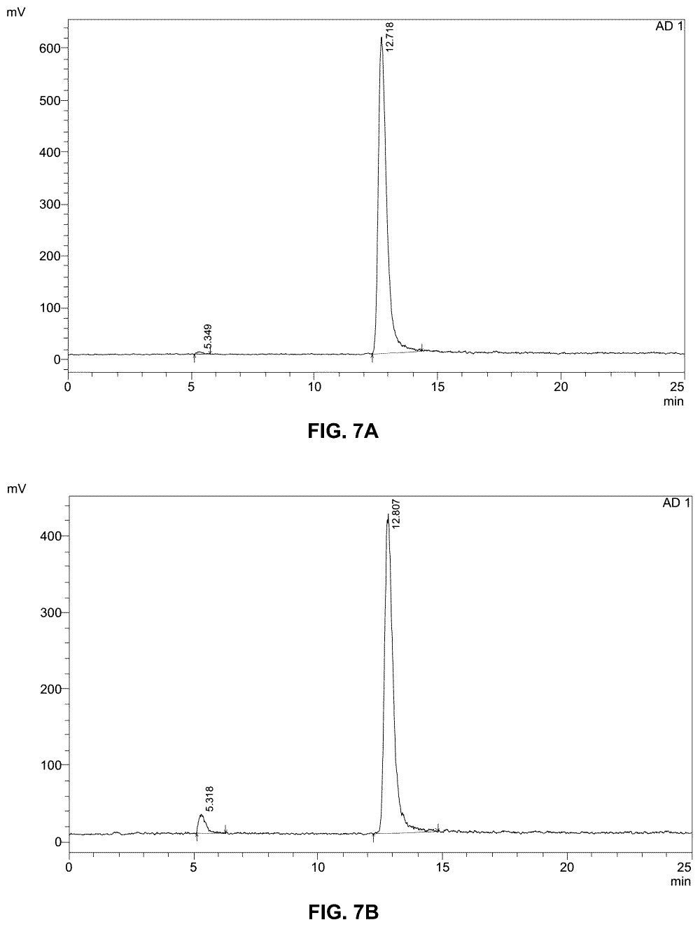

FIG. 7A and FIG. 7B show HPLC radio-chromatograms of experiment 2 at 0 and 71 hours post EOS, respectively.

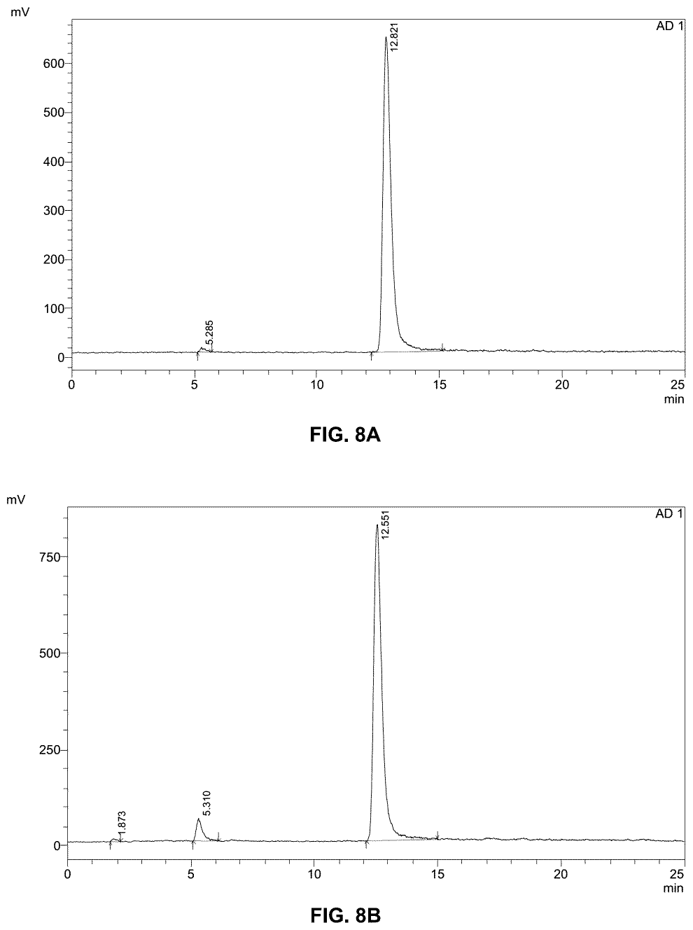

FIG. 8A and FIG. 8B show HPLC radio-chromatograms of experiment 3 at 0 and 90 hours post EOS, respectively.

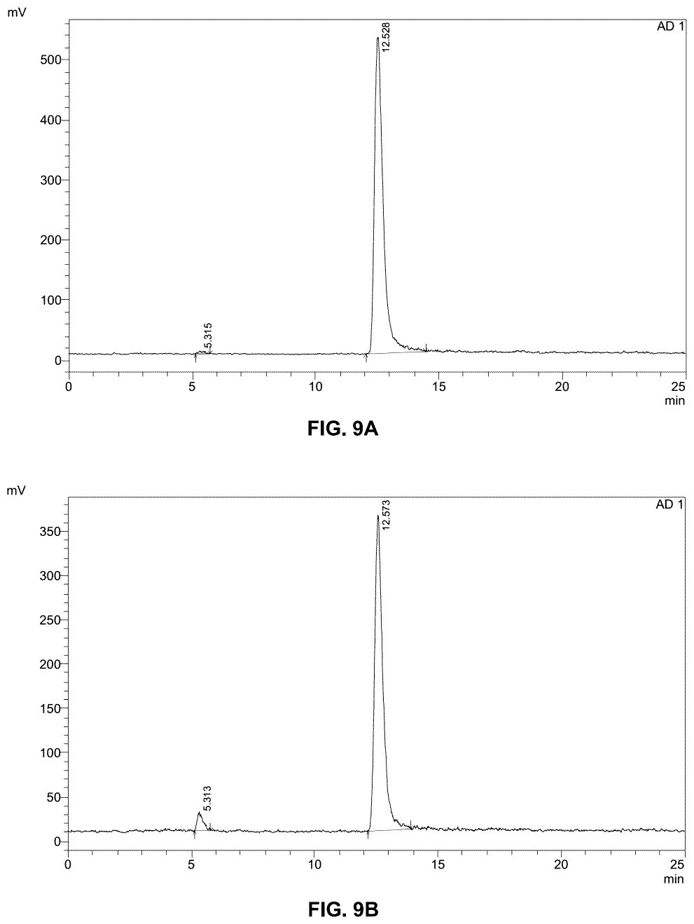

FIG. 9A and FIG. 9B show HPLC radio-chromatograms of experiment 4 at 0 and 92 hours post EOS, respectively.

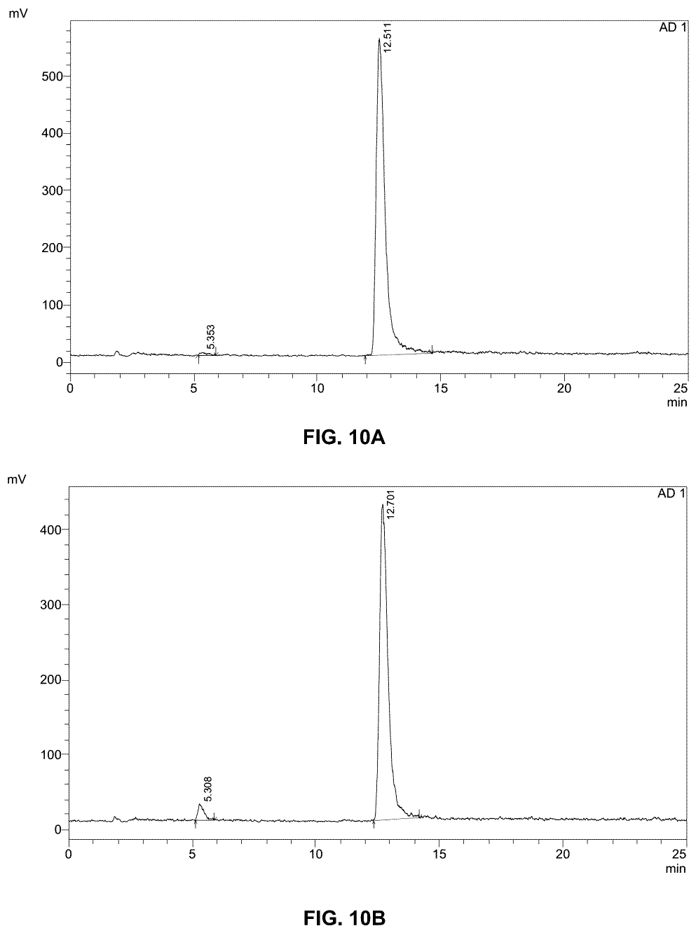

FIG. 10A and FIG. 10B show HPLC radio-chromatograms of experiment 5 at 0 and 71 hours post EOS, respectively.

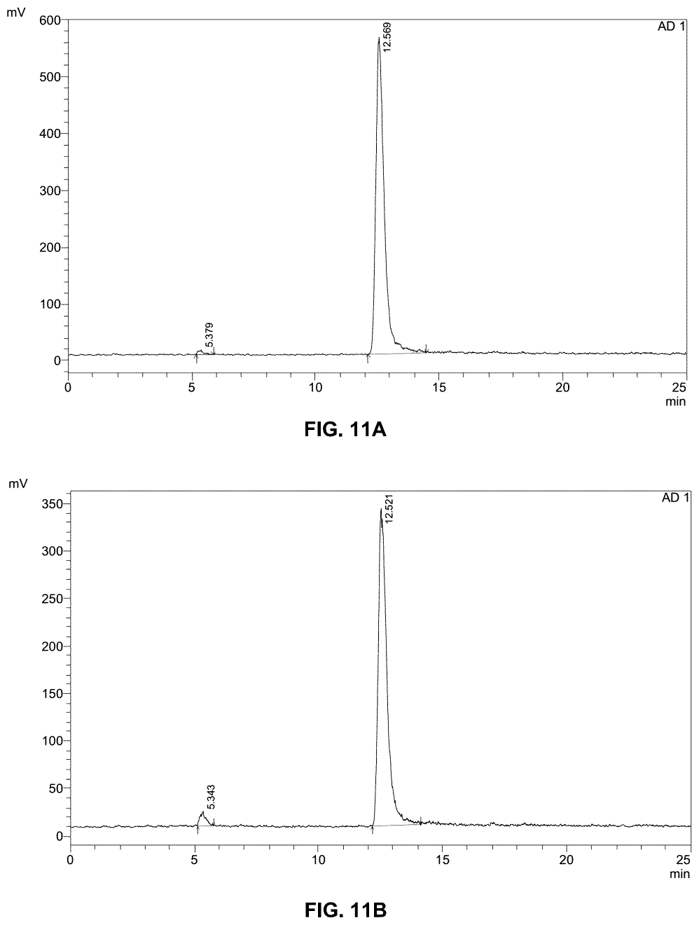

FIG. 11A and FIG. 11B show HPLC radio-chromatograms of experiment 6 at 0 and 93 hours post EOS, respectively.

The radiochemical stability results for each experiment at different time points are provided in Tables 5-10.

TABLE 5
Experiment 1
Radiochemical purity by HPLC (hours post EOS)

| T = 0 h | T = 24 h | T = 46 h | T = 64 h | T = 71 h |
|---|---|---|---|---|
| 99.0% | 96.5% | 93.3% | N/A | 91.2% |

TABLE 6
Experiment 2
Radiochemical purity by HPLC (hours post EOS)

| T = 0 h | T = 24 h | T = 46 h | T = 64 h | T = 71 h |
|---|---|---|---|---|
| 99.4% | 98.0% | 96.7% | 95.3% | 95.2% |

TABLE 7
Experiment 3
Radiochemical purity by HPLC (hours post EOS)

| T = 0 h | T = 24 h | T = 46 h | T = 64 h | T = 72h | T = 90 h |
|---|---|---|---|---|---|
| 99.1% | 97.7% | 96.5% | N/A | N/A | 94.5% |

TABLE 8
Experiment 4
Radiochemical purity by HPLC (hours post EOS)

| T = 0h | T = 19h | T = 46 h | T = 71 h | T = 92 h |
|---|---|---|---|---|
| 99.4% | 98.3% | 97.5% | 96.8% | 96.0% |

TABLE 9
Experiment 5
Radiochemical purity by HPLC (hours post EOS)

| T = 0 h | T = 25 h | T = 47 h | T = 71 h |
|---|---|---|---|
| 99.2% | 98.4% | 97.3% | 96.5% |

TABLE 10
Experiment 6
Radiochemical purity by HPLC (hours post EOS)

| T = 0 h | T = 20 h | T = 44 h | T = 69 h | T = 93 h |
|---|---|---|---|---|
| 99.1% | 98.7% | 98.0% | 97.4% | 97.0% |

In the example, formulation composition pH had a considerable effect on the radiochemical stability of ¹⁷⁷Lu-PSMA I&T and more specifically in the formation of radiochemical impurity eluting at about 5.2 minutes as exemplified in FIGS. 6A-11B. In high RAC solutions, the decrease in radiochemical purity over time at pH 4.5 was two times slower than at pH 7.

Further decrease of formulation pH to 3.5 did not show measurable improvement in radiochemical stability compared to pH 4.5 solution. It is possible that ascorbic acid solution at pH 4.5, being near pKa value of ascorbic acid, already possesses sufficient quantity of protons to serve as inhibitors against radiolysis of ¹⁷⁷Lu-PSMA I&T and reduce formation of the radiochemical impurity eluting at about 5.2 minutes.

Incorporation of solution pH of 4.5 in the lower RAC formulation further improved radiochemical stability of ¹⁷⁷Lu-PSMA I&T. In the lower RAC formulation, change in formulation pH from 5 to 4.5 had similar effect on radiochemical stability as increasing ascorbic acid concentration from 21 mg/ml to 31 mg/ml.

The radiochemical purity of a 10 ml high RAC ¹⁷⁷Lu-PSMA I&T formulation composition comprising 42.5 mg/ml ascorbic acid is at least about 99% at 0 hours post EOS and at least about 93.3% at 46 hours post EOS, as measured by HPLC. The radiochemical purity decreases as the pH of the formulation is increased from pH 4.5.

The radiochemical purity of a 20 ml low RAC ¹⁷⁷Lu-PSMA I&T formulation comprising 31 mg/ml ascorbic acid is at least about 99.1% at 0 hours post EOS as measured by HPLC. The rate in which the radiochemical purity decreases over time is slower for low RAC ¹⁷⁷Lu-PSMA-I&T formulations having a pH of 4.5 compared to a pH of 5.

The results demonstrate that, formulation composition pH of 5 or below can substantially reduce formation of radiochemical impurity eluting at about 5.2 minutes and therefore enhance radiochemical stability of ¹⁷⁷Lu-PSMA I&T compared to formulation compositions at pH above 5. In addition, radiochemical stability of ¹⁷⁷Lu-PSMA I&T can further be improved by incorporation of lower solution RAC. In the example, ¹⁷⁷Lu-PSMA I&T solution showed highest radiochemical stability in low RAC solution of pH 4.5 and ascorbic acid concentration of 31 mg/ml. This formulation is considered a preferred composition to minimize formation of radiochemical impurities and to maintain radiochemical stability of ¹⁷⁷Lu-PSMA I&T above 95.0% for 72 hours or longer.

### Example 4: Effects of Treatment with Both ¹⁷⁷Lu-PSMA-617 and ¹⁷⁷Lu-PSMA-I&T Compared to Third Line Treatment

### Dosimetry for ¹⁷⁷Lu-PSMA I&T

There was no major difference in absorbed dose estimates between ¹⁷⁷Lu-PSMA I&T and ¹⁷⁷Lu-PSMA-617. The specific known dosimetry for ¹⁷⁷Lu-PSMA I&T is presented below.

For normal organs, the mean whole-body effective dose for all cycles was 0.41±0.18 Sv (0.06 Sv/GBq). The mean absorbed organ doses were 5.3±1.6 Gy (0.72 Gy/GBq) for the kidneys; 0.89±0.42 Gy (0.12 Gy/GBq) for the liver; and 4.0±1.1 Gy (0.55 Gy/GBq) for the parotid, 4.8±2.8 Gy (0.64 Gy/GBq) for the submandibular, and 27±10 Gy (3.8 Gy/GBq) for the lacrimal glands.

No substantial difference for absorbed doses of normal organs were observed when comparing them with respect to cycle number (Table 13). The mean organ masses underlying these absorbed dose estimates were 1.595±307 g (range, 1,165-2,373 g) for the liver, 153±29.9 g (range, 88.4-218.7 g) for the kidneys, 19.1±5.7 g (range, 8.0-35.6 g) for the parotid, 8.2±1.9 g (range, 4.2-14.3 g) for the submandibular, and 0.45±0.12 g (range, 0.25-0.78 g) for the lacrimal glands. For paired organs, masses from both sides were summed.

For tumor lesions, all lesions received a mean dose per cycle of 23±20 Gy (3.3 Gy/GBq). Mean absorbed doses for bone, lymph node, liver, and lung metastases were 26±20 Gy (3.4 Gy/GBq), 24±16 Gy (3.2 Gy/GBq), 8.5±4.7 Gy (1.28 Gy/GBq), and 13±7.4 Gy (1.7 Gy/GBq).

The values (mean, SD, and ranges) for the corresponding absorbed doses per GBq for normal organs and tumor lesions are presented in the tables below, respectively.

TABLE 11
Effective Dose for Whole Body in Sv/GBq and Absorbed
Doses for NormalOrgans in Gy/GBq.

| Cycles | Whole | | | Parotid | Submandibular | Lacrimal |
|---|---|---|---|---|---|---|
| investigated | body | Kidneys* | Liver | glands* | glands* | glands* |
| Overall | | | | | | |
| (n = 34) | | | | | | |
| Mean ± SD | 0.06± | 0.72 ± 0.21 | 0.12 ± 0.06 | 0.55± | 0.64 ± 0.40 | 3.8 ± 1.4 |
| Range | 0.02-0.11 | 0.33-1.22 | 0.05-0.26 | 0.25-0.84 | 0.24-1.70 | 1.68-7.03 |
| First cycle | | | | | | |
| (n = 15) | | | | | | |
| Mean ± SD | 0.05± | 0.71 ± 0.25 | 0.12 ± 0.07 | 0.56± | 0.54 ± 0.29 | 3.8 ± 1.5 |
| Range | 0.02-0.11 | 0.33-1.22 | 0.05-0.26 | 0.25-0.84 | 0.25-1.35 | 1.68-7.03 |
| Second cycle | | | | | | |
| (n [symbol] ) | | | | | | |
| Mean ± SD | 0.06± | 0.75 ± 0.19 | 0.13 ± 0.05 | 0.59± | 0.71 ± 0.42 | 4.1 ± 1.1 |
| Range | 0.03-0.11 | 0.44-1.01 | 0.08-0.21 | 0.47-0.80 | 0.40-1.70 | 2.44-5.56 |
| Third cycle | | | | | | |
| (n = [symbol] | | | | | | |
| Mean ± SD | 0.06± | 0.73 ± 0.22 | 0.12 ± 0.06 | 0.49± | 0.83 ± 0.59 | 3.5 ± 1.4 |
| Range | 0.03-0.10 | 0.42-0.93 | 0.07-0.21 | 0.32-0.64 | 0.33-1.68 | 2.41-5.04 |
| Fourth cycle | | | | | | |
| (n [symbol] | | | | | | |
| Mean ± SD | 0.05± | 0.69 ± 0.18 | 0.11 ± 0.04 | 0.50± | 0.66 ± 0.53 | 3.4 ± 1.7 |
| Range | 0.03-0.11 | 0.45-0.95 | 0.07-0.17 | 0.32-0.58 | 0.24-1.58 | 1.75-5.35 |

[symbol]

indicates data missing or illegible when filed

TABLE 12
Absorbed Doses for Tumor Lesions in Gy/GBq

| | All | Bone | Lymph node | Liver | Lung |
|---|---|---|---|---|---|
| Cycles investigated | metastases | metastases | metastases | metastases | metastases |
| Overall (n) | 93 | 74 | 8 | 8 | 3 |
| Mean ± SD | 3.2 ± 2.6 | 3.4 ± 2.7 | 3.2 ± 2.2 | 1.2 ± 0.67 | 1.75 ± 0.92 |
| Range | 0.22-12.03 | 0.22-12.03 | 1.63-8.46 | 0.47-2.59 | 0.94-2.68 |
| First cycle (n) | 41 | 33 | 5 | 2 | 1 |
| Mean ± SD | 3.5 ± 2.9 | 3.8 ± 3.1 | 2.6 ± 0.89 | 1.7 | 2.7 |
| Range | 0.22-12.03 | 0.22-12.03 | 1.63-3.76 | 0.85-2.59 | |
| Second cycle (n) | 26 | 21 | 2 | 2 | 1 |
| Mean ± SD | 3.3 ± 2.5 | 3.4 ± 2.4 | 5.2 | 0.94 | 1.3 |
| Range | 0.70-8.46 | 1.03-9.59 | 1.98-8.46 | 0.70-1.17 | |
| Third cycle (n) | 14 | 10 | 1 | 2 | 1 |
| Mean ± SD | 2.7 ± 2.3 | 3.2 ± 2.5 | 2.6 | 0.95 | 0.94 |
| Range | 0.94-7.99 | 1.11-7.99 | 18.87 | 0.47-1.42 | |
| Fourth cycle (n) | 12 | 10 | | 2 | |
| Mean ± SD | 2.4 ± 2.2 | 2.7 ± 2.3 | | 1.13 | |
| Range | 0.74-7.60 | 1.04-7.60 | | 0.74-1.51 | |

There is a clear trend toward a lower absorbed dose with an increasing number of the cycle. Mean absorbed dose per lesion was 26±21 Gy (3.5 Gy/GBq) for the first, 24±19 Gy (3.3 Gy/GBq) for the second, 20±18 Gy (2.7 Gy/GBq) for the third, and 18±17 Gy (2.4 Gy/GBq) for the fourth cycle. A similar trend can be seen for the subgroup of bone metastases. No reliable comparison is possible for lymph node, liver, and lung metastases because of a low sample number. See the below table for the effective half-lives and mean absorbed doses of ¹⁷⁷Lu-PSMA I&T.

¹⁷⁷Lu-PSMA RLT, the study included both ¹⁷⁷Lu-PSMA-617 and ¹⁷⁷Lu-PSMA I&T, had better effects and caused less adverse effects than third-line treatment. Twelve studies including 669 patients reported ¹⁷⁷Lu-PSMA RLT. Overall, 44% of the patients had a maximum decline of PSA of ≥50% following treatment with ¹⁷⁷Lu-PSMA RLT. The treatment with ¹⁷⁷Lu-PSMA-617 and ¹⁷⁷Lu-PSMA for imaging and therapy (I&T) had mainly transient adverse effects. Sixteen studies including 1338 patients reported third-line treatment. Overall, 21% of the patients had a best decline of PSA of ≥50% following third-line treatment. After third-line treatment with enzalutamide and cabazitaxel, adverse effects caused discontinuation of treatment for 10% to 23% of the patients. ¹⁷⁷Lu-PSMA RLT gave a best PSA decline ≥50% more often than third-line treatment (mean 44% versus 22%, p=0.0002, t-test). ¹⁷⁷Lu-PSMA RLT gave objective remission more often than third-line treatment (overall 31 of 109 patients versus 43 of 275 patients, p=0.004, χ2 test). Median survival was longer after ¹⁷⁷Lu-PSMA RLT than after third-line treatment, but the difference was not statistically significant (mean 14 months versus 12 months, p=0.32, t-test). Adverse effects caused discontinuation of treatment more often for third-line treatment than for ¹⁷⁷Lu-PSMA RLT (22 of 66 patients versus 0 of 469 patients, p<0.001, χ2 test).

The aim of this investigational medical product dossier (IMPD) is to provide a scientific and ethical platform for this useful treatment with ¹⁷⁷Lu-PSMA I&T to initially pursue its application, preferably in academic centers and under controlled investigational protocols. There are not existing guidelines except for ¹⁷⁷Lu-PSMA-617. The information in this IMPD is based on the newest literature and existing best experience from the nuclear medicine centers who have been treating PC patients with this modality.

The synthesis is a one-step labelling process with injections grade ethanol and water used as the only solvents. Therefore, no residual solvents are present. Radiochemical impurities are quantified by chromatographical methods (HPLC and TLC). Radiochemical purity may not be less than 95.0%.

### Example 5: A MULTI-CENTER, OPEN-LABEL, RANDOMIZED PHASE 3 TRIAL COMPARING THE SAFETY AND EFFICACY OF ¹⁷⁷Lu-PSMA I&T

A Multi-Center, Open-Label, Randomized Phase 3 Trial was conducted comparing the safety and efficacy of the composition comprising ¹⁷⁷Lu-PSMA I&T versus hormone therapy in patients with Metastatic Castration-Resistant Prostate Cancer.

This study aims to identify and characterize the safety and efficacy of using ¹⁷⁷Lu-PSMA I&T in the treatment of adult male human patients (men) with metastatic Castration-Resistant Prostate Cancer (mCRPC) who progress despite treatment with one course of standard of care hormone therapy.

¹⁷⁷Lu-PSMA I&T is a radioactive therapeutic agent that specifically targets the prostate specific membrane antigen proteins that are expressed on metastatic prostate cancer cells.

In this study, ¹⁷⁷Lu-PSMA I&T is provided as a sterile, filtered radiopharmaceutical solution that contains a microdose of ¹⁷⁷Lu-PSMA I&T in an aqueous ascorbic acid and ethanol solution.

Patients randomized to be treated in accordance with the standard of care hormone therapy for mCRPC will be treated either with abiraterone acetate in combination with prednisone, or enzalutamide based on the Investigator's independent medical judgment.

Abiraterone acetate is indicated in combination with prednisone for the treatment of patients with metastatic castration-resistant prostate cancer (mCRPC) or metastatic high-risk castration-sensitive prostate cancer (mCSPC). Abiraterone acetate is converted in vivo to abiraterone, an androgen biosynthesis inhibitor, that inhibits 17 α-hydroxylase/C17,20-lyase (CYP17). This enzyme is expressed in testicular, adrenal, and prostatic tumor tissues and is required for androgen biosynthesis.

Enzalutamide is an androgen receptor inhibitor indicated for the treatment of patients with CRPC or mCSPC (Metastatic Castration-Sensitive Prostate Cancer). Enzalutamide has been shown to competitively inhibit androgen binding to androgen receptors and, consequently, inhibits nuclear translocation of androgen receptors and their interaction with DNA.

### A. Study Objectives and Endpoints

The primary objective of this study is to prospectively assess the efficacy of ¹⁷⁷Lu-PSMA I&T on the improvement of radiographic progression-free survival (rPFS) as determined by PCWG3-modified RECIST 1.1 in men with metastatic Castration-Resistant Prostate Cancer (mCRPC) compared to standard of care hormone therapy. The endpoint of this objective is the time from randomization to radiographic progression as determined by Prostate Cancer Working Group 3 (PCWG3) criteria as assessed by blinded independent central review.

The secondary objective is to assess if ¹⁷⁷Lu-PSMA I&T improves overall survival (OS) in patients with mCRPC compared to those treated with standard of care hormone therapy. The endpoint of this objective is the time from randomization to death by any cause.

Other secondary objectives and endpoints include:
Objective: evaluate the improvement in overall survival (OS) in men with mCRPC treated with ¹⁷⁷Lu-PSMA I&T compared to hormone therapy; Endpoint: time from randomization to the second radiographic progression as determined by PCWG3 or RECIST 1.1 by BICR (Blinded Independent Central Review) after crossover.
Objective: assess changes in the time to second radiographic progression for patients who crossover from the standard of care hormone therapy arm to the ¹⁷⁷Lu-PSMA I&T treatment arm; Endpoint: time from randomization to the second radiographic progression as determined by PCWG3 or RECIST 1.1 by BICR after crossover,
Objective: identify changes in progression-free survival (PFS, composite) following ¹⁷⁷Lu-PSMA I&T radioligand therapy compared to standard of care hormone therapy; Endpoint: time from randomization to progression (PFS, composite) based on the following events, whichever occurs first: PCWG3 or RECIST progression, clinical/symptomatic progression and/or pain progression, or death due to any cause as determined by investigator.
Objective: identify changes in progression-free survival 2 (PFS2, composite) following ¹⁷⁷Lu-PSMA I&T radioligand therapy compared to standard of care hormone therapy; Endpoint: time from randomization to the second progression (PFS, composite) based on the following events, whichever occurs first: PCWG3 or RECIST progression, clinical/symptomatic progression and/or pain progression, or death due to any cause as determined by investigator,
Objective: assess changes in PSA50 (Response Rate of Patients with a reduction of PSA ≥50% from Baseline) response rate following ¹⁷⁷Lu-PSMA I&T radioligand therapy compared to standard of care hormone therapy; Endpoint: PSA50 response rate, defined as a confirmed reduction of PSA from baseline of ≥50%,
Objective: determine the impact of ¹⁷⁷Lu-PSMA I&T compared to standard of care hormone therapy on skeletal symptoms; Endpoint: time from randomization to first symptomatic skeletal event (SSE-free survival),
Objective: determine the impact of ¹⁷⁷Lu-PSMA I&T compared to standard of care hormone therapy on radiographic soft-tissue progression; Endpoint: time from randomization to radiographic soft tissue progression (rSTP) as measured by RECIST 1.1 by BICR,
Objective: assess changes in use of chemotherapy following ¹⁷⁷Lu-PSMA I&T compared to standard of care hormone therapy; Endpoint: time from randomization to first use of chemotherapy, and
Objective: evaluate the impact on quality-of-life following ¹⁷⁷Lu-PSMA I&T radioligand therapy to hormone therapy; Endpoint: Quality of Life improvement based on EORTC QLQ-C30 questionnaire.

Exploratory objectives and endpoints are:
Objective: evaluate any differences in objective response rate and disease control rate with ¹⁷⁷Lu-PSMA I&T compared to standard of care hormone therapy; Endpoint: objective response rate based on PCWG3 criteria and disease control rate (DCR=complete/partial response and stable disease),
Objective: evaluate changes in time to PSA progression following ¹⁷⁷Lu-PSMA I&T radioligand therapy compared to standard of care; Endpoint: time from randomization to PSA progression, defined as a rise in PSA of ≥25% from the post-treatment nadir, and
Objective: assess the duration of response (DoR) in patients who achieve complete or partial response following ¹⁷⁷Lu-PSMA I&T radioligand therapy compared to hormone therapy; Endpoint: the time from complete or partial response to radiographic progression.

### B. Study Design

### (i) Overview and Rationale

This is an open-label, randomized, multicenter Phase 3 study of ¹⁷⁷Lu-PSMA I&T radioligand therapy compared to hormone therapy in men with mCRPC who have been previously treated with androgen receptor (AR)-directed therapy. The hormone therapy regimens for this study are enzalutamide or abiraterone with prednisone based on NCCN guidelines. Selection of the specific regimen will be based on a switch from the patient's previous ADRT for patients randomized to receive standard of care. Based on the published literature, ¹⁷⁷Lu-PSMA I&T radioligand therapy has encouraging anti-tumor activity in men with mCRPC and is associated with a favorable safety profile.

The study consists of a Screening Phase, a Treatment Phase, and a Post-Treatment Follow-up Phase. The study employs a 2:1 randomization into the following treatment groups: (1) ¹⁷⁷Lu-PSMA I&T radioligand therapy, or (2) Standard of Care hormone therapy. Standard of care hormone treatment options are abiraterone with prednisone or enzalutamide, with the specific choice based on the Investigator's clinical judgment. Patients randomized to the hormone therapy arm will be given the option to crossover to the radioligand therapy arm upon documentation of radiographic progression.

Patients will be followed for safety and efficacy as per the schedule of activities and will remain on study treatment until documented radiographic progression occurs as assessed by a blinded independent central review (BICR) or the development of unacceptable toxicity. Patients discontinuing treatment due to documented radiographic progression will enter the Long-term Follow-up Phase. Patients discontinuing treatment prior to documented radiographic progression will continue to have scheduled disease assessments every three months until documented radiographic progression.

In addition, a sub-study is conducted under this protocol in patients randomized to receive ¹⁷⁷Lu-PSMA I&T radioligand therapy to evaluate the pharmacokinetic and radiation dosimetry (as discussed in Example 4 above).

Due to the nature of the treatments, the identity of the test and control treatments will be known to the Investigators, research staff, and patients. Blinding of this study is not feasible. Immediately following Investigator determination of progressive disease for progressing patients and following the completion of the study for non-progressing patients, blinded radiographic images will be read and interpreted by a panel of up to three trained independent radiologists without access to clinical information or treatment groups for assessing the overall response rate to treatment.

The severity of AEs and SAEs will be graded based upon the subject's symptoms in accordance with the Common Terminology Criteria for Adverse Events (CTCAE), Version 5.0. AEs that are not defined in the CTCAE current version should be evaluated for severity in accordance with the following scale:
Grade 1=Mild: transient or mild discomfort, no limitation in activity, no medical intervention/therapy required
Grade 2=Moderate: mild to moderate limitation in activity, some assistance may be needed, no or minimal medical intervention/therapy required
Grade 3=Severe: marked limitation in activity, some assistance usually required, medical intervention/therapy required, hospitalization possible
Grade 4=Life-threatening: extreme limitation in activity, significant assistance required, significant medical intervention/therapy required, hospitalization or hospice care probable
Grade 5=Death: the event results in death

It is important to distinguish between serious and severe AEs. Severity is a measure of intensity, whereas seriousness is defined by the criteria outlined in Section 10.3. An AE of severe intensity may not be considered serious. Seriousness, not severity, serves as a guide for defining regulatory obligations.

### (ii) Selection of the Primary Endpoint

Metastatic castration-resistant prostate cancer is generally considered a late stage in the natural progression of prostate cancer. Although mCRPC is usually associated with an unfavorable prognosis, many patients experience a more indolent disease progression resulting in a larger range of overall survival with approximately 15% of men with mCRPC surviving beyond 5 years (Moreira et al., Clin Genitourin Cancer, 2017; 15(1): 60-66).

In consideration of the relatively large range of overall survival in men with mCRPC, radiographic progression-free survival using RECIST 1.1 (soft tissue lesion status), and Prostate Cancer Working Group 3 (PCWG3) criteria (bone lesion status), as assessed by blinded independent central review, was chosen as the primary endpoint for this study. Overall survival is a secondary endpoint of the study, and patients will be followed for overall survival for a period of 5 years after enrollment.

(iii) Study Outcomes

The primary efficacy outcome is radiographic progression-free survival. Once radiographic progression is confirmed by the BIRC, study treatments will be discontinued. Patients will then enter the Follow-up Phase of the trial to assess overall survival for a 5-year follow-up period from the date of enrollment in the study.

Safety will be assessed until the end of treatment and one-month follow-up period by assessing the following safety parameters: adverse events, vital signs, changes in concomitant medications/therapies, changes in physical examination, and clinical laboratory measurements.

### C. Patient Selection

### (i) Study Population

The study population will include patients with mCRPC with progressive disease based on PCWG3 modified RECIST 1.1 criteria.

### (ii) Inclusion Criteria Include:

1. Male 18 years or older.
2. Histologically or pathologically confirmed prostate adenocarcinoma without predominant small cell component.
3. Progressive disease by one or more of the following criteria:
a. Serum/plasma PSA progression defined as 2 consecutive increases in PSA over a previous reference value measured at least 1 week apart with a minimum start value of >2 ng/mL.
b. Progression of measurable disease (RECIST 1.1) or presence of at least two new bone lesions (PCWG3 criteria)
4. Previous treatment with next-generation androgen receptor (AR)-directed therapy (e.g. abiraterone, enzalutamide, apalutamide, darolutamide).
a. Must have received no more than one previous AR-directed therapy.
b. Must have been administered ARAT (abiraterone, enzalutamide, darolutamide, or apalutamide) in the castration-sensitive or castration-resistant setting.
c. Must have progressed while on ARAT.
5. PSMA-PET scan (e.g., [⁶⁸Ga]Ga-PSMA-11 or [¹⁸F]DCFPyL) positive as determined by central reader.
6. Effective castration with serum testosterone level of <50 ng/dL and plan to continue with chronic medical or surgical castration.
7. Patients with HIV that are healthy and with a low risk of acquired immune deficiency syndrome related outcomes may participate in the study at the investigators' discretion.
8. Patients with HBV and HCV may also participate if symptoms are sufficiently managed.
9. Life expectancy of at least 6 months as assessed by investigator.
10. Willing to initiate ARAT therapy determined by investigator.
   (iii) Exclusion Criteria Include:
1. Prior treatment with radioligand therapy including other lutetium-labeled compounds.
2. Prior treatment with radium-223 (Xofigo) within the past 12 weeks.
3. Prior chemotherapy treatment for castration-sensitive or castration-resistant prostate cancer (docetaxel or cabazitaxel).
4. Eastern Cooperative Oncology Group (ECOG) performance status (PS) ≥2.
5. Patients with known HRR (Haploid relative risk) gene-mutation who have not been previously treated with olaparib or rucaparib.
6. Other concurrent cytotoxic chemotherapy, immunotherapy, radioligand therapy, or investigational therapy.
7. Inadequate organ and bone marrow function as evidenced by:
a. Hemoglobin <8 g/dL.
b. Absolute neutrophil count <1.5×109/L.
c. Platelet count <100×109/L.
d. AST/SGOT and/or ALT/SGPT >3.0×ULN (wherein: “AST” is Aspartate aminotransferase, “SGOT” is Serum glutamic-oxaloacetic transaminase, “SGPT” is Serum glutamic-pyruvic transaminase, and “ALT” is Alanine aminotransferase).
e. Total bilirubin >2×ULN (Upper Limit of Normal) unless patient has known Gilbert's syndrome and then may be 3×ULN.
f. Creatinine clearance (CrCl)<50 mL/min based on the Cockcroft-Gault equation.
g. Albumin ≥2.75 g/dL
8. Patients who undergo a transfusion for the sole purpose of meeting eligibility for this study.
9. Use of an investigational therapeutic drug within the last 4 weeks prior to start of study treatment or scheduled to receive one during the study period.
10. Known CNS metastasis unless received therapy, asymptomatic and neurologically stable.
11. Patients receiving zoledronic acid for bone-targeted therapy must be on stable dose for 4 weeks prior to randomization.
12. Patients with active significant cardiac disease
13. Participants with symptomatic cord compression or clinical/radiological findings indicating impending spinal cord compression.
14. Patients with a superscan seen on baseline bone scan as determined by investigator.
15. Active malignancy other than low-grade non-muscle-invasive bladder cancer and non-melanoma skin cancer.
16. Previous use of G-CSF (Granulocyte-colony stimulating factor) for persistent neutropenia after standard of care treatment.
17. Participants with active Covid19. Recovered patients may be included when completely recovered (no symptoms at least 28 days before study medication and a negative Covid test within 72 hours).

### D. Investigational Product Dosage, Route of Administration, and Dosing Schedule

### (i) ¹⁷⁷Lu-PSMA I&T

The medicinal product is a sterile filtered radiopharmaceutical solution that contains a microdose of ¹⁷⁷Lu-PSMA I&T formulated in an aqueous solution containing ascorbic acid and ethanol. The product contains a standard activity of approximately 200 mCi at the time of expiry with a standard concentration of approximately 27 mCi/mL at end of production; therefore, the final volume of the dose vial is adjusted to between 7.0 and 10.0 mL in order to provide the required amount of radioactivity at the date and time of infusion. ¹⁷⁷Lu-PSMA I&T injection is supplied as a sterile solution in a single-dose vial. The septum is sealed with a crimped aluminum cap. The glass vial containing the radiopharmaceutical is kept in a lead shielded container until use. ⁷⁷Lu-PSMA I&T is stored at 25° C.; excursions between 15° C.-30° C. are permitted. The shield label will state the expiration date and time for each vial shipped to the clinical sites.

### (ii) Standard of Care Hormone Treatments

Standard of care hormone treatments are:
Abiraterone Acetate with Prednisone: Abiraterone acetate is a CYP 17 inhibitor used in combination with prednisone or methylprednisone. Prednisone is a glucocorticoid. Glucocorticoids are adrenocortical steroids that are readily absorbed from the gastrointestinal tract.
Enzalutamide is an androgen receptor inhibitor.
   (iii) Preparation

All infusion solutions will be prepared and dispensed by the site prior to administration. The preparation of solutions should be performed under aseptic conditions, and final solutions should be inspected visually for particulate matter. If an insoluble precipitate is observed, the solution should be discarded.

¹⁷⁷Lu-PSMA I&T injection solution is administered as supplied. The radioactivity in the vial should be measured in a calibrated radiation dose calibrator prior to, and after administration to the patient. The administered dose should then be calculated and recorded.

### (iv) Dosing and Administration

Patients will be randomized 2:1 to receive either ¹⁷⁷Lu-PSMA I&T radioligand therapy or standard of care hormone therapy.

¹⁷⁷Lu-PSMA I&T Administration: Patients randomized to receive radioligand therapy will receive a single intravenous radioactive dose of 200 mCi (7.4 GBq)±10% of ¹⁷⁷Lu-PSMA I&T at the beginning of each treatment cycle.

An intravenous line should be established prior to administration of ¹⁷⁷Lu-PSMA I&T. The ¹⁷⁷Lu-PSMA I&T will be injected as a slow bolus over a minimum of 10 to 15 minutes using the site's standard radioligand therapy administration procedures. The patient should be encouraged to void as frequently as possible and drink two liters of liquid daily for two days following ¹⁷⁷Lu-PSMA I&T administration.

Cooling of the patients' salivary glands should be performed by placing ice packs over the parotid and submandibular glands for 30 minutes prior to and up to 4 hours after the injection of ¹⁷⁷Lu-PSMA I&T to reduce the risk of salivary gland radiation injuries.

A six-week treatment cycle will be used for injection of ¹⁷⁷Lu-PSMA I&T for 4 treatment cycles or until radiographic progression of disease is determined based on the BIRC assessment of radiographic images (maximum 18 weeks of treatment). Alternatively an eight-week treatment cycle may be used for injection of ¹⁷⁷Lu-PSMA I&T for 6 treatment cycles or until radiographic progression of disease is determined based on the BIRC assessment of radiographic images (maximum 18 weeks of treatment).

The dosing cycle for ¹⁷⁷Lu-PSMA I&T may be extended based on evaluation of dose-limiting toxicity experienced by a patient. Dose-limiting toxicity is defined as grade 3 or 4 bone marrow toxicity or grade 2 or greater salivary gland toxicity. For grade 3 or 4 bone marrow toxicity, dosing may resume when improvement to grade 2 or better is observed. For grade 2 or greater salivary gland toxicity, dosing may resume upon improvement to grade 1 toxicity.

In addition, the ¹⁷⁷Lu-PSMA I&T dose should be held and/or reduced to 160 mCi (5.9 GBq)±10% if dose-limiting toxicity is noted. The following dose hold/reductions should be implemented accordingly:
For grade ≥3 Anemia: Hold dose until return to baseline or Grade ≤2 and then reduce to 160 mCi (5.9 GBq) for next cycle
For grade ≥2 Neutropenia: Hold until return to baseline or Grade ≤1
For grade ≥2 Thrombocytopenia: Hold until return to baseline or Grade ≤1 and reduce dose to 160 mCi (5.9 GBq) for next cycle
For grade ≥3 Non-platelet hematological toxicity: Hold until return to baseline or Grade ≤2 and reduce dose to 160 mCi (5.9 GBq) for the next cycle

For cases of acute renal toxicity that is Grade 3 or higher, dose should be held for next cycle. Dose may be reinstated upon return to baseline or Grade ≤2 but reduced to 160 mCi (5.9 GBq) for all remaining doses.

Dosing cycle extensions of an additional six weeks and dose reduction modifications to 160 mCi (5.9 GBq) for remaining cycles should be initiated for any other Grade 3 or higher non-hematological toxicity related to ¹⁷⁷Lu-PSMA I&T as determined by the Investigator. Dosing cycle and dose level may be returned to every six weeks when toxicity returns to a Grade 2 or below.

For all grade 3 and 4 AEs, patients will only be able to have a 1-time reduction in dose. If the event persists, the patient would need to permanently discontinue from study treatment.

For grade 2 AEs, only 2 dose reductions are permitted. If the event persists the patient would need to permanently discontinue from study treatment.

Abiraterone Acetate with Prednisone: The dose of abiraterone acetate should be administered per package insert. Per Abiraterone package insert, for patients with baseline moderate hepatic impairment, the starting dose of abiraterone should be reduced to 250 mg daily. For patients who develop hepatoxicity during treatment, dosing with abiraterone acetate should be suspended until recovery. Retreatment may be initiated at a reduced dose. Abiraterone acetate treatment should be discontinued in patients that develop severe hepatotoxicity.

Enzalutamide: The dose of enzalutamide is 160 mg (four 40 mg capsules) administered orally once daily. Capsules should be swallowed whole and can be taken with or without food. Per enzalutamide package insert, if a patient experiences a ≥Grade 3 toxicity or an intolerable side effect, dosing should be withheld for one week or until symptoms improve to ≤Grade 2, then resumed at the same or a reduced dose (80 or 80 mg) if warranted.

### (v) Duration of Study Treatment

Patients will be treated until radiographic progressive disease, clinical/symptomatic progression, unacceptable/unmanageable toxicity, or patient's refusal of further study treatment (i.e., withdrawal of consent). All patients will be followed while on study treatment and after completion of study treatment during follow-up period: until death, the study cut-off date (which is a minimum of 22 weeks post-enrollment), or withdrawal of consent, whichever comes first. Long-term follow-up with all patients will be five years from enrollment or until death or loss to follow-up.

### E. Study Procedures and Evaluations

Trial evaluations and time points are summarized in the Schedule of Activities in Tables 13 and 14.

TABLE 13
Schedule of Events for ¹⁷⁷Lu-PSMA-I&T Arm.

| | | | | | | | | | | Post- | | |
|---|---|---|---|---|---|---|---|---|---|---|---|---|
| | | | | | | | | | | treatment | | |
| | | Treatment Phase | | | | | | | | Phase | | |
| | Screening | Cycle 1 | | Cycle 2 | | Cycle 3 | | Cycle 4 | | EOT | LTFU | |
| Week (±7 days) | −4 to 0 | 0 | 4 | 6 | 8 | 12 | 16 | 18 | 20 | 22 | 24-256 | US |
| Informed Consent | X | | | | | | | | | | | |
| Demographics | X | | | | | | | | | | | |
| Inclusion/Exclusion | X | | | | | | | | | | | |
| Criteria | | | | | | | | | | | | |
| Medical History | X | | | | | | | | | | | |
| PSMA PET | X | | | | | | | | | | | |
| CT and Bone Scan^{a} | X | | | | X | | X | | | | X^{a} | |
| ¹⁷⁷Lu-PSMA-I&T | | X | | X | | X | | X | | | | |
| Treatment | | | | | | | | | | | | |
| Hematology | X | X | X | | X | X | X | | X | X | | |
| Blood Chemistry | X | X | X | | X | X | X | | X | X | | |
| PSA | X | X | | | X | | X | | | X | | |
| ECOG | X | X | | | | | | | | | | |
| Physical Exam | X | X | X | | | | | | | X | | |
| Vital Signs | X | X | X | | | | | | | X | | |
| 12-Lead ECG | X | X | | | | | | | | | | |
| Concomitant | X | X | X | X | X | X | X | X | X | X | X | |
| Medications | | | | | | | | | | | | |
| EORTC | | X | X | | | X | X | | X | X | | |
| FACT-P | | X | X | | X | X | X | | X | X | | |
| BPI-SF | | X | X | | X | X | X | | X | X | | |
| Adverse Events | X | X | X | X | X | X | X | X | X | X | X | X |
| Progression^{b} | | | | | X | | X | | X | | X | |
| Survival | | | | | | | | | | | X | |

EOT = End of Treatment;
LTFU = Long-Term Follow Up;
US = Unscheduled
^{a}CT/Bone scan will be performed every 8 weeks throughout treatment until week 24, and then, if no progression, every 12 weeks continued in LTFU.
^{b}Per section 8.5, progression assessments to be conducted every 12 weeks throughout LTFU until radiographic evidence of disease progression is noted

TABLE 14
Schedule of Events for Standard of Care Hormone Therapy Arm.

| | Screening | Treatment Phase | | | | | | EOT | LTFU | |
|---|---|---|---|---|---|---|---|---|---|---|
| Week (±7 days) | −4 to 0 | 0 | 4 | 8 | 12 | 16 | 20 | 22 | 24-256 | US |
| Informed Consent | X | | | | | | | | | |
| Demographics | X | | | | | | | | | |
| Inclusion/ | X | | | | | | | | | |
| Exclusion | | | | | | | | | | |
| Criteria | | | | | | | | | | |
| Medical History | X | | | | | | | | | |
| PSMA PET | X | | | | | | | | | |
| CT and Bone Scan^{a} | X | | | X | | X | | | X^{a} | |
| Abiraterone or | | Daily | | | | | | | | |
| Enzalutamide | | | | | | | | | | |
| Treatment | | | | | | | | | | |
| Hematology | X | X | X | X | X | X | X | X | | |
| Blood Chemistry | X | X | X | X | X | X | X | X | | |
| PSA | X | X | | X | | X | | X | | |
| BCOG | X | X | | | | | | | | |
| Physical Exam | X | X | X | | | | | X | | |
| Vital Signs | X | X | X | | | | | X | | |
| 12-Lead ECG | X | | | | | | | | | |
| Concomitant | X | X | X | X | X | X | X | X | X | |
| Medications | | | | | | | | | | |
| EORTC | | X | X | X | X | X | X | X | | |
| FACT-P | | X | X | X | X | X | X | X | | |
| BPI-SF | | X | X | X | X | X | X | X | | |
| Adverse Events | X | X | X | X | X | X | X | X | X | X |
| Progression^{b} | | | | X | | X | X | | X | |
| Survival | | | | | | | | | X | |

EOT = End of Treatment;
LTFU = Long-Term Follow Up;
US = Unscheduled
^{a}CT/Bone scan will be performed every 8 weeks throughout treatment until week 24, and then, if no progression, every 12 weeks continued through LTFU.
^{b}Progression assessments to be conducted every 12 weeks throughout LTFU until radiographic evidence of disease progression is noted

### (i) Clinical Assessments

Clinical assessments include demographics, medical history, physical examination, vital signs, performance status, adverse events, concomitant medications/therapies, tumor assessments, and Blinded Independent Central Review (BJCR).

For performance status, the Eastern Cooperative Oncology Group (ECOG) performance status scale will be used and will be assessed at Screening and every subsequent clinic visit as shown below in Table 15:

TABLE 15
Eastern Cooperative Oncology Group (ECOG) performance status scale/

| Grade | Activity Level |
|---|---|
| 0 | Fully active, able to carry on all pre-disease performance without restriction. |
| 1 | Restricted in physically strenuous activity but ambulatory and able to carry |
| | out work of light or sedentary nature (e.g., light housework, office work). |
| 2 | Ambulatory and capable of all self-care but unable to carry out any work |
| | activities, up and about more than 50% of waking hours. |
| 3 | Capable of only limited self-care, confined to bed or chair more than 50% of |
| | waking hours. |
| 4 | Completely disabled cannot carry on any self-care, totally confined to bed or |
| | chair. |
| 5 | Dead |

Tumor assessments will be performed according to the assessment calendar, regardless of treatment delays resulting from toxicity. Care must be taken in scheduling tumor assessments to prevent the introduction of bias based on treatment delays.

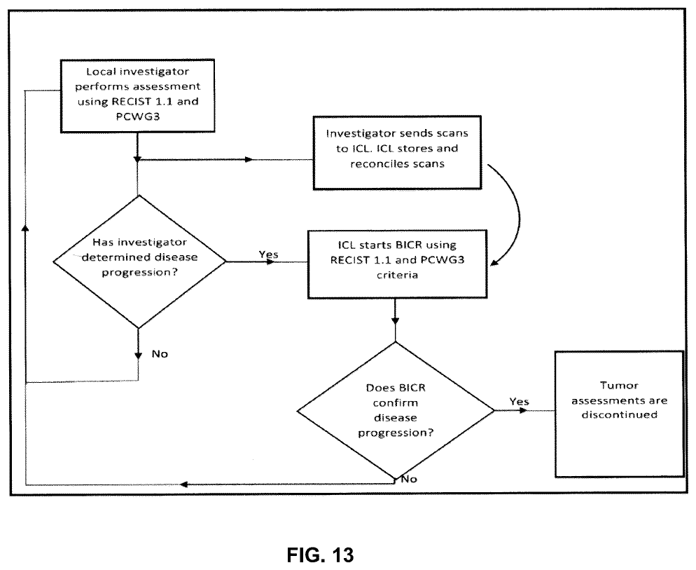

Patients will be evaluated for tumor response by CT imaging plus a bone scan at Screening and every 8 weeks (±1 week) following the start of treatment through week 24 of the study. Thereafter in patients without radiographic progression noted during the 24-week period, CT imaging and bone scans will be performed every 12 weeks (±1 week) until radiographic progression is determined. The schedule for scans will be based on calendar and not the start of a treatment cycle. Evaluations will include CT scans of chest, abdomen, pelvis, and brain (only as clinically warranted based on symptoms/findings). The Investigator, sub-Investigator, or qualified Site personnel will read the bone scans and CT images to assess whether radiographic progression has been noted. If the Investigator determines a patient's metastatic prostate cancer has progressed, the bone scans and CT images for the patient will immediately be transmitted to the Imaging Core Laboratory (ICL) for review by the Blinded Independent Central Review (BICR) to confirm radiographic progression as shown in FIG. 13.

No change in treatment should be made by the Investigator until after confirmation of disease status is received from the BICR.

### Blinded Independent Central Review (BICR)

Screening: All scans (CT, bone, and PSMA PET), will be submitted to a third-party imaging core lab (ICL) for independent review of patient eligibility (within 3 days of receipt of imaging scans that pass quality assessment). Following confirmation by the BICR, the patient can be randomized into the study provided all other eligibility criteria have been met.
Radiographic Disease Progression: The investigator will assess the CT and bone scans to assess disease progression based upon RECIST 1.1 and PCWG3 criteria. If the investigator determines the disease progression has occurred, the BIRC, consisting of a panel two independent radiologists qualified to assess bone scans and CT images, will independently assess disease progression in accordance with RECIST 1.1 and PCWG3 criteria. Confirmation of radiographic disease progression requires agreement between the two blinded readers. If agreement between the two readers does not occur, a third reader will be utilized to adjudicate. The BICR will complete the confirmation of disease progression whenever possible within 72 hours of receipt of the image set from the Investigator demonstrative of radiographic progression. No alteration in the clinical management of the patient should be initiated by the Investigator prior to receipt of the BICR confirmation of radiographic progression. FIG. 13 presents the baseline and on-treatment disease status evaluation and treatment decisions.

### (ii) Patient-Reported Outcomes

Patient-reported outcomes will be determined using the European Organization for Research and Treatment of Cancer Quality of Life Questionnaire (EORTC QLQ-C30), the Functional Assessment of Cancer Therapy—Prostate (FACT-P) questionnaire, and the Brief Pain Inventory—Short Form (BPI-SF) questionnaire.

The EORTC QLQ-C30 is a questionnaire of thirty quality of life (QoL) questions developed to assess the QoL of cancer patients. The QoL questionnaire has been included in over 3,000 Phase 3 cancer clinical trials as an efficacy endpoint. The EORTC questionnaire will be administered to the patients at baseline, during treatment as provided in the Schedule of Events in Table 15, and at the end of treatment.

The Functional Assessment of Cancer Therapy—Prostate (FACT-P) is a health-related Quality of Life questionnaire with 39 prostate cancer-specific questions that evaluates: Physical Well-Being, Functional Well-Being, Emotional Well-Being, Social Well-Being, and Additional Concerns or Prostate Cancer Subscale specific to prostate cancer. Higher FACT-P scores correspond to better quality of life. The FACT-P questionnaire will be administered to the patients at baseline, during treatment, and at the end of treatment.

The Brief Pain Inventory—Short Form (BPI-SF) is a short survey to assess overall pain and symptoms experienced by the participant. The BPI-SF questionnaire will be administered to the patients at baseline, during treatment, and at the end of treatment.

### F. Study Assessments by Visit

### (i) Screening

Screening must be completed within 28 days prior to randomization into the study. The screening visit includes (a) drawing blood samples and submit to central laboratory for determining PSA level and baseline clinical laboratory assessments, (b) obtaining CT of the brain, chest, abdomen, and pelvis plus bone scan and submit to BICR within 72 hours to confirm patient eligibility, (c) obtaining PSMA-PET scan with FDA-approved radiotracer ([⁶⁸Ga]Ga-PSMA-11 or [¹⁸F]DCFPyL). PSMA-PET positivity is required for inclusion in the trial. PSMA-PET positivity is defined as PSMA-PET uptake greater than that of liver in one or more metastatic lesions of any size in any organ system, (d) recording medical history, including history of prostate cancer, diagnosis date, and prior treatments, (e) recording concomitant medications, (f) performing a complete physical examination, (g) performing and record vital signs and ECOG performance status grade, (h) performing 12-lead ECG, and (i) if patient is confirmed eligible by the Medical Monitor for enrollment in the study, randomizing patient in accordance with IVRS system and proceed to Cycle 1, Day 1 visit.

### (ii) Treatment Day 1

The following assessments will be conducted on Treatment Day 1:
administering EORTC QLQ-C30, FACT-P, and BPI-SF questionnaires prior to treatment for baseline determination.
recording any changes to concomitant medications, and any adverse events noted from time of screening.
performing abbreviated physical examination
performing and recording vital signs and ECOG performance status.
collecting blood for clinical laboratory assessments.
administering study drug in the clinic. For patients enrolled into the standard of care arm of the study, initiate the Investigator's assigned standard of care hormone treatment:
Abiraterone acetate with prednisone: The initial dose for abiraterone acetate is 1000 mg (four 250 mg tablets) administered once daily. The first dose of abiraterone acetate in combination with prednisone will be administered at the clinic. The time and date of the initiation of abiraterone therapy will be recorded.
Enzalutamide: The initial dose for enzalutamide is 160 mg (four 40 mg capsules) administered once daily. The first dose of enzalutamide will be administered at the clinic. The time and date of the initiation of enzalutamide therapy will be recorded.
Patients enrolled in the radioligand therapy arm should be administered an initial radioactive dose of 200 mCi (7.4 GBq) of ¹⁷⁷Lu-PSMA-I&T infused over a minimum of 10 to 15 minutes. The dose and time period for ¹⁷⁷Lu-PSMA-I&T infusion will be recorded.
performing 12-lead ECG for patients enrolled in the ¹⁷⁷Lu-PSMA-I&T after first dose.
   (iii) On-Study Treatments

¹⁷⁷Lu-PSMA I&T: Patients randomized into the radioligand therapy arm of the study will receive ¹⁷⁷Lu-PSMA I&T infusions on a 6-week infusion cycle at a dose of 200 mCi (7.4 GBq) until radiographic progression is confirmed by the BICR or until the patient experiences toxicity requiring discontinuation of treatment or withdraws their consent to participate in the study. A maximum 4 cycles of ¹⁷⁷Lu-PSMA I&T infusions may be given to a patient in this study.

Abiraterone and Enzalutamide Standard of Care Arms: Patients randomized to standard of care and being treated with either abiraterone or enzalutamide will be treated daily with the standard of care dose regimen for these drugs according to their prescribing information. Patients in this treatment group will continue receiving the standard of care treatment until radiographic progression is confirmed by the BICR or until the patient experiences toxicity requiring discontinuation of treatment or withdraws their consent to participate in the study.

### (iv) Ongoing Assessments

Ongoing assessments will be conducted for all patients enrolled in the study every 4 weeks for patients randomized to receive ¹⁷⁷Lu-PSMA I&T and patients receiving abiraterone or enzalutamide standard of care.

### (v) Progression Assessments

Assessments will be conducted on all patients enrolled in the study starting 8 weeks+1 week from the initial treatment and continuing through Week 24, and thereafter every 12 weeks until radiographic evidence of disease progression is noted. The assessments consist of (i) obtaining CT chest, abdomen, and pelvis images plus bone scan and submit to ICL (Imaging Core Laboratory) for BICR to evaluate, and (ii) obtaining plasma samples for PSA and other clinical laboratory assessments.

### (vi) End of Treatment Visit

The End of Treatment (EOT) visit will be conducted at one-month (±7 days) after the last infusion of ¹⁷⁷Lu-PSMA I&T. Patients receiving abiraterone acetate or enzalutamide can continue receiving daily treatment up until the EOT visit. The following assessments will be conducted: administering EORTC QLQ-C30, FACT-P, and BPI-SF questionnaires; recording any changes to concomitant medications; recording any adverse events noted from time of screening; performing abbreviated physical examination; performing and recording vital signs and ECOG performance status; and collecting blood for clinical laboratory assessments including PSA and other clinical laboratory assessments.

(vii) Crossover

Patients in the standard of care hormone therapy arm may crossover to receive ¹⁷⁷Lu-PSMA I&T based on the following criteria:
Must have documented radiographic progression by PCWG3-modified RECIST 1.1 by BICR while on the standard of care hormone therapy
Must not have started any other anti-cancer medications or therapy.
Participants with inadequate organ and bone marrow function as defined below will not be eligible for crossover:
Absolute neutrophil count <1.5×109/L
Platelet count <100×109/L.
Hemoglobin <8 g/dL.
AST/SGOT and/or ALT/SGPT >3.0×ULN.
Total bilirubin >2×ULN unless patient has known Gilbert's syndrome and then may be 3×ULN.
Creatinine clearance (CrCl)<50 mL/min based on the Cockcroft-Gault equation.
Albumin ≥2.75 g/dL

If patients are not eligible for crossover, then they should instead complete the End-of-Study Visit and go into Long Term Follow-up.

(viii) Long-Term Follow-Up

Long-term patient follow-up will continue up to 5 years after the initial treatment in this study or until patient death or loss to follow-up. The following information will be collected:
Survival status, development of symptomatic disease progression, initiation of any new systemic anti-cancer therapies, progression on first subsequent therapy, and medical resource utilization every 4 months.
In addition, if patients discontinue study treatment prior to documented disease progression, obtain CT of the chest, abdomen, and pelvis plus bone scan and submit to BICR to evaluate disease progression every 12 weeks until documentation of radiographic disease progression.

### G. Statistical Considerations and Analytical Plan

### (i) Sample Size

It is hypothesized that treatment of patients with ¹⁷⁷Lu-PSMA I&T will increase the radiographic Progression Free Survival (rPFS) from 6 months in the standard of care group to 10 months. Thus, a target Hazard Ratio (HR) under the alternative hypothesis of 0.60 is reasonable to expect for this Phase 3 study. Utilizing a 2:1 randomization, an estimated 237 progression events across both treatment arms will provide a 95% power to detect a statistically significant treatment effect using a two-sided log-rank test at an overall significance level of α=0.05. Given the anticipated accrual rates and follow times, these 237 progression events will occur in an estimated 269 patients.

It is also hypothesized that the treatment of patients with ¹⁷⁷LuPSMA I&T will increase the Overall Survival (OS) from 18 months in the standard of care group to 25 months. Thus, a target Hazard Ratio (HR) under the alternative hypothesis of 0.70 is reasonable to expect for this Phase 3 study.

Utilizing the larger HR (associated with OS) and a 2:1 randomization, an estimated 352 events across both treatment arms will provide a 95% power to detect a statistically significant treatment effect using a two-sided log-rank test at an overall significance level of α=0.0. Given the anticipated accrual rates and follow times, these 352 deaths will occur in an estimated 400 patients.

### (ii) Planned Interim Analysis

The secondary outcome variable Overall Survival (OS) will have two interim analyses and a final analysis conducted. The first interim analyses will be conducted after approximately 25% (90) of the deaths have been observed and the second interim analysis will be conducted after approximately 75% (264) deaths have been observed. Then final analysis will be conducted after all planned 352 deaths are observed. It is anticipated the first interim analysis for OS will be conducted at the time when all 237 progression events will have been observed for the primary endpoint analysis.

The interim analyses and the final analysis for OS will be conducted using a two-sided log-rank test at a nominal significance level adjusted utilizing the standard O'Brien-Fleming spending function for a. The first interim analysis will have α=0.0006, the second interim analysis will have α=0.0151 and the final analysis of OS will be using a nominal significance level of α=0.047. These p-values satisfy the O'Brien-Fleming spending function for a cumulative α=0.05.

If the test of OS meets the nominal significance level at any of the interim analyses, the study will be deemed to be positive and patient accrual may stop, following review and approval by the Data Monitoring Committee (DMC).

(iii) Analysis Populations

A total of 400 patients are planned for this study.
Intention-to-treat (ITT) population: all randomized patients classified according to the treatment arms into which they were randomized, regardless of the actual treatment received.
Safety Population: all treated patients classified according to the actual treatment received, regardless of random assignment.
Cross over Population: All patients in the ITT population but where the analysis utilizes the information the patients were crossed over on treatment from the standard of care to ¹⁷⁷Lu-PSMA I&T. This will be utilized for the final OS analysis.

Other efficacy datasets (per protocol, evaluable, etc.) may be defined in the SAP (Statistical Analysis Plan), but the primary analyses of efficacy will be on the ITT population.

### (iv) Analysis of Efficacy

The primary analysis of efficacy will use the ITT population. Unless stated otherwise, the analysis of secondary endpoints will be based on data collected during the randomized treatment period.

a. Radiographic Progression-Free Survival

rPFS is time from randomization to the first documented radiographic progressive disease or death due to any cause. The rPFS time of any living patient with no radiographic documented progression, or any patient starting other anti-cancer systemic therapies, will be censored at the date of last evaluable disease assessment on-study. The rPFS time of patients with no evaluable disease assessment on-study will be censored at randomization. The frequency of rPFS will be performed approximately every 8 weeks from time of first treatment through Week 24 and then every 12 weeks thereafter.

Distributions of rPFS times will be estimated using the Kaplan-Meier product-limit method. The median rPFS times with two-sided 95% CIs will be estimated for each treatment group. A log-rank test will be used as the primary analysis for comparison of treatment effects. A Cox proportional hazards model will be used to estimate the HR, and its two-sided 95% CI (Confidence interval).

b. Overall Survival

OS time is from randomization to death due to any cause. OS will be followed for 5 years post-enrollment in the study or until death or loss to follow-up. Patient data for the standard of care group will not be censored at the time of crossover.

Distributions of OS times will be estimated using the Kaplan-Meier product-limit method. The median OS times with two-sided 95% CIs will be estimated for each treatment group. A log-rank test will be used as the primary analysis for comparison of treatment effects. A Cox proportional hazards model will be used to estimate the hazard ratio, and its two-sided 95% CI.

Supplementary analyses may be performed if a sufficient proportion of patients crossover to ¹⁷⁷Lu-PSMA-I&T. The Rank Preserving Structural Failure Time (RPSFT) (Robins et al 1991) methodology will be utilized in this analysis.

c. Second Radiographic Progression Free Survival

rPFS2 is time from randomization to the second documented radiographic progressive disease or death due to any cause. rPFS2 is) defined as the time from randomization to the second radiographic progression (using PCWG3 criteria as assessed by blinded independent central review [BICR]) or death in participants who crossover from the standard of care hormone therapy arm to treatment with ¹⁷⁷Lu-PSMA I&T.

Distributions of rPFS2 times will be estimated using the Kaplan-Meier product-limit method. The median rPFS 2 times with two-sided 95% CIs will be estimated for each treatment group. A log-rank test will be used as the primary analysis for comparison of treatment effects. A Cox proportional hazards model will be used to estimate the HR, and its two-sided 95% CI.

d. Progression Free Survival

Progression is defined as the first occurrence of PCWG3 progression, clinical/symptomatic progression and/or pain progression, or death due to any cause. The time to progression will be assessed for all patients. Any patient with no documented progression, or any patient starting other anti-cancer systemic therapies, will be censored at the date of last evaluable disease assessment on-study.

Distributions of PFS will also be estimated using the Kaplan-Meier product-limit method. The median PFS times with two-sided 95% CIs will be estimated for each treatment group. A log-rank test will be used as the primary analysis for comparison of treatment effects. A Cox proportional hazards model will be used to estimate the HR (Hazard ratio) and its two-sided 95% CI.

Supplementary analyses may be performed if a sufficient proportion of patients crossover to ¹⁷⁷Lu-PSMA-I&T. The Rank Preserving Structural Failure Time methodology will be utilized in this analysis.

e. Second Progression-Free Survival

Progression-free survival 2, is the time from randomization to progression based on the following events, whichever occurs first: RECIST 1.1 progression, PCWG3 progression, clinical/symptomatic progression and/or pain progression, or death due to any cause as assessed by the Investigator.

Distributions of PFS2 will also be estimated using the Kaplan-Meier product-limit method. The median PFS2 times with two-sided 95% CIs will be estimated for each treatment group. A log-rank test will be used as the primary analysis for comparison of treatment effects. A Cox proportional hazards model will be used to estimate the HR and its two-sided 95% CI.

Supplementary analyses may be performed if a sufficient proportion of patients' crossover to ¹⁷⁷Lu-PSMA I&T. The Rank Preserving Structural Failure Time methodology will be utilized in this analysis.

f. PSA50 Response Rate

PSA50 is defined as the response rate of patients who achieve a reduction of ≥50% in PSA from the baseline PSA assessment. The PSA50 in each treatment arm, with corresponding exact 95% CIs, will be computed. In addition, the difference in response rates, along with a 95% CI, will be determined. The primary test of treatment effect between ¹⁷⁷Lu-PSMA I&T and standard of care will be a Cochran-Mantel-Haenszel (CMH) general association chi-square test, controlling for the randomization strata. Relative risk with the two-sided 95% CI will be calculated.

g. Time to First Symptomatic Skeletal Event

The Time to First Symptomatic Skeletal (or SSE-free survival) event is defined as the occurrence of either bone-directed radiotherapy to relieve bony pain, new symptomatic pathologic fractures, spinal cord compression, or tumor-related orthopedic surgery. The time to SSE will be assessed for all patients. Any patient with no documented event, or any patient starting other anti-cancer systemic therapies, will be censored at the date of last evaluable disease assessment on-study.

Distributions of SSE will also be estimated using the Kaplan-Meier product-limit method. The median SSE times with two-sided 95% CIs will be estimated for each treatment group. A log-rank test will be used as the primary analysis for comparison of treatment effects. A Cox proportional hazards model will be used to estimate the HR and its two-sided 95% CI.

Supplementary analyses may be performed if a sufficient proportion of patients crossover to ¹⁷⁷Lu-PSMA I&T. The Rank Preserving Structural Failure Time methodology will be utilized in this analysis.

h. Time to Soft Tissue Progression

The Time to first radiographic soft tissue progression (STP) is defined as the occurrence of radiographic progression in soft tissue noted by PCWG3 modified RECIST 1.1. The time to STP will be assessed for all patients. Any patient with no documented event, or any patient starting other anti-cancer systemic therapies, will be censored at the date of last evaluable disease assessment on-study.

Distributions of STP will also be estimated using the Kaplan-Meier product-limit method. The median STP times with two-sided 95% CIs will be estimated for each treatment group. A log-rank test will be used as the primary analysis for comparison of treatment effects. A Cox proportional hazards model will be used to estimate the HR and its two-sided 95% CI.

Supplementary analyses may be performed if a sufficient proportion of patients crossover to ¹⁷⁷Lu-PSMA I&T. The Rank Preserving Structural Failure Time methodology will be utilized in this analysis.

i. Time to Chemotherapy

The Time to Chemotherapy (TTC) is defined as time from randomization to the initiation of chemotherapy or death, whichever comes first. The time to Chemotherapy will be assessed for all patients. Any patient with no documented event, or any patient starting other anti-cancer systemic therapies, will be censored at the date of last evaluable disease assessment on-study.

Distributions of TTC will also be estimated using the Kaplan-Meier product-limit method. The median TTC times with two-sided 95% CIs will be estimated for each treatment group. A log-rank test will be used as the primary analysis for comparison of treatment effects. A Cox proportional hazards model will be used to estimate the HR and its two-sided 95% CI.

Supplementary analyses may be performed if a sufficient proportion of patients crossover to ¹⁷⁷Lu-PSMA I&T. The Rank Preserving Structural Failure Time methodology will be utilized in this analysis.

j. Quality of Life

The EORTC QLQ-C30 is a questionnaire of 30 QoL questions developed to assess the QoL of cancer patients. It has been translated and validated into 81 languages including English and Mandarin Chinese. Version 3.0 of the QLQ-C30 will be used in this Phase 3 study. The QoL questionnaire has been used as an efficacy endpoint in over 3,000 Phase 3 cancer clinical trials.

The EORTC questionnaires will be given to the patients at baseline, Day 8 of each cycle, and at the EOT. There are numerous statistical techniques that have been presented in the literature to analyze QoL data. The statistical methods to be used will be included in the SAP.

k. Objective Response Rate

Objective response rate (ORR) to protocol treatment will be evaluated by RECIST version 1.1, and the best overall response will be classified as CR, PR, stable disease, progressive disease (PD), and not evaluable (NE). ORR is defined as the proportion of patients who achieve either a CR or a PR. CT, and bone scans as applicable, at baseline and during study will be reviewed by independent radiologists at a central imaging laboratory to determine objective response, date of response, and progression.

The ORR in each treatment arm, with corresponding exact 95% CIs, will be computed. In addition, the difference in response rates, along with a 95% CI, will be determined. The primary test of treatment effect between ¹⁷⁷Lu-PSMA I&T and standard of care will be a Cochran-Mantel-Haenszel (CMH) general association chi-square test, controlling for the randomization strata. Relative risk with the two-sided 95% CI will be calculated.

A further analysis of the ORR may be performed utilizing the Cross over population.

l. Disease Control Rate

Disease Control Rate (DCR) to protocol treatment will be evaluated by RECIST version 1.1, and the best overall response will be classified as CR (Complete response), PR (Partial response), stable disease, progressive disease (PD), and not evaluable (NE). DCR is defined as the proportion of patients who achieve disease control.

The DCR in each treatment arm, with corresponding exact 95% CIs, will be computed. In addition, the difference in response rates, along with a 95% CI, will be determined. The primary test of treatment effect between ¹⁷⁷Lu-PSMA I&T and standard of care will be a Cochran-Mantel-Haenszel (CMH) general association chi-square test, controlling for the randomization strata. Relative risk with the two-sided 95% CI will be calculated.

A further analysis of the DRR may be performed utilizing the Cross over population.

m. Duration of Response

In patients who achieve the best objective response of CR or PR, DoR is time from the first observation of CR or PR (whichever status occurs first) to the first documented progressive disease. DoR of any patient with no documented progression, or any patient starting other anti-cancer systemic therapies, will be censored at the date of last evaluable disease assessment on-study.

Distributions of DoR will also be estimated using the Kaplan-Meier product-limit method. The median DoR times with two-sided 95% CIs will be estimated for each treatment group. A log-rank test will be used as the primary analysis for comparison of treatment effects. A Cox proportional hazards model will be used to estimate the HR and its two-sided 95% CI.

Supplementary analyses may be performed if a sufficient proportion of patients' crossover to ¹⁷⁷Lu-PSMA I&T. The Rank Preserving Structural Failure Time methodology will be utilized in this analysis.

n. Time to PSA Progression

The time to PSA progression as assessed by a ≥25% increase in PSA from the post-treatment nadir or, if no nadir, the baseline PSA assessment. The time to PSA progression of any patient with no documented progression, or any patient starting other anti-cancer systemic therapies, will be censored at the date of last evaluable disease assessment on-study.

Distributions of time to PSA progression will also be estimated using the Kaplan-Meier product-limit method. The median time to PSA progression with two-sided 95% CIs will be estimated for each treatment group. A log-rank test will be used as the primary analysis for comparison of treatment effects. A Cox proportional hazards model will be used to estimate the HR, and its two-sided 95% CI.

Supplementary analyses may be performed if a sufficient proportion of patients crossover to ¹⁷⁷Lu-PSMA I&T. The Rank Preserving Structural Failure Time methodology will be utilized in this analysis.

### (vi) Clinical Laboratory Evaluations

Clinical laboratory results may be collected pretreatment through 28 days after the last dose of any study therapy. All clinically significant laboratory abnormalities namely tests that result in treatment modification and/or require intervention, may be recorded as AEs.

### H. End of Trial

Patients may remain on study treatment until BICR-confirmed disease progression, development of unacceptable toxicity, or withdrawal of consent. Patients discontinuing study treatment may enter the Long-term Follow-up Phase and remain on study until death, loss of follow-up, or withdrawal of consent, whichever comes first.

With an estimated accrual duration of 12-24 months, it is assumed that human patients may be followed for a minimum of approximately 34 weeks beyond Last Patient In (LPI) for the primary endpoint of radiographic progression free survival, to 5 years beyond LPI for the secondary endpoint of OS. This corresponds to total projected study duration of approximately 6-7 years.

### Example 6: PHARMACOKINETICS AND RADIATION DOSIMETRY SUB-STUDY

### A. Objectives

The objective of this study is to evaluate the plasma pharmacokinetic and radiation dosimetry of ¹⁷⁷Lu-PSMA I&T radioligand therapy in a subset of 30 patients.

This sub-study is designed to evaluate the plasma pharmacokinetic profile and radiation dosimetry of ¹⁷⁷Lu-PSMA I&T radioligand therapy over 4 treatment cycles.

### B. Study Design and Patient Population

Representative batch certification of the batches of ¹⁷⁷Lu-PSMA I&T used in the study is provided in Table 18.

TABLE 16
Representative certificate of analysis of ¹⁷⁷Lu-PSMA I&T

| | | |
|---|---|---|
| Characteristics | Specification | Result |

Release Tests

| Appearance | | Clear, colorless or slightly yellow | Pass |
|---|---|---|---|
| pH | | 4.0-5.0 ± 0.5 | 4.5 ± |
| Ascorbic acid concentration | | 15-35 mg/ml ± 5 mg/ml | 0.5 Pass |
| Radionuclidic identification | | Gamma ray energy | 113 keV ± |
| | | [symbol] | 10 keV |
| | | No other significant peaks with | 209 keV ± |
| | | gamma | 12 keV |
| Radiochemical Identity | | The radiometric RT of the test | Pass |
| | | sample | |
| Radiochemical | by HPLC | ≥95.0% as ¹⁷⁷Lu-PSMA | 98.2% ± 1.8% |
| purity | by TLC | 177Lu-colloid ≤ 5,0% | 0% |

| Chemical purity | Lu-PSMA-I&T concentration ≤ | 1.4 ug/ml ± |
|---|---|---|
| | 6.0 μg/ml | 0.8 μg/ml |
| Ethanol content (w/w) | 2.5%-4.5% ± 1.5% | 3.7% ± 0.7% |
| ¹⁷⁷Lu-PSMA-I&T | Reported | 168 GBq/μmol ± |
| Specific activity | | 50 GBq/μmol |
| Endotoxins | <175 EU/20 mL vial | Pass |

Post Release Tests

| | | |
|---|---|---|
| Sterility | Sterile | Pass |
| Radionuclidic purity | ≥99.9% of the total activity of | Pass |
| | [symbol] | |

[symbol]

indicates data missing or illegible when filed

The radioactivity concentration (RAC), concentration of unchelated PSMA (PSMA I&T), and concentration of chelated PSMA (M-PSMA I&T) is provided in Table 19 for specific batches. The radioactivity concentration may be from 564 to 616 MBq/mL±20 MBq/mL.

TABLE 17
Properties of specific batches used in the study.

| | RAC | PSMA I&T | M-PSMA I&T |
|---|---|---|---|
| Batch | (MBq/mL)* | (μg/mL) | (μg/mL) |
| LKMI2316A-1 | 585 | 3.7 | 1.8 |
| LKMI2316G-1 | 588 | 3.9 | 1.6 |
| LKMI2317G-1 | 585 | 3.8 | 2.4 |
| LKMI2318B-1 | 586 | 3.7 | 1.6 |
| LKMI2320G-1 | 564 | 2.7 | 0.9 |
| LKMI2322A-1 | 616 | 2.5 | 1.1 |
| LKMI2322G-3 | 610 | 2.2 | 1.3 |
| LKMI2323A-2 | 596 | 2.0 | 1.4 |
| LKMI2323B-1 | 588 | 2.4 | 1.5 |
| LKMI2326B-1 | 589 | 2.1 | 0.8 |
| LKMI2328A-1 | 597 | 1.9 | 1.4 |
| LKMI2328G-2 | 594 | 2.8 | 0.7 |
| LKMI2329A-1 | 591 | 2.2 | 0.9 |
| LKMI2329B-1 | 588 | 2.4 | 0.7 |

*Measured at the end of production

The plasma pharmacokinetic profile and radiation absorbed dose of ¹⁷⁷Lu-PSMA I&T radioligand therapy will be assessed in a subset of 30 patients enrolled that will be participating in the study of Example 5. The subset of 30 patients will undergo the same screening procedures as the main protocol of Example 5 in order to be randomized into the study, following the same Inclusion/Exclusion criteria of Example 5, with the following additional enrollment criteria:

### Inclusion Criteria

Enrollment in the main study (Example 5).
Separate informed consent for participation in the sub-study.
Willing to undergo planar and/or SPECT/CT imaging at 4 hours, 24 hours, 48 hours, and 6-8 days after each ¹⁷⁷Lu-PSMA I&T treatment cycle (Main study Cycles 1-6).

### Exclusion Criteria

Unable to undergo SPECT/CT imaging as required in the sub-study protocol.

### C. Methods

The plasma pharmacokinetic profile will be determined in the subset of 30 patients enrolled in this study by acquiring plasma samples prior to and at approximately 1, 4, 24, and 48 hours and 6-8 days after the completion of each ¹⁷⁷Lu-PSMA I&T infusion. The time of completion of ¹⁷⁷Lu-PSMA I&T infusion and the actual times of sample collection will be recorded. Radioactivity will be assayed in plasma samples using a calibrated well counter, and the percent of the injected dose will be calculated after correcting the radioactive decay half-life of 6.647 days for ¹⁷⁷Lu.

Planar whole body scintigraphic images will be acquired at approximately 4, 24, and 48 hours and at 6-8 days following completion of each ¹⁷⁷Lu-PSMA I&T infusion. SPECT/CT images of the upper abdomen, kidneys, and salivary glands will be acquired at approximately 24 hours and 6-8 days following each ¹⁷⁷Lu-PSMA I&T infusion. The actual start time and date for all scintigraphic imaging will be recorded on the CRF.

Imaging data will be submitted to a core imaging lab for processing. For tumor dosimetry calculations, regions of interests (ROIs) showing negligible overlap with high physiologic uptake or other positive lesions on scintigraphy will be selected. Background ROIs will be drawn from outside of the body. At a minimum, ROIs will be selected for the whole body, kidneys, liver, parotid glands, submandibular glands, and lacrimal glands, as well as tumor ROIs and other organs demonstrating significant uptake of the ¹⁷⁷Lu-PSMA I&T.

Following the administration/infusion of a dose of a ¹⁷⁷Lu-PSMA I&T solution, the human patient(s) may undergo single photon emission computed tomography (SPECT)/computed tomography (CT) imaging, e.g., at four timepoints (4 h, 24 h, 48 h 168 h). Image data may be analyzed to compute time integrated activity coefficients (TIAC) in each organ of interest and/or with appreciable and meaningful ¹⁷⁷Lu-PSMA I&T activity above background which included kidneys, bladder, liver, lumbar vertebrae L2-L4, lacrimal glands, salivary glands, intestine, whole body, and combinations thereof. To compute subject-specific organ doses, organ-level TIAC data may be entered into Organ Level Internal Dose Assessment, OLINDA 2.2.3 and resulting organ doses for each target organ (e.g., kidney) and whole body effective dose may be collated and averaged.

### D. Schedule of Activities Specific to the Pharmacokinetic and Radiation Dosimetry Sub-Study

### (i) Screening

Verify inclusion/exclusion criteria are met
Obtain informed consent for participation in sub-study
   (ii)¹⁷⁷Lu-PSMA I&T Infusion Cycles 1-4
Obtain plasma samples prior to and at approximately 1, 4, 24, and 48 hours and 6-8 days after the completion of each ¹⁷⁷Lu-PSMA I&T infusion as shown in Table 20.
Obtain planar whole body scintigraphic images at approximately 4, 24, and 48 hours and 6-8 days following completion of each ¹⁷⁷Lu-PSMA I&T infusion.
Obtain SPECT/CT images at approximately 24 hours and 6-8 days following completion of each ¹⁷⁷Lu-PSMA I&T infusion.

TABLE 18
Schedule of time points to take samples.

| Activities | | Time After Each ¹⁷⁷Lu-PSMA | | | | | |
|---|---|---|---|---|---|---|---|
| Specific to | | I&T Infusion (Cycles 1-4) | | | | | |
| Sub-study | Screening | Prior to | 1 hr | 4 hr | 24 hr | 48 hr | 6-8 days |
| Informed | X | | | | | | |
| consent | | | | | | | |
| Plasma PK | | X | X | X | X | X | X |
| [symbol] | | | | | | | |
| Whole | | | | X | X | X | X |
| Body | | | | | | | |
| Planar | | | | | | | |
| Imaging | | | | | | | |
| SPECT/CT | | | | | X | | X |
| Imaging | | | | | | | |

[symbol]

indicates data missing or illegible when filed

### E. Statistical Methods

### (i) Pharmacokinetic Parameters

The following PK parameters derived from whole blood radiation counts of ¹⁷⁷Lu-PSMA-I&T infusion will determined as appropriate:
AUC_{0-24; 0-last}: area under the whole blood radiation counts time curve (0 to 24 hours, 0 to last) calculated by use of the trapezoidal rule
AUC_{0-∞}: area under the whole blood radiation counts time curve from AUC₀₋₂₄ or AUC₀₋ₗₐₛₜ (calculated by use of the trapezoidal rule)+area under the curve estimated by Cpₙ/kₑₗ, where: Cpₙ=last observed whole blood radiation count at time n
kₑₗ=elimination rate constant, calculated from the log-linear terminal portion of the whole blood radiation counts time curve
Cₘₐₓ: observed maximum whole blood radiation count
Tₘₐₓ: time to Cₘₐₓ
kₑₗ: elimination rate constant, calculated from the log-linear terminal portion of the whole blood radiation counts time curve
t_{1/2}: apparent elimination half-life, calculated as 1n2/kₑₗ
CL: total body clearance for extravascular administration at steady-state, calculated as Dose/AUC₀₋₂₄
V_{d}: volume of distribution

### (ii) ¹⁷⁷Lu-PSMA I&T Infusion Radiation Counts

C(t): The radiation counts measured at time=t were tabulated for time and each patient. Whole blood radiation counts below the limit of quantitation were treated as zero (0).
   (iii) Area Under the Curve

The following characteristics will be calculated for all complete profiles, if not stated otherwise:
AUC_{0-24, 0-last}: The area under the whole blood radiation counts C(t) from administration will be calculated by use of the trapezoidal rule.

### Example 7: Biodistribution and Dosimetry Results after Administration of ¹⁷⁷Lu-PSMA I&T in the Dosimetry Sub-Study Described in Example 6

The aim of this study was to present the biodistribution and dosimetry results following the administration of ¹⁷⁷Lu-PSMA-I&T in a subset of 6 patients enrolled in the dosimetry sub-study of Example 6. Specific focus was on estimating the cumulative absorbed renal dose after 6 treatment cycles of ¹⁷⁷Lu-PSMA I&T.

Six subjects were administered a target dose of 7.4 GBq (mean 7.52±0.16 GBq) of ¹⁷⁷Lu-PSMA-I&T. Following the infusion, subjects underwent single photon emission computed tomography (SPECT)/computed tomography (CT) imaging at four timepoints (4 h, 24 h, 48 h, 168 h). Image data were analysed to compute time integrated activity coefficients (TIAC) in each organ of interest and/or with appreciable and meaningful ¹⁷⁷Lu-PSMA-I&T activity above background which included kidneys, bladder, liver, lumbar vertebrae L2-L4, lacrimal glands, salivary glands, intestine and whole body. To compute subject-specific organ doses, organ-level TIAC data were entered into Organ Level Internal Dose Assessment, OLINDA 2.2.3 and resulting organ doses for each target organ and whole-body effective dose were collated and averaged.

### A. Data Acquisition

Patients received SPECT/CT imaging (anatomical coverage included the salivary gland and extended through the pelvis) following the injection of 7.4 (+/−10%) GBq of ¹⁷⁷Lu-PSMA-I&T (detailed injected doses are reported). All patients completed the first treatment cycle and the acquisition of images was carried out without any protocol deviation.

TABLE 19
Injected doses

| Subject ID | Injected Dose ¹⁷⁷Lu-PSMA-I&T (GBq) |
|---|---|
| 25-005 | 7.58 |
| 25-007 | 7.56 |
| 25-008 | 7.21 |
| 80-001 | 7.67 |
| 80-004 | 7.52 |
| 80-007 | 7.54 |
| Mean | 7.52 |
| SD | 0.16 |

### B. Image Pre-Processing and Segmentation

The imaging data was analyzed using Invicro's VivoQuant software, a validated software used in a 21 CFR § 11-compliant workflow. The general quantification approach was based on the principles detailed in MIRD Pamphlet No. 16 (Siegel J, Thomas S, Stubbs J, et al. MIRD pamphlet no. 16: Techniques for quantitative radiopharmaceutical biodistribution data acquisition and analysis for use in human radiation dose estimates. J Nucl Med. 1999; 40:37S-61S and MIRD Primer for Absorbed Dose Calculations, Revised Ed. J Nucl Med. 1991), No 23 (Dewaraja Y K, Frey E C, Sgouros G, et al. MIRD pamphlet No. 23: quantitative SPECT for patient-specific 3-dimensional dosimetry in internal radionuclide therapy. J Nucl Med. 2012; 53(8):1310-1325), and No 26 (Ljungberg M, Celler A, Konijnenberg M W, et al. MIRD Pamphlet No. 26: Joint EANM/MIRD Guidelines for Quantitative ¹⁷⁷Lu SPECT Applied for Dosimetry of Radiopharmaceutical Therapy. J Nucl Med. 2016; 57(1):151-162), as appropriate.

Each SPECT image was co-registered with its corresponding CT. Image calibration was performed to convert the counts measured on the SPECT images to activity (Bq) (Ljungberg et al. MIRD Pamphlet No. 26: Joint EANM/MIRD Guidelines for Quantitative ¹⁷⁷Lu SPECT Applied for Dosimetry of Radiopharmaceutical Therapy. J Nucl Med. 2016; 57(1):151-162). The same SPECT/CT system was used in each patient for the entirety of the dosimetry study.

Regions of interest (kidneys, bladder, liver, lumbar vertebrae L2-L4, lacrimal glands, salivary glands and whole body) were drawn on each dataset by a trained image analyst. Lumbar vertebrae L2-L4 was delineated on the CT images (Hindorf C, Glatting G, Chiesa C, Linden O, Flux G, Committee ED. EANM Dosimetry Committee guidelines for bone marrow and whole-body dosimetry. Eur J Nucl Med Mol Imaging. 2010; 37(6):1238-1250) to derived red marrow doses. For kidneys, an ROI was drawn on the SPECT image to include all the uptake seen in the kidneys. Similarly for lacrimal glands, all the uptake observed on the SPECT image at the lacrimal glands location was segmented.

Time-activity curves of the activity in each ROI as a function of time were plotted. These curves were normalized by the known injected activity to arrive at units of fraction of injected activity. Calculations of time-integrated activity coefficient (TIAC, in units of hours) were performed for each region of interest listed above.

### C. Dosimetry Analysis

Clearance of the tracer was derived from a data-appropriate fit of each organ TAC. The curve fit was optimized using the Akaike information criterion (AIC) and checked visually by an image scientist. Depending on the shape of the TAC, the data was fitted with the sum of one, two or three exponentials or using a rise and fall model. TIACs were calculated through analytical integration of the curve fit extrapolated to infinite. If no appropriate fit was found, the AUC was estimated using the sum of the trapezoidal integration of imaging measurements and physical decay from the last timepoint onward (the fraction of injected activity at t=0 was set to zero for organs and to 1 for total body). The remainder activity was determined by subtracting the cumulative organ TIACs from the whole body TIAC.

Organ TIACs were inputted into Organ Level Internal Dose Assessment, OLINDA 2.2.3 for the computation of organ and whole-body absorbed doses and effective dose (computed using ICRP-103 weighing factors). ICRP-89 derived male phantom was used for dose estimation. Organs and body weights were scaled based on height and mass using the effective mass as described by Wahl et al.⁵ Kidney volumes were measured for each patient on the CT images and used for the kidneys mass of the ICRP-89 phantom assuming a density of 1 g/ml.

Lacrimal glands absorbed dose was computed using the OLINDA 2.2.3 sphere model which assumes local deposition of the dose only. A lacrimal gland volume of 0.680 cm³ was used for all patients, based on the results published by Bingham et al. (Calculated CT volumes of lacrimal glands in normal Caucasian orbits. Ophthal Plast Reconstr Surg. 2013; 29:157-159).

### D. Dosimetry Results—Cycle 1 Results

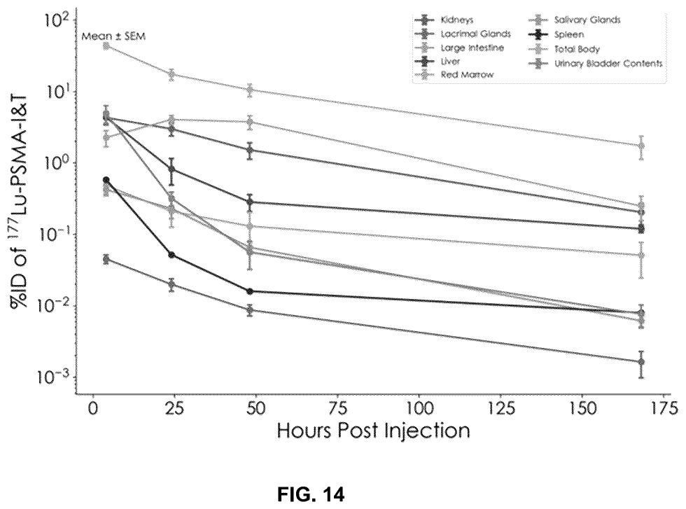

Physiologic uptake was predominantly seen in the kidneys, urinary bladder, and salivary glands. ¹⁷⁷Lu-PSMA-I&T was primarily excreted through the kidneys and via urinary excretion in the bladder. In four patients, uptake in the intestines was seen while no uptake was seen in the other 2 patients analyzed. Time activity curves for all ROI delineated are reported in FIG. 14, Whole body time activity curves showed an initial rapid clearance with low activity retention at 4 h, followed by a second, slower clearance.

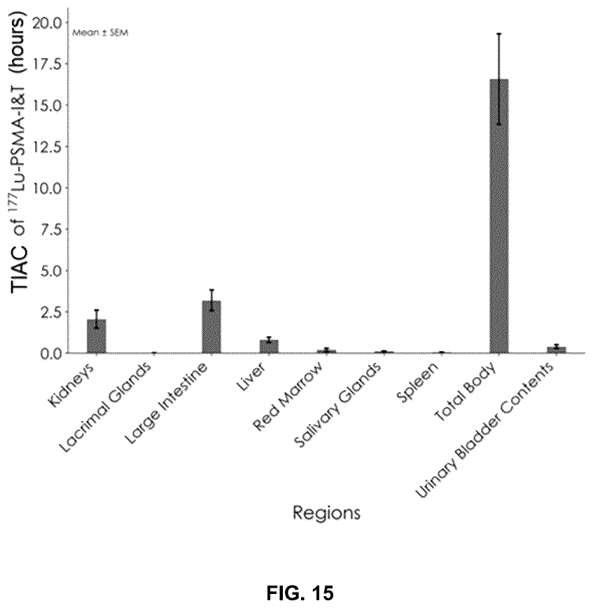

FIG. 15 reports the mean TIACs for the source organs. Whole-body time integrated activity coefficient were between 8.2 and 27.6 hours with a mean of 16.6 hours (SD 6.7), as shown in Table 22. Mean whole body effective dose was 0.0068 mSv/MBq (SD 0.033). Whole body effective doses are shown in Table 23.

TABLE 20
Whole body time integrated activity
coefficients (hours) per patient, mean and SD

| Subject | 25- | 25- | 25- | 80- | 80- | 80- | | |
|---|---|---|---|---|---|---|---|---|
| ID | 005 | 007 | 008 | 001 | 004 | 007 | Mean | SD |
| Whole | 12.6 | 19.7 | 14.2 | 27.6 | 8.2 | 17.2 | 16.6 | 6.7 |
| Body | | | | | | | | |
| TIAC | | | | | | | | |
| (hrs) | | | | | | | | |

TABLE 21
Effective dose for all six patients.

| Subject ID | Effective Dose (mSv/MBq) cycle 1 |
|---|---|
| 25-005 | 3.11E−02 |
| 25-007 | 1.11E−01 |
| 25-008 | 6.97E−02 |
| 80-001 | 8.11E−02 |
| 80-004 | 2.68E−02 |
| 80-007 | 9.03E−02 |

Organs with the highest absorbed doses were the lacrimal glands (0.77±0.25), kidneys (0.46±0.23 Gy/GBq), and the urinary bladder wall (0.37±0.20 Gy/GBq). Absorbed doses to the part of the large intestines (left colon, right colon and rectum) were respectively: 0.39±0.32, 0.21±0.17, 0.37±0.31 Gy/GBq). Dose to the salivary glands was 0.10±0.06 Gy/GBq. Absorbed doses to the kidneys (in Gy and Gy/GBq) are reported for each patient included in the dosimetry sub-study in Table 24 and average doses to all target organs are reported in Table 25.

TABLE 22
Absorbed doses to the kidneys in Gy and Gy/GBq.

| | | Kidneys | Kidneys |
|---|---|---|---|
| | Injected Dose ¹⁷⁷Lu- | Absorbed | Absorbed |
| Subject ID | PSMA-I&T (GBq) | Dose (Gy) | Dose (Gy/GBq) |
| 25-005 | 7.58 | 4.87 | 0.64 |
| 25-007 | 7.56 | 2.56 | 0.34 |
| 25-008 | 7.21 | 5.82 | 0.81 |
| 80-001 | 7.67 | 2.03 | 0.27 |
| 80-004 | 7.52 | 1.68 | 0.22 |
| 80-007 | 7.54 | 3.62 | 0.48 |
| Mean | 7.52 | 3.43 | 0.46 |
| SD | 0.16 | 1.65 | 0.23 |

Note:
one gray (Gy) is a measurement of radiation and is the international system of units (SI) equivalent of 100 rads, which is equal to an absorbed dose of 1 Joule/kilogram.

TABLE 23
Absorbed doses in Gy/GBq in OLINDA target organs.

Absorbed Dose (Gy/GBq)

| Target Organ | Mean | SD |
|---|---|---|
| Adrenals | 0.02 | 0.01 |
| Brain | 0.01 | 0.01 |
| Esophagus | 0.01 | 0.01 |
| Eyes | 0.01 | 0.01 |
| Gallbladder Wall | 0.02 | 0.01 |
| Left colon | 0.39 | 0.32 |
| Small Intestine | 0.05 | 0.03 |
| Stomach Wall | 0.01 | 0.01 |
| Right colon | 0.21 | 0.17 |
| Rectum | 0.37 | 0.31 |
| Heart Wall | 0.01 | 0.01 |
| Kidneys | 0.46 | 0.23 |
| Liver | 0.03 | 0.02 |
| Lungs | 0.01 | 0.01 |
| Pancreas | 0.02 | 0.01 |
| Prostate | 0.02 | 0.01 |
| Salivary Glands | 0.10 | 0.06 |
| Red Marrow | 0.02 | 0.02 |
| Osteogenic Cells | 0.02 | 0.01 |
| Spleen | 0.02 | 0.01 |
| Testes | 0.01 | 0.01 |
| Thymus | 0.01 | 0.01 |
| Thyroid | 0.01 | 0.01 |
| Urinary Bladder Wall | 0.37 | 0.02 |
| Total Body | 0.02 | 0.01 |

### E. Kidney Absorbed Dose Projected to 6 Cycles

Kidneys were identified as the main organ at risk. To determine the safety of delivering 6 treatment cycles, projection of the cumulative absorbed dose to the kidneys after 6 treatment cycles was calculated by multiplying the number of cycles by the absorbed dose at cycle 1. With an injected dose of 7.4 GBq per cycle, the estimated mean absorbed dose for the kidneys after 6 cycles is 20.4 Gy±10.2 Gy (Table 26).

TABLE 24
Absorbed doses to the kidneys extrapolated to 6 cycles (in
Gy/GBq, and assuming an administered dose of 7.4 Gy)

| | | | Kidneys | Extrapolation |
|---|---|---|---|---|
| | Injected | | Absorbed | of Absorbed |
| | Dose | | Dose | Dose to 6 |
| | ¹⁷⁷Lu- | Kidneys | (Gy) | Cycles (Gy) |
| | PSMA- | Absorbed | Assuming | Assuming |
| Subject | I&T | Dose | 7.4 GBq | 7.4 GBq |
| ID | (MBq) | (Gy/GBq) | per cycle | per cycle |
| 25-005 | 7.58 | 0.64 | 4.8 | 28.5 |
| 25-007 | 7.56 | 0.34 | 2.5 | 15.0 |
| 25-008 | 7.21 | 0.81 | 6.0 | 35.8 |
| 80-001 | 7.67 | 0.27 | 2.0 | 11.8 |
| 80-004 | 7.52 | 0.22 | 1.7 | 9.9 |
| 80-007 | 7.54 | 0.48 | 3.6 | 21.3 |
| mean | 7.52 | 0.46 | 3.4 | 20.4 |
| SD | 0.16 | 0.23 | 1.7 | 10.2 |

### F. Conclusion and Discussion

Numerous studies have shown that ¹⁷⁷Lu-PSMA I&T is safe and well tolerated. Dosimetry results from cycle 1 of the dosimetry sub-study were in line with published dosimetry reports using the same ligand (Schuchardt et, al. Prostate-Specific Membrane Antigen Radioligand Therapy Using ¹⁷⁷Lu-PSMA I&T and ¹⁷⁷Lu-PSMA-617 in Patients with Metastatic Castration-Resistant Prostate Cancer: Comparison of Safety, Biodistribution, and Dosimetry. J Nucl Med. 2021; 63:1199-807 and Herrmann K. et al. Multi-cycle dosimetry of [177Lu]Lu-PSMA-617 for the treatment of metastatic castration-resistant prostate cancer: results from the VISION trial sub-study. Journal of Nuclear Medicine June 2022, 63 (supplement 2) 2626). Most common reported adverse events are mild and transient xerostomia, fatigue, anaemia, neutropenia, and thrombocytopenia Therefore, while normal organs receiving the highest absorbed doses are the lacrimal glands and the salivary glands, kidneys are identified as the dose limiting organs.

Preliminary kidney dosimetry results of this trial are comparable with the results from other dosimetry studies of ¹⁷⁷Lu-PSMA I&T and ¹⁷⁷Lu-PSMA-617, a similar ligand, in patients with metastatic castration-resistant prostate cancer. Specifically, the VISION trial reported a mean renal absorbed dose of 0.43±0.16 Gy/GBq from dosimetry analysis on 29 patients⁹ and Violet et al. (Dosimetry of ¹⁷⁷Lu-PSMA-617 in Metastatic Castration-Resistant Prostate Cancer: Correlations Between Pretherapeutic Imaging and Whole-Body Tumor Dosimetry with Treatment Outcomes. J Nucl Med. 2019 April; 60(4):517-523) calculated a mean renal absorbed dose of 0.39±0.16 Gy/GBq. Other dosimetry studies estimated higher kidney absorbed doses of 0.73±0.33 Gy/GBq and 0.8 Gy/GBq. However, these studies were done using 2D planar images instead of SPECT images and the overlay of liver and bowel activity could have led to higher absorbed doses.

Cumulative absorbed dose to the kidneys after six treatment cycles was projected by assuming that kidneys accumulate radiation damages from cycle to cycle and that kidneys absorbed dose will have minimal variability between cycles. Studies looking at dosimetry variations from cycle to cycle have found little variations in kidney absorbed doses, and that extrapolation dosimetry results from the first cycle was highly predictive of the cumulative absorbed dose to the kidneys at the end of treatment. Based on our results, the projected cumulative absorbed dose to the kidneys at 6 cycles will be 20.4±10.2 Gy. The mean projected dose is below the commonly accepted absorbed dose limit of 23 Gy, which comes from external beam radiation therapy and is considered conservative when applied to targeted radionuclide therapy. In the Vision trial, the projected cumulative absorbed dose to the kidneys at 6 cycles was 19±7.3 Gy and no renal toxicities were observed.

Dosimetry results suggest that ¹⁷⁷Lu-PSMA I&T treatment with 6 cycles is possible without the risk of kidney toxicities.

All references cited herein are hereby incorporated by reference. The foregoing is offered primarily for purposes of illustration. It will be readily apparent to those skilled in the art that further drugs can be included, and that the components, additives, proportions, methods of formulation, methods of use, and other parameters described herein can be modified further or substituted in various ways without departing from the spirit and scope of the invention.

### Example 7A: Evaluate the Safety and Efficacy of ¹⁷⁷Lu-PSMA-I&T Radioligand Therapy Compared to Hormone Therapy in Men with Metastatic Castration-Resistant Prostate Cancer (mCRPC)

Eclipse is a Phase 3, open-label, multicenter, randomized trial evaluating the safety and efficacy of ¹⁷⁷Lu-PSMA-I&T radioligand therapy compared to hormone therapy in men with metastatic castration-resistant prostate cancer (mCRPC). ¹⁷⁷Lu-PSMA-I&T is a radioactive therapeutic agent that specifically targets the prostate specific membrane antigen (PSMA) that is expressed on both primary and metastatic prostate cancer cells.

A sub-study was conducted to evaluate the PK and radiation dosimetry of ¹⁷⁷Lu-PSMA-I&T, in which patients underwent PK sampling and SPECT/CT imaging after the first and third ¹⁷⁷Lu-PSMA I&T infusion.

### Methods

Data Acquisition: All participating sites underwent a rigorous site setup process during which all dose calibrators, scanners, and gamma counters used in the sub-study were calibrated. Sites were allowed to enroll patients in the sub-study only after the completion and approval of all site setup processes by Invicro.

Patients underwent SPECT/CT imaging, with anatomical coverage typically extending from the salivary glands to the pelvis, following the administration of a targeted 7.4±10% GBq of ¹⁷⁷Lu-PSMA-I&T at cycle 1. SPECT/CT imaging was performed at four timepoints: 4 h, 24 h, 48 h, and 168 h post-injection. Additionally, a subset of patients imaged at Cycle 1 (16 out of 27), were also imaged at Cycle 3 (Table 7 in Appendix 1).

Image acquisition and reconstruction protocols were standardized across all imaging timepoints and patients at each site. Furthermore, each site consistently utilized the same SPECT/CT scanner throughout the duration of the study to ensure uniformity.

In general, raw projection data was acquired into a 128×128 matrix with medium energy general purpose (MEGP) collimation using a step and shoot acquisition mode, acquiring 60 projections per detector (180 degrees rotation per detector), 20 seconds per projection, with an acquisition zoom of 1.0, and a 20% (±10%) energy window centered over 208 keV.

A low-dose CT scan was performed prior to beginning the SPECT scan for attenuation correction. Sites were instructed to use the Institutional standard parameters for acquisition of the low-dose CT. Institutional standard reconstruction parameters were used. Scatter correction was required to be applied using a 20% (±10%) scatter window centered over 170 keV.

PK plasma samples were collected at pre-Infusion, 1 h, 4 h, 24 h, 48 h, and 168 h, and counted for radioactivity. Blood samples were first centrifuged to extract plasma, and then the radioactivity in plasma was counted in a gamma counter.

For a detailed summary of site qualification and set up and data acquisition procedures, refer to the Technical Operations Manual (TOM) (Appendix 2).

### Image Analysis Methods

### Image Pre-Processing and Segmentation

The imaging data was analysed using Invicro's VivoQuant software, a validated software used in a 21 CFR § 11-compliant workflow. The general quantification approach was based on the principles detailed in the Medical Internal Radiation Dose (MIRD) Pamphlet No. 16,¹ No 23,2 and No 26,3 as appropriate.

SPECT images were calibrated in units of Becquerel (Bq) using a calibration factor derived from a source of known activity, measured during site set up with the same acquisition and reconstruction parameters employed for patient imaging. A patient-specific calibration factor was also derived at each clinical time point after acquisition of the patient SPECT/CT. As a quality control measure, if the patient-specific calibration factor differed by more than 10% from the site set up calibration factor, an investigation was conducted.

Volume of interest (VOI) (kidneys, bladder, liver, lumbar vertebrae L2-L4, lacrimal glands, salivary glands [parotid and submandibular glands], and whole body) were delineated on each dataset by a trained image analyst. The GI tract and spleen were delineated only in a subset of patients where these organs displayed visible uptake. Organs were segmented either by drawing the entire contour of the organ, or by placing a sphere in a representative region of the organ and then multiplying by the organ's effective mass (based on patient's height and weight).⁴ Segmentation methods for each organ are specified in Table 1 and were consistently applied to each patient.

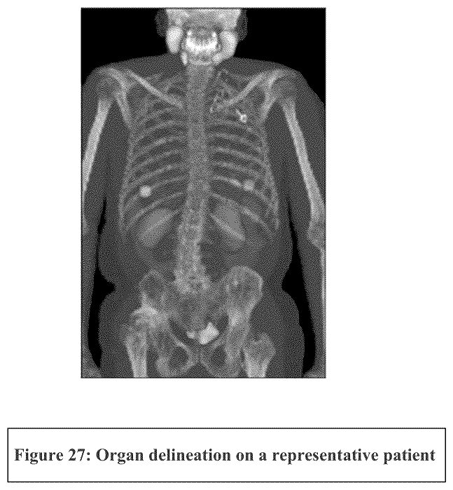

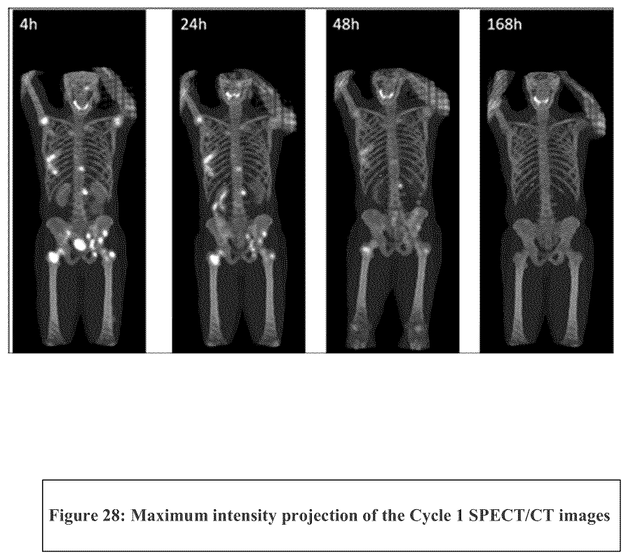

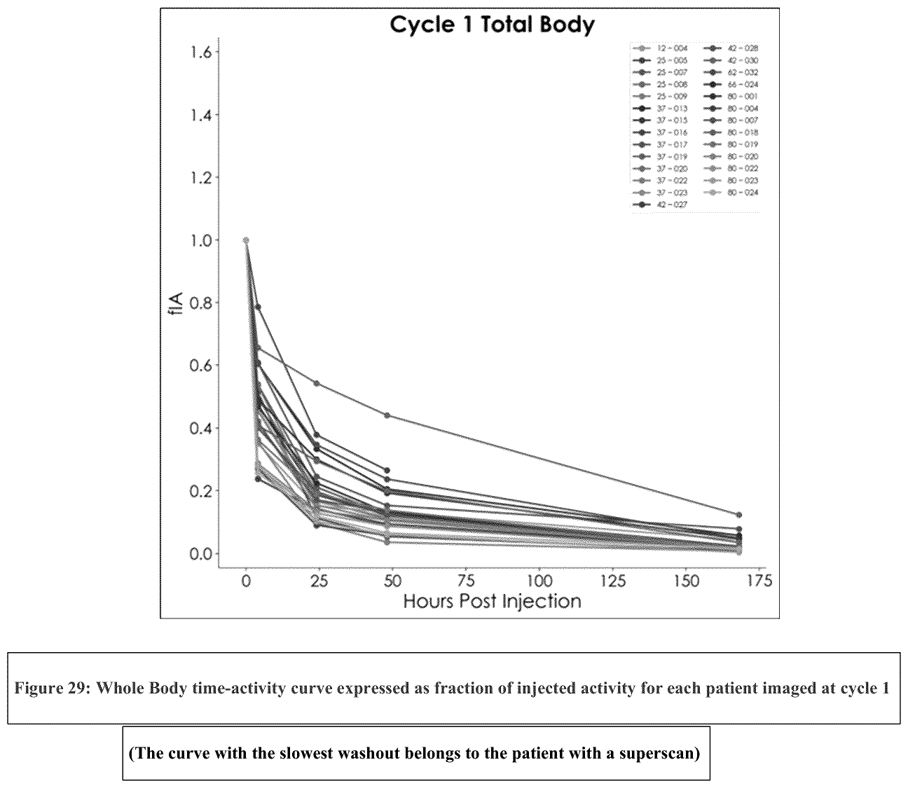

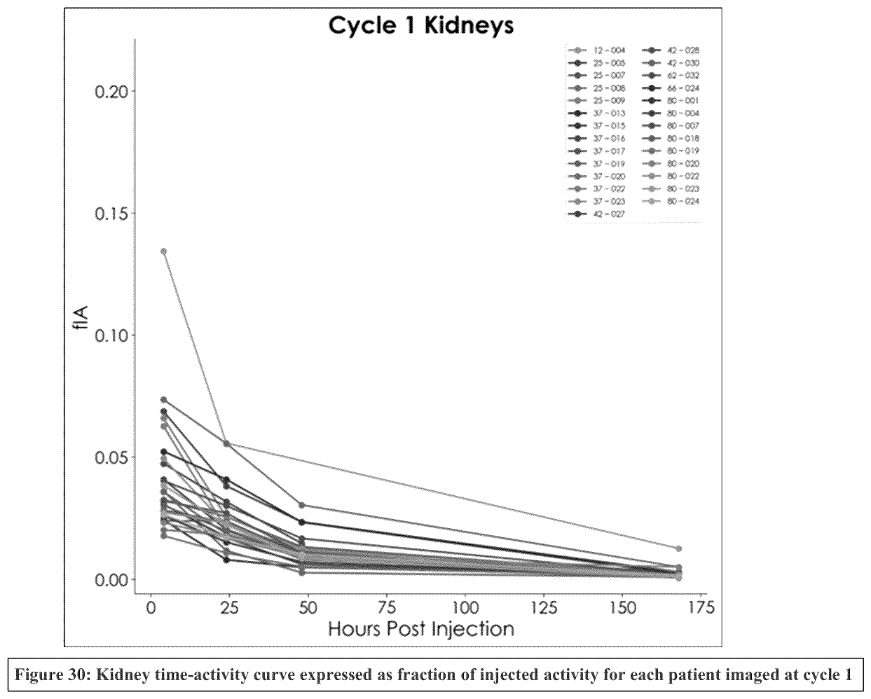

A maximum intensity projection (MIPs) CT image with segmented regions is shown for a representative patient in FIG. 27.

TABLE 25
Organ VOIs and delineation method

| Organ | Method |
|---|---|
| Bladder | CT and SPECT Uptake |
| Liver | Sphere based off CT |
| Spleen | Sphere based off CT |
| Kidney | SPECT Uptake |
| L2-L4 | CT |
| Salivary Glands | SPECT Uptake |
| GI Tract | SPECT Uptake |
| Lacrimal Glands | SPECT Uptake |

### Plasma Quantitative Analysis

Whole blood samples were collected as detailed in the protocol Schedule of Events i.e. pre-injection, and then at 1 h, 4 h, 24 h, 48 h, and 168 h post-injection. The samples were first centrifuged to separate plasma, and the radioactivity in the plasma was then measured using a gamma counter. Plasma concentrations were subsequently fitted with a bi-exponential function to derive outcome parameters such as the half-life and clearance rate of the radiopharmaceutical in the plasma.

### Normal Organ Dosimetry Analysis

Clearance of the radiopharmaceutical was derived from a data-appropriate fit of each organ's TAC. Depending on the TAC's shape, the data was fitted with the sum of one, two, or three exponentials, or using a rise and fall model. Fitting was performed with in-house Python software. TIACs were calculated through analytical integration of the curve fit extrapolated to infinity. If no appropriate fit was found, the area under the curve (AUC) was estimated using the sum of the trapezoidal integration of imaging measurements and physical decay from the last timepoint onward (the fraction of injected activity at t=0 was set to zero for organs and to 1 for the whole body). A whole-body TAC was derived from delineating the imaged body. The remainder activity was determined by subtracting the cumulative organ TIACs from the whole body TIAC.

Organ TIACs were input into OLINDA 2.2.3 for the computation of organ and whole-body absorbed doses, using International Commission on Radiological Protection (ICRP)-103 weighing factors. The ICRP-89 derived male phantom was used for dose estimation. Organs and body mass as defined by ICRP-89 were scaled based on height and mass using patient effective mass.⁴ The voiding bladder model as implemented in OLINDA was used to determine bladder TIAC, assuming a voiding interval of 4 hours. The human alimentary tract model (HAT model) described in ICRP 100 and implemented in OLINDA was used to compute doses to the small intestine, right colon, left colon and rectum for patients showing clearance through the GI tract. The fraction of injected activity (fIA) cleared via the GI tract was determined by taking the peak fIA in the GI tract over all the imaging timepoints. Red marrow dose estimation was derived from segmenting the Lumbar Vertebrae L2-L4 as seen on CT. The Red Marrow mass in L2-L4 was assumed to be 6.7% of the total red marrow mass.5

### Single Time Point Dosimetry Analysis Cycle 3

To evaluate potential changes in the tracer's kinetics and uptake after multiple treatment cycles, 16 of the 27 patients included in the dosimetry sub study, were imaged at both cycle 1 and cycle 3. Among these, at cycle 3, three patients were imaged at all 4 timepoints and the other 13 were imaged at one or two timepoints only (24 h, 48 h or both 24 h and 48 h) (Table 7 in Appendix 1). For these 13 patients, the TACs obtained at cycle 1 were used for each source organ and scaled to the normalized activity value(s) measured at Cycle 3. For patients imaged at two timepoints, the TACs were preferably scaled using the average scaling factor between the two timepoints. If the two timepoints yielded significantly different scaling factors, the more conservative timepoint was preferred, provided the dose estimation derived was reasonable.

### Data Acquisition Quality Control

Quantitative accuracy of each SPECT image was controlled using a ¹⁷⁷Lu reference standard of known activity. Before a patient was scanned, a reference standard was prepared by injecting 100 μCi (taken from the patient dose vial) into a 100 mL saline bag. The saline bag's radioactivity was subsequently imaged immediately after each imaging timepoint using the same acquisition and reconstruction parameters. The number of counts in the images attributed to the reference standard activity was measured, and the calibration factor was obtained by dividing the number of counts by the known activity in the reference standard at the time of imaging. This calibration factor is expected to be the same (<10% difference compared to the value determined at site setup) for all images acquired with the same SPECT/CT system. For patients where the calibration factor differed by more than 10% compared to the site set-up, an investigation was launched to determine the cause of this variation, and if it reflected a difference in patient data acquisition and/or reconstruction.

Gamma-counted plasma samples were quality controlled using the same ¹⁷⁷Lu reference standard by measuring 0.5 ml of the 100 mL in the saline bag. For each patient, triplicates were made and measured right before all plasma samples were counted. A long-lived isotope was also counted to ensure that the protocol was properly followed by each site. Similarly to the imaging standard, when the gamma counter efficiency factor differed by more than 10% compared to that at site set-up, an investigation was launched to determine the cause of this variation, and if it reflected a difference in data acquisition.

### Results

All twenty-seven (27) patients enrolled in the sub-study had SPECT/CT images acquired for dosimetry purposes at the first cycle. Sixteen out of the 27 patients were additionally imaged at cycle 3 as described in Section 4.4.1. The activity injected into each patient at cycles 1 and 3 is shown in Table 2.

TABLE 26
177Lu-PSMA-I&T Injected Activity at cycle 1 and 3

| Patient ID | Cycle 1 (GBq) | Cycle 3 (GBq) |
|---|---|---|
| 12-004 | 7.93 | 7.97 |
| 25-005 | 7.58 | 7.51 |
| 25-007 | 7.56 | 7.44 |
| 25-008 | 7.21 | 7.35 |
| 25-009 | 7.71 | 7.72 |
| 37-013 | 7.42 | NA |
| 37-015 | 7.49 | 7.53 |
| 37-016 | 7.46 | 7.50 |
| 37-017 | 7.59 | 7.53 |
| 37-019 | 7.35 | 7.40 |
| 37-020 | 7.19 | 7.37 |
| 37-022 | 7.48 | NA |
| 37-023 | 7.53 | NA |
| 42-027 | 7.56 | NA |
| 42-028 | 7.62 | NA |
| 42-030 | 7.56 | NA |
| 62-032 | 7.48 | NA |
| 66-024 | 7.26 | NA |
| 80-001 | 7.67 | NA |
| 80-004 | 7.52 | 7.22 |
| 80-007 | 7.54 | 7.14 |
| 80-018 | 7.31 | 7.16 |
| 80-019 | 7.43 | 7.58 |
| 80-020 | 7.39 | 7.42 |
| 80-022 | 7.32 | 7.48 |
| 80-023 | 7.44 | NA |
| 80-024 | 7.49 | NA |
| Mean | 7.48 | 7.46 |
| SD | 0.16 | 0.20 |

Dosimetry analysis included all acquired and/or evaluable imaging. Patient 37-016 did not have SPECT/CT imaging conducted at the 168 h timepoint, and thus the results were based on only 3 timepoints for this patient. For patient 12-004, the 48 h scan presented unrealistically high values (the whole-body fraction of injected activity at 48 h was above that at 4 h and 24 h, despite no additional activity was administered to the patient after the initial ¹⁷⁷Lu-PSMA-I&T injection). In addition, the image quality at 48 h was unusually poor for unknown reasons. Therefore, this timepoint was excluded from further analysis, and dosimetry was performed using the 4 h, 24 h and 168 h SPECT images.

### Cycle 1 Biodistribution

Based on the images and dosimetry data, physiologic uptake of ¹⁷⁷Lu-PSMA-I&T was predominantly observed in the kidneys, urinary bladder, lacrimal glands, GI tract, and salivary glands. ¹⁷⁷Lu-PSMA-I&T was primarily excreted through the urine, as indicated by visible accumulation in the urinary bladder. In 25 patients, uptake in the intestines was observed, while no appreciable uptake was found in 2 of the patients analysed (80-004 and 25-005). Salivary glands were not in the SPECT/CT field of view for 1 patient (12-004), and lacrimal glands were outside the field of view for 5 patients (12-004, 25-009, 42-027, 80-018, 66-024). ¹⁷⁷Lu-PSMA-I&T distribution in a patient imaged over time is represented in FIG. 28.

The individual time-activity curves for each source organ, expressed as fIA at the different timepoints are shown in FIGS. 3-10. Note: the time-activity curves in these figures are not decay corrected to time of injection.

All patients showed similar whole-body clearance except for patient 42-030 who had a visibly slower clearance. This slower clearance was likely due to the patient's diffuse bone metastatic disease, confirmed by exposure measurements performed by the site 24 hours after investigational product (IP) injection. An ionization chamber (Ludlum 9DP*) was used for the measurement, performed 1 meter from the patient. After each cycle, this patient had dose rates approximately three times higher than those of the other two patients from the same site:
patient 42-027: 7 uSv/h; 6 uSv/h; 5 uSv/h.
patient 42-028: 6 uSv/h; 8 uSv/h; 5 uSv/h.
patient 42-030: 16 uSv/h; 18 uSv/h; 17 uSv/h.

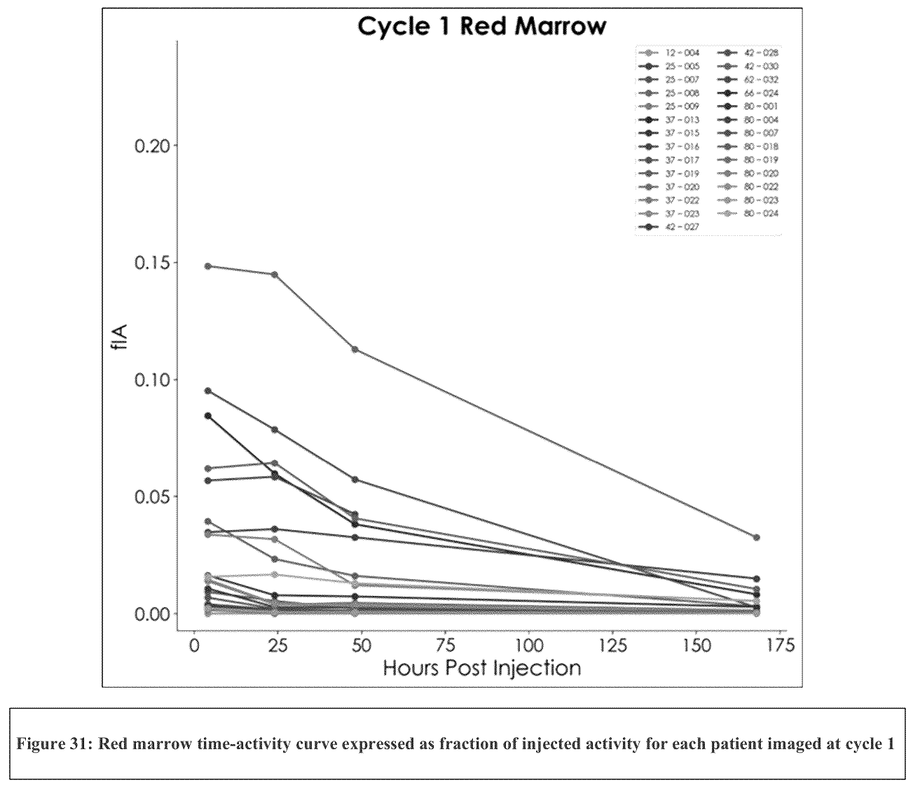

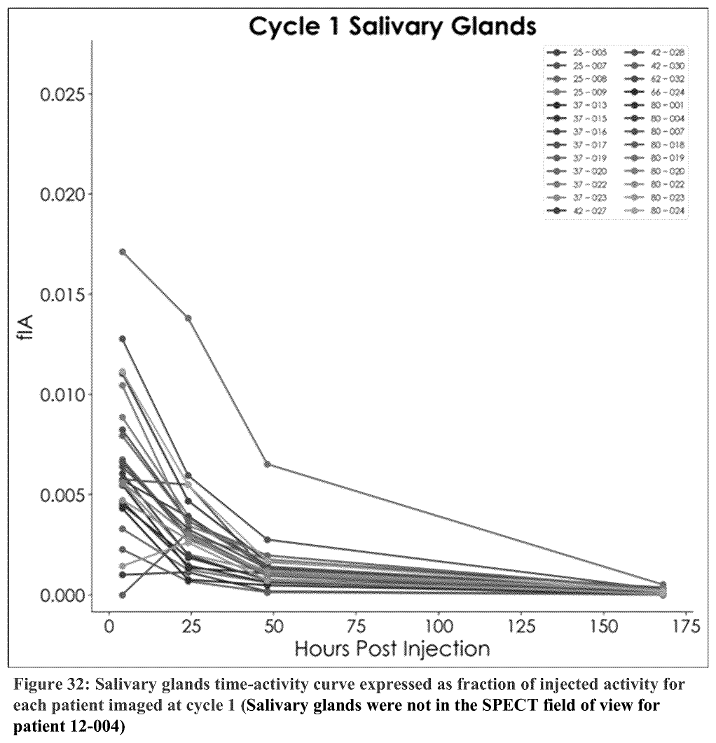

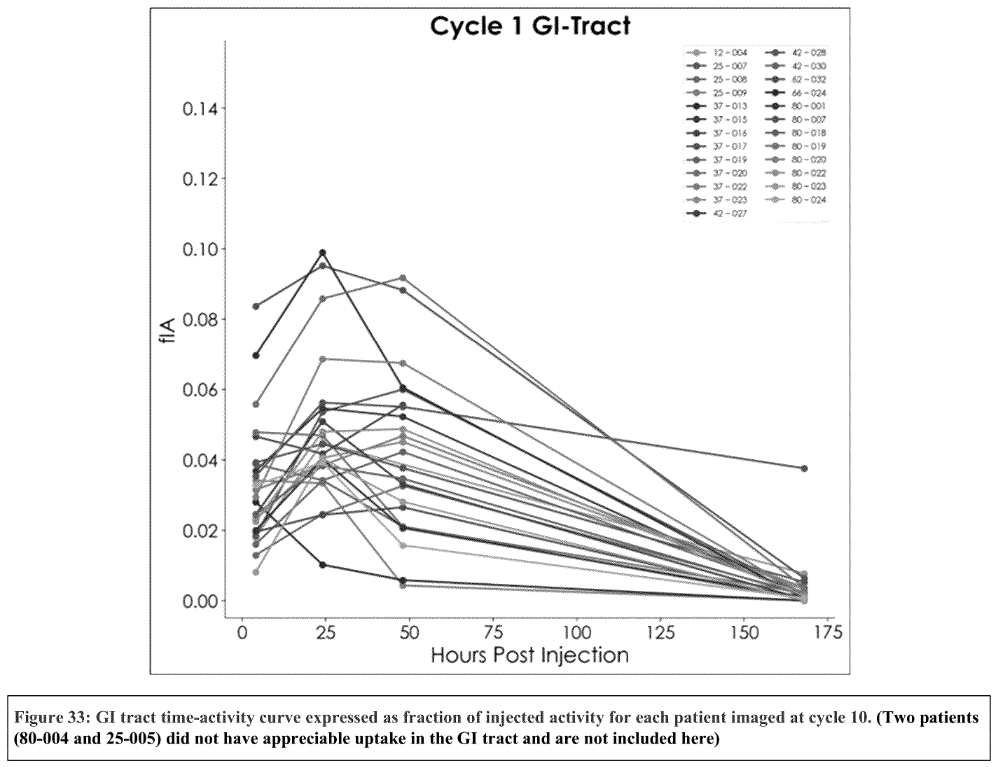

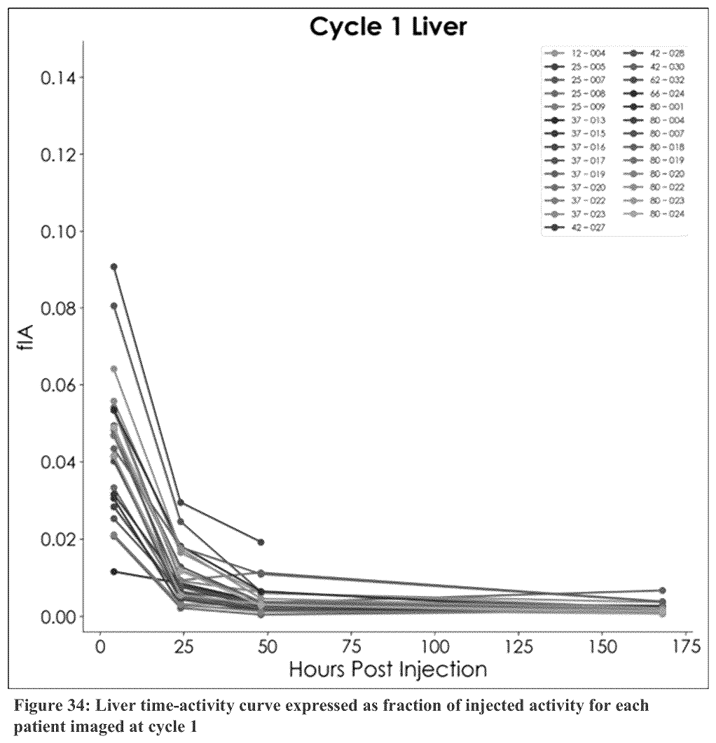

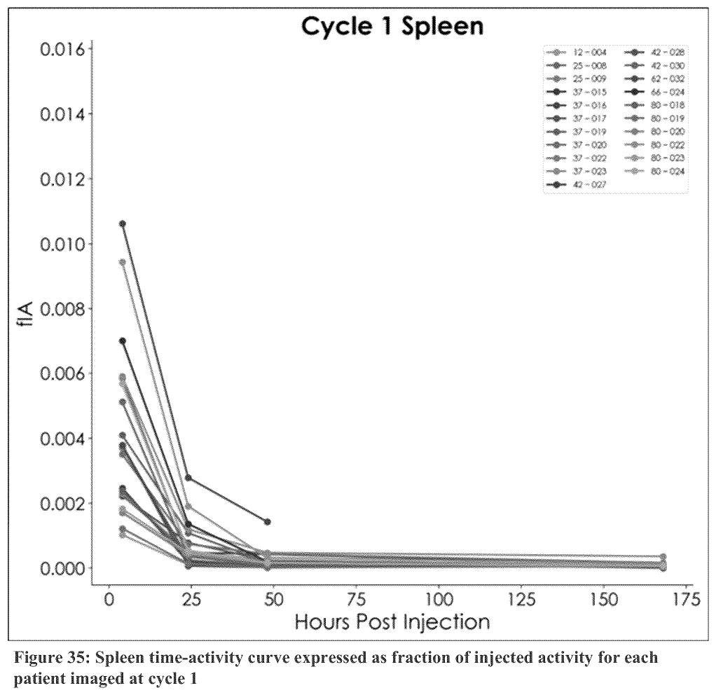

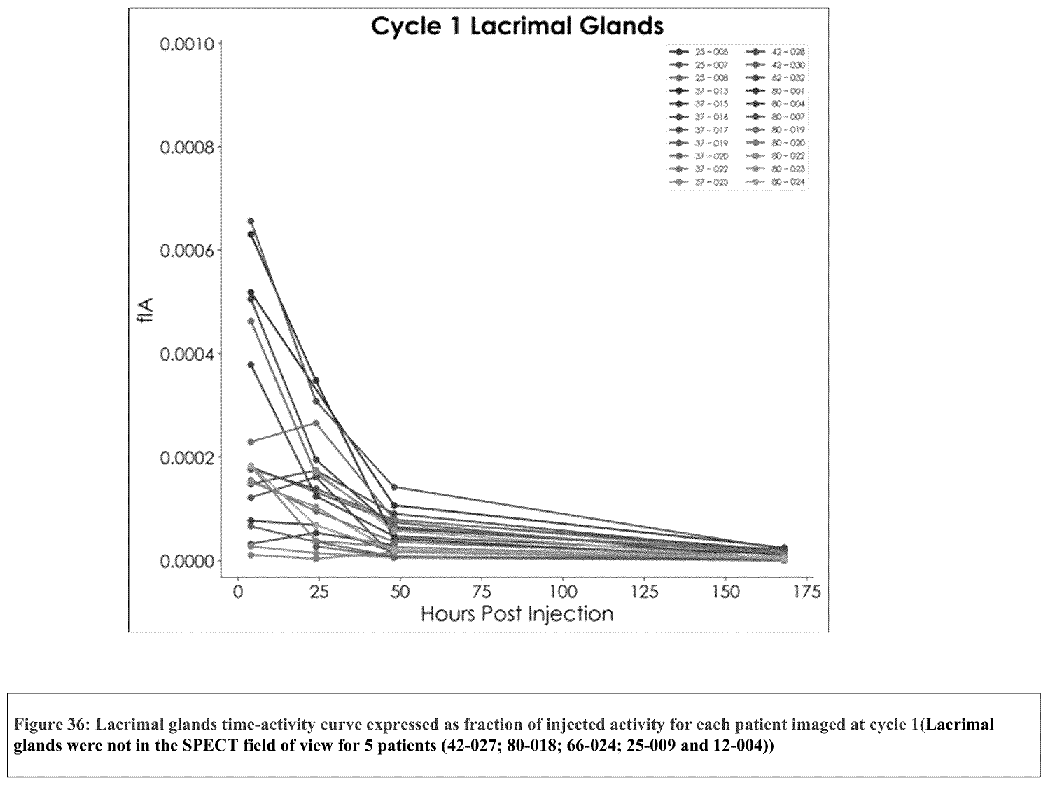

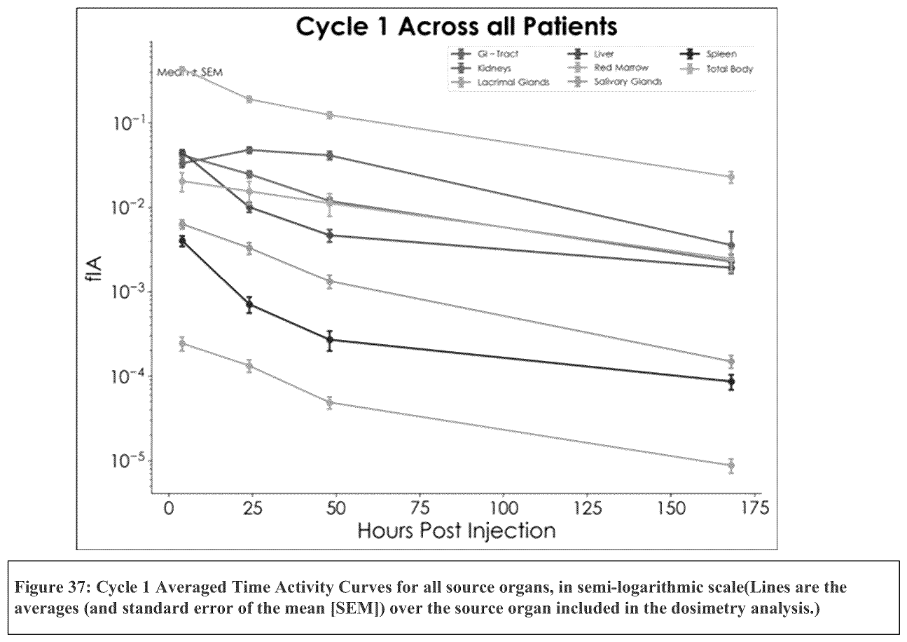

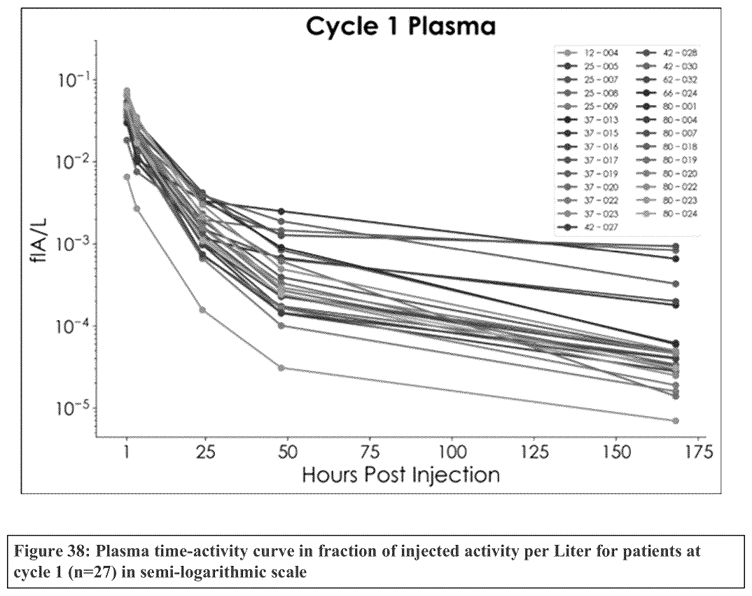

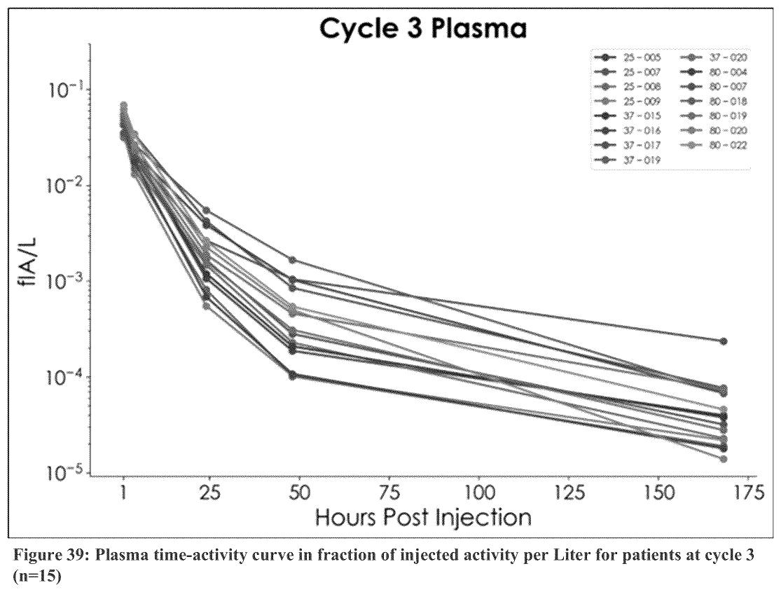

Given the significant bone involvement, this patient was determined to have superscan by the site Principal Investigator (PI), which is an exclusion criterion. In line with the high bone involvement in this patient, the estimated bone marrow uptake was also high, as shown in FIG. 31. Consequently, even if the dosimetry and PK analysis were performed, the biodistribution and dose values of this patient were removed from all aggregated data in this report (mean, standard deviation, and average curves).

The whole-body time-activity curves were fitted with bi-exponential equation and the two components were used for the distribution and elimination half-life. The mean half-lives (excluding patient 42-030) were distribution half-life of 2.16±1.30 hours and the elimination half-life of 46.29±23.83 hours.

Regarding the TACs in the various organs (kidney, liver, spleen, salivary glands, lacrimal glands, GI tract), the profiles were rather similar among the different patients, with some expected variability. Unsurprisingly, the variability is more pronounced in the GI tract.

### Cycle 1 Dosimetry

Mirroring the biodistribution data, the absorbed radiation dose per gram of tissue was higher in the organs responsible for the elimination of the tracer, i.e. kidneys (0.41±0.15 Gy/GBq), urinary bladder (0.41±0.05 Gy/GBq) and some parts of the GI tract, i.e. left colon (0.47±0.31 Gy/GBq) and rectum (0.44±0.30 Gy/GBq). Salivary and lacrimal glands also showed moderate absorbed radiation dose (0.19±0.16 and 0.40±0.36 Gy/GBq, respectively). To note for patient 12-004 the Salivary Glands were not in the field of view of the scans so no Salivary glands TIAC could be computed, this patient is excluded from the Salivary glands absorbed dose aggregate metrics.

The mean absorbed dose for bone marrow was 0.08±0.12 Gy/GBq). The high variability of the marrow dose is due to some patients having unusually high doses to the bone marrow (42-027, 62-032, 80-018, 80-024, 37-013, 37-016). In two out of these six patients (42-027 and 62-032), the high marrow dose estimation was likely due to the presence of an isolated metastasis in the vertebrae that were segmented to obtain the image-based marrow dosimetry (L2-L4). Because of the methodology of bone marrow dose estimation, the presence of a metastatic lesion within the VOI, leads to an overestimation of the bone marrow absorbed radiation dose. In the other four patients, the images showed a diffuse bone metastatic disease, resulting in a higher and more diffused radioactivity distribution in the bone tissue, with consequently higher marrow dose estimation. Excluding these six specific cases and the patient with a superscan, the remaining 20 patients would have had a mean red marrow absorbed dose of 0.02±0.02 Gy/GBq.

Average doses to all target organs are reported in Table 27 and individual doses are reported in Appendix 1.

TABLE 27
Average Cycle 1 Absorbed Dose Estimates (n = 26)

| | Absorbed Dose (Gy/GBq) | | | | | |
|---|---|---|---|---|---|---|
| Target Organ | Mean | SD | min | max | median | COV |
| Adrenals | 0.02 | 0.02 | 0.01 | 0.08 | 0.02 | 0.71 |
| Brain | 0.01 | 0.02 | 0.00 | 0.07 | 0.01 | 1.06 |
| Esophagus | 0.02 | 0.02 | 0.00 | 0.07 | 0.01 | 1.02 |
| Eyes | 0.01 | 0.02 | 0.00 | 0.07 | 0.01 | 1.06 |
| Gallbladder | 0.02 | 0.02 | 0.00 | 0.07 | 0.01 | 0.89 |
| wall | | | | | | |
| Heart wall | 0.02 | 0.02 | 0.00 | 0.07 | 0.01 | 1.02 |
| Kidneys | 0.41 | 0.15 | 0.17 | 0.81 | 0.37 | 0.36 |
| Left colon | 0.47 | 0.31 | 0.01 | 1.53 | 0.41 | 0.67 |
| Liver | 0.04 | 0.02 | 0.01 | 0.10 | 0.03 | 0.56 |
| Lungs | 0.01 | 0.02 | 0.00 | 0.07 | 0.01 | 1.03 |
| Osteogenic | 0.05 | 0.07 | 0.00 | 0.29 | 0.02 | 1.40 |
| cells | | | | | | |
| Pancreas | 0.02 | 0.02 | 0.00 | 0.07 | 0.01 | 0.92 |
| Prostate | 0.02 | 0.02 | 0.01 | 0.07 | 0.01 | 0.84 |
| Rectum | 0.44 | 0.30 | 0.01 | 1.46 | 0.39 | 0.67 |
| Red Marrow | 0.08 | 0.12 | 0.00 | 0.49 | 0.03 | 1.56 |
| Right Colon | 0.25 | 0.17 | 0.01 | 0.82 | 0.22 | 0.66 |
| Salivary | 0.19 | 0.16 | 0.03 | 0.80 | 0.15 | 0.82 |
| Glands | | | | | | |
| Small | 0.05 | 0.03 | 0.01 | 0.15 | 0.04 | 0.59 |
| intestine | | | | | | |
| Spleen | 0.03 | 0.02 | 0.01 | 0.11 | 0.02 | 0.76 |
| Stomach Wall | 0.02 | 0.02 | 0.00 | 0.07 | 0.01 | 0.99 |
| Testes | 0.01 | 0.02 | 10.00 | 0.07 | 0.01 | 1.02 |
| Thymus | 0.01 | 0.02 | 0.00 | 0.07 | 0.01 | 1.05 |
| Thyroid | 0.01 | 0.02 | 0.00 | 0.07 | 0.01 | 1.06 |
| Urinary | 0.41 | 0.05 | 0.33 | 0.50 | 0.41 | 0.12 |
| Bladder wall | | | | | | |
| Lacrimal | 0.40 | 0.36 | 0.04 | 1.19 | 0.26 | 0.90 |
| Glands | | | | | | |
| Total Body | 0.02 | 0.02 | 0.01 | 0.09 | 0.02 | 0.74 |

### Data Quality Control Results

The calibration factors of the individual patients were similar (<10% difference) to those determined at site setup, with the exception of two patients at cycle 1 from one site.

These two patients (80-018 and 80-023) showed discrepancies in both calibration and efficiency factors, with differences of approximately +135% and −45%, respectively, from the reference values determined at site setup (Table 8 in Appendix 1). Since the same discrepancy was found in the calibration factor for the SPECT and the efficiency factor for the gamma counter, we concluded that these discrepancies may have been due to errors in the measurement of the standard reference value. After quality control of the acquisition and reconstruction parameters, we concluded that the whole-body images of these two patients were acquired correctly, and the issue appeared limited to the measurement of the calibration factors. This conclusion was corroborated by the dosimetry results of these two patients, which were well within the range of the other patients.

### Cycle 1 PK Modelling

Plasma PK modelling results are reported in Table 4. Plasma data was fitted with a bi-exponential curve. As shown in FIG. 12 the plasma concentration decreased over time with a similar profile in all patients, initially with a faster decline (mean distribution half-life was 1.89±0.34 h) followed by a slower, prolonged decrease. The mean elimination half-life was 14.70±10.10 hours. Patient 42-030 demonstrated an outlier half-life value of 113.96 h. which aligns with the patient's whole body and bone marrow TACs, in which the radioactivity was taken up by the numerous bone metastases.

The time of peak concentration always corresponds to the time of the first post-administration blood sample taken one hour after injection. Likely, the peak value occurred shortly after injection, but was not captured by the sampling schedule applied in this study. Therefore, the peak concentration value reflects the value measured at the time of the first sample, and not the true peak value in the plasma. Notably, the time to peak for patient 80-007 is at 127 minutes, rather than one hour after injection, due to the first post-administration sample in that patient being acquired with a delay.

While the elimination phase of the plasma profile appears to be prolonged in most patients, it should be noted that the concentration levels at later timepoints are very low. Indeed, the clearance values (13.06±16.50 L/h) indicate a rather rapid clearance of most of the injected dose from the body. Part of the injected activity is captured in the target-expressing tissues and tumors, as indicated by the moderate-to-high distribution volume.

TABLE 28
Cycle 1 PK plasma modelling results

| | | | | Time | | | | |
|---|---|---|---|---|---|---|---|---|
| | | | Peak | of | | | | |
| | Distri- | Elimi- | Con- | Peak | | | | |
| | bution | nation | cen- | Con- | | | AUC | |
| | Half- | Half- | tration | cen- | | | Total | Clearance |
| | Life | Life | Value | tration | Vd | Vc | (fIA/L × | Rate |
| Patient | (h) | (h) | (fIA/L) | (min) | (L) | (L) | min) | (L/h) |
| 25-007 | 2.22 | 10.35 | 0.05 | 56.00 | 54.60 | 14.74 | 34.60 | 3.66 |
| 80-007 | 2.21 | 23.96 | 0.05 | 127.00 | 165.33 | 11.55 | 33.81 | 4.78 |
| 80-024 | 1.67 | 16.65 | 0.05 | 47.00 | 296.49 | 15.53 | 19.09 | 12.34 |
| 42-028 | 1.14 | 9.61 | 0.04 | 53.00 | 148.91 | 14.35 | 15.16 | 10.74 |
| 80-018 | 2.14 | 24.34 | 0.03 | 60.00 | 136.62 | 21.87 | 30.05 | 3.89 |
| 37-016 | 1.88 | 15.32 | 0.05 | 51.00 | 93.01 | 15.83 | 27.64 | 4.21 |
| 37-023 | 1.93 | 9.32 | 0.04 | 60.00 | 134.00 | 16.82 | 20.58 | 9.97 |
| 37-022 | 1.69 | 10.58 | 0.05 | 58.00 | 273.75 | 13.76 | 19.49 | 17.94 |
| 12-004 | 2.13 | 10.59 | 0.01 | 61.00 | 1365.03 | 111.78 | 3.07 | 89.38 |
| 80-019 | 2.19 | 12.43 | 0.04 | 60.00 | 112.27 | 17.80 | 25.59 | 6.26 |
| 80-020 | 1.69 | 10.02 | 0.06 | 50.00 | 139.07 | 11.45 | 25.58 | 9.62 |
| 37-017 | 2.31 | 10.46 | 0.04 | 63.00 | 115.14 | 20.41 | 21.49 | 7.63 |
| 42-030 | 1.81 | 113.96 | 0.02 | 61.00 | 463.99 | 38.22 | 26.70 | 2.82 |
| 80-022 | 1.66 | 9.19 | 0.07 | 57.00 | 62.71 | 10.76 | 31.83 | 4.73 |
| 37-019 | 1.78 | 12.50 | 0.04 | 55.00 | 383.51 | 18.90 | 15.14 | 21.26 |
| 80-001 | 1.35 | 53.30 | 0.03 | 52.00 | 222.08 | 22.65 | 28.67 | 2.89 |
| 80-004 | 1.77 | 10.35 | 0.04 | 68.00 | 308.54 | 15.26 | 17.09 | 20.65 |
| 25-008 | 2.12 | 9.28 | 0.04 | 60.00 | 108.16 | 21.10 | 19.85 | 8.08 |
| 80-023 | 1.92 | 8.16 | 0.07 | 60.00 | 67.50 | 9.92 | 34.96 | 5.73 |
| 37-020 | 2.39 | 10.35 | 0.04 | 71.00 | 118.24 | 17.87 | 24.13 | 7.92 |
| 25-009 | 1.66 | 8.92 | 0.03 | 60.00 | 227.51 | 22.51 | 13.46 | 17.68 |
| 37-013 | 1.30 | 35.48 | 0.04 | 51.00 | 540.13 | 14.72 | 17.93 | 10.55 |
| 37-015 | 2.03 | 8.68 | 0.05 | 52.00 | 177.78 | 16.91 | 18.79 | 14.19 |
| 42-027 | 2.42 | 11.79 | 0.03 | 65.00 | 291.02 | 25.15 | 15.54 | 17.10 |
| 62-032 | 1.46 | 19.98 | 0.04 | 33.00 | 307.91 | 17.72 | 19.50 | 10.68 |
| 66-024 | 2.05 | 11.28 | 0.06 | 40.00 | 64.25 | 12.87 | 37.86 | 3.95 |
| 25-005 | 2.04 | 9.41 | 0.05 | 69.00 | 187.12 | 15.24 | 21.09 | 13.78 |
| Mean | 1.89 | 14.70 | 0.04 | 59.19 | 234.64 | 20.29 | 22.77 | 13.06 |
| stdev | 0.34 | 10.10 | 0.01 | 16.20 | 257.46 | 19.07 | 8.04 | 16.50 |
| min | 1.14 | 8.16 | 0.01 | 33.00 | 54.60 | 9.92 | 3.07 | 2.89 |
| max | 2.42 | 53.30 | 0.07 | 127.00 | 1365.03 | 111.78 | 37.86 | 89.38 |
| median | 1.93 | 10.52 | 0.04 | 59.00 | 157.12 | 16.33 | 20.84 | 9.79 |
| COV | 0.18 | 0.69 | 0.31 | 0.27 | 1.10 | 0.94 | 0.35 | 1.26 |

### Cycle 3 Biodistribution and Dosimetry Results

A subgroup of 16 out of the 27 patients underwent and additional dosimetry assessment at Cycle 3. Among these, three patients had dosimetry performed based on images taken at 4 timepoints, while the remaining patients had dosimetry performed at 1 or 2 imaging timepoints only, as described in Section 4.4.1.

Similarly to Cycle 1, the highest uptake and organ absorbed radiation dose in Cycle 3 was predominantly seen in the kidneys, urinary bladder, lacrimal glands, GI tract, and salivary glands (individual and aggregate values for the TIACs of the target organs are in Appendix 1).

The organ absorbed radiation doses were compared to the mean organ absorbed radiation doses from Cycle 1 for the same subset of patients, as shown in Table 5. The dosimetry estimations obtained at cycle 3 align closely with those from cycle 1. At this timepoint, the kidneys had the highest dose (0.49±0.19 Gy/GBq) but, for most organs, the estimated absorbed dose values were similar to or lower compared to cycle 1. The average absorbed dose for the kidneys and urinary bladder wall was slightly higher at cycle 3 compared to cycle 1, but this difference remains within the expected variability range.

For the three patients whose dosimetry at cycle 3 was calculated using 4 image timepoints, the organ absorbed radiation dose values are also within the variability ranges observed at Cycle 1.

In conclusion, the dosimetry estimation performed at cycle 3 supports the approach of predicting cumulative absorbed dose based on extrapolation from cycle 1 data in this patient population

TABLE 29
Mean, SD, Min and Max Organ
Absorbed Radiation Dose estimates for
the 16 patients imaged at cycle 1 and 3

| | Absorbed Dose in | | | | Absorbed Dose in | | | |
|---|---|---|---|---|---|---|---|---|
| | Cycle 1 (Gy/GBq) | | | | Cycle 3 (Gy/GBq) | | | |
| Target | (N = 16) | | | | (N = 16) | | | |
| Organ | Mean | SD | min | max | Mean | SD | min | max |
| Adrenals | 0.02 | 0.02 | 0.01 | 0.08 | 0.02 | 0.01 | 0.01 | 0.03 |
| Brain | 0.01 | 0.02 | 0.00 | 0.07 | 0.01 | 0.00 | 0.00 | 0.02 |
| Esophagus | 0.01 | 0.02 | 0.00 | 0.07 | 0.01 | 0.01 | 0.00 | 0.02 |
| Eyes | 0.01 | 0.02 | 0.00 | 0.07 | 0.01 | 0.00 | 0.00 | 0.02 |
| Gallbladder | 0.02 | 0.02 | 0.00 | 0.07 | 0.01 | 0.01 | 0.00 | 0.02 |
| wall | | | | | | | | |
| Heart wall | 0.01 | 0.02 | 0.00 | 0.07 | 0.01 | 0.01 | 0.00 | 0.02 |
| Kidneys | 0.41 | 0.17 | 0.17 | 0.81 | 0.49 | 0.19 | 0.19 | 0.85 |
| Left colon | 0.52 | 0.37 | 0.01 | 1.53 | 0.41 | 0.21 | 0.07 | 0.94 |
| Liver | 0.04 | 0.03 | 0.0 | 0.10 | 0.06 | 0.04 | 0.00 | 0.15 |
| Lungs | 0.01 | 0.02 | 0.00 | 0.07 | 0.01 | 0.00 | 0.00 | 0.02 |
| Osteogenic | 0.04 | 0.05 | 0.00 | 0.19 | 0.02 | 0.02 | 0.00 | 0.09 |
| cells | | | | | | | | |
| Pancreas | 0.02 | 0.02 | 0.00 | 0.07 | 0.01 | 0.01 | 0.00 | 0.02 |
| Prostate | 0.02 | 0.02 | 0.01 | 0.07 | 0.01 | 0.01 | 0.01 | 0.02 |
| Rectum | 0.50 | 0.35 | 0.01 | 1.46 | 0.39 | 0.20 | 0.06 | 0.90 |
| Red Marrow | 0.05 | 0.08 | 0.00 | 0.28 | 0.03 | 0.04 | 0.00 | 0.17 |
| Right Colon | 0.28 | 0.20 | 0.01 | 0.82 | 0.22 | 0.12 | 0.04 | 0.51 |
| Salivary | 0.17 | 0.18 | 0.03 | 0.80 | 0.20 | 0.16 | 0.04 | 0.69 |
| Glands | | | | | | | | |
| Small | 0.06 | 0.04 | 0.01 | 0.15 | 0.04 | 0.02 | 0.01 | 0.10 |
| intestine | | | | | | | | |
| Spleen | 0.03 | 0.03 | 0.01 | 0.11 | 0.03 | 0.03 | 0.01 | 0.12 |
| Stomach | 0.02 | 0.02 | 0.00 | 0.07 | 0.01 | 0.01 | 0.00 | 0.02 |
| Wall | | | | | | | | |
| Testes | 0.0 | 0.02 | 0.00 | 0.07 | 0.01 | 0.00 | 0.00 | 0.02 |
| Thymus | 0.01 | 0.02 | 0.00 | 0.07 | 0.01 | 0.00 | 0.00 | 0.02 |
| Thyroid | 0.01 | 0.02 | 0.00 | 0.07 | 0.01 | 0.00 | 0.00 | 0.02 |
| Urinary | 0.40 | 0.04 | 0.33 | 0.47 | 0.45 | 0.08 | 0.36 | 0.70 |
| Bladder wall | | | | | | | | |
| Lacrimal | 0.39 | 0.36 | 0.04 | 1.19 | 0.29 | 0.20 | 0.10 | 0.73 |
| Glands^{a} | | | | | | | | |
| Total Body | 0.02 | 0.02 | 0.01 | 0.09 | 0.02 | 0.01 | 0.01 | 0.03 |

^{a}Lacrimal glands absorbed dose was calculated using OLINDA sphere model that assume local deposition of the dose.

### Cycle 3 PK Modelling

Blood samples were obtained for 15 out of 16 patients at cycle 3. Patients 12-004 had blood taken only at the 24 h timepoint and, therefore, was not included in the plasma PK analysis. Consistent with Cycle 1, the time-course of plasma concentrations was similar among the patients. The ranges of PK parameters obtained at cycle 3 were similar to those from Cycle 1 data (Table 6). See Table 30. Cycle 3 PK plasma modeling results (n=15)

| | | | Peak | Time | | | | |
|---|---|---|---|---|---|---|---|---|
| | | | Con- | of | | | | |
| | Distri- | Elimi- | cen- | Peak | | | AUC | |
| | bution | nation | tration | Con- | | | Total | |
| | Half- | Half- | Value | cen- | | | (fIA/ | Clearance |
| | Life | Life | (fIA/ | tration | | | L × | Rate |
| Patient | (h) | (h) | L) | (min) | Vd (L) | Vc (L) | min) | (L/h) |
| 25-007 | 3.17 | 10.67 | 0.06 | 60.00 | 51.46 | 15.13 | 39.62 | 3.34 |
| 80-007 | 2.61 | 18.37 | 0.04 | 60.00 | 152.16 | 18.12 | 28.52 | 5.74 |
| 80-018 | 2.22 | 14.16 | 0.05 | 61.00 | 61.59 | 16.40 | 36.09 | 3.01 |
| 37-016 | 2.07 | 12.51 | 0.04 | 60.00 | 73.36 | 17.66 | 29.33 | 4.07 |
| 80-019 | 1.69 | 11.70 | 0.05 | 68.00 | 129.70 | 11.98 | 26.16 | 7.68 |
| 80-020 | 1.88 | 10.92 | 0.06 | 61.00 | 106.80 | 11.38 | 29.32 | 6.78 |
| 37-017 | 2.30 | 9.33 | 0.04 | 66.00 | 105.05 | 21.49 | 20.20 | 7.81 |
| 80-022 | 2.06 | 9.92 | 0.07 | 61.00 | 70.92 | 10.66 | 36.74 | 4.95 |
| 37-019 | 1.64 | 8.16 | 0.04 | 55.00 | 171.89 | 16.42 | 16.57 | 14.60 |
| 80-004 | 2.07 | 9.10 | 0.04 | 60.00 | 239.78 | 16.53 | 19.01 | 18.27 |
| 25-008 | 2.55 | 9.11 | 0.03 | 65.00 | 121.21 | 24.53 | 17.88 | 9.22 |
| 37-020 | 2.28 | 10.32 | 0.04 | 59.00 | 126.73 | 17.80 | 23.21 | 8.52 |
| 25-009 | 1.85 | 9.75 | 0.03 | 60.00 | 361.81 | 20.07 | 13.86 | 25.73 |
| 37-015 | 2.11 | 9.63 | 0.05 | 59.00 | 158.71 | 13.50 | 24.55 | 11.43 |
| 25-005 | 2.07 | 9.75 | 0.05 | 55.00 | 191.15 | 14.30 | 22.70 | 13.59 |
| 37-017 | 2.30 | 9.33 | 0.04 | 66.00 | 105.05 | 21.49 | 20.20 | 7.81 |
| Mean | 2.17 | 10.89 | 0.05 | 60.67 | 141.49 | 16.40 | 25.58 | 9.65 |
| stdev | 0.39 | 2.56 | 0.01 | 3.52 | 79.92 | 3.82 | 7.72 | 6.24 |
| min | 1.64 | 8.16 | 0.03 | 55.00 | 51.46 | 10.66 | 13.86 | 3.01 |
| max | 3.17 | 18.37 | 0.07 | 68.00 | 361.81 | 24.53 | 39.62 | 25.73 |
| median | 2.07 | 9.92 | 0.04 | 60.00 | 126.73 | 16.42 | 24.55 | 7.81 |
| COV | 0.18 | 0.23 | 0.22 | 0.06 | 0.56 | 0.23 | 0.30 | 0.65 |

### Conclusions

The biodistribution, PK, and dosimetry analysis of a population of patients injected with ¹⁷⁷Lu-PSMA-I&T shows that the organs with moderate to high physiological uptake and absorbed dose are primarily those involved in the elimination of the radiopharmaceutical and/or those expressing PSMA. These include kidneys (0.41±0.15 Gy/GBq), urinary bladder (0.41±0.05 Gy/GBq), salivary and lacrimal glands (0.19±0.16 and 0.40±0.36 Gy/GBq, respectively) and some parts of the GI tract (left colon, 0.47±0.31 Gy/GBq, and rectum, 0.44±0.30 Gy/GBq).

The mean red marrow absorbed radiation dose was 0.08±0.12 Gy/GBq. The high variability observed for this parameter, calculated using the image-based method, is attributed to the presence of diffuse bone metastatic disease in some patients, leading to an increased bone uptake of ¹⁷⁷Lu-PSMA-I&T, and consequently higher absorbed radiation dose in the marrow. Additionally, few patients had metastases in the lumbar region (L2-L4), where the image for bone marrow dosimetry is obtained, potentially leading to overestimation of the marrow absorbed dose in these cases.

PK results demonstrated consistent elimination profile of the radiopharmaceutical from plasma across patients. In particular, the plasma kinetic showed a monotonically decreasing trend across all patients, fitting well with a bi-exponential model.

Overall, the dosimetry data for the main organs of interest were within the expected ranges, as compared to values published in the literature for ¹⁷⁷Lu-PSMA-I&T, derived from various studies, employing different dosimetry methodologies.⁶⁻⁸

Notably, cycle 3 data showed dosimetry and PK values similar to those from cycle 1. This consistency suggests that the values obtained at cycle 1 can be reliably used to extrapolate the cumulative absorbed dose of subsequent cycles.

### Example 8: Comparative Study for Extension of Product Expiration from 48 Hours to 74 Hours for ¹⁷⁷Lu-PSMA I&T

The objective of this study was to extend the expiry period of ¹⁷⁷Lu-PSMA I&T Injection from 48 hours to 72 hours. The 72-hour expiry was achieved by modifying the drug product composition, by reducing the strength of the ¹⁷⁷Lu-PSMA I&T from 1 GBq/mL to 0.5 GBq/mL and maintaining a more restrictive pH of the formulated drug product in the range of 4 to 5. The total patient dose was unchanged, which remains at 7.4 GBq ¹⁷⁷Lu-PSMA I&T. In order to deliver the desired radioactive dose, the volume of drug was increased from 8-10 mL to 15-20 mL. Thus, the primary container closure system was changed from a 10 mL to a 20 mL vial, while maintaining the same glass quality, the same elastomeric closure, and the same aluminum crimp seal (Table 31).

In this study, the stability of “Drug Product B” was studied and resulted in extended expiration period of [¹⁷⁷Lu]Lu-PSMA I&T Injection of “Drug Product A”.

### A. Description and Composition of the Drug Product

TABLE 31
Description of Drug Product

| Drug Product A | Drug Product B |
|---|---|
| The drug product was packaged | The drug product was packaged |
| in a 10 mL sterile glass vial | in a 20 mL sterile glass vial |
| with a Fluorotec coated 4432.50 | with a Fluorotec coated 4432.50 |
| chlorobutyl pharmaceutical-grade | chlorobutyl pharmaceutical-grade |
| rubber stopper secured in place | rubber stopper secured in place |
| with an aluminum crimp seal. | with aluminum crimp seal. |

Table 32 provides a listing of the qualitative and quantitative composition of [¹⁷⁷Lu]Lu-PSMA I&T Injection, which had undergone a composition change to increase the shelf-life (i.e., an expiration extension), which was achieved by reducing the strength (radioactive concentration) from 1 GBq/mL to 0.5 to 0.6 GBq/mL, using a more stringent pH, and increasing the fill volume to 20 mL/vial. The number of vials produced per batch was adjusted in order to produce the number of doses required for therapeutic administration. The drug product qualitative and quantitative composition of [¹⁷⁷Lu]Lu-PSMA I&T are provided in Table 26.

TABLE 32
Drug Product Qualitative and Quantitative Composition of [¹⁷⁷Lu]Lu -PSMA I&T

| | Drug Product A | | Drug Product B | | |
|---|---|---|---|---|---|
| | Quantity | In 1 dose 7.4 | Quantity | In 1 dose 7.4 | |
| Component | per mL | GBq (10 mL) | per mL | GBq (20 mL) | Function |
| ¹⁷⁷Lu-PSMA I&T | 1 | | 0.5 | | Radioactive |
| (GBq) | | | | | pharmaceutical |
| | | | | | ingredient |
| Typical volume | | 8-9 (Max | | 15 (15-20 | |
| for 1 dose (mL) | | 10 mL) | | 10 mL)** | |
| ¹⁷⁷Lu-PSMA I&T | <12 | <80 | <6 | <80 | |
| and PSMA-I&T | | | | | |
| (μg) | | | | | |
| Ascorbic acid | 42.5 | Formulation: | 31 | Formulation: | Stabilizing agent |
| (mg) | (20-70) | 340-425 | (15-35) | 340-425 | |
| | | Specification: | | Specification: | |
| | | 200-700 | | 200-700* | |
| Disodium EDTA | 21 | 168-210 | 15.5 | <310 | Metal ion |
| (μg) | | | | | scavenger, |
| | | | | | present as |
| | | | | | excipient in |
| | | | | | formulated |
| | | | | | ascorbic acid |
| | | | | | injection solution |
| Sodium | A | QS | A | | Present as |
| Bicarbonate | sufficient | | sufficient | | excipient in |
| | quantity | | quantity | | formulated |
| | | | | | ascorbic acid |
| | | | | | injection solution |
| NaOH | A | QS | A | | Present as |
| | sufficient | | sufficient | | excipient in |
| | quantity | | quantity | | formulated |
| | | | | | ascorbic acid |
| | | | | | injection solution |
| Hydrochloric acid | N/A | N/A | A | N/A | pH adjustment |
| (Max addition of | | | sufficient | | |
| 30% HC1 is | | | quantity | | |
| 468 μ1/100 m1 | | | | | |
| final formulation, | | | | | |
| i.e. should not | | | | | |
| have any relevant | | | | | |
| impact on | | | | | |
| chloride ion | | | | | |
| quantities) | | | | | |
| Ethanol (μL) | 50-80 (5- | Formulation: | | Formulation: | Stabilizing agent |
| | 8% v/v) | 600-750 | | 563-750 | (radiolysis) |
| | | Specification: | | Specification: | |
| | | 500-800 (5- | | 375-900 (2.5 | |
| | | 8% v/v) | | to 4.5% v/v) | |
| Water for | Sufficient | QS | Sufficient | QS | Vehicle |
| Injection | to make 1 | | to make 1 | | |
| | mL | | mL | | |
| pH*** | | 5-8 | | 4-5 | |

*North American medical authorities have set a maximum adult dose of 2,000 mg per day, so 700 mg is acceptable.
**The recommended dose of [¹⁷⁷Lu]Lu-PSMA I&T is 7.4 GBq (200 mCi)/administration.
Depending on the actual radioactive strength of each batch, the volume dispensed into the 20 mL vial may vary between ~15-20 mL.
***The updated pH range is within the range is within the range of other FDA-approved USP grade injectable solutions (ex: 5% dextrose injection solution).

The changes associated with the expiration extension did not change the identity, quality, or purity of any of the components of the drug substance or drug product. It did change the strength of the API in the final drug product, established a narrower control range for pH, and modified the concentration of the two main excipients, ascorbic acid and ethanol. These changes were made to extend the expiry period of the drug product.

With these changes associated with the expiration extension, the batch size was scaled to provide the number of therapy doses, plus the overage needed to provide quality control test samples for both microbiological and chemistry tests and to provide a reserve sample for each batch. In this context, all components and excipients were scaled to the measured amount of radioactive Lu-177 precursor labeling solution.

### B. Manufacturing Process

This expiration extension study did not involve any substantive changes to the manufacturing process, equipment, or reagents used. The reformulated product was formulated to a lower radioactive concentration (strength), more stringent pH limits applied, and the same excipients used, but in different strengths from the original formulation. As the manufacturing chemistry of the drug substance remained unchanged, the changes only applied to the formulation of the final drug product solution.

The pH was the only critical step that was updated. The pH of the final drug product solution was controlled to the narrower range of 4.0 to 5.0 from 5.0 to 8.0. This was accomplished by the controlled addition of hydrochloric acid.

The stability of the product was evaluated over the assigned shelf life at storage temperatures ranging from 2° C.-40° C. In addition, microbial bioburden studies were performed on three other batches of ¹⁷⁷Lu-PSMA I&T injection. All batches met pre-determined acceptance criteria through the extended 72-hour expiration time point. The product composition was changed, to decrease the target strength from 1 GBq/mL to between 0.5 and 0.6 GBq per mL. The pH range was also controlled to the narrower range of 4.0 to 5.0 from 5.0 to 8.0, and due to the lower strength formulation, the dispensed volume was increased from 10 to 20 mL/vial. In addition, the strength of ascorbic acid and ethanol was decreased. This lower strength product was subjected to process validation studies that confirmed that the 72-hour expiration date had been achieved.

A media fill study was successfully executed on three test batches to support aseptic fill operations corresponding to changes in drug product volume (increase) and vial size.

There are no changes to the excipients utilized in the drug product formulation, which remain as (Ethanol, Ascorbic Acid, and Water for Injection). With drug product composition change, it did not result in the creation of any new impurities or an increase in existing impurities. Only the concentrations/volume of existing excipients were adjusted to achieve a larger volume.

Table 33 below lists Drug Product A and Drug Product B tests and acceptance criteria.

TABLE 33
Tests and Acceptance Criteria for Drug Product A and
Drug Product B of [¹⁷⁷Lu]Lu-PSMA-I&T injection.

| | Acceptance Requirement | |
|---|---|---|
| Test | Drug Product A* | Drug Product B* |
| Appearance | Clear and colorless to | Clear and colorless to |
| | yellow solution, no visible | yellow solution, no visible |
| | particles | particles |
| pH | 5.0-8.0 | 4.0-5.0 |
| Ascorbic Acid | 20-70 mg/mL | 15-35 mg/mL |
| Radionuclidic identification | Energy from ¹⁷⁷Lu main | Energy from ¹⁷⁷Lu main |
| | peaks: 113 ± 10 keV and | peaks: 113 ± 10 keV and |
| | 208 ± 12 keV No other | 208 ± 12 keV No other |
| | significant peaks with | significant peaks with |
| | gamma energy >100 keV | gamma energy >100 keV |
| | are detected | are detected |
| Chemical purity | Lu-PSMA I&T - | Lu-PSMA I&T - |
| | concentration ≤12.0 μg/mL | concentration ≤6.0 μg/mL |
| Radiochemical Identity | The radiometric RT of the | The radiometric RT of the |
| | test sample = UV RT of | test sample = UV RT of |
| | Reference Standard ±5%. | Reference Standard ±5%. |
| Radiochemical purity by HPLC | ≥95.0% | ≥95.0% |
| method | | |
| Radiochemical purity by TLC | ¹⁷⁷Lu-colloid ≤5.0% | ¹⁷⁷Lu-colloid ≤5.0% |
| method | | |
| Radionuclidic purity | ≥99.9% of the total activity | ≥99.9% of the total activity |
| | of ¹⁷⁷Lu at the end of the | of ¹⁷⁷Lu at the end of the |
| | shelf life | shelf life |
| Endotoxin | <175 EU/10 mL vial* | <175 EU/10 mL vial* |
| | | <8.75 EU/mL |
| Ethanol | 5.0% to 8.0% (v/v) | 2.5% to 4.5% (v/v) |
| Sterile filter integrity | ≥3.45 bar (Millex GV) | ≥3.32 bar (Pall, Mini |
| | | Kleepak)** |
| Sterility*** | Sterile | Sterile |

*Dose = maximum volume to be administered per dose is defined as 10 mL (200 mCi) for original formulation and 20 mL for the new formulation (200 mCi)
**There was no change to the sterilizing filter.
***The sterility test samples are initiated post-release.

### C. Analytical Procedures

Due to the decrease in strength (radioactive concentration) of the product (per mL), the sample injection volume was increased from 50 μL to 100 μL for some of the drug product tests.

Due to this change, the following method verification studies were performed:
Determination of Colloidal Impurities in Lu-177-PSMA Injection by TLC
Bioburden Microbial Growth
Endotoxin Testing
Sterility Testing
Determination of Ethanol by Gas Chromatography

The decrease in strength (radioactive concentration) resulted in a slower rate of radiolysis, such that the radiochemical purity at 72 hours post-end-of-synthesis remained above 95%. In effect, this did not result in the creation of any new impurities or an increase in existing impurities

### D. Container Closure System

The vial in the primary container closure system was changed from a 10 mL glass pharmaceutical-grade injection vial to a 20 mL vial of the same pharmaceutical grade and neck finish. It was sealed with the current fluoro coated bromobutyl elastomeric closure secured in position with an open-top aluminum crimp. Neither the elastomeric closure nor crimp was changed (Table 34).

TABLE 34
Container Closure Comparison of
Drug Product A and Drug Product B

| Component | Drug Product A | Drug Product B |
|---|---|---|
| Vial, 10 mL | Schott (Glass Vial) | NA |
| | Adelphi (Sterile, air-filled) | |
| Vial, 20 mL | NA | Curium/US code 5935 |
| | | 2703 Wagner Place |
| | | St. Louis, Missouri |
| | | 63042, USA |
| Elastomeric | West Pharmaceutical | West Pharmaceutical |
| Closure | Services 1028 Innovation | Services 1028 Innovation |
| | Way Kinston, NC 28504 | Way Kinston, NC 28504 |
| Aluminum Crimp | GI Pharma | GI Pharma |

### E. Stability

The radiochemical purity and chemical properties of the 0.5 GBq/mL [¹⁷⁷Lu]Lu-PSMA-I&T injection solution (inclusive of pH, impurities, and visual properties) were tested on three process validation and four separate stability batches over a time span of 72 hours from the end-of-synthesis time. All batches utilized the ¹⁷⁷Lu precursor labeling solution. Stability samples from all batches of [¹⁷⁷Lu]Lu-PSMA-I&T injection drug product were stored at room temperature, with selected samples also stored inverted and some samples stored at elevated temperatures. Initial tests were generally completed within 7 hours post end of synthesis, and stability-indicating tests were repeated at 24, 48, and 72 hours post EOS. No deviations from specification acceptance criteria were observed for samples tested up to and including 72 hours for samples formulated at a strength of −0.5 GBq/mL. Based on these supportive stability data, an expiration time of 72 hours post-end-of-synthesis was assigned. The changes to the shelf life of the drug product is provided in Table 35.

TABLE 35
Shelf Life comparison of Drug Product A and Drug Product B

| Attribute | Drug Product A | Drug Product B |
|---|---|---|
| Shelf-Life (Expiry) | 48 Hrs Post End of | 72 Hrs Post End of |
| | Synthesis | Synthesis |

### F. Conclusion

The 72-hour expiry was achieved by modifying the drug product composition, by reducing the strength of the [¹⁷⁷Lu]Lu-PSMA I&T from 1 GBq/mL to 0.5 GBq/mL and maintaining a more restrictive pH of the formulated drug product in the range of 4 to 5.

### Example 9: A Multi-Center, Open-Label, Randomized, Phase 3 Trial Comparing the Safety and Efficacy of ¹⁷⁷Lu-PSMA-I&T Versus Hormone Therapy in Patients with Metastatic Castration-Resistance Prostate Cancer

Twelve subjects were administered a target activity of 7.4 GBq (mean 7.56±0.17 GBq) of [¹⁷⁷Lu]Lu-PSMA-I&T. Following the infusion, subjects underwent single photon emission computed tomography (SPECT)/computed tomography (CT) imaging at four timepoints (4 hours, 24 hours, 48 hours, and 168 hours). Image data was analyzed to compute time integrated activity coefficients (TIAC) in each organ of interest defined a priori in the independent review charter and in organs showing appreciable and meaningful [¹⁷⁷Lu]Lu-PSMA I&T activity. Segmented organs included kidneys, bladder, liver, lacrimal glands, salivary glands, intestine, lumbar vertebrae L2-L4, and whole body. To compute subject-specific organ doses, organ-level TIAC data were entered into Organ Level Internal Dose Assessment (OLINDA 2.2.3) and resulting organ doses for each target organ and whole-body effective dose were collated and averaged.

### Data Acquisition

Patients received SPECT/CT imaging (anatomical coverage included the salivary gland in most of the patients and extended through the pelvis) following the injection of 7.4 (±10%) GBq of [¹⁷⁷Lu]Lu-PSMA-I&T (detailed injected doses are reported in Table 36). All patients completed the first treatment cycle, and the acquisition of images was carried out. To note, for patient 37-016 the 168 hours SPECT/CT timepoint wasn't acquired, whereas patient 12-004 48 hours scan was presenting unrealistically high values, and the 48 hours scan was therefore excluded from further analysis.

TABLE 36
Injected ¹⁷⁷Lu-PSMA-I&T Activity

| Subject ID | Injected Dose [¹⁷⁷Lu]Lu-PSMA-I&T (GBq) |
|---|---|
| 25-005 | 7.58 |
| 25-007 | 7.56 |
| 25-008 | 7.21 |
| 25-009 | 7.71 |
| 80-001 | 7.67 |
| 80-004 | 7.52 |
| 80-007 | 7.54 |
| 37-013 | 7.42 |
| 37-015 | 7.49 |
| 37-016 | 7.49 |
| 37-017 | 7.59 |
| 12-004 | 7.93 |
| Mean | 7.56 |
| SD | 0.17 |

### Image Pre-Processing and Segmentation

The imaging data was analyzed using Invicro's VivoQuant software, a validated software used in a 21 CFR § 11-compliant workflow. The general quantification approach was based on the principles detailed in the Medical Internal Radiation Dose (MIRD) Pamphlet No. 16, No 23, and No 26, as appropriate.

Each SPECT image was co-registered with its corresponding CT. Image calibration was performed to convert the counts measured on the SPECT images to activity (Bq). The same SPECT/CT system was used in each patient for the entirety of the dosimetry study.

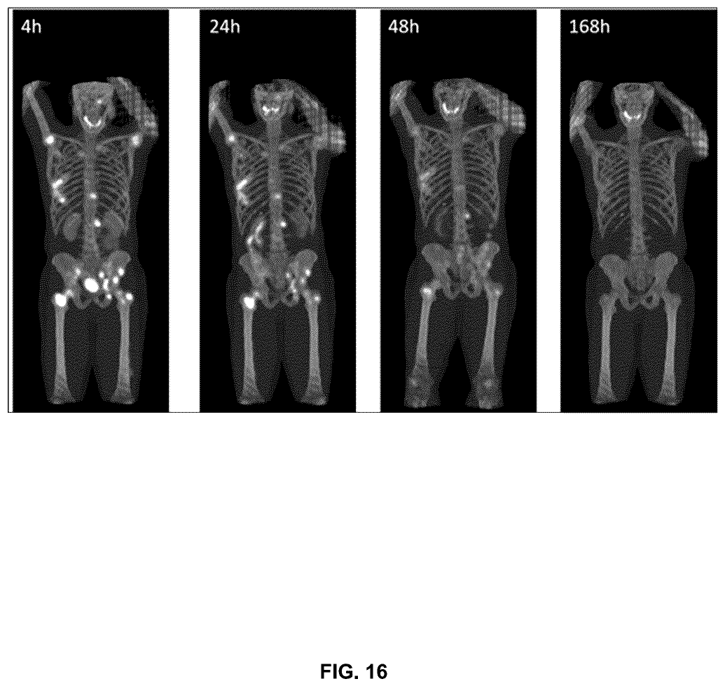

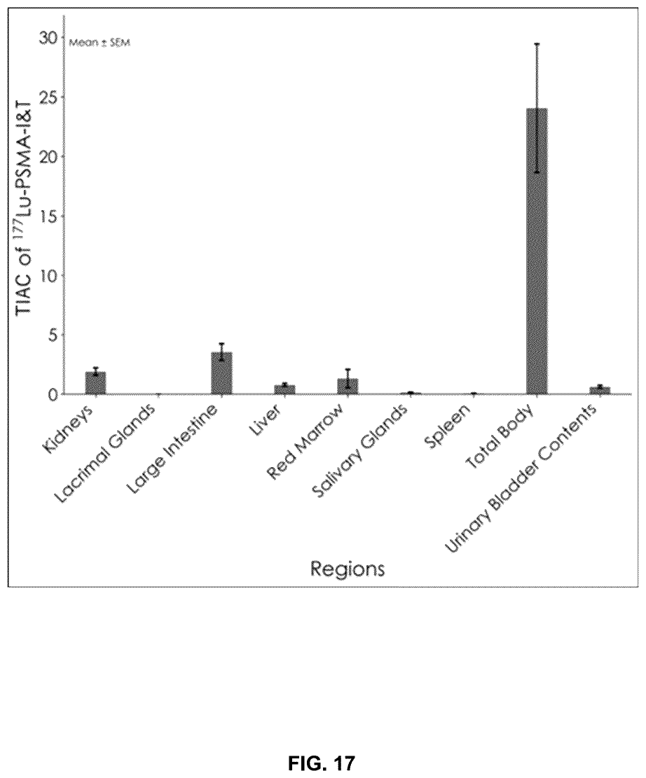

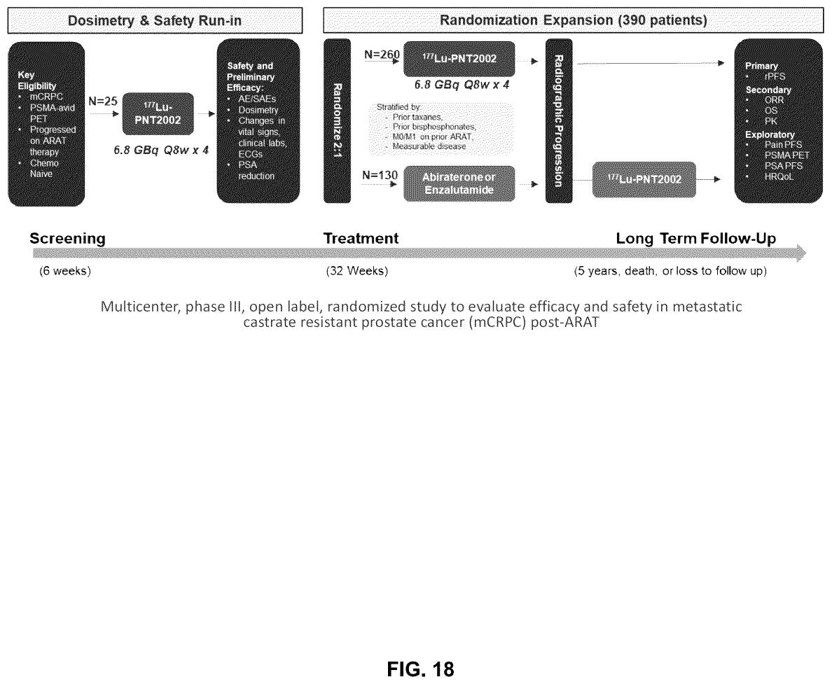

Regions of interest (ROI) (kidneys, bladder, liver, lumbar vertebrae L2-L4, lacrimal glands, salivary glands (parotid and submandibular glands), and whole body) were drawn on each dataset by a trained image analyst. Intestines and spleen were delineated only in a subset of subjects where the organs displayed visible uptake. Lumbar vertebrae L2-L4 was delineated for all patients on the CT images to derive red marrow doses.⁴ For kidneys, a ROI was drawn on the SPECT image encompassing all the uptake seen in the kidneys. Similarly for lacrimal glands, all the uptake observed on the SPECT image at the lacrimal glands' location was segmented. [¹⁷⁷Lu]Lu-PSMA-I&T distribution in a patient imaged over time is represented in FIG. 16.

Time-activity curves of the activity in each ROI as a function of time were plotted. These curves were normalized by the known injected activity to arrive at units of fraction of injected activity. Calculations of time-integrated activity coefficient (TIAC, in units of hours) were performed for each region of interest listed above.

### Dosimetry Analysis

Clearance of the tracer was derived from a data-appropriate fit of each organ time activity curve (TAC). Depending on the shape of the TAC, the data was fitted with the sum of one, two or three exponentials or using a rise and fall model. TIACs were calculated through analytical integration of the curve fit extrapolated to infinite. If no appropriate fit was found, the area under the curve (AUC) was estimated using the sum of the trapezoidal integration of imaging measurements and physical decay from the last timepoint onward (the fraction of injected activity at t=0 was set to zero for organs and to 1 for whole body). The remainder activity was determined by subtracting the cumulative organ TIACs from the whole body TIAC.

Organ TIACs were inputted into Organ Level Internal Dose Assessment, OLINDA 2.2.3 for the computation of organ and whole-body absorbed doses and effective dose (computed using ICRP-103 weighing factors). ICRP-89 derived male phantom was used for dose estimation. Organs and body weights as defined by ICRP-89 were scaled based on height and mass using the patient effective mass. Kidney volumes were measured for each patient on the CT images and used for the kidneys mass of the ICRP-89 phantom assuming a density of 1 g/ml. The voiding bladder model as implemented into OLINDA was used to determine bladder TIAC, as well as the HAT model for patients showing clearance through the gastrointestinal tract.

Lacrimal glands absorbed dose was computed using the OLINDA 2.2.3 sphere model which assumes local deposition of the dose only. A lacrimal gland volume of 0.680 cm³ was used for all patients.

### Dosimetry Results

Physiologic uptake was predominantly seen in the kidneys, urinary bladder, and salivary glands. [¹⁷⁷Lu]Lu-PSMA-I&T was primarily excreted through the kidneys and via urinary excretion in the bladder. In ten patients, uptake in the intestines was seen while no appreciable uptake was found in 2 of the patients analyzed. Salivary glands were not in the field of view for 1 patient (12-004), and lacrimal glands were not in the field of view for 2 patients (12-004 and 25-009). Time activity curves for all ROI delineated are reported in FIG. 14. Whole body time activity curves showed an initial rapid clearance with low activity retention at 4 h, followed by a second, slower clearance.

FIG. 14 reports the mean TIACs for the source organs. Whole-body time integrated activity coefficients were between 8.2 and 69.7 hours with a mean of 24.1±18.7 hours, as shown in Table 37. Mean whole body effective dose was 0.095±0.048 mSv/MBq. Whole body effective doses are reported for all patients in Table 37.

TABLE 37
Whole body time integrated activity coefficients (hours) per pa

| Subject ID | Whole Body TIAC (hrs) |
|---|---|
| 25-005 | 12.6 |
| 25-007 | 19.7 |
| 25-008 | 14.2 |
| 25-009 | 8.5 |
| 80-001 | 27.6 |
| 80-004 | 8.2 |
| 80-007 | 17.2 |
| 37-013 | 41.7 |
| 37-015 | 14.4 |
| 37-016 | 69.7 |
| 37-017 | 43.7 |
| 12-004 | 11.1 |
| Mean | 24.1 |
| SD | 18.7 |

TABLE 38
Effective Dose for All Six Patients

| Subject ID | Effective Dose (mSv/MBq) Cycle 1 |
|---|---|
| 25-005 | 0.03 |
| 25-007 | 0.11 |
| 25-008 | 0.07 |
| 25-009 | 0.06 |
| 80-001 | 0.08 |
| 80-004 | 0.03 |
| 80-007 | 0.09 |
| 37-013 | 0.12 |
| 37-015 | 0.11 |
| 37-016 | 0.16 |
| 37-017 | 0.19 |
| 12-004 | 0.07 |
| Mean | 0.09 |
| SD | 0.05 |

Organs with the highest absorbed doses were the lacrimal glands (0.67±0.33 Gy/GBq), left colon (0.54±0.42), rectum (0.52±0.40), kidneys (0.43±0.18 Gy/GBq), and the urinary bladder wall (0.38±0.03 Gy/GBq). Absorbed doses to the rest of the large intestines (right colon) was 0.30±0.23 Gy/GBq. Dose to the salivary glands was 0.13±0.08 Gy/GBq, and dose to the red marrow was 0.08±0.15 Gy/GBq. Average doses to all target organs are reported in the Table 34.

TABLE 39
Absorbed doses (Gy/GBq) in Target Organs included
in the ICRP-89 Male Phantom and lacrimal glands
region, calculated using OLINDA sphere model.

Absorbed Dose (Gy/GBq)

| Target Organ | Mean | SD |
|---|---|---|
| Adrenals | 0.03 | 0.02 |
| Brain | 0.02 | 0.02 |
| Esophagus | 0.02 | 0.02 |
| Eyes | 0.02 | 0.02 |
| Gallbladder Wall | 0.02 | 0.02 |
| Left colon | 0.54 | 0.42 |
| Small Intestine | 0.06 | 0.04 |
| Stomach Wall | 0.02 | 0.02 |
| Right colon | 0.30 | 0.23 |
| Rectum | 0.52 | 0.40 |
| Heart Wall | 0.02 | 0.02 |
| Kidneys | 0.43 | 0.18 |
| Liver | 0.04 | 0.02 |
| Lungs | 0.02 | 0.02 |
| Pancreas | 0.02 | 0.02 |
| Prostate | 0.02 | 0.02 |
| Salivary Glands | 0.13 | 0.08 |
| Red Marrow | 0.08 | 0.15 |
| Osteogenic Cells | 0.05 | 0.09 |
| Spleen | 0.03 | 0.03 |
| Testes | 0.02 | 0.02 |
| Thymus | 0.02 | 0.02 |
| Thyroid | 0.02 | 0.02 |
| Urinary Bladder Wall | 0.38 | 0.03 |
| Total Body | 0.03 | 0.02 |
| Lacrimal glands^{a} | 0.67 | 0.33 |

^{a}Lacrimal glands absorbed dose was calculated using OLINDA sphere model that assume local deposition of the dose.

Absorbed doses to the kidneys (in Gy and Gy/GBq) are reported for each patient included in the dosimetry sub-study in Table 40.

TABLE 40
Absorbed doses to the kidneys in Gy and Gy/GBq

| | Kidneys Absorbed | Kidneys Absorbed |
|---|---|---|
| Subject ID | Dose (Gy) | Dose (Gy/GBq) |
| 25-005 | 4.87 | 0.64 |
| 25-007 | 2.56 | 0.34 |
| 25-008 | 5.82 | 0.81 |
| 25-009 | 2.26 | 0.29 |
| 80-001 | 2.03 | 0.27 |
| 80-004 | 1.68 | 0.22 |
| 80-007 | 3.62 | 0.48 |
| 37-013 | 2.00 | 0.27 |
| 37-015 | 2.36 | 0.32 |
| 37-016 | 3.99 | 0.54 |
| 37-017 | 3.08 | 0.41 |
| 12-004 | 4.48 | 0.57 |
| Mean | 3.23 | 0.43 |
| SD | 1.32 | 0.18 |

Kidneys were identified as the main organ at risk. To determine the safety of delivering 6 treatment cycles, projection of the cumulative absorbed dose to the kidneys after 6 treatment cycles was calculated by multiplying the number of cycles by the absorbed dose at cycle 1, assuming an injected dose of 7.4 GBq.

With an injected dose of 7.4 GBq per cycle, the estimated mean absorbed dose for the kidneys after 6 cycles is 19.3 Gy±8.0 Gy (min: 9.9 Gy, max: 35.83 Gy). Kidney absorbed dose for each subject analyzed after the first treatment cycle, along with the projected kidney dose after 6 cycles is detailed in Table 36.

TABLE 41
Absorbed doses to the kidneys extrapolated to 6 cycles (in
Gy/GBq, and assuming an administered dose of 7.4 Gy)

| | | Kidneys | |
|---|---|---|---|
| | | Absorbed Dose | Extrapolation of |
| | Kidneys | (Gy) Assuming | Absorbed Dose to 6 |
| | Absorbed Dose | 7.4 GBq per | Cycles (Gy) Assuming |
| Subject ID | (Gy/GBq) | cycle | 7.4 GBq per cycle |
| 25-005 | 0.64 | 4.8 | 28.5 |
| 25-007 | 0.34 | 2.5 | 15.0 |
| 25-008 | 0.81 | 6.0 | 35.8 |
| 25-009 | 0.29 | 2.2 | 13.0 |
| 80-001 | 0.27 | 2.0 | 11.8 |
| 80-004 | 0.22 | 1.7 | 9.9 |
| 80-007 | 0.48 | 3.6 | 21.3 |
| 37-013 | 0.27 | 2.0 | 12.0 |
| 37-015 | 0.32 | 2.3 | 14.0 |
| 37-016 | 0.54 | 4.0 | 23.8 |
| 37-017 | 0.41 | 3.0 | 18.0 |
| 12-004 | 0.57 | 4.2 | 25.1 |
| Mean | 0.43 | 3.2 | 19.3 |
| SD | 0.18 | 1.3 | 8.0 |

### Conclusion

The first 12 patients received their first dose of IP in the Eclipse sub-study and 11 of them underwent SPECT/CT at 4 imaging time points as per protocol, while one subject (37-016) only had 3 SPECT/CT, without the scan at 168 h. Dosimetry analysis was successfully run for all of them. Results demonstrate a mean whole-body half-life of 24.1±18.7 hours and a mean effective dose of 0.095±0.048 mSv/MBq. Mean absorbed dose of selected organs are as follows: kidneys (0.43±0.18 Gy/GBq), urinary bladder wall (0.38±0.03 Gy/GBq), large intestines (left colon, right colon, and rectum: 0.54±0.42, 0.30±0.23, 0.52±0.40 Gy/GBq, respectively), and salivary glands (0.13±0.08 Gy/GBq). Mean absorbed dose to the lacrimal glands is 0.67±0.33 Gy/GBq. Based on these results, the projected cumulative absorbed dose to the kidneys after 6 cycles will be 19.3±8.0 Gy.

### Example 10: Study Evaluating Metastatic Castrate Resistant Prostrate Cancer (mCRPC) Treatment Using [¹⁷⁷Lu]Lu PSMA-I&T Prostrate-Specific Membrane Antigen (PSMA) Therapy

Key subject criteria eligibility for inclusion in the study are described below:
Male age ≥18 years;
Progressive mCRPC based on at least one of the following per PCWG3:
   Serum/plasma PSA progression
   Soft-tissue progression
   Bone progression;
Prior treatment with only one ARAT (abiraterone, enzalutamide, darolutamide, apalutamide) in either the CSPC or CRPC setting;
Ineligible or averse to chemotherapy;
PSMA PET scan positive (18F-DCFPyL or 68Ga-PSMA11) per central review; and
ECOG Performance Status: 0-1

Subjects excluded from the study based on the following:
Any prior cytotoxic chemotherapy for CRPC (e.g. cabazitaxel or docetaxel); chemotherapy for hormone-sensitive prostate cancer is allowed (last dose >1 year prior to consent;
Liver metastases >1 cm;
Superscan on bone scan;
CNS metastases; or
Contraindications to the use of planner ARAT therapy.

Radiation dosimetry of [¹⁷⁷Lu]Lu-PSMA-I&T was calculated in 27 patients with mCRPC based on biodistribution data from planar whole-body conjugate imaging at 1, 24, 48, 72, and 168 hours and, for 7 of them, SPECT/CT imaging at 48-72 hours post injection of their first cycle of [¹⁷⁷Lu]Lu-PSMA-I&T (6.8±10% GBq).

Regions of interest were drawn on the planar images.

Volumes of interest were constructed on the SPECT and CT

images for kidneys.

For each subject, kinetic data were modeled to determine

normalized number of disintegrations in organs and tissues, from which the absorbed dose estimates were computed using the MIRD method.

Table 37 summarizes the dose estimates summary for selected organs (mGy/MBq or Gy/GBq). As illustrated in Table 42, organs receiving the

largest absorbed doses were the lacrimal glands at 1.2 Gy/GBq, followed by the kidneys at 0.73 Gy/GBq.

TABLE 42
[¹⁷⁷Lu]Lu -PSMA-I&T dose estimates summary
statistics for selected organs (mGy/MBq or Gy/GBq)

| | Mean | Min | Max | SD | CV |
|---|---|---|---|---|---|
| Kidneys | 0.73 | 0.22 | 1.8 | 0.33 | 45% |
| Left Colon | 0.52 | 0.013 | 1.5 | 0.281 | 54% |
| Salivary Glands | 0.34 | 0.14 | 1.5 | 0.27 | 80% |
| Red Marrow | 0.034 | 0.014 | 0.11 | 0.019 | 58% |
| Spleen | 0.16 | 0.022 | 0.80 | 0.17 | 113% |
| Heart Wall | 0.036 | 0.017 | 0.15 | 0.025 | 71% |
| Lacrimal Glands | 1.2 | 0.38 | 6.7 | 1.2 | 97% |
| Liver | 0.051 | 0.025 | 0.24 | 0.042 | 82% |
| Thyroid | 0.17 | 0.011 | 0.77 | 0.15 | 90% |
| Bladder Wall | 0.33 | 0.14 | 0.36 | 0.041 | 13% |

The average dose to the salivary glands and red marrow was 0.34 Gy/GBq and 0.034 Gy/GBq, respectively.

For a cumulative administered activity of 27.2 GBq, i.e. four cycles of 6.8 GBq, the kidneys would receive a cumulative absorbed dose of 19.9 Gy, and the red marrow, 0.91 Gy.

SPECT/CT vs planar-based kidney dosimetry was consistent across most subjects (±20%) where SPECT/CT images were available with a mean kidney absorbed dose difference of 1%.

Table 43A and 43B show a comparison of the dosimetry results of different studies.

TABLE 43A
[¹⁷⁷Lu]Lu-PSMA-I&T dose estimates summary statistics for
selected organs (mGy/MBq or Gy/GBq)—Head-to-Head
Comparison of Eclipse to Splash and/or Pluvicto.

| | Splash | | Pluvicto Spc | | Eclipse | |
|---|---|---|---|---|---|---|
| | Mean | | Mean | | Mean | |
| Organs | Gy/GBq | SD | Gy/GBq | SD | Gy/GBq | SD |
| Heart Wall | 0.04 | 0.03 | 0.17 | 0.12 | 0.02 | 0.02 |
| Kidneys | 0.73 | 0.33 | 0.43 | 0.16 | 0.43 | 0.18 |
| Lacrimal gland | 1.20 | 1.20 | 2.1 | 0.47 | 0.67 | 0.33 |
| Left colon | 0.52 | 0.28 | 0.58 | 0.14 | 0.54 | 0.42 |
| Liver | 0.051 | 0.04 | 0.09 | 0.04 | 0.04 | 0.02 |
| Rectum | | | 0.56 | 0.14 | 0.52 | 0.40 |
| Red Marrow | 0.03 | 0.02 | Not included | | 0.08 | 0.15 |
| Right colon | | | 0.32 | 0.08 | 0.30 | 0.23 |
| Salivary glands | 0.34 | 0.27 | 0.63 | 0.36 | 0.13 | 0.08 |
| Spleen | 0.16 | 0.17 | 0.07 | 0.03 | 0.03 | 0.03 |
| Thyroid | 0.17 | 0.15 | 0.26 | 0.37 | 0.02 | 0.02 |
| Urinary bladder wall | 0.33 | 0.04 | 0.32 | 0.03 | 0.38 | 0.03 |

Note:
superior and unexpected dosimetry results of Eclipse embodiment compared to Splash and/or Pluvicto.

TABLE 43B^{a}
Results from 26 patients after Cycle 1 of 177Lu-PSMA I&T

| | Absorbed Dose Mean Gy/GBq (SD) | | | | |
|---|---|---|---|---|---|
| Organs | ECLIPSE | Pluvicto | | SPLASH | |
| Kidneys | 0.41 (0.15) | 0.43 | (0.16) | 0.73 | (0.33) |
| Lacrimal | 0.40 (0.36) | 2.1 | (0.47) | 1.2 | (1.2) |
| Glands | | | | | |
| Salivary | 0.19 (0.16) | 0.63 | (0.36) | 0.34 | (0.27) |
| Glands | | | | | |
| Liver | 0.04 (0.02) | 0.090 | (0.044) | 0.051 | (0.042) |
| Left Colon | 0.47 (0.31) | 0.58 | (0.14) | 0.52 | (0.281) |
| Right Colon | 0.25 (0.17) | 0.32 | (0.078) | NP | |
| Bone Marrow | 0.08 (0.12) | NP | | 0.034 | (0.019) |
| Spleen | 0.03 (0.02) | 0.067 | (0.027) | 0.16 | (0.17) |
| Thyroid | 0.01 (0.02) | 0.26 | (0.37) | 0.17 | (0.15) |
| Bladder | 0.41 (0.05) | 0.32 | (0.027) | 0.33 | (0.041) |
| Heart | 0.02 (0.02) | 0.17 | (0.12) | 0.036 | (0.025) |

^{a}Variability in dose may be due to: methodology, variability in patient disease status, dosimetry never to be written in stone

### Example 11: Parameters Evaluated for Compositions

A summary of the parameters, range evaluated, and the established proven acceptable range (PAR) during development is presented in Table 44 below.

| Parameter | Range Evaluated | | Target | |
|---|---|---|---|---|
| Batch Size at | 50-15,200 | mCi | 4-15 | Ci |
| Radiolabeling | | | | |
| Drug Substance | pH 3.0-5.75 | | >5 | |
| Drug Substance - | 0.04-0.53M | | Concentration of Reaction | |
| Sodium Ascorbate | (7.9-105.0 mg/mL) | | Mixture: ≥0.2M (40 mg/mL) | |
| Concentration | | | | |
| Radiolabeling | 55-95° | C. | 75° | C.^{a} |
| Reactor Temperature | | | | |
| Setpoint | | | | |
| Reaction Time | 0-45 | min | 10 | min |
| Drug Substance - | 0.1-1.2 | μg/mCi | 0.6 | μg/mCi |
| PSMA I&T to | | | | |
| activity ratio at | | | | |
| calibration | | | | |
| Reaction RAC | 91-333 | mCi/mL | Variable based on batch size | |
| Drug Product - | 2.8-35 | mg/mL | 33 mg/mL on the ascorbic | |
| Sodium Ascorbate | | | acid basis | |
| Content | | | | |
| Drug Product - | 0-7% | | 0% | |
| Ethanol Content | | | | |
| Drug Product - | 0-0.15 | mg/mL | 0.1 | mg/mL |
| DTPA Content | | | | |
| Drug Product - pH | 3.55-13.14 | | 4.0-4.5 | |
| Drug Product | 7.57-22.6 | mCi/mL | 18.2 mCi/mL at time of | |
| Radioactivity | | | formulation | |
| Concentration | | | | |
| Drug Product Vial | 25R Schott Vial with 20 mm Stopper (Serum, Nova Pure RP | | | |
| | 1343; 4023/50G (Item: 19700361) | | | |
| Static Product Hold | 0-72 | h | 72 | h |
| in Filters | | | | |
| Product Bulk | Hold 0-96 h | | Stable up to 96 h | |
| | 5.2-11.5 Ci | | | |
| Terminal Sterilization | Yes/No | | No | |
| Reaction Buffer | −5%-+5% | | −2%-+2% | |
| Weight Tolerance | | | | |
| Range - Sodium | | | | |
| Ascorbate | | | | |
| Dilution Buffer | −5%-+5% | | −2%-+2% | |
| Weight Tolerance | | | | |
| Range - Sodium | | | | |
| Ascorbate & | | | | |
| Ascorbic Acid | | | | |
| Intermediate | 0-4 | hours | ≤1 | hr |

| | | |
|---|---|---|
| Formulation Hold | | |
| Reaction Buffer | 0-4 Weeks prior to use | 0-3 weeks; |
| Preparation Age | | Fresh recommended |
| Dilution Buffer | 0-4 Weeks prior to use | 0-4 weeks; |
| Preparation Age | | Fresh recommended |

^{a}Internal reaction temperature; Equipment set-points may differ

### Example 12—Impact of pH on the Extent of Lu-177 Incorporation During Radiolabeling

The purpose of this study was to determine whether changing the pH of the dilution buffer used to dilute the Lu-PSMA I&T reaction solution to the final volume would impact the stability of the drug product formulation at a 200 rig/vial PSMA I&T concentration.

Approach: The impact of pH on the stability of the drug product was evaluated by preparing eight formulations at pH values between 3.6 and 13.1 and measuring their pH, radiochemical purity (RCP), and PSMA I&T content at release and expiry. Once the effect of pH was established over a wide range, the impacts to drug product stability of pH values closer to expected conditions was further elucidated by holding four of the formulations with pH values between 3.6 and 5.0 for an additional 4 days. After this additional period, the appearance, pH, RCP, and PSMA I&T content was measured to further understand the impacts of pH on drug product stability.

MATERIALS & EQUIPMENT: A detailed list of all raw materials and equipment used in the preparation and evaluation of the formulations in this report can be found in the batch records for Lot #8017-QF-3 (Attachment 1) and 8017-QF-3-E2 (Attachment 2) and the accompanying analytical records (Attachment 3 and 4).

### Procedure

Radiosynthesis and Formulation: A detailed description of the procedure is provided in the batch records for Lot #8017-QF-3 (Attachment 1) and 8017-QF-3-E2 (Attachment 2). In brief, the contents of eight vials containing approximately 250 mCi of [¹⁷⁷Lu]LuCl₃ were transferred to eight (8) individual 10 mL tubing vials, each containing 200 μg of PSMA I&T in 1.2 mL of Reaction Buffer (0.2 M sodium ascorbate). Reaction vials were heated for at least 10 min at 75° C. to allow for radiolabeling of the PSMA I&T with the [¹⁷⁷Lu]Lu³⁺ to occur. Reaction vials were then cooled for at least 5 min before transferring contents to 30 mL molded vials and diluted with 13.1 mL dilution buffer to achieve the target values listed in Table 45. To achieve the final desired pH values, the pH of sample 8017-QF-3-E2-D was adjusted using 40 μL of 1M NaOH to a final measured pH of 5.0. Actual measured concentrations and properties of the prepared formulations at release, T0+72 h, and T0+168 h are reported in the Results and Discussion section.

TABLE 45
Target Values for the Formulations Used in this Report

| | Final Drug Product Formulation (Targets) | | | | | | |
|---|---|---|---|---|---|---|---|
| | | PSMA | | Sodium | | Total | |
| | | I&T | | Ascor- | DTPA | Vol- | RAC¹ |
| | | Quantity | Ethanol | bate | mg/ | ume | (mCi/ |
| ID | pH | (μg/mL) | (% v/v) | mg/ml) | mL) | mL) | mL) |
| 8017-QF-3-A | 4.0 | 13.5 | 3.5 | 20 | 0.075 | 14.8 | 16.9 |
| 8017-QF-3-B | 5.2 | | | | | | |
| 8017-QF-3-C | 12.0 | | | | | | |
| 8017-QF-3-D | 14.0 | | | | | | |
| 8017-QF-3- | 3.5 | 13.5 | 3.5 | 20 | 0.075 | 14.8 | 16.9 |
| E2-A | | | | | | | |
| 8017-QF-3- | 4.0 | | | | | | |
| E2-B | | | | | | | |
| 8017-QF-3- | 4.6 | | | | | | |
| E2-C | | | | | | | |
| 8017-QF-3- | 5.0 | | | | | | |
| E2-D | | | | | | | |

¹RAC: Radioactivity Concentration at the end of formulation

Sample Analysis: The pH, radioactivity concentration (RAC), PSMA I&T content, and radiochemical purity (RCP) of the formulations were determined using a variety of analytical techniques described in the following sub-sections. A detailed record of the steps performed for each analysis can be found in Attachment 1-4.

pH: The pH of the sample was measured with a pH meter and probe calibrated on the day of analysis using three buffers at pH values of 4.01, 7.00, and 10.01.

### Radioactive Concentration

The activity of each formulation vial was determined on a Capintec CRC-15R Dose Calibrator adjusted to the appropriate Lu-177 isotope setting.

### PSMA I&T Content and Radiochemical Purity (RCP)

Both the determination of the assay for PSMA I&T and related substances and the RCP of the formulations were determined via HPLC analysis. Details regarding the chromatographic method are summarized in Table 46. The system suitability of the instrument was established by triplicate injections of a 15 ppm PSMA I&T standard prior to sample analysis and a single 15 ppm check standard every 10 sample injections. A five-point standard curve of PSMA I&T concentrations between 0.5 and 25 ppm was used to determine the PSMA I&T concentration in the formulations.

TABLE 46
HPLC Method and Instrument Conditions
Instrument Parameters

| Column | Phenomenex Luna C18(2) Column- | | | | | |
|---|---|---|---|---|---|---|
| | 5 μm-150 × 4.6 mm | | | | | |
| Flow Rate | 1.0 mL/min | | | | | |
| Mobile Phase | Mobile Phase A: Water (0.1% TFA) | | | | | |
| | Mobile Phase B: Acetonitrile (0.1% TFA) | | | | | |
| Column Temperature | 35° C. | | | | | |
| Injection Volume | 100 μL for PSMA I&T quantification, | | | | | |
| | variable for RCP analysis^{a} | | | | | |
| Run Time | 30 min | | | | | |
| UV Wavelength | 220 nm | | | | | |
| Radioactive Detector | Beckman Coulter Rad Detector | | | | | |
| Radioactivity Detector | Variable^{a} | | | | | |
| Setting | | | | | | |
| Gradient | Time (min) | 10 | 20 | 26 | 27 | 30 |
| | % Mobile | 85 | 75 | 75 | 85 | 85 |
| | Phase A | | | | | |
| | % Mobile | 15 | 25 | 25 | 15 | 15 |
| | Phase B | | | | | |

^{a}Adjusted based on sample radioactivity concentration to maximize use of detector's full scale

Results: The results of the analytical testing for the eight formulations prepared in this report are summarized in Table 47.

TABLE 47
Analytical results from the analysis of the formulations
prepared for this report.

| | | | | PSMA | | |
|---|---|---|---|---|---|---|
| | | | | I&T and | | |
| | | | | Related | | |
| | | | | Sub- | | |
| | | | | stances | | RAC² |
| | Time- | | | Content | RCP | mCi/ |
| ID | point | Appearance¹ | pH | (μg/mL) | (%) | mL) |
| 8017- | T0 h | Clear, Colorless, | 4.30 | 12.1 | 98.7 | 14.2 |
| QF- | | Particulate Free | | | | |
| 3-A | T0 + 72 h | ND | 4.24 | 11.6 | 96.5 | ND³ |
| 8017- | T0 h | Clear, Colorless, | 5.30 | 13.0 | 98.4 | 14.3 |
| QF- | | Particulate Free | | | | |
| 3-B | T0 + 72 h | ND | 5.28 | 11.8 | 94.0 | ND³ |
| 8017- | T0 h | Clear, Light | 11.35 | 2.7 | 98.2 | 13.8 |
| QF- | | Orange, | | | | |
| 3-C | | Particulate Free | | | | |
| | T0 + 72 h | ND | 10.23 | 11.3 | 90.5 | ND³ |
| 8017- | T0 h | Clear, Orange, | 13.14 | 0.5 | 4.4 | 14.4 |
| QF- | | Particulate Free | | | | |
| 3-D | T0 + 72 h | ND | 13.08 | 0.7 | 3.1 | ND³ |
| 8017- | T0 h | Clear, Colorless, | 3.55 | 14.7 | 99.5 | 15.5 |
| QF- | | Free | | | | |
| 3-E2-A | T0 + 72 h | Clear, Colorless | 3.52 | 13.2 | 96.9 | ND³ |
| | | Solution, No | | | | |
| | | Visible Particles | | | | |
| | T0 + 168 h | Clear, Colorless | 3.47 | 13.1 | 93.7 | ND³ |
| | | Solution, No | | | | |
| | | Visible Particles | | | | |
| 8017- | T0 h | Clear, Colorless, | 4.09 | 15.5 | 99.2 | 15.7 |
| QF- | | Particulate Free | | | | |
| 3-E2-B | T0 + 72 h | Clear, Colorless | 4.10 | 13.5 | 96.5 | ND³ |
| | | Solution, No | | | | |
| | | Visible Particles | | | | |
| | T0 + 168 h | Clear, Colorless | 4.07 | 13.6 | 93.6 | ND³ |
| | | Solution, No | | | | |
| | | Visible Particles | | | | |
| 8017- | T0 h | Clear, Colorless, | 4.50 | 15.0 | 98.4 | 14.6 |
| QF- | | Particulate Free | | | | |
| 3-E2-C | T0 + 72 h | Clear, Colorless | 4.53 | 13.8 | 96.5 | ND³ |
| | | Solution, No | | | | |
| | | Visible Particles | | | | |
| | T0 + 168 h | Clear, Colorless | 4.52 | 13.9 | 93.7 | ND³ |
| | | Solution, No | | | | |
| | | Visible Particles | | | | |
| 8017- | T0 h | Clear, Colorless, | 4.97 | 15.5 | 99.0 | 14.7 |
| QF- | | Particulate Free | | | | |
| 3-E2-D | T0 + 72 h | Clear, Colorless | 4.99 | 14.4 | 95.4 | ND³ |
| | | Solution, No | | | | |
| | | Visible Particles | | | | |
| | T0 + 168 h | Clear, Colorless | 4.97 | 13.6 | 91.6 | ND³ |
| | | Solution, No | | | | |
| | | Visible Particles | | | | |

¹Appearance results are equivalent between batches except for QF-3-C and QF-3-D but wording varies based on both the discretion of the analyst and where the results were recorded (i.e., batch record, notebook or datasheet);
²Radioactivity Concentration at the end of formulation;
³RAC was not measured at stability timepoints since it is not considered a stability-indicating attribute;
ND: Not Determined

### 8. Discussion

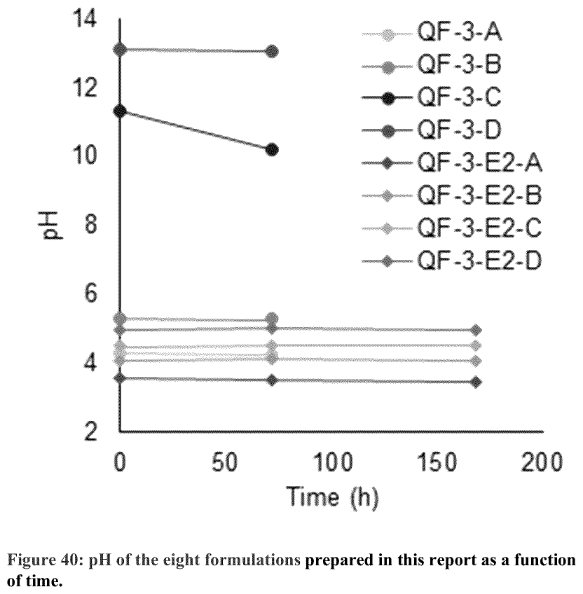

To determine the impacts of pH on the stability of the drug product, eight formulations were prepared at various pH values. The pH of the samples remained relatively stable over the course of the testing period with a max change of 0.1 pH units from the initial value for any of the formulations except for the sample at a starting pH of 11.4 (FIG. 40). The pH of the formulation at 11.4 dropped to a value of 10.2 after 72 h. The decrease in the pH due to the degradation of various species present in the formulation changing the composition and therefore the solution pH.

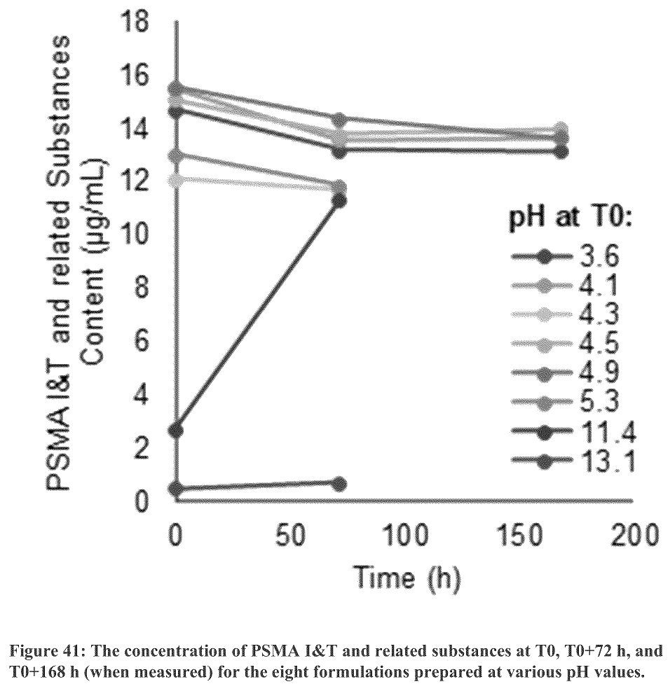

Next, the stability of the drug product as evaluated by the RCP of the formulations at expiry was assessed as a function of the starting pH. The RCP of most formulations at release (TO) was as expected with an average value of 98.8±0.5% except for the formulation at a starting pH of 13.1 which had a starting RCP of 4.4% (Table 47). As none of the other formulations had this low of an RCP value including the formulation at a starting pH of 11.4, the low RCP of 4.4% is expected to be due to a phenomenon occurring specifically at extremely high pH values of at least 13.1. When the PSMA I&T content of the sample at a pH of 13.1 was evaluated, it was much lower at release compared to the target value of 13.5 ppm that the other formulations prepared in this study were close to (FIG. 41). Therefore, it is expected that the high pH of this formulation caused the PSMA I&T to rapidly degrade immediately after preparation.

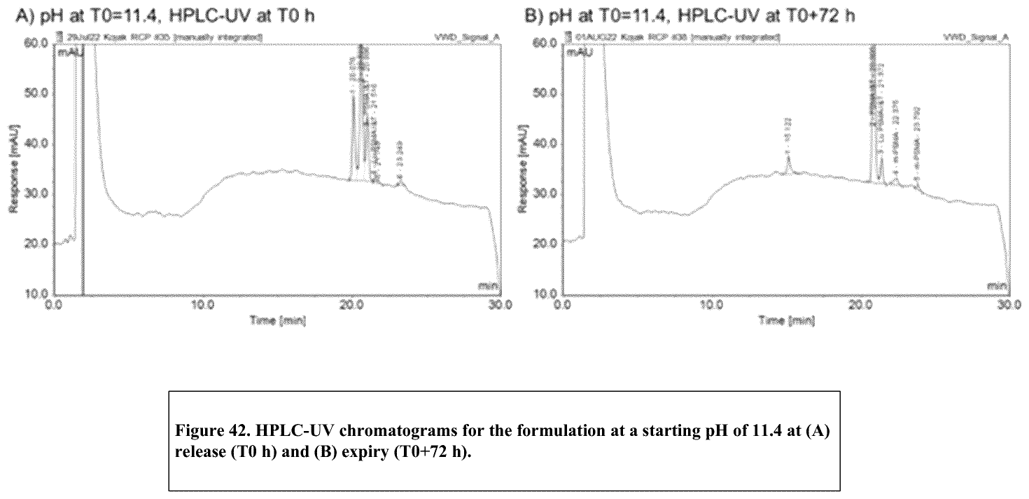

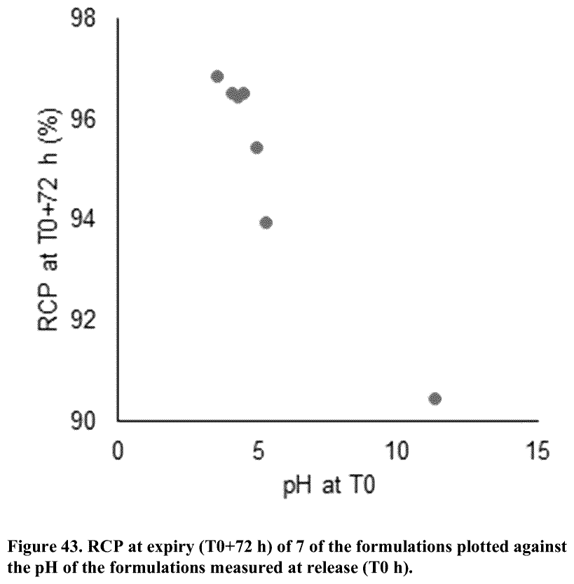

During the HPLC analysis of the PSMA I&T content, the PSMA I&T content for the sample at a starting pH of 11.4 had a PSMA I&T content of 2.7 μg/mL at release, but at expiry, the PSMA I&T content was measured to be 11.3 μg/mL (FIG. 41). The value measured at expiry was much closer to the expected value of 13.5 μg/mL and much higher than the value measured at release. Since it is improbable that the PSMA I&T content increased during this time, we hypothesize that there was an issue in the preparation or measurement of this formulation that caused the PSMA I&T content to increase from release to expiry. From the HPLC-UV chromatograms, there were two additional peaks present for the sample at release that eluted near the PSMA I&T peak (FIG. 42A) that were not present in the chromatogram at expiry (FIG. 42B). The cause of these peaks in the release chromatogram and the measured increase in PSMA I&T content between release and expiry was not investigated as the RCP of the sample was lower at expiry than other formulations (FIG. 43) so this formulation pH would not be used going forward.

The difference in the RCP values for the seven formulations below an initial pH of 12 become clearer at expiry (T0+72 h) with RCP values between 96.9% and 90.5% (FIG. 7-4). The decreasing RCP values at expiry trended with increasing pH with the lowest RCP being measured for the sample at a starting pH of 11.4. Therefore, it is expected that keeping the formulation at lower pH values will improve the stability of the formulation at expiry.

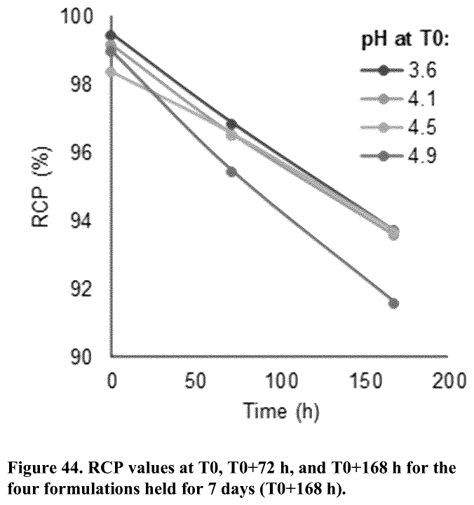

To further assess the impact of pH on the stability of the formulation, four of the formulations at pH values between 3.6 and 4.9 were tested after an additional 4 days (FIG. 44). The three formulations at pH values between 3.6 and 4.5 had a very small spread of RCP values at 168 h with an average value of 93.7±0.1%. In contrast, the formulation at a starting pH of 4.9 had an RCP 2.1% lower than the other three formulations. This data indicates that pH has no measurable impact on the RCP of the formulation when between pH values of 3.6 and 4.5 even at 7 days after preparation, but the RCP decreased when the pH was increased by just 0.4 pH units outside of this range.

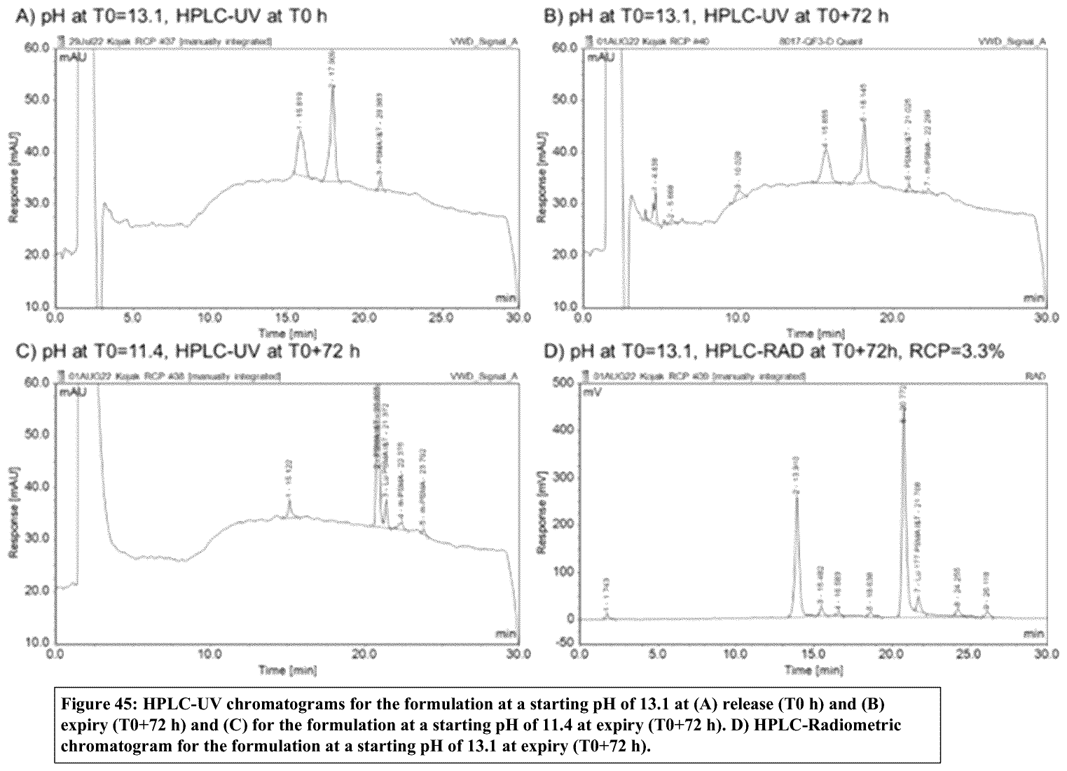

The presence of degradation products can be assessed using the HPLC-UV and HPLC-Radiometric chromatograms of the individual samples and can provide additional information about how formulation pH impacts stability. For the formulation at a starting pH of 13.1, the HPLC-UV chromatogram at release displayed two large impurity peaks (FIG. 45A) that after an additional 72 h were now accompanied by many smaller peaks throughout the chromatogram (FIG. 45B). In contrast, the sample with the next highest initial pH of 11.4 had only a small organic impurity peak at expiry (FIG. 45C) which demonstrates the difference in degradation between the formulation at a pH of 11.4 compared to 13.1. Similarly, the HPLC-Radiometric chromatogram at expiry for the formulation at an initial pH of 13.1 had only a small peak associated with the [¹⁷⁷Lu]Lu-PSMA I&T (FIG. 45D). There were also many large and small peaks scattered throughout the chromatogram associated with PSMA I&T degradation products still chelated to the [¹⁷⁷Lu]Lu³⁺ indicating that many of the radiometric impurities were not unchelated [¹⁷⁷Lu]Lu³⁺ but were related to the incomplete degradation of the [¹⁷⁷Lu]Lu-PSMA I&T complex.

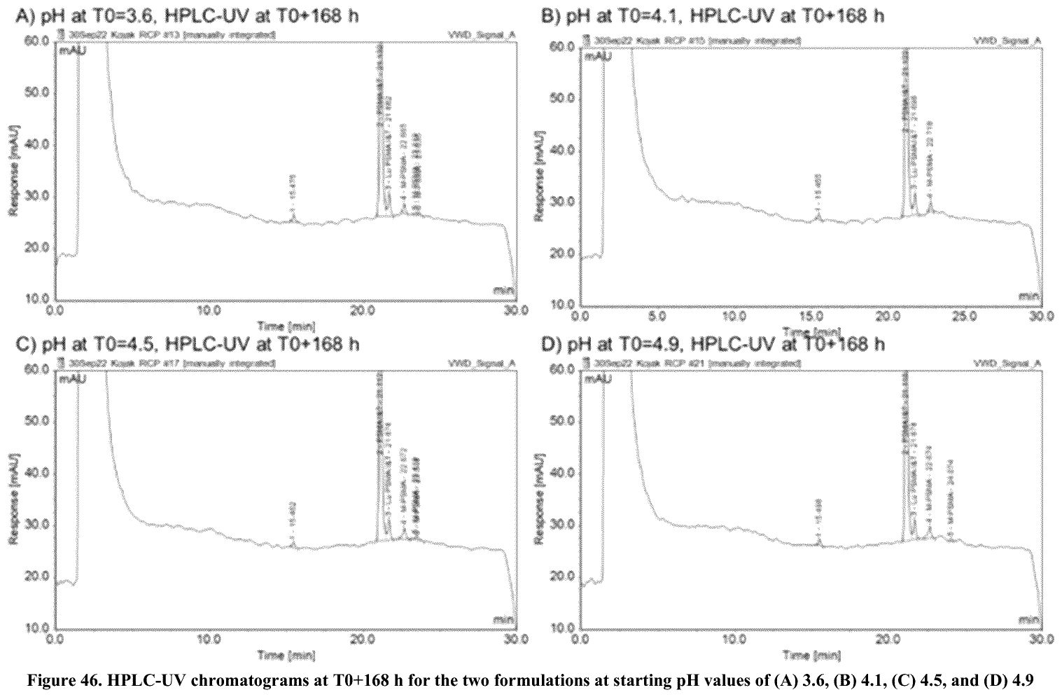

In contrast to the large number of peaks observed in the formulation at a starting pH of 13.1, the HPLC-UV chromatograms for the formulations at pH values between 3.6 and 4.9 only had a small peak close to 15.5 min even at 7 days after preparation (FIG. 46). Due to the lack of variability in the presence of organic impurities in the HPLC-UV chromatograms for these four formulations, it is expected that the organic impurity content of the formulation is largely independent of the pH between pH values of 3.6 and 4.9.

### Conclusion

In conclusion, the pH of the dilution buffer used to prepare the final formulation will impact the drug product stability if the final pH is above 4.5. For formulations prepared at final pH values above 4.5, the pH was found to negatively correlate with the RCP of the formulation with extreme degradation (i.e., RCP=4.4%) observed for the formulation at a pH of 13.1. The three formulations that had a starting pH between 3.6 and 4.5 had comparable RCP values and similar organic impurity profiles to each other at both 3 and 7 days after preparation. As such, future formulations should be prepared within the pH range of 3.6 to 4.5 to maximize stability of the formulation with regards to pH.

There is no difference at time of release for samples prepared at a pH between 4.30 and 5.30. Likewise, no difference was observed in samples prepared at a pH between 3.55 and 4.50.

The solution pH of the reaction mixture does affect the radiolabeling of the drug substance. At pH's less than about 4.25, about 4.15, or about 4.00, the RCY is negatively impacted. It may be recommended to keep the pH of the target solution above 4.0, above 4.1, above 4.25, above 4.2, above 4.3, above 4.4, above 4.5, above 4.6, above 4.7, above 4.75 above 4.8, above 4.9, or above 5.0, however development data suggest that a range of approximately 4.5 to 5.75 may also produce acceptable results.

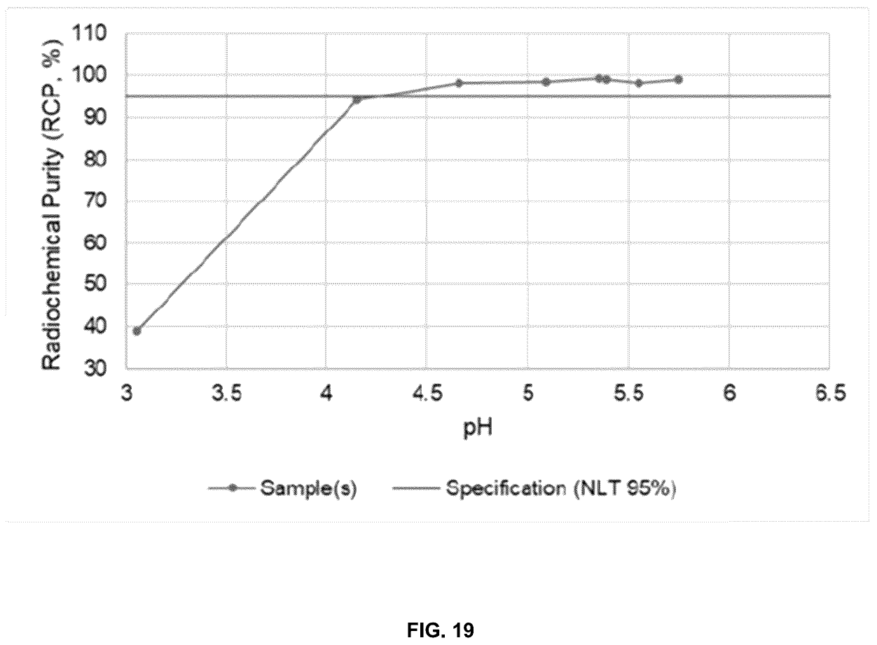

### FIG. 19—RCP as a Function of Radiolabeling pH

### Example 13—RCP as a Function of Sodium Ascorbate Concentration (Reaction Buffer)

Despite an outlier at a reaction mixture concentration of 0.46M sodium ascorbate, in reactions performed with low radioactivity (i.e., 50 mCi), the data support the conclusion that the concentration of sodium ascorbate in the reaction mixture does not have an impact on the extent of Lu-177 incorporation (e.g., including across the range of 0.04M to 0.53M).

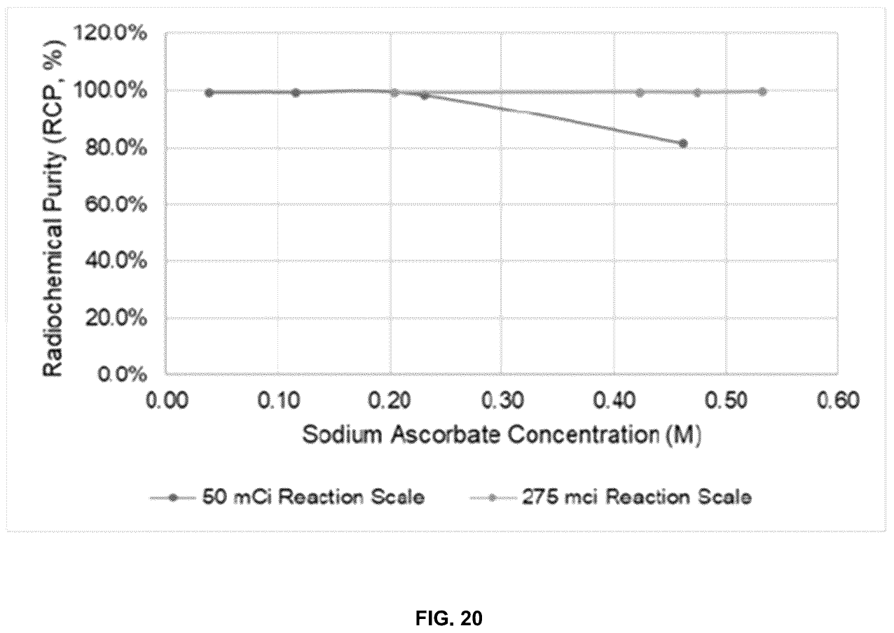

### FIG. 20—RCP as a Function of Sodium Ascorbate Concentration (Reaction Buffer)

### Example 14—Impact of Reaction Temperature and Time on Radiochemical Yield (RCY)

The reaction temperature for producing ¹⁷⁷Lu-PSMA I&T may occur at a temperature from about 55° C. to about 95° C., from about 55° C. to about 85° C., from about 55° C. to about 75° C., from about 55° C. to about 70° C., from about 60° C. to about 85° C., from about 65° C. to about 85° C., from about 70° C. to about 80° C., from about 65° C. to about 75° C. The reaction may occur from about 0 minutes to about 60 minutes, about 0 minutes to about 45 minutes, about 0 minutes to about 30 minutes, about 0 minutes to about 15 minutes, about 0 minutes to about 10 minutes, or about 0 minutes to about 5 minutes. The reaction temperature for producing ¹⁷⁷Lu-PSMA I&T may occur at an internal temperature from about 65° C. to 75° C. for 5 minutes to about 15 minutes, about 70° C. to 75° C. for 5 minutes to about 15 minutes, about 72° C. to 75° C. for 5 minutes to about 15 minutes, about 75° C. for 5 minutes to about 15 minutes, about 75° C. for 10 minutes. In one aspect, short reaction times (5-10 minutes) at temperatures of 75° C. or greater may result in Lu-177 incorporation of 95% or greater, 95.5% or greater, 96% or greater, 96.5% or greater, 97% or greater, 97.5% or greater, 98% or greater, 98.5% or greater, 99% or greater, or 99.5% or greater.

However, in some aspects, at temperatures greater than 75° C., degradation of the peptide is increased. This is seen by the double cyclization peaks after the main product peak when the drug substance is analyzed via HPLC. Note that there are minimal peaks seen when a reaction temperature of 55° C. is used, but this temperature requires extending the radiolabeling time. Therefore, in some aspects, to minimize generating impurities during the reaction step of the process and minimize the reaction time, a target internal temperature of 75° C. for 10 minutes is recommended with 80° C. as an upper limit.

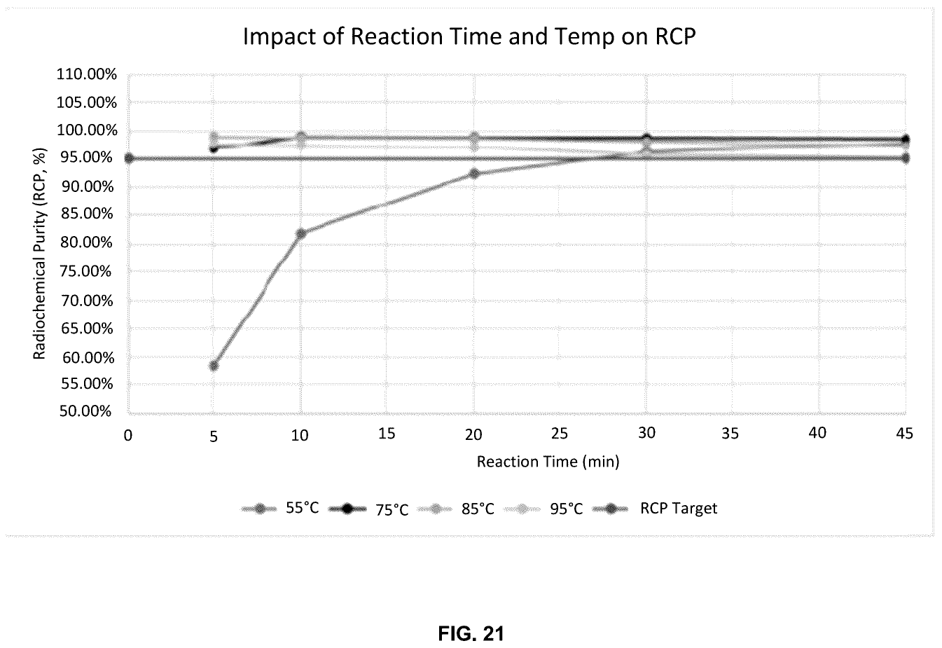

### FIG. 21—Impact of Reaction Time and Temp on RCP

### Example 15—Purification Impact the Drug Product's Radiochemical Yield and Radiochemical Purity

Purification resulted in a lower radiochemical yield at time of synthesis, and a drug product with lower RCP after 72 h.

TABLE 48

| | Release | Expiry |
|---|---|---|
| Sample ID | (T0) | (T0 + 72 h) |
| Not Purified | 99.51% | 95.99% |
| Purified | 98.40% | 94.05% |

### Example 16—PSMA I&T: Lu-177 Ratio and Radiochemical Yield

### Introduction

The radiotherapeutic drug product, lutetium Lu-177 PSMA I&T solution, is currently being evaluated as a treatment for metastatic castration-resistant prostate cancer. The main component of the drug product is the β- emitter Lu-177, which when chelated to PSMA I&T ligand, delivers the radionuclide to the cancer cells located in the prostate.

Several variables must be considered when optimizing conditions for radiosynthesis. These variables include, but are not limited to, stoichiometry of starting materials, reaction temperature, and time.

### Purpose

The purpose of this study is to determine the effects of the ratio of PSMA I&T to Lu-177 on the incorporation of [¹⁷⁷Lu]Lu³⁺ (i.e, RCP) to form the [¹⁷⁷Lu]Lu-PSMA I&T drug substance.

### Approach

The effects of the ratio of PSMA I&T to [¹⁷⁷Lu]Lu³⁺ on the radiochemical yield (RCY) of the lutetium Lu-177 PSMA I&T drug substance was examined by comparing the RCY of several radiolabeling experiments performed using different ratios of PSMA I&T relative to [¹⁷⁷Lu]Lu³⁺ during radiosynthesis.

### Test Materials & Equipment

### Materials and Equipment

Traceability to all raw materials and equipment used in these studies is provided through the manufacturing batch records contemporaneously completed during study execution. The lot numbers referenced in this report are summarized in Table 49.

| Data Set | Lot Number |
|---|---|
| 1 | 8017-QP3-E1 |
| 2 | 8017-QP3-E2 |

### Procedure

### Reaction Conditions

For specific details regarding manufacturing steps, please consult the batch records provided in the attachments. In general, the manufacturing process was performed as follows: lutetium Lu-177 PSMA I&T was prepared by heating a solution of [¹⁷⁷Lu]lutetium chloride ([¹⁷⁷Lu]LuCl₃) and PSMA I&T in a sodium ascorbate buffer (target pH 5.25) for approximately 10 minutes at 75° C. Since the objective of this experiment was to examine the impact of the ratio of PSMA I&T to [¹⁷⁷Lu]Lu³⁺ on the manufacturing process, formulation details are not discussed.

In these experiments, radiochemical purity (RCP) was measured after the end of synthesis and formulation of the drug substance.

The details of lutetium Lu-177 PSMA I&T drug substance referenced in this report are summarized in Table 50.

TABLE 50
Batch Record Summary

| | | | Lu-177 | PSMA | PSMA |
|---|---|---|---|---|---|
| Data | | | Activity | Quantity | I&T:Lu |
| Set | Lot No. | Experiment | (mCi) | (μg) | (μg:mCi) |
| 1 | 8017-QP3-E1 | 1-A | 61.9 | 5 | 0.08 |
| | | 1-B | 61.9 | 15 | 0.24 |
| | | 1-C | 61.8 | 40 | 0.65 |
| | | 1-D | 61.6 | 60 | 0.97 |
| 2 | 8017-QP3-E2 | 2-A | 36.6 | 15 | 0.41 |
| | | 2-B | 36.6 | 22.5 | 0.61 |
| | | 2-C | 36.6 | 30 | 0.82 |
| | | 2-D | 36.5 | 40 | 1.10 |

### Sample Analysis

The samples prepared were analyzed by high-performance liquid chromatography (HPLC) to determine the radiochemical purity (RCP). The details of the method are briefly described below.

### Radiochemical Purity (HPLC)

Radiochemical purity was evaluated by HPLC analysis. Details regarding the chromatographic method are summarized in Table 51.

Instrument Parameters

| Column | Phenomenex Luna C18(2) Column- | | | | | |
|---|---|---|---|---|---|---|
| | 5 μm-150 × 4.6 mm | | | | | |
| Flow Rate | 1.0 mL/min | | | | | |
| Mobile Phase | Mobile Phase A: Water (0.1% TFA) | | | | | |
| | Mobile Phase B: Acetonitrile (0.1% TFA) | | | | | |
| Column Temperature | 35° C. | | | | | |
| Injection Volume | PSMA I&T quantification: 100 μL, | | | | | |
| | RCP analysisª: variable | | | | | |
| Run Time | 30 min | | | | | |
| UV Wavelength | 220 nm | | | | | |
| Radioactive Detector | Beckman Coulter Rad Detector | | | | | |
| Radioactivity Detector | Variable^{a} | | | | | |
| Setting | | | | | | |
| Gradient | Time (min) | 0 | 20 | 26 | 27 | 30 |
| | % Mobile | 85 | 75 | 75 | 85 | 85 |
| | Phase A | | | | | |
| | % Mobile | 15 | 25 | 25 | 15 | 15 |
| | Phase B | | | | | |

### Results and Discussion

The purpose of this experiment was to evaluate the impact of the ratio of PSMA I&T to [¹⁷⁷Lu]Lu³⁺ during radiolabeling, and to explore the design space to determine the appropriate ratio for the process. In this study, the ratio of PSMA I&T to [¹⁷⁷Lu]Lu³⁺ ranged from 0.1 to 1.2 for reactions that contained approximately 40-60 mCi of [¹⁷⁷Lu]Lu³⁺.

[¹⁷⁷Lu]Lutetium PSMA I&T solution prepared as part of the pivotal phase 3 clinical trials targeted 120 μg of PSMA I&T for every 200 mCi dose. This translates to a simple ratio of 0.6 μg:mCi, which is at the mid-point of the range examined in this study.

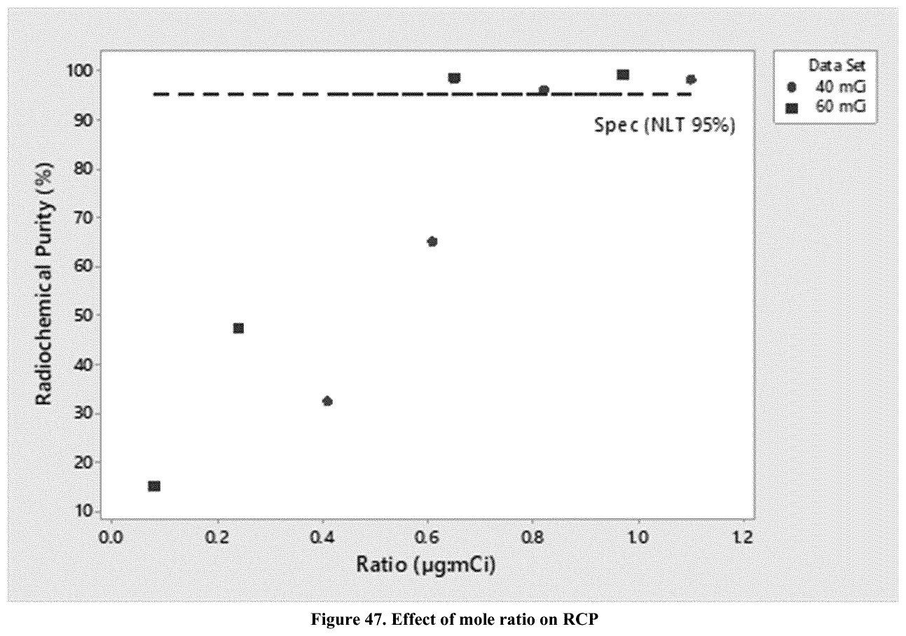

As summarized in Table 52, the results obtained for [¹⁷⁷Lu]Lu³⁺ incorporation are reflected in the radiochemical purity results (RCP), and are represented graphically in FIG. 47

TABLE 52
Data Summary Radiochemical Yield (%
RCY) as a Function of PSMA I&T Ratio

| | PSMA | | | RCP |
|---|---|---|---|---|
| | I&T:Lu- | Radiolabelling | | (%, Lu-177 |
| Experiment | 177 (μg:mCi) | Activity (mCi) | pH | PSMA I&T) |
| 1-A | 0.08 | 61.9 | 5.05 | 14.62 |
| 1-B | 0.24 | 61.9 | 5.05 | 47.03 |
| 1-C | 0.65 | 61.8 | 5.04 | 98.57 |
| 1-D | 0.97 | 61.6 | 5.03 | 99.10 |
| 2-A | 0.41 | 36.6 | 4.90 | 32.27 |
| 2-B | 0.61 | 36.6 | 4.91 | 64.70 |
| 2-C | 0.82 | 36.6 | 4.92 | 96.07 |
| 2-D | 1.10 | 36.5 | 4.90 | 98.18 |

The two data sets were created using [¹⁷⁷Lu]Lutetium supplied by two vendors. The lower the ratio of PSMA I&T to Lu-177, the lower the radiochemical yield. The results summarized in Table 52 indicate that ratios of PSMA I&T to [¹⁷⁷Lu]Lu³⁺ of 0.65 or higher resulted in the average radiochemical yield (RCY) of 97.98% with a relative standard deviation of 1.33%. For each data set, a sharp decrease in RCP was observed. For reactions performed at approximately 60 mCi, RCP decreased at a ratio of 0.24. For reactions performed at 40 mCi, the same decrease occurred at a ratio of 0.6. This has been observed in other experiments where the RCP can be lower for smaller-scale experiments. When lower ratios were examined, the radiochemical yield of the resulting product decreased to 39.7%, which is unacceptable for the desired labeling process.

As the ratio of PSMA I&T to [¹⁷⁷Lu]Lu³⁺ decreases, two (2) factors may explain the observed decrease in radiochemical yield. First, the kinetics of the radiolabeling reaction could be impacted by the more diluted reaction conditions. Secondly, due to the propensity of the DOTAGA-moiety of the PSMA I&T ligand to bind with a wide variety of metals, the lower ratio of ligand to lutetium means there is a greater susceptibility for competing metals to bind instead of [¹⁷⁷Lu]Lu³⁺, thus resulting in a lower radiochemical purity drug substance

### Conclusion

The studies described examined the impact of the ratio of PSMA I&T to [¹⁷⁷Lu]Lu³⁺ on the percent of lutetium incorporated into PSMA I&T drug substance, as measured by the radiochemical yield of the derived product. The results suggest that a PSMA I&T to [¹⁷⁷Lu]Lu³⁺ ratio of at least 0.65 provided greater than 95% [¹⁷⁷Lu]lutetium incorporation for reactions performed with at least 60 mCi of [¹⁷⁷Lu]Lu³⁺ at the time of radiolabeling. Above this threshold, the ratio of PSMA I&T to [¹⁷⁷Lu]Lu³⁺, has a negligible impact on the radiochemical yield of the drug substance.

The data presented suggest that increasing the PSMA I&T ratio relative to [¹⁷⁷Lu]Lu³⁺ could be advantageous. Future studies should examine this further. However, changes to the amount of ligand could impact the applicability of the clinical trial data relative to the commercial product.

The clinical formulation may use about 80 μg/dose (±5 μg/dose). Increasing the PSMA ratio may impact the validity of our clinical trial data. A concentration of 80 μg PSMA/dose (0.6 μg/mCi at time of administration, 200 mCi/dose) may be targeted for the process. In another embodiment, 0.6 μg/mCi±0.1 μg/mCi at time of administration may be targeted for the process. In yet another embodiment, 200 mCi/dose±25 mCi/dose at time of administration may be targeted for the process.

### FIG. 21—RCP as a Function of PSMA:Lu Ratio

### Example 17—Impact of Sodium Ascorbate Concentration on Drug Product Stability

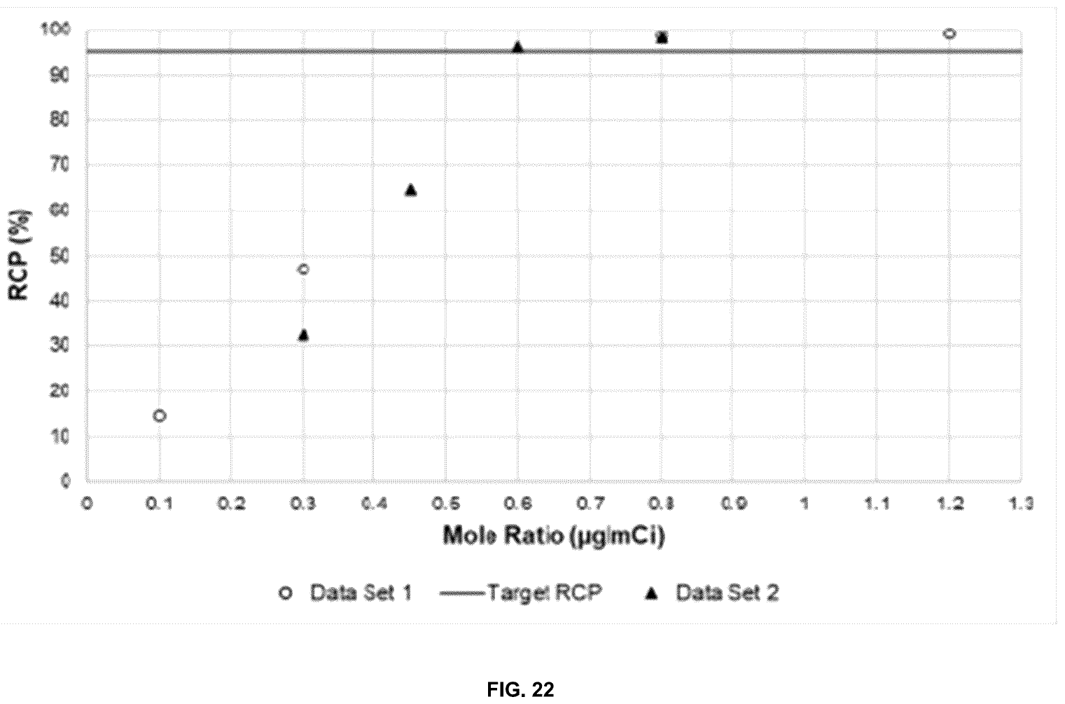

### FIG. 22—RCP as a Function of Ascorbate Content (T0+3d)

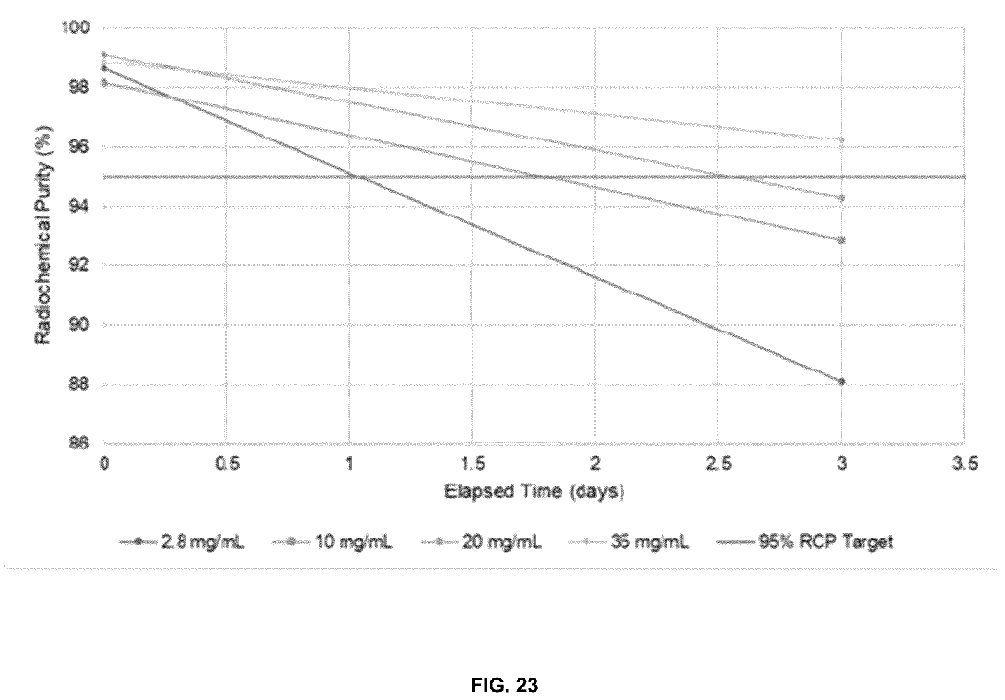

### FIG. 23—CP as a Function of Ascorbate Content (T0+7d)

### Example 18—the Impact of DTPA Content on Drug Product Stability

Purpose: The purpose of this experiment is to evaluate the effect of DTPA concentration on the stability of lutetium Lu 177 PSMA I&T injection.

Approach: To evaluate the effect of DTPA concentration on the stability of the drug product, four (4) formulations containing 0, 10, 75, and 150 ppm DTPA were prepared and changes in radiochemical purity (RCP) and presence of organic impurities were evaluated at expiry (T0+72 h) as a function of DTPA concentration.

### PROCEDURE: Radiosynthesis and Final Formulation

A detailed description of the procedure is provided in the batch record for Lot #8017-QF-2-E3 (Attachment 1). In brief, the contents of four vials containing approximately 250 mCi of [¹⁷⁷Lu]LuCl3 were transferred to four (4) individual 10 mL tubing vials, each containing 200 μL of 1 mg/mL PSMA I&T and 1 mL of Reaction Buffer (0.2 M sodium ascorbate, pH=5.25). Reaction vials were heated for 10 min at 75° C. to allow for radiolabeling of the PSMA I&T with the [¹⁷⁷Lu]Lu³⁺ to occur. Reaction vials were then cooled for 5 min before transferring contents to 30 mL molded vials and diluted with 13.1 mL dilution buffer to achieve the target values listed in Table 53. Actual measured concentrations and properties of the prepared formulations at release and T0+72 h are reported in the Results and Discussion section.

TABLE 53
Target Values for the Formulations Used in this Report.
¹RAC: Radioactivity Concentration at the end of formulation

| | Final Drug Product Formulation (Targets) | | | | | | |
|---|---|---|---|---|---|---|---|
| | | PSMA | Ethanol | DTPA | Total | RAC¹ | |
| | Sodium | I&T | (% | (mg/ | Volume | mCi/ | |
| ID | Ascorbate | Quantity | v/v) | ml) | (mL) | mL) | pH |
| QF-2-E3-A | 20 | 13.5 | 3.5 | 0 | 14.8 | 16.9 | 5.0 |
| QF-2-E3-B | | | | 0.010 | | | |
| QF-2-E3-C | | | | 0.075 | | | |
| QF-2-E3-D | | | | 0.150 | | | |

¹RAC: Radioactivity Concentration at the end of formulation

### Sample Analysis

The DTPA content, free Lu-177, PSMA I&T content, and RCP of the formulations were determined using a variety of analytical techniques described in the following sub-sections. A detailed record of the steps performed for each analysis can be found in Attachment 1 and 2.

### DTPA Content

Details regarding the chromatographic method for determining DTPA content are summarized in Table 54. A five-point standard curve of DTPA concentrations between 5 and 20 ppm was used to determine DTPA concentrations in samples. The system suitability of the instrument was confirmed by a single 15 m DTPA check standard every 10 sample injections.

TABLE 54
HPLC Method and Instrument Conditions
Instrument Parameters

| Column | Phenomenex Kinetex C18 | |
|---|---|---|
| | Column - 2.6 pm - 150 - [symbol] | |
| Flow Rate | 0.2 | mL/min |
| Mobile Phase | Mobile Phase: 7.5 mM | |
| | Dodecyltriethyl Ammonium | |
| | Phosphate, [symbol] | |
| Column Temperature | 40° | C. |
| Injection Volume | 50 | pL |
| Run Time | 80 | min |
| UV Wavelength | 260 | nm |

[symbol]

indicates data missing or illegible when filed

### Free Lu-177

The presence of free Lu-177 in the formulations was determined by thin layer chromatography (TLC). A 10 pL aliquot of each sample was diluted to 0.3 mCi/mL using a 20 mg/mL sodium ascorbate solution before spotting a 5 pL portion onto a 1.5×11.5 cm iTLC silica gel strip at 1 cm from the bottom of the strip. Strips were then placed into a development chamber containing approximately 2 mL of mobile phase (methanol:water:ammonium hydroxide, 20:20:1, v/v/v) and developed until the solvent front had migrated to a height of 9 cm. Strips were then removed from the development chamber and analyzed using a Bioscan B-AR-2000-1. Bioscan parameters are listed in Table 55.

TABLE 55
Bioscan B-AR-2000-1 Instrument Conditions
Instrument Parameters

| Max Time | 5 | min |
|---|---|---|
| Max Count | 200000 | |
| Channels | 256 | |
| Collimator Width | 10 | mm |

| | |
|---|---|
| Collimator Type | High |
| | [symbol] |
| Electronic | Normal |
| [symbol] | |

[symbol]

indicates data missing or illegible when filed

### PSMA I&T Content and Radiochemical Purity (RCP)

Both the determination of the assay for the PSMA I&T and related substances and the RCP of the formulations were determined via HPLC analysis. Details regarding the chromatographic method are summarized in Table 56. The system suitability of the instrument was established by triplicate injections of a 15 ppm PSMA I&T standard prior to sample analysis and a single 15 ppm check standard every 10 sample injections. A five-point standard curve of PSMA I&T concentrations between 0.5 and 25 ppm was used to determine the PSMA I&T concentration in the formulations.

TABLE 56
HPLC Method and Instrument Conditions
Instrument Parameters

| Column | Phenomenex Luna C18(2) Column- | | | | | |
|---|---|---|---|---|---|---|
| | 5 μm-150 × [symbol] | | | | | |
| Flow Rate | 1.0 mL/min | | | | | |
| Mobile Phase | Mobile Phase A: Water (0.1% TFA) | | | | | |
| | Mobile Phase B: Acetonitrile (0.1% TFA) | | | | | |
| Column Temperature | 35° C. | | | | | |
| Injection Volume | 100 μL for PSMA I&T quantification, | | | | | |
| | variable for RCP | | | | | |
| Run Time | 30 min | | | | | |
| UV Wavelength | 220 nm | | | | | |
| Radioactive Detector | Beckman Coulter Rad Detector | | | | | |
| Radioactivity Detector | Variable^{a} | | | | | |
| [symbol] | | | | | | |
| Gradient | Time (min) | 0 | 20 | 26 | 27 | 30 |
| | % Mobile | 85 | 75 | 75 | 85 | 85 |
| | Phase A | | | | | |
| | % Mobile | 15 | 25 | 25 | 15 | 15 |
| | Phase B | | | | | |

^{a}Adjusted based on sample radioactivity concentration to maximize use of detector's full scale [symbol]
indicates data missing or illegible when filed

### Results and Discussion

The results of the analytical testing for the four formulations prepared in this report are summarized in Table 57.

TABLE 57
Analytical results from the analysis of the formulations
prepared for this report.

| | | | | | PSMA | | |
|---|---|---|---|---|---|---|---|
| | | | | | I&T | | |
| | | | | | and | | |
| | | | | | Related | | |
| | | DTPA | | | Sub- | | |
| | | Con- | | | stances | | RAC1 |
| | Time- | tent | | | Content | RCP | (mCi/ |
| ID | point | (ppm) | Appearance | pH | (μg/mL) | (%) | mL) |
| QF-2- | T0 | 5 | Clear, Colorless, | 5.17 | 16.66 | 98.39 | 14.0 |
| E3-A | | | Particulate Free | | | | |
| | T72 | 0 | Clear, Colorless | 5.15 | 16.2 | 95.53 | ND³ |
| | | | Solution | | | | |
| QF-2- | T0 | 9 | Clear, Colorless, | 5.17 | 17.08 | 98.37 | 11.0 |
| E3-B | | | Particulate Free | | | | |
| | T72 | 0 | Clear, Colorless | 5.18 | 16.85 | 94.53 | ND³ |
| | | | Solution | | | | |
| QF-2- | T0 | 85 | Clear, Colorless, | 5.19 | 17.18 | 98.41 | 13.2 |
| E3-C | | | Particulate Free | | | | |
| | T72 | 41 | Clear, Colorless | 5.21 | 16.78 | 95.26 | ND³ |
| | | | Solution | | | | |
| QF-2- | T0 | 165 | Clear, Colorless, | 5.20 | 14.23 | 98.17 | 12.5 |
| E3-D | | | Particulate Free | | | | |
| | T72 | 104 | Clear, Colorless | 5.25 | 13.69 | 94.69 | ND³ |
| | | | Solution | | | | |

1Radioactivity Concentration at the end of formulation;
²Appearance results are equivalent between batches but wording is dependent on who performs the testing and where the results were recorded;
³RAC was not measured at stability timepoints since it is not considered a stability-indicating attribute;
ND: Not Determined

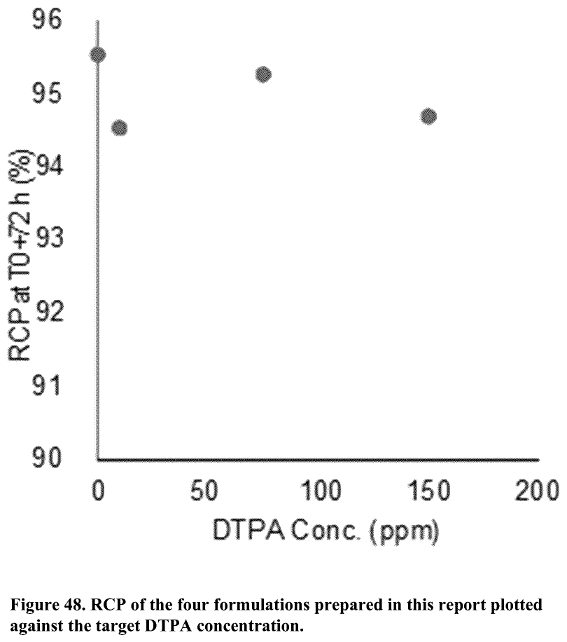

To determine the effect of DTPA on the stability of the [¹⁷⁷Lu]Lu-PSMA I&T, the RCP of four (4) formulations containing different concentrations of DTPA was measured 72 h after preparation (FIG. 48). The RCP at expiry did not show an observable trend relative to increasing DTPA concentration. Statistically, the RCP values are similar for each DTPA concentration level with a standard deviation of only 0.5% between the four values. The lack of trend and variability in the RCP results as a function of DTPA concentration for the four formulations at 72 h indicate that the RCP does not trend with the DTPA concentration.

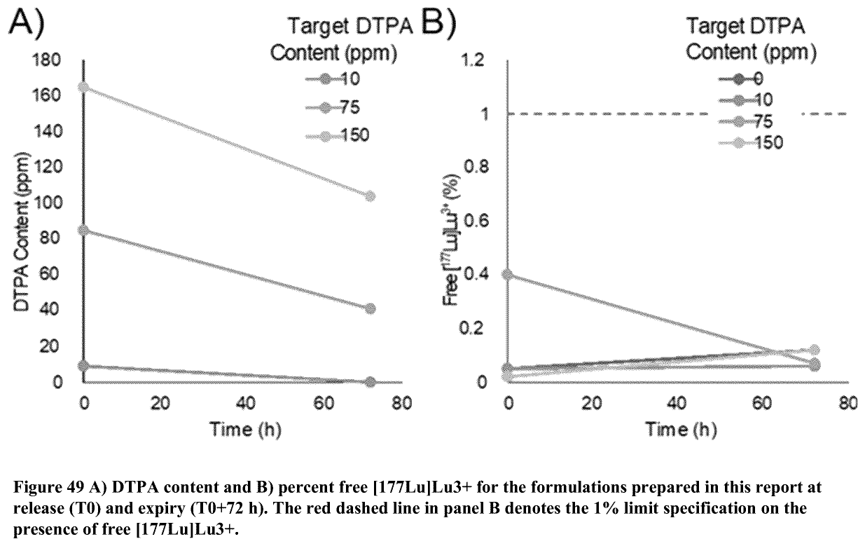

In addition to the radiolysis of PSMA I&T and decrease in RCP, the radioactive decay of the lutetium-177 can also degrade the DTPA leaving free [¹⁷⁷Lu]Lu³⁺ without a metal scavenger. During the storage of the formulations, the DTPA concentration decreased for the three formulations that contained DTPA (FIG. 49A). This decrease in the measured DTPA concentration could be due to the inability of the HPLC method to measure DTPA chelated to free [¹⁷⁷Lu]Lu³⁺ or radiolysis of the DTPA. As degradation of the DTPA could lead to free [¹⁷⁷Lu]Lu³⁺ not having a metal scavenger to bind to, the presence of free [¹⁷⁷Lu]Lu³⁺ was analyzed (FIG. 49B). For all samples evaluated, the free [¹⁷⁷Lu]Lu³⁺ content remained stable during the testing period and therefore the extent of the change observed is not impactful to the overall quantity of free [¹⁷⁷Lu]Lu³⁺ in the drug product.

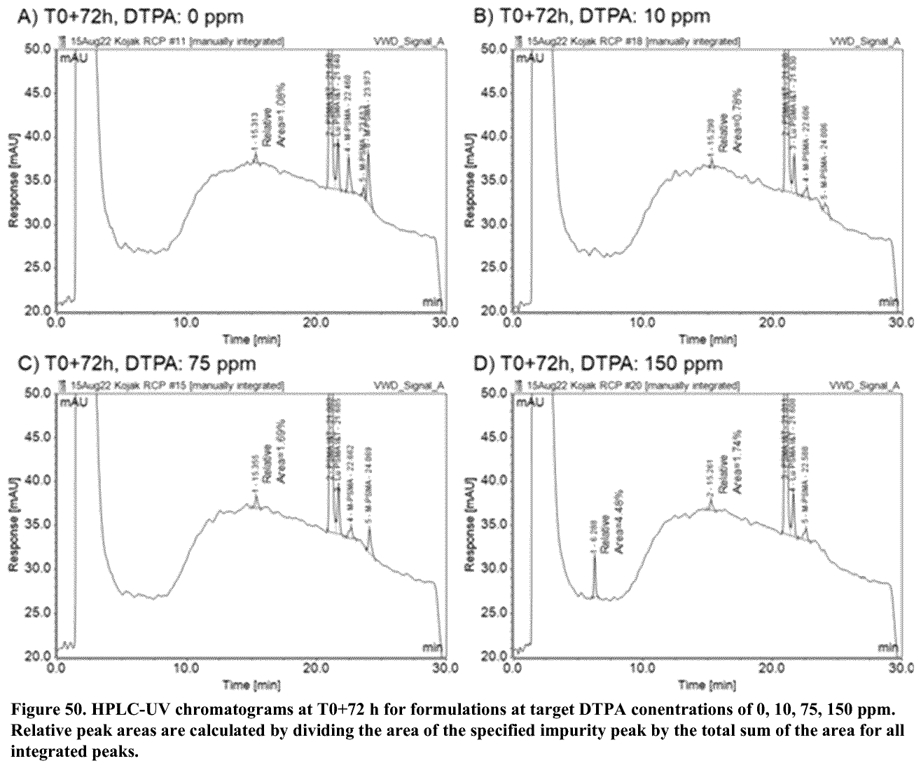

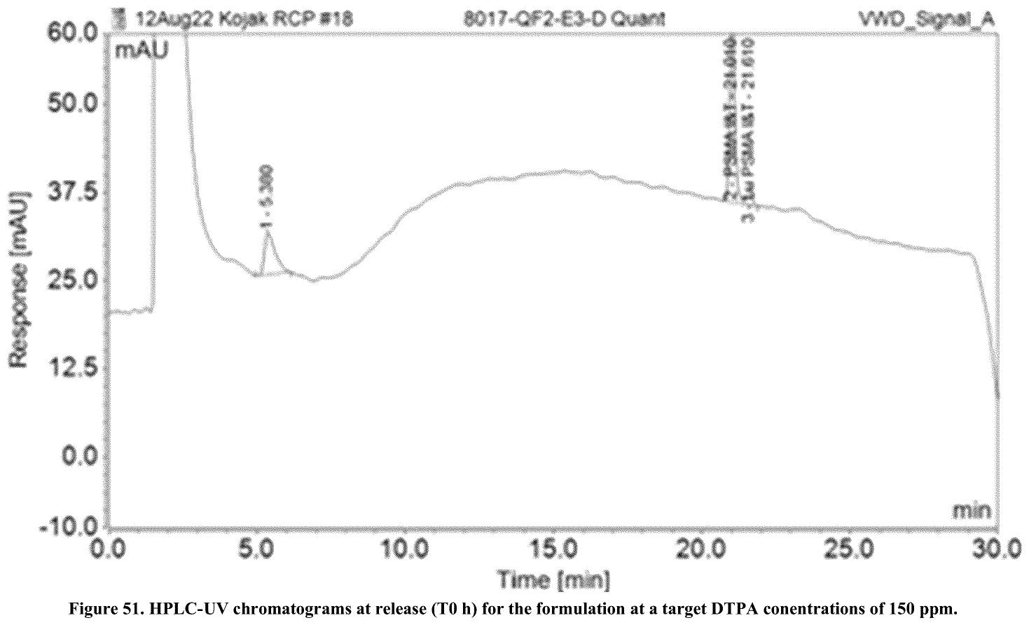

Additionally, the degradation of DTPA and impurities in the DTPA raw material could lead to the presence of organic impurities found in the HPLC-UV chromatograms for each formulation at 72 h after preparation (FIG. 50). These peaks typically appear in the chromatograms prior to the elution of the [¹⁷⁷Lu]Lu-PSMA I&T peak (RRT<1). For all of the formulations evaluated at T0+72 h, there was an organic impurity peak present at a relative retention time of 0.73. Since this peak was present in all four (4) chromatograms including the formulation containing no DTPA, it is unlikely that this organic impurity peak is related to the DTPA content. There was also a large peak present in the chromatograms at RRT=0.30 for the formulation containing 150 ppm DTPA that was not present in the other chromatograms (FIG. 50D). As this peak was also present in the HPLC-UV chromatogram for the 150 ppm DTPA formulation at release (FIG. 51), it is expected that this impurity is unrelated to the DTPA content and instead an organic impurity introduced during the preparation of the 150 ppm DTPA formulation. Additionally, the size of each of the organic impurities peaks is small relative to the peak areas of the PSMA I&T related peaks and therefore not expected to be at a high concentration in the formulation (Table 58).

TABLE 58
Relative peak areas for the two organic impurity peaks for the HPLC-UV
chromatograms for each formulation at T0 + 72 h. Relative peak
areas are calculated by dividing the area of the specified impurity
peak by the total sum of the area for all integrated peaks.

| DT | Relative | Relative |
|---|---|---|
| 0 | ND | 1.08 |
| 10 | ND | 0.78 |
| 75 | ND | 1.69 |
| 150 | 4.48 | 1.74 |

### Conclusion

During this study, it was determined that the stability of the drug product as measured by the RCP and presence of organic impurities was independent of DTPA content at all tested concentrations (i.e., 0 to 150 ppm). Future formulation and development work will contain 100 ppm (i.e., 0.1 mg/mL) DTPA to match Pluvicto (lutetium Lu 177 vipivotide tetraxetan), which is a [¹⁷⁷Lu]Lu-PSMA-617 based drug product currently on the market.

DTPA is a scavenger of any un-bound Lu-177 within the drug product. It is included in the formulation for patient safety, to prevent unnecessary uptake in the bones. Development studies show no significant impact to the drug product stability based on the content included in the formulation. Therefore, the target quantity of DTPA in the final formulation of the present invention may be about 0.1 mg/mL±0.05 mg/mL.

### Example 20—the Impact of RAC on Drug Product Stability

There is a clear dependency of the radiochemical purity of the drug product as a function of the radioactivity concentration at time of formulation. In one aspect, a concentration of 11.9 mCi/mL represents the limit observed in this study to meet specifications (i.e., greater than or equal to 95%) after an elapsed time of 72 hours. In another aspect, a concentration of 11.9 mCi/mL to about 18.2 mCi/mL at time of synthesis is required to have a drug product stability (e.g., an RAC) of greater than or equal to 95% at 72 hours. In another aspect, a concentration of at least 18.2 mCi/mL (±15%, ±10%, or ±5%) at time of synthesis is required to have a drug product stability (e.g., an RAC) of greater than or equal to 95% at 72 hours. In yet another aspect, a concentration of at least 17.0 mCi/mL (±15%, ±10%, or ±5%) at time of synthesis is required to have a drug product stability (e.g., an RAC) of greater than or equal to 95% at 72 hours.

### Example 21—RadioHPLC Chromatogram

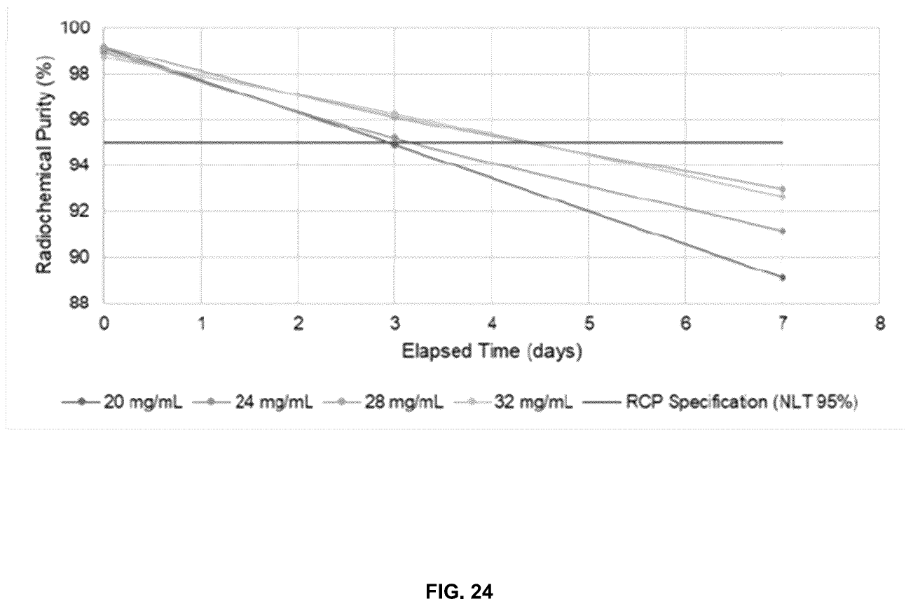

### FIG. 24—RadioHPLC Chromatogram of ¹⁷⁷Lu-PSMA I&T Composition at Formation/Production (i.e., T0), Wherein PSMA Content is about 80 μg

### Example 22—the Impact of Terminal Sterilization

Terminal sterilization of drug product was evaluated by sterilizing 2 samples via an autoclave using the following cycle: 121° C. for 15 min hold; 15.3 psi. The drug product was sampled and tested before and after autoclaving on the day of manufacture. The target for radiochemical purity at release is greater than 95%. Based on the data presented below, the drug product lutetium ¹⁷⁷Lu-PSMA-I&T injection is not compatible with terminal sterilization. The compositions described herein are therefore contemplated without terminal sterilization (i.e., in the absence of terminal sterilization).

TABLE 59

| Sample ID | RCP (%) |
|---|---|
| Pre-Sterilization | 99.33 |
| Post Sterilization | 40.10 |

### Example 23—RCP as a Function of Reaction Buffer Age

In some embodiments, the drug product lutetium [¹⁷⁷Lu]Lu-PSMA-I&T comprises an RCP of 95% or greater for at least 1 week, at least 2 weeks, at least 3 weeks, or at least 3.5 weeks. Provided herein are drug product lutetium [¹⁷⁷Lu]Lu-PSMA-I&T injection compositions with radiochemical yields of 95% or greater for solutions up to 3 weeks old. In other embodiments, it may be recommended that solutions are prepared as close to use as possible to ensure the highest radiochemical yield at time of synthesis.

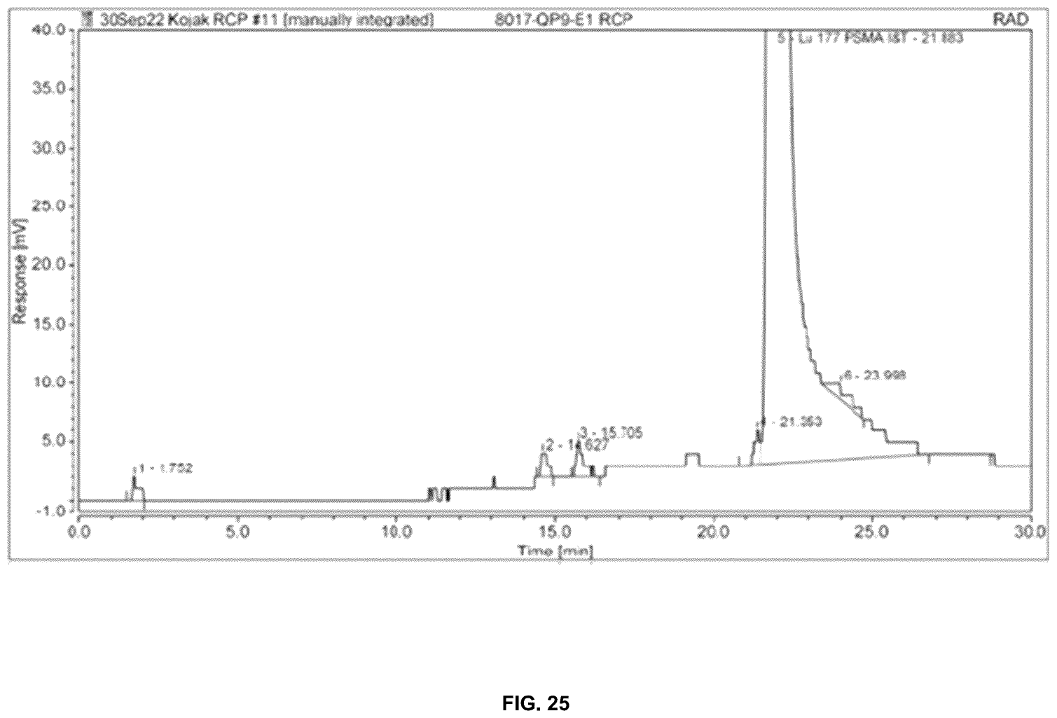

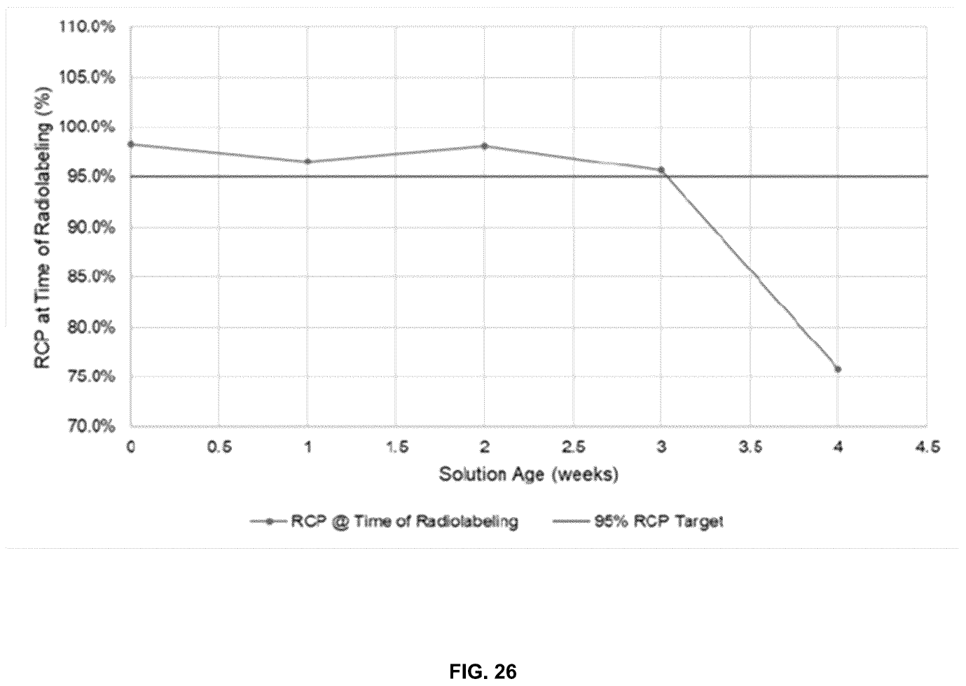

### FIG. 25—RCP as a Function of Reaction Buffer Age

### Example 24: Imaging Center Training and Set Up

### Technical Operations Manual Spectict & Gamma Counter Data

Always ensure consistent scan parameters are used across time points. The same scanner must be used for all assessments within and across all patients, unless otherwise approved by Invicro or the study sponsor.

Acquire images according to the trial-specific process standards within and according to the protocol's Schedule of Activities. Image acquisition and documentation should not deviate from the clinical trial protocol; however, the T0M may provide expanded information and processes_

All SPECUCT scans and gamma counter data, and corresponding documents must be submitted to Invicro preferably within 24 hours, but no more than 3 business days following the final imaging and blood collection time point for each patient per cycle. All gamma counter data must be submitted to Invicro within 24 hours of the final blood collection timepoint for the cycle per patient.

Respond promptly to queries generated by Invicro (preferably within 24 hours but no more than 5 days).

Confirm that all technologists or other personnel who will be performing scans for this protocol are property trained on the study-specific acquisition and reconstruction parameters, as well as data submission and query handling procedure&

Regulatory compliance: All site personnel must comply with the International Council for Harmonization Efi(R2) guidelines on Good Clinical Practice (GCP) when documenting acquisition, archiving images, and submitting image data to Invicro.

Confidentiality: Follow regional privacy practices to de-identify (ie, redact) all patient information (name, medical record number, etc) before submitting image data to Invicro. Submitters should pay particular attention to data that are likely to include protected health information such as DICOM headers, CT dose reports, image overlays, and screenshots.

### Schedule of Activities

Your imaging center may scan and collect samples from the first patient following completion of all training and approval requirements. After the scans from the first patient are approved, scanning and sample collection for the remainder of the patients may begin, according to the protocol Schedule of Activities, as summarized in Table 60.

TABLE 60
PK Sub-study Schedule of Activities

| Time Post- | Study Drug | Blood Sampling | SP ECT:GT |
|---|---|---|---|
| Injection′ | Administration ° | for PK | Imaging |
| Pre-injection | | X | |
| Time = 0 | X | | |
| 1 hour (±15 min) | | X | |
| 4 hours (±30 min) | — | X | X |
| Tam Post- | Study Drug | Blood Sampling | SP ECT.:CT |
| Injection′ | Administration″ | for PK | Imaging |
| 24 hours (±2 hours) | | X | X |
| 43 hours (±2 hours) | | X | X |
| 7 ± 1 days | | X | X |

a Tines shown are relative to inLu-PSMA-I&T infusion. When assessments occur in the same window, the order of the assessments is Blood Sampling then SPECTICT Imaging. The actual time and date of each assessment will be recorded.
b During Treatment Cycles 1-4, patients will receive a single infusion of InLu-PSMA-I&T. Twenty percent of patents (first 6 patients) to be enrolled in the sub-study, will undergo PK sampling and SPECTICT imaging after the first and third ¹⁷⁷Lu-PSMA I&T infusion (Week 0 and Week 12). Data generated from those 6 patents at 4 selected time points at cycle 1 (4 hours, 1, 2, 7 + 1 − 1 days). and at cycle 3 (24 and/or 48 hours per investigator's choice), will be analyzed to determine if differences in drug kinetics or uptake are observed between week 0 and Week 12. If only one SPECTICT timepoint is chosen by the investgatorfor cycle 3, the 24 hour tmepoint is preferred. If no significant differences are observed between cycle 1 and cycle 3 for the set of the 6 patents, PK sampling and SPECTICT triaging will be pertained after the first cycle only (Week 0) for the remaining 24 patients.

### Image Data Submission Timelines

All SPEC-MCI scans and gamma counter data, and corresponding documents must be submitted to Invicro preferably within 24 hours, but no more than 3 business days following the final imaging and blood collection time point for each patient per cycle.

Imaging center setup: Participating imaging centers will be required to complete Invicro's setup procedures prior to conducting study patient imaging. The purpose of these procedures is to ensure each imaging center participating in the study meets the high level of standard required by Invicro and the study sponsor, provide technologists and supporting staff with training on study imaging requirements, assess the performance of the imaging equipment to be used for the study, and ensure the protocols to be followed for patient imaging are property established.

The following procedures are included as part of setup.

### Technical Assessment Questionnaire

The initial step in the setup process is the centers completion of a technical

assessment questionnaire. This form provides Invicro with contact information for key individuals, address information, specific scanner and computer system capabilities, and other specifications necessary for satisfactory completion of the study. Invicro will provide a brief overview of the setup process to the site and imaging center at the time of sending the questionnaire.

The completed questionnaire is reviewed by Invicro for initial assessment of the imaging centers technical capabilities. If the center meets the technical standards required to perform as a participating center, Invicro will contact the center informing them of the next steps necessary for completion of the qualification process for the study described in the following paragraphs.

In certain cases where Invicro has recently worked with, or is currently working with, an imaging center on another study and has already collected a questionnaire, the requirement for the center to complete a new questionnaire may be waived.

### Technical Site Training

The imaging center training for this trial will be conducted via teleconference.

The teleconference training will be among the imaging center staff, the clinical site personnel, and an Invicro setup specialist. During this meeting the overall objectives and rationale for the imaging component of the clinical trial are reviewed. Individual responsibilities for obtaining the imaging measures and a walk-through of the communication flow, logistics, and potential problems (eg, scanner availability, etc,) at the site are discussed and questions addressed—

Imaging centers may be required to acquire and transfer phantom data to Invicro, The phantom data will be analyzed and used to assess scanner performance. Details regarding phantom preparation, acquisition, and reconstruction, will be provided in a separate document_ Please note, the acquisition and transfer of phantom data may be waived if Invicro has recent phantom data on record from the participating imaging center.

In addition, imaging centers will be required to build the study-specific protocol in their scanner and may be asked to submit screenshots of the relevant parameters to Invicro for review and approval.

Please now, at least one primary staff member (technologist, physicist, or physician) responsible for the conduct of study imaging IMISE he Trained by Invicro. It is the responsibility of the imaging center to ensure all additional personnel are trained peer-to-peer by the primary staff member initially trained by Invicro and are supplied with a copy of this T0M, as well as all other study materials. All additional training activities must be documented, with a copy of this training documentation provided ro the referring clinical sires). Please contact Invicro for assistance whh additional training or questions.

Approval to scan First Patient: Following completion of the technical setup procedures, an ‘Approval to Scan’ notification and a detailed technical site setup report summarizing the dose, acquisition and reconstruction parameters, image archival, and the approved method of image transfer will be sent to the imaging center, with a copy to the clinical site and sponsor designees. The approval notification and technical report should be archived.

Please Now: This ‘Approval to Scan’ notification indicates that the imaging center is now ready ro scan their tirsr study patient only.

Approval to scan all subsequent patients: After the first study patient has been imaged, the imaging center will be instructed to transfer the images to Invicro within 24 hours of acquisition for OC review. Once the patient images have passed QC, the imaging center and clinical coordinator will be notified via email that they have been approved to continue imaging additional patients. Sites will be notified within approximately 5 business days of receipt of the first patient data {barring no queries). Should the first patient scan not pass OC review, Invicro will communicate with the imaging and clinical personnel regarding how to resolve the issue prior to moving forward with additional patient imaging

All references cited herein are hereby incorporated by reference. The foregoing is offered primarily for purposes of illustration. It will be readily apparent to those skilled in the art that further drugs can be included, and that the components, additives, proportions, methods of formulation, methods of use, and other parameters described herein can be modified further or substituted in various ways without departing from the spirit and scope of the invention. Please Now: The imaging center MUS1 wait for the ‘Approval to Scan Subsequent Patient’ notcation in the study.

Please Now: The clinical coordinator should NOT schedule a second patient for imaging until lovicro has reviewed the first patient and it has passed CC.

Technical Binder: The imaging center will receive a link to an electronic technical binder containing all essential imaging study documents including, but not limited to, a copy of this T0M, the training presentation, radiopharmaceutical information, and key Invicro contact information. These documents are to be printed and made available to any staff which will need access to this information. Additionally, Invicro recommends each imaging center file all study related communications (eg, emails or faxes).

Please Now: Study related documents may be updated throughout the Trial and will be distributed to your imaging center

Scan identification: It is very important that the imaging center uses a standard file naming convention so that all scans can be easily identified by Invicro. The patient naming convention will be provided by the clinical site personnel in accordance with the study specific requirements_

The site number is assigned to the clinical sites in the trial, not the imaging centers. Therefore, imaging centers that are receiving referrals from several clinical sites will need to use the clinical site number associated with the site the patient is being referred from, on the data submission form and in the naming of the scans when data is transmitted to Invicro. The clinical study site numbers in this trial are comprised of 2 digits.

In this study, patients will be assigned a 5-digit Patient ID. This Patient ID is composed of the 2-digit site number (represented as XX), and 3 digits unique to the patient (represented by YYY). The naming conventions are summarized in Table 61.

| | Scan File Naming | Numbers for Inclusion on iPACS |
|---|---|---|
| Visit | Convention | Submission Form |
| All Visits | 5-digit. screening ID | 5-digit screening ID |
| | (XX-YYY) | (XX-YYY) |

Please note: the 5-digit Patient ID number should he populated in both the Patient Name and ID Gelds with the Date of Birth arbitrarily populated with 01, 1 an4970 when setting up an acquisition on the scanner or during the de-identilication of the scan file

### ⁷⁷Lu-PSMA-I&T Administration

The study drug, InLu-PSMA-I&T, is a novel radiotherapeutic that will be characterized for efficacy, and dosimetry for the treatment of mCRPC_ Patients participating in the PK and Radiation Dosimetry Sub-study will have 4 injections of inLu-PSMA-I&T, followed by SPECTiCT and blood collection at certain timepoints depending on the cycle as detailed in Table 1.

Important Reminders for all sties:

infil-PSMA-iai is a radioactive drug and should be handled with appropriate safety measures to minimize radiation exposure_ Radiophamiaceuticals should be received, used, and 1 administered only by authorized persons in designated clinical settings, and handled with strict adherence to local regulation&

The responsible clinical physician for the study should confirm the patients suitability for undergoing the imaging study in accord with the inclus ioniexclus ion criteria in clinical protocol.

Refer to Study Procedure Manual and the Myesiigarors Brochure for lwa-PSMA-I&T for nar derails on dose ordering, receipt, handling, administration, and desirucubnidecay.

### Mi_u-PSMA-18T Dose Ordering

IThLu-PSMA-I&T doses will be produced and supplied from a central manufacturing site.

The order for ¹⁷⁷LN-PSM4-I&T will be placed by the imaging center at least 2 weeks prior to the week of planned dosing. Imaging center staff must be aware of production schedules, approximate time of dose delivery and product expiration time to facilitate scheduling with the clinical staff.

### 'Ntu-PSMA-1.ST Dosing Information

### Dose Calibrator Calibration

Before scheduling patients to be injected with ¹⁷⁷Lu-PSW-I&T, your imaging center must confirm that the dose calibrator to be used has been accurately calibrated to measure Lu-177_ The close calibrator dial setting established for this isotope as part of site setup must be used for all patient injections. Invicro will work with imaging center personnel to review the calibration procedure.

### InLu-PSI/¼ A-I&T Dose Preparation

InLu-PSMA-I&T is a ready-to-use, sterile formulation in a single-dose vial. The glass vial containing the radiopharmaceutical is kept in a lead shielded container until use, and the shield label will state the expiration date and time for each vial shipped. ¹⁷⁷Lu-PSMA-1&T injection solution is administered as supplied_ The radioactivity in the vial should be measured in a calibrated radiation dose calibrator prior to, and after administration to the patient. The administered dose will be automatically calculated.

### Patient Preparation

Prior to ¹⁷Thu-PSIgIA-I&T administration, the following will be performed: Cooling of the patients' salivary glands may be performed by placing ice packs over the parotid and submandibular glands for 30 minutes prior to and up to 4 hours after the injection of ¹⁷⁷Lu-PSMA-I&T to reduce the risk of salivary gland radiation injuries. The patient should be encouraged to void frequently. The patient should be encouraged to drink 2 liters of liquid daily for 2 days following ¹⁷Lu-PSMA-I&T administration. An intravenous (IV) line will be inserted for administration of ^{1P}Lu-PSMA-18tT, according to site standard procedures.

### ¹⁷⁷Lu-PSMA-I&T Dose Administration

Patients will receive a single IV dose of 200 mCi (7.4 GBq)±10% of ¹⁷Lu-PSMA-I&T as an infusion over a minimum of 10 minutes, using the sites standard radioligand therapy administration procedures_

IThLu-PShIA-I&T will be administered every 6 weeks for 4 cycles or until radiographic progression of disease. Based on evaluation of dose-limiting toxicities for the patient, the dosing cycle for ¹⁷Lu-PSMA-I&T may be extended. Additionally, the ¹ThLu-PSMA-I&T dose should be held and/or reduced to 160 mCi (5_9 GBq)±10% if dose-limiting toxicity if noted_

See the study protocol for full derails on the evaluation of dose-limiting roxichies and the impact on ⁱⁿLu-PSAM-f&T dose.

### Imaging Protocol

### Reference Standard

Prepare a reference standard by injecting approximately 100 uCi of ¹⁷Lu-PSMA-I&T from the residual dose into a 100 nil saline bag_ The reference standard will be acquired immediately following each patient SPECTICT scan as a separate scan using the same acquisition and reconstruction parameters as are used for the patient scan.

Please note: The same reference standard must be used for elf SPECT/CT scans acquired for a given patient Cycle. The reference standard must therefore be appropriately labeled with the P2fiela ID prior to storage. The reference standard should be held for decay as per local regulations.

### SPECTICT Imaging

The patient should be informed about the total acquisition time and positioned for maximum comfort, lying supine on the scanner table with their head in a head holder.

SPECTICT anatomical coverage should start with the salivary glands fully in the field of view (FOY) and extend through the pelvis. Coverage of the kidneys must be prioritized in situations where two or three FOVs are unable to be obtained due to patient comfort.

In general, raw projection data will be acquired into a 128×128 matrix with MEGP collimation using a step and shoot acquisition mode, acquiring 60 projections per detector (180 degrees rotation per detector), 20 seconds per projection, with an acquisition zoom of 1.0, and a 20% (±10%) energy window centered over 208 key.

To optimize image quality, detector heads should be as close as possible, and remain consistent for each time point of a given patient. A low-dose CT scan will be performed prior to beginning the SPECT scan for attenuation correction. Institutional standard parameters may be used for the low-dose CT. Institutional standard reconstruction parameters may be used if approved for use by Invicro. Scatter correction is required to be applied using a 20% (±10%) scatter window centered over 170 key.

The start time of each SPECTICT scan must be recorded for later entry into the iPACS submission form when submitting to Invicro.

Please note: A reference standard of known acriviry of Lu-177 must be acquired immediately following each patient SPECT/CT scan as a separate scan using Me same acquisition (only IFC) V required) and reconsuircdon parameters as are used for the patient scan.

Please rote: trivia° will determine and approve the exact parameters to be used for this study as pan of the setup procedures prior ro patient imaginq.

Please note: frivicro will collect both the raw projection and conrecred reconstructions for each rime point.

### Pharmacokinetic (PK) Sample for Radioactivity Measurement

PK plasma samples will be collected according to the schedule displayed in Table 60 and counted for radioactivity. For each sample, collect sufficient sample volume for triplicate aliquots to meet the measurement requirements of your institution's gamma counter and perform measurements according to standard procedure& Blood will be processed and plasma will be measured on the gamma counter, as described in the following subsections.

### Blood Sample Preparation

All blood samples drawn must be aliquoted 3 times, labeled appropriately, and stored per standard procedure for later measurement of plasma in the qualified gamma counter. All samples will be counted as a group for each patient for each Cycle, no later than 24 hours following the final sample collection.

Each aliquot of plasma must be 0.5 mL in volume for gamma counting. Sufficient volume at each CitIM point mast be collected w achieve 0.5 mL for each of the 3 required aliquots per sample type in addition to other required PK analyses.

For each blood sample time point, label the tubes to be used for gamma counter measurements with the appropriate subject ID, sample time point, sample type, and aliquot number.

For each aliquoted sample, measure and record the weight of the labeled empty tube and again measure and record the weight of the tube plus the aliquoted sample. Tube weights should be recorded in grams up to 3 decimal places, if possible, in the Gamma Counter Workbook (see Section 73). Weight measurements must be performed using a laboratory grade scale approved for the weight ranges expected for this study_

### Plasma Preparation for Gamma Counting

The following procedure is intended for plasma gamma counting. Please note, Invicro does not guarantee that plasma obtained with this procedure will be suitable for any other assay. Please refer to the instructions provided within the Technical eBinder (Section 3) from the manufacturer for blood draw and Removal of BD Hemogardw

Collect 5 ml of blood in a BD PPT system (Plasma Protein Preservation Tube-draw volume of 5 ml).

After collection of whole blood in the BD PPTTM Tube, immediately and gently invert the BD PPT′″ Tube 8-10 times. After mixing, the whole blood specimen may be stored up to six (6) hours at room temperature until centrifugation—

Centrifuge at 18-25° C. at 1100 g for 10 min. The resulting supernatant is designated plasma.

Following centrifugation, immediately aliquot the liquid component (plasma):

To obtain an undiluted plasma sample, remove the BD Hemogard nu Closure (See Instructions for Removal of BD Hemogard™ Closure Section) and aliquot 0.5 ml of plasma into the tubes used for gamma counting using a transfer pipette. NOTE: When using a transfer pipette be sure NOT to disturb the gel bather with the tip of the pipette.

Repeat 2 times to get 3 tubes containing 0.5 ml of plasma.

Note, for each aliquoted sample, measure and record the weight of the labeled empty tube and again measure and record the weight of the tube plus the aliquoted sample. Tube weights should be recorded in grams up to 3 decimal places, if possible, in the Gamma Counter Workbook (see Section 73). Weight measurements must be performed using a laboratory grade scale approved for the weight ranges expected for this study.

The aliquots can be stored ar room iemperarure until gamma counted. All samples will be counted as a group for each pagenr for each Cycle, no Parer Than 24 tours following the final sample collection.

Gamma Counter Measurements: All patient samples must be counted for 60 seconds, with a counting energy window of 102-229 keV.

ecay Correction and Background Subtraction functions should be turned Off.

As noted in the Section 6.1.2 above, the 3, 0.5 mL aliquots drawn from the reference standard during initial preparation must also be counted with the samples. In addition, 3 empty (blank) tubes must be placed in the gamma counter when the samples are measured to account for background. Lastly, a long-lived gamma counter reference source (ie, Cs-137) must also be included.

The completed Gamma Counter Workbook, and all gamma counter output files (PDFs,.td., etc.) must be submitted to Invicro at the time of each data transfer by selecting the Gamma Counter Submission Form (see Appendix 2). The Workbook must be completed electronically and checked for accuracy prior to transferring to invicra

Please note: When measuring multiple samples in one single run, ro avoid cross calk between sample types, please place and count the above-mentioned samples (blanks, reference standard samples, and long-lived reference source) in separare racks, it is imperative that all samples be properly positioned as recorded in the Gamma Counter Workbook provided and counted as a group for each parkin for each Cycle, no later than 24 hours fallowing the final sample collection.

Please note: Variarions from the above procedures will be discussed and documented as parr of tnvicro's imaging center servo.

Gamma Counter Data Documentation: The following file types will be required to be transferred to Invicro with each data submission: gamma counter output file(s) (if available) and the Gamma Counter Workbook.

Gamma counter output file(s) The output file is the direct raw output from the gamma counter containing the counts-per-minute (CPM) values for each sample and can be submitted in the site's standard format (eg, .PDF, .txt., etc.).

Please notify invicro if your gamma counter does not provide an output tile.

Gamma Counter Workbook—The Gamma Counter Workbook is an electronic tabular format file provided to each site by Invicro for standardized data submission. Template Workbooks to be completed for each patient are located within the Technical Binder (see Section 3) provided by Invicro with your Approval notification (see Section 23). The Gamma Counter Workbook must be completed electronically by each site and allows for interpretation of the output file(s) and captures the sample counting results.

The Gamma Counter Workbook records the sample type (blood, blank, long-lived reference source, etc.), the sample aliquot number {1, 2, or 3), the weight (in grams) of each empty tube and the weight of the tube plus the sample, the time each sample was collected (24 hr clock), the time each sample was counted (24 hr clock), the total counts and the CPM result.

Following data entry into the Workbook, review the Verify inputs tab. The Verify Inputs tab is meant to identify any data entry discrepancies or sample outliers. The tube weight measurements and CPIA values will automatically populate on the Verify Inputs tab with the values recorded on the Key Fife Mb.

Please now: Values higher than 10 (70 in the Coefficient of Variation column of the Verity Inputs rah should be double-checked to ensure the data enrry was performed con-ectiy. Outlier samples should be recounted ro verify the accuracy of the measurement. To avoid queries, please document the reason for any identified ° when's) and for confirm the data has been verified as accurate.

All gamma counter output files and Gamma Counter Workbooks MUSE be archived following submission ro Mulct®.

Patient Safety and Adverse Events: Each patient needs to be monitored for adverse events (rkEs) while they are at the imaging center. An AE is defined as any unfavorable and unintended sign including an abnormal laboratory finding, symptom or disease associated with the use of a medical treatment or procedure, regardless of whether it is considered related to the medical treatment or procedure, that occurs during the course of the study.

The clinical site Principal Investigator (PI) is responsible for reporting AEs to the study sponsor and should follow AE reporting procedures described in the study protocol. Although not anticipated to happen frequently, AEs may occur during the patient's visit to the imaging center, and therefore, it is very important for the imaging centers to contact their respective clinical study site personnel immediately (ie, PI or study coordinator) if they become aware of any AEs during the imaging procedures. The clinical study coordinator should be contacted by phone and the time of that call documented along with specific information related to the adverse event.

Please note: Archive the original document completed for the adverse event hi the Technical BWder and send a copy to the clinical coordinator.

### Image Data Archiving and Submission:

Data Submission Checklist: All imaging metadata and data files should be sent through iPACS. Modality specific electronic submission forms are required to be completed within iPACS for each data submission. Please reference Section 1.3.2 of this document, and the clinical protocol for details on the Schedule of Activities.
Projection and Reconstructed SPECT image data
Gamma Counter Workbook and all associated gamma counter outputs
Completed information contained in the iPACS submission form

Prior to submission, scans should be assessed for completeness, artifact and/or patient motion and any relevant infomnation documented accordingly in the iPACS submission form.

Quality control and calibrations: QC measures are to be performed according to the center and the camera manufacturers standard procedures as implemented in the center's DC program for the SPECT/CT camera and close calibrator.

Scanner Quarry Assurance: The SPECT camera should have and maintain up-to-date flood uniformity corrections (intrinsic and extrinsic) and center of rotation (CDR) corrections. Invicro recommends that calibrations are to be completed on a schedule based on manufacturer recommendations and requirements. In addition, a daily Co^{s} flood scan should be done at the beginning of the day the scanning is to be completed. The ⁵⁷Co flood scan should be visually inspected for abnormalities. If there is a possibility that the abnormality could impact the quality of the SPECT scan the study patients visit should be rescheduled. Verification that the daily CC scan has been performed should be documented on the iPACS submission form.

Software upgrades, changes in hardware and any other manipulations or changes to the imaging camera should be recorded on the iPACS submission form.

Dose Calibrator: The dose calibrator and gamma counter approved for study use must be calibrated in accordance with nationally recognized standards and/or manufacturers recommendation, at the time of install, after any mai ntenan ceirepair procedure that could affect performance and routinely throughout the course of the study_ QC checks must be performed in accordance with nationally recognized standards and for manufacturers recommendation. A record of your CC schedule should be retained. All routine CC measures performed are to be documented on Invicro's technical assessment questionnaire as part of setup.

Phantom Data: Invicro may request a phantom scan to be acquired on the SPECT/CT scanner intended for use in the study as part of the initial setup and qualification procedures or at any time throughout the course of a study. Phantoms may be requested if imaging centers are new to Invicro, an imaging center prefers to use a scanner which Invicro has not previously qualified, or the phantom data previously collected by Invicro does not meet the requirements of the study. A phantom acquisition may also be requested should a qualified SPECTICT scanner undergo significant maintenance during the study, or if scanner performance issues are noticed during QC review of study patient data.

In addition, as calibration factors will be derived from the phantom performed as part of setup, periodic phantoms may be requested to assess the stability of the SPECTICT scanners performance

Scanner replacements and upgrades: If your center will be replacing or upgrading your SPECT scanner or acquisition software, it is critical that you inform Invicro prior to the replacement or upgrade occurring, so that Invicro can take the necessary steps to ensure the continuity of the imaging outcome measures in this longitudinal research study. Invicro may need to revisit the imaging center to acquire another phantom.

Numerous examples are provided herein to enhance the understanding of the present disclosure. A specific set of statements are provided as follows.

Statement 1: A composition comprising 177Lu-PSMA I&T; wherein the molar ratio of the PSMA I&T to 177Lu is from 4.4:1.0 to 7.6:1.0, and the composition is suitable for administration to a human patient in need thereof for at least 72 hours after formulation.

Statement 2: The composition of statement 1, wherein the composition has a radiochemical purity (RCP) of 95% or greater at administration, 97% or greater at administration, or 97.5% or greater at administration.

Statement 3: A composition comprising 177Lu-PSMA I&T; wherein the molar ratio of the PSMA I&T to 177Lu is from 4.4:1.0 to 7.6:1.0, and the composition is suitable for administration to a human patient in need thereof for at least 120 hours after formulation.

Statement 4: The composition of statement 3, wherein the composition has a radiochemical purity (RCP) of 95% or greater at administration.

Statement 5: The composition of statement 1, wherein the composition has a radiochemical purity (RCP) of 98.0% or greater, 98.5% or greater, 99.0% or greater, or 99.5% or greater at administration.

Statement 6: The composition of statement 1, wherein the molar ratio of the PSMA I&T to 177Lu is from 7.0:1.0 to 7.6:1.0, 7.1:1.0 to 7.5:1.0, or 7.2:1.0 to 7.4:1.0.

Statement 7: The composition of statement 1, wherein the molar ratio of the PSMA I&T to 177Lu is from 6.0:1.0 to 7.0:1.0, 6.1:1.0 to 6.9:1.0, 6.2:1.0 to 6.8:1.0, 6.3:1.0 to 6.7:1.0, or 6.4:1.0 to 6.6:1.0.

Statement 8: The composition of statement 1, wherein the molar ratio of the PSMA I&T to 177Lu is from 5.0:1.0 to 6.0:1.0, 5.1:1.0 to 5.9:1.0, 5.2:1.0 to 5.8:1.0, 5.3:1.0 to 5.7.0:1.0, or 5.4:1.0 to 5.6:1.0.

Statement 9: The composition of statement 1, wherein the molar ratio of the PSMA I&T to 177Lu is from 4.4:1.0 to 5.0:1.0, 4.5:1.0 to 4.9:1.0, or 4.6:1.0 to 4.8:1.0.

Statement 10: The composition of statement 1, wherein the composition has an (RCP) of 95% or greater at 72 hours after formulation, 96 hours after formulation, or 120 hours after formulation.

Statement 11: The composition of statement 1, further comprising a buffering agent or solvent.

Statement 12: The composition of statement 1, wherein the PSMA I&T content is 30 μg/dose to 110 μg/dose, 30 μg/dose to 100 μg/dose, or 30 μg/dose to 90 μg/dose.

Statement 13: The composition of statement 1, wherein the PSMA I&T content is 95 μg/dose±15%, ±10%, or ±5%, 90 μg/dose±15%, ±10%, or ±5%, 85 μg/dose±15%, ±10%, or ±5%, 80 μg/dose±15%, ±10%, or ±5%, 75 μg/dose±15%, ±10%, or ±5%, 70 μg/dose±15%, ±10%, or ±5%, 60 μg/dose±15%, ±10%, or ±5%, 55 μg/dose±15%, ±10%, or ±5%, 50 μg/dose±15%, ±10%, or ±5%, 45 μg/dose±15%, ±10%, or ±5%, or 40 μg/dose±15%, ±10%.

Statement 14: The composition of statement 1, wherein the Fe metal content is ≤0.05 μg/GBq, ≤0.03 μg/GBq, ≤0.01 μg/GBq, or below the detectable limit.

Statement 15: The composition of statement 1, wherein the Cu metal content is ≤0.05 μg/GBq, ≤0.03 μg/GBq, ≤0.01 μg/GBq, or below the detectable limit.

Statement 16: The composition of statement 1, wherein the Zn metal content is ≤0.05 μg/GBq, ≤0.03 μg/GBq, ≤0.01 μg/GBq, or below the detectable limit.

Statement 17: The composition of statement 1, wherein the Pb metal content is ≤0.05 μg/GBq, ≤0.03 μg/GBq, ≤0.01 μg/GBq, or below the detectable limit.

Statement 18: The composition of statement 1, wherein the composition has a radiochemical purity (RCP) of 95% or greater at 72 hours after production.

Statement 19: The composition of statement 1, wherein the composition has a radiochemical purity (RCP) of 97% or greater at 72 hours after production.

Statement 20: The composition of statement 1, wherein the composition has a radiochemical purity (RCP) of 97.5% or greater at 72 hours after production.

Statement 21: The composition of statement 1, wherein the composition has a radiochemical purity (RCP) of 98.0% or greater, 98.5% or greater, 99.0% or greater, or 99.5% or greater at 72 hours after production.

Statement 22: The composition of statement 1, wherein the composition has a radiochemical purity (RCP) of 90% or greater, 91% or greater, 92% or greater, 93% or greater, 94% or greater, 95% or greater at 7 days after production.

Statement 23: The composition of statement 1, wherein the composition has a radiochemical purity (RCP) of 96.0% or greater, 96.5% or greater, 97.0% or greater, 97.5% or greater, 98.0% or greater, 98.5% or greater, 99.0% or greater, or 99.5% or greater at 7 days after production.

Statement 24: The composition of statement 1, wherein the composition is produced as part of a 4 Ci to 10 Ci batch scale.

Statement 25: The composition of statement 1, wherein the composition is produced as part of a 4 Ci to 15 Ci batch scale.

Statement 26: A radiopharmaceutical kit comprising a predetermined amount of the composition of statement 1.

Statement 27: A method of treating cancer in a patient in thereof comprising administering to the human patient the pharmaceutical composition of statement 1.

Statement 28: The method of statement 27, wherein the patient is treatment naïve.

Statement 29: The method of statement 27, wherein the patient is not treatment naïve.

Statement 30: The method of statement 27, wherein the pharmaceutical composition is administered to the cancer patient as a first line therapy or as a regimen.

Statement 31: A composition comprising 177Lu-PSMA I&T; wherein the molar ratio of the PSMA I&T to 177Lu is from 5.0:1.0 to 7.6:1.0, and the composition is suitable for administration to a human patient in need thereof for at least 96 hours after formulation.

Statement 32: The composition of statement 31, wherein the composition has a radiochemical purity (RCP) of 95% or greater at administration, 97% or greater at administration, or 97.5% or greater at administration.

Statement 33: A composition comprising 177Lu-PSMA I&T; wherein the molar ratio of the PSMA I&T to 177Lu is from 5.0:1.0 to 7.6:1.0, and the composition is suitable for administration to a human patient in need thereof for at least 120 hours after formulation.

Statement 34: The composition of statement 33, wherein the composition has a radiochemical purity (RCP) of 95% or greater at administration.

Statement 35: The composition of statement 31, wherein the composition has a radiochemical purity (RCP) of 98.0% or greater, 98.5% or greater, 99.0% or greater, or 99.5% or greater at administration.

Statement 36: The composition of statement 31, wherein the molar ratio of the PSMA I&T to 177Lu is from 7.0:1.0 to 7.6:1.0, 7.1:1.0 to 7.5:1.0, or 7.2:1.0 to 7.4:1.0.

Statement 37: The composition of statement 31, wherein the molar ratio of the PSMA I&T to 177Lu is from 6.0:1.0 to 7.0:1.0, 6.1:1.0 to 6.9:1.0, 6.2:1.0 to 6.8:1.0, 6.3:1.0 to 6.7:1.0, or 6.4:1.0 to 6.6:1.0.

Statement 38: The composition of statement 31, wherein the molar ratio of the PSMA I&T to 177Lu is from 5.0:1.0 to 6.0:1.0, 5.1:1.0 to 5.9:1.0, 5.2:1.0 to 5.8:1.0, 5.3:1.0 to 5.7.0:1.0, or 5.4:1.0 to 5.6:1.0.

Statement 39: The composition of statement 31, wherein the molar ratio of the PSMA I&T to 177Lu is about 5.1:1.0 to about 5.9:1.0.

Statement 40: The composition of statement 31, wherein the composition has an (RCP) of 95% or greater at 72 hours after formulation, 96 hours after formulation, or 120 hours after formulation.

Statement 41: The composition of statement 31, further comprising a buffering agent or solvent.

Statement 42: The composition of statement 31, wherein the PSMA I&T content is 30 μg/dose to 110 μg/dose, 30 μg/dose to 100 μg/dose, or 30 μg/dose to 90 μg/dose.

Statement 43: The composition of statement 31, wherein the PSMA I&T content is 95 μg/dose±15%, ±10%, or ±5%, 90 μg/dose±15%, ±10%, or ±5%, 85 μg/dose±15%, ±10%, or ±5%, 80 μg/dose±15%, ±10%, or ±5%, 75 μg/dose±15%, ±10%, or ±5%, 70 μg/dose±15%, ±10%, or ±5%, 60 μg/dose±15%, ±10%, or ±5%, 55 μg/dose±15%, ±10%, or ±5%, 50 μg/dose±15%, ±10%, or ±5%, 45 μg/dose±15%, ±10%, or ±5%, or 40 μg/dose±15%, ±10%.

Statement 44: The composition of statement 31, wherein the Fe metal content is ≤0.05 μg/GBq, ≤0.03 μg/GBq, ≤0.01 μg/GBq, or below the detectable limit.

Statement 45: The composition of statement 31, wherein the Cu metal content is ≤0.05 μg/GBq, ≤0.03 μg/GBq, ≤0.01 μg/GBq, or below the detectable limit.

Statement 46: The composition of statement 31, wherein the Zn metal content is ≤0.05 μg/GBq, ≤0.03 μg/GBq, ≤0.01 μg/GBq, or below the detectable limit.

Statement 47: The composition of statement 31, wherein the Pb metal content is ≤0.05 μg/GBq, ≤0.03 μg/GBq, ≤0.01 μg/GBq, or below the detectable limit.

Statement 48: The composition of statement 31, wherein the composition has a radiochemical purity (RCP) of 95% or greater at 72 hours after production.

Statement 49: The composition of statement 31, wherein the composition has a radiochemical purity (RCP) of 97% or greater at 72 hours after production.

Statement 50: The composition of statement 33, wherein the composition has a radiochemical purity (RCP) of 97.5% or greater at 120 hours after production.

Statement 51: The composition of statement 33, wherein the composition has a radiochemical purity (RCP) of 98.0% or greater, 98.5% or greater, 99.0% or greater, or 99.5% or greater at 120 hours after production.

Statement 52: The composition of statement 31, wherein the composition has a radiochemical purity (RCP) of 90% or greater, 91% or greater, 92% or greater, 93% or greater, 94% or greater, 95% or greater at 7 days after production.

Statement 53: The composition of statement 31, wherein the composition has a radiochemical purity (RCP) of 96.0% or greater, 96.5% or greater, 97.0% or greater, 97.5% or greater, 98.0% or greater, 98.5% or greater, 99.0% or greater, or 99.5% or greater at 7 days after production.

Statement 54: The composition of statement 31, wherein the composition is produced as part of a 4 Ci to 10 Ci batch scale.

Statement 55: The composition of statement 31, wherein the composition is produced as part of a 4 Ci to 15 Ci batch scale.

Statement 56: A radiopharmaceutical kit comprising a predetermined amount of the composition of statement 31.

Statement 57: A method of treating cancer in a patient in thereof comprising administering to the human patient the pharmaceutical composition of statement 31.

Statement 58: The method of statement 57, wherein the patient is treatment naïve.

Statement 59: The method of statement 57, wherein the patient is not treatment naïve.

Statement 60: The method of statement 57, wherein the pharmaceutical composition is administered to the cancer patient as a first line therapy or as a regimen.

Statement 61: A composition comprising 177Lu-PSMA I&T and ascorbic acid; wherein the molar ratio of the PSMA I&T to 177Lu is from 4.4:1.0 to 7.6:1.0, the composition has a radioactivity of about 13.3 to about 13.5 mCi, and the composition is suitable for administration to a human patient in need thereof for at least 72 hours after formulation.

Statement 62: A composition comprising 177Lu-PSMA I&T and DTPA; wherein the molar ratio of the PSMA I&T to 177Lu is from 4.4:1.0 to 7.6:1.0, the composition has a radioactivity of about 13.3 to about 13.5 mCi, and the composition is suitable for administration to a human patient in need thereof for at least 72 hours after formulation.

Statement 63: A method of treating or reducing the incidence of a cancer using an radiochemical composition, the method comprising administering a radiochemical composition into a human patient in need thereof, the radiochemical composition comprising 177Lu-PSMA I&T in a solution having a pH of 3.5 to 5.0, wherein the molar ratio of the PSMA I&T to 177Lu is from 4.4:1.0 to 7.6:1.0, wherein the solution has a radiochemical purity of more than 95% when administered, and wherein the composition is suitable for administration to a human patient in need thereof for at least 72 hours after formulation.

Statement 64: The method of statement 63, wherein the pH is from 3.5 to 4.5.

Statement 65: The method of statement 63, wherein the administering is via injection.

Statement 66: The method of statement 63, wherein the radiochemical composition further comprises ascorbic acid.

Statement 67: The method of statement 63, wherein the solution comprises about 10 mg/ml to about 50 mg/ml ascorbic acid.

Statement 68: The method of statement 63, wherein the radiochemical composition further comprises DTPA.

Statement 69: The method of statement 63, wherein the administering occurs over 1 to 6 cycles of treatment.

Statement 70: The method of statement 63, wherein the administering occurs over 7 or more cycles of treatment.

Statement 71: The method of statement 63, wherein the administering occurs over 8 or more cycles of treatment.

Statement 72: The method of statement 63, wherein the method of treating cancer prolongs a survival of the patient.

Statement 73: The method of statement 63, wherein the method of treating cancer increases progression-free survival of said patient.

Statement 74: The method of statement 63, the cancer is metastatic castration-resistant prostate cancer (mCRPC).

Statement 75: The method of statement 63, wherein the compound comprises <6 μg of Lu-PSMA I&T per mL of solution.

Statement 76: The method of statement 63, wherein the compound comprises <8 μg of Lu-PSMA I&T per mL of solution.

Statement 77: The method of statement 63, wherein the compound comprises <10 μg of Lu-PSMA I&T per mL of solution.

Statement 78: The method of statement 63, wherein the solution comprises about 13 μg to about 18 μg disodium EDTA per mL of solution.

Statement 79: The method of statement 63, wherein the solution comprises a complete absence of EDTA.

Statement 80: The method of statement 63, wherein the solution comprises about 1% to about 5% ethanol (v/v).

Statement 81: The method of statement 63, wherein upon administration of the composition to a patient, the patient maintains low or no levels of hematotoxic and nephrotoxic toxicity, and wherein the prostate-specific antigen decline is more than about 50%.

Statement 82: A method of treating a human patient with cancer, the method comprising:
administering a radiopharmaceutical composition comprising 177Lu-PSMA I&T and ascorbic acid in a solution having a pH of 3.5 to 6.0, wherein the molar ratio of the PSMA I&T to 177Lu is from 4.4:1.0 to 7.6:1.0, and wherein the solution has a radiochemical purity of ≥95% when administered.

Statement 83: The method of statement 82, further comprising imaging the patient using PSMA-PET to document and confirm the patient is cancer positive prior to administering the radiopharmaceutical composition.

Statement 84: The method of statement 82, wherein the patient has improved radiographic progression free survival (rPFS).

Statement 85: The method of statement 82, wherein the patient has an rPFS of about 6 months to 12 months.

Statement 86: The method of statement 82, wherein the patient has improved overall survival (OS).

Statement 87: The method of statement 82, wherein the patient has an OS of about 18 months to 25 months.

Statement 88: The method of statement 82, wherein the patient has improved second radiographic progression free survival (rPFS 2).

Statement 89: The method of statement 82, wherein the patient has improved progression free survival.

Statement 90: The method of statement 82, wherein the patient has improved second progression-free survival.

Statement 91: The method of statement 82, wherein the patient has an improved PSA50 response rate.

Statement 92: The method of statement 82, wherein the patient has an improved time to first symptomatic skeletal event (SSE).

Statement 93: The method of statement 82, wherein the patient has an improved time to soft tissue progression (STP).

Statement 94: The method of statement 82, wherein the patient has an improved time to chemotherapy (TTC).

Statement 95: The method of statement 82, wherein the patient has improved results on a Quality of Life Questionnaire.

Statement 96: The method of statement 82, wherein the pH of the composition is from 3.5 to 4.5.

Statement 97: The method of statement 82, wherein the administering is via injection.

Statement 98: The method of statement 82, wherein the composition further comprises ascorbic acid.

Statement 99: The method of statement 82, wherein the solution comprises about 10 mg/ml to about 50 mg/ml ascorbic acid.

Statement 100: The method of statement 82, wherein the composition further comprises DTPA.

Statement 101: The method of statement 82, wherein the administering occurs over 1 to 6 cycles of treatment.

Statement 102: The method of statement 82, wherein the administering occurs over 7 or more cycles of treatment.

Statement 103: The method of statement 82, wherein the composition has a radioactivity of less than 635 MBq/ml.

Statement 104: A method of treating a patient with mCRPC, the method comprising:
administering a radiopharmaceutical composition comprising 177Lu-PSMA I&T and ascorbic acid in a solution having a pH of 3.5 to 6.0, wherein the molar ratio of the PSMA I&T to 177Lu is from 4.4:1.0 to 7.6:1.0, and wherein the solution has a radiochemical purity of ≥more than 95% when administered.

Statement 105: The method of statement 104, further comprising imaging the patient using PSMA-PET to document and confirm the patient is mCRPC positive prior to administering the radiopharmaceutical composition.

Statement 106: The method of statement 104, wherein the patient has improved radiographic progression free survival (rPFS).

Statement 107: The method of statement 104, wherein the patient has an rPFS of about 6 months to 12 months.

Statement 108: The method of statement 104, wherein the patient has improved overall survival (OS).

Statement 109: The method of statement 104, wherein the patient has an OS of about 18 months to 25 months.

Statement 110: The method of statement 104, wherein the patient has improved second radiographic progression free survival (rPFS 2).

Statement 111: The method of statement 104, wherein the patient has improved progression free survival.

Statement 112: The method of statement 104, wherein the patient has improved second progression-free survival.

Statement 113: The method of statement 104, wherein the patient has an improved PSA50 response rate.

Statement 114: The method of statement 104, wherein the patient has an improved time to first symptomatic skeletal event (SSE).

Statement 115: The method of statement 104, wherein the patient has an improved time to soft tissue progression (STP).

Statement 116: The method of statement 104, wherein the patient has an improved time to chemotherapy (TTC).

Statement 117: The method of statement 104, wherein the patient has improved results on a Quality of Life Questionnaire.

Statement 118: The method of statement 104, wherein the pH is from 3.5 to 4.5.

Statement 119: The method of statement 104, wherein the administering is via injection.

Statement 120: The method of statement 104, wherein the composition further comprises ascorbic acid.

Statement 121: The method of statement 104, wherein the solution comprises about 10 mg/ml to about 50 mg/ml ascorbic acid.

Statement 122: The method of statement 104, wherein the composition further comprises DTPA.

Statement 123: The method of statement 104, wherein the administering occurs over 1 to 6 cycles of treatment.

Statement 124: The method of statement 104, wherein the administering occurs over 7 or more cycles of treatment.

Statement 125: The method of statement 104, wherein the composition has a radioactivity of less than 635 MBq/ml.

Statement 126: A radiopharmaceutical kit, comprising:
a vial containing at least a single dose of a 177Lu-PSMA I&T solution for injection to a human patient in need thereof, wherein the injection comprises a 7.5 GBq±0.1 GBq dose 7.4 GBq±0.1 GBq dose, or 7.3 GBq±0.1 GBq dose, the dose has a radiochemical purity that is ≥95% at the time of administration, and wherein the molar ratio of the PSMA I&T to 177Lu is from 4.4:1.0 to 7.6:1.0.

Statement 127: The radiopharmaceutical kit of statement 126, wherein the vial is a sterile pyrogen free glass vial of Type 1 glass with a fluorocoated bromobutyl rubber septum.

Statement 128: The radiopharmaceutical kit of statement 126, wherein the septum is sealed with a crimped aluminum capsule.

Statement 129: The radiopharmaceutical kit of statement 126, further comprising a lead shielded transport container, wherein the glass vial is kept in a lead shielded container during transportation.

Statement 130: The radiopharmaceutical kit of statement 126, wherein the lead shielded transport container complies to type A requirements (IAEA standards).

Statement 131: The radiopharmaceutical kit of statement 126, wherein the vial contains multiple doses.

Statement 132: The radiopharmaceutical kit of statement 126, wherein the 177Lu-PSMA I&T solution has a volume from about 10 ml to about 20 ml, from about 20 ml to about 30 ml, from about 30 ml to about 40 ml, from about 40 ml to about 50 ml, from about 50 ml to about 60 ml, from about 60 ml to about 70 ml, from about 70 ml to about 80 ml, from about 80 ml to about 90 ml, or from about 90 ml to about 100 ml.

Statement 133: The radiopharmaceutical kit of statement 126, wherein the 177Lu-PSMA I&T solution has a strength of about 0.1 GBq/ml, about 0.2 GBq/ml, about 0.3 GBq/ml, about 0.4 GBq/ml, about 0.5 GBq/ml, about 0.6 GBq/ml, about 0.7 GBq/ml, about 0.8 GBq/ml, about 0.9 GBq/ml, about 1.0 GBq/ml, about 1.1 GBq/ml, about 1.2 GBq/ml, about 1.3 GBq/ml, about 1.4 GBq/ml, about 1.5 GBq/ml, about 1.6 GBq/ml, about 1.7 GBq/ml, about 1.8 GBq/ml, about 1.9 GBq/ml, or about 2.0 GBq/ml.

Statement 134: The radiopharmaceutical kit of statement 126, wherein the 177Lu-PSMA I&T solution is suitable for administration to a human patient in need thereof more than 72 hours after formulation, more than 96 hours after formulation, or more than 100 hours after formulation.

Statement 135: The radiopharmaceutical kit of statement 126, wherein the 177Lu-PSMA I&T solution has a pH of 3.5 to 4.5.

Statement 136: A method of diagnosing or treating a tumor of a patient in need thereof, the method comprising administering by injection a radiopharmaceutical composition comprising 177Lu-PSMA I&T and ascorbic acid at a pH of 3.5 to 6.0 in solution, wherein the molar ratio of the PSMA I&T to 177Lu is from 4.4:1.0 to 7.6:1.0, the radiochemical purity of the composition is ≥95% at the time of administration, and wherein 20 hours after injection the radiopharmaceutical composition has an activity of at least 20% IA to 30% IA in the whole body.

Statement 137: The method of statement 136, wherein 40 hours after injection the radiopharmaceutical composition has an activity of at least 10% IA to 20% IA in the whole body.

Statement 138: The method of statement 136, wherein 60 hours after injection the radiopharmaceutical composition has an activity of at least 5% IA to 10% IA in the whole body.

Statement 139: A method of diagnosing or treating a tumor of a patient in need thereof, the method comprising administering to the patient by injection a radiopharmaceutical composition comprising 177Lu-PSMA I&T and ascorbic acid at a pH of 3.5 to 6.0 in solution, wherein the molar ratio of the PSMA I&T to 177Lu is from 4.4:1.0 to 7.6:1.0, the radiochemical purity of the composition is ≥95% at the time of administration, and wherein less than 20 hours after injection the radiopharmaceutical composition has an activity of at least 8% IA to 10% IA in the kidneys.

Statement 140: The method of statement 139, wherein 20 hours after injection the radiopharmaceutical composition has an activity of at least 3% IA to 8% IA in the kidneys.

Statement 141: The method of statement 139, wherein 40 hours after injection the radiopharmaceutical composition has an activity of at least 1% IA to 5% IA in the kidneys.

Statement 142: The method of statement 139, wherein 60 hours after injection the radiopharmaceutical composition has an activity of at least 1% IA to 5% IA in the kidneys.

Statement 143: A method of diagnosing or treating a tumor in a patient in need thereof, the method comprising administering to the patient by injection a radiopharmaceutical composition comprising 177Lu-PSMA I&T and ascorbic acid at a pH of 3.5 to 4.5 in solution, wherein the molar ratio of the PSMA I&T to 177Lu is from 4.4:1.0 to 7.6:1.0, the radiochemical purity of the composition is ≥95% at the time of administration, and wherein less than 20 hours after injection the radiopharmaceutical composition has an activity of at least 0.7% IA to 1% IA in the parotid glands.

Statement 144: The method of statement 143, wherein 20 hours after injection the radiopharmaceutical composition has an activity of at least 0.3% IA to 0.8% IA in the parotid glands.

Statement 145: The method of statement 143, wherein 40 hours after injection the radiopharmaceutical composition has an activity of at least 0.2% IA to 0.5% IA in the parotid glands.

Statement 146: The method of statement 143, wherein 60 hours after injection the radiopharmaceutical composition has an activity of at least 0.1% IA to 0.3% IA in the parotid glands.

Statement 147: A method of diagnosing or treating a tumor in a patient in need thereof, the method comprising administering to the patient by injection a radiopharmaceutical composition comprising 177Lu-PSMA I&T and ascorbic acid at a pH of 3.5 to 6.0 in solution, wherein the molar ratio of the PSMA I&T to 177Lu is from 4.4:1.0 to 7.6:1.0, the radiochemical purity of the composition is ≥95% at the time of administration, and wherein less than 20 hours after injection the radiopharmaceutical composition has an activity of at least 0.2% IA to 0.5% IA in lymph node lesions of the patient.

Statement 148: The method of statement 147, wherein 20 hours after injection the radiopharmaceutical composition has an activity of at least 0.1% IA to 0.3% IA in the lymph node lesions.

Statement 149: The method of statement 147, wherein 40 hours after injection the radiopharmaceutical composition has an activity of at least 0.08% IA to 0.2% IA in the lymph node lesions.

Statement 150: The method of statement 147, wherein 60 hours after injection the radiopharmaceutical composition has an activity of at least 0.05% IA to 0.1% IA in the lymph node lesions.

Statement 151: A method of diagnosing or treating a tumor in a patient in need thereof, the method comprising administering to the patient by injection a radiopharmaceutical composition comprising 177Lu-PSMA I&T and ascorbic acid at a pH of 3.5 to 6.0 in solution, wherein the molar ratio of the PSMA I&T to 177Lu is from 4.4:1.0 to 7.6:1.0, the radiochemical purity of the composition is ≥95% at the time of administration, and wherein less than 20 hours after injection the radiopharmaceutical composition has an activity of at least 0.1% IA to 0.4% IA in bone lesions of the patient.

Statement 152: The method of statement 151, wherein 20 hours after injection the radiopharmaceutical composition has an activity of at least 0.1% JA to 0.2% JA in the bone lesions.

Statement 153: The method of statement 151, wherein 40 hours after injection the radiopharmaceutical composition has an activity of at least 0.05% JA to 0.1% JA in the bone lesions.

Statement 154: The method of statement 151, wherein 60 hours after injection the radiopharmaceutical composition has an activity of at least 0.02% JA to 0.05% JA in the bone lesions.

Statement 155: A method of diagnosing or treating a tumor in a patient in need thereof, the method comprising administering to the patient by injection a radiopharmaceutical composition comprising 177Lu-PSMA I&T and ascorbic acid at a pH of 3.5 to 6.0 in solution, wherein the molar ratio of the PSMA I&T to 177Lu is from 4.4:1.0 to 7.6:1.0, the radiochemical purity of the composition is ≥95% at the time of administration, and wherein the radiopharmaceutical composition has an effective half-life of about 30 hours to 40 hours in the whole body of the patient.

Statement 156: The method of statement 155, wherein the effective half-life of the radiopharmaceutical composition is about 25 hours to 35 hours in the kidneys of the patient.

Statement 157: The method of statement 155, wherein the effective half-life of the radiopharmaceutical composition is about 20 hours to 30 hours in the parotid glands of the patient.

Statement 158: The method of statement 155, wherein the effective half-life of the radiopharmaceutical composition is about 45 hours to 55 hours in bone lesions of the patient.

Statement 159: The method of statement 155, wherein the effective half-life of the radiopharmaceutical composition is about 35 hours to 45 hours in lymph node lesions of the patient.

Statement 160: A method of diagnosing or treating a tumor in a patient in need thereof, the method comprising administering to the patient by injection a radiopharmaceutical composition comprising 177Lu-PSMA I&T and ascorbic acid at a pH of 3.5 to 6.0 in solution, wherein the molar ratio of the PSMA I&T to 177Lu is from 4.4:1.0 to 7.6:1.0, the radiochemical purity of the composition is ≥95% at the time of administration, and wherein a mean absorbed dose of the radiopharmaceutical is about 0.01 mGy/MBq to 0.5 mGy/MBq in the whole body of the patient.

Statement 161: The method of statement 160, wherein the mean absorbed dose of the radiopharmaceutical composition is about 0.5 mGy/MBq to 1.0 mGy/MBq in the kidneys of the patient.

Statement 162: The method of statement 160, wherein the mean absorbed dose of the radiopharmaceutical composition is about 1 mGy/MBq to 1.5 mGy/MBq in the parotid glands of the patient.

Statement 163: The method of statement 160, wherein the mean absorbed dose of the radiopharmaceutical composition is about 2.5 mGy/MBq to 3.5 mGy/MBq in bone lesions of the patient.

Statement 164: The method of statement 160, wherein the mean absorbed dose of the radiopharmaceutical composition is about 3.5 mGy/MBq to 4.5 mGy/MBq in lymph node lesions of the patient.

Statement 165: A radiopharmaceutical kit, comprising:
a vial containing at least a single dose of a 177Lu-PSMA I&T solution for injection to a human patient in need thereof, wherein the injection comprises a ≥7.1 GBq dose, the molar ratio of the PSMA I&T to 177Lu is from 4.4:1.0 to 7.6:1.0, and the radiochemical purity of the dose is ≥95%.

Statement 166: A method comprising administering to a human patient in need thereof a radiopharmaceutical composition comprising 177Lu-PSMA I&T in solution, wherein the molar ratio of the PSMA I&T to 177Lu is from 4.4:1.0 to 7.6:1.0, the radiochemical purity of the composition is ≥95% at the time of administration, and wherein a mean whole body effective dose of the radiopharmaceutical composition is about 0.001 mSv/MBq to 0.1 mSv/MBq.

Statement 167: The method of statement 166, wherein the composition comprises a pH of 3.5 to 4.5.

Statement 168: The method of statement 166, wherein the composition comprises ascorbic acid, ethanol, or a combination thereof.

Statement 169: The method of statement 166, wherein the administering is by injection.

Statement 170: The method of statement 166, wherein the composition comprises a 7.4 GBq±0.30 GBq dose of 177Lu-PSMA I&T.

Statement 171: The method of statement 166, wherein the projected cumulative absorbed dose to the kidneys at 6 cycles will be 20.4±10.2 Gy.

Statement 172: A method comprising administering to a human patient in need thereof a radiopharmaceutical composition comprising 177Lu-PSMA I&T, wherein the molar ratio of the PSMA I&T to 177Lu is from 4.4:1.0 to 7.6:1.0, and the radiochemical purity of the composition is ≥95% at the time of administration, and wherein a mean absorbed dose of the radiopharmaceutical is about 0.1 Gy/MBq to 1.0 Gy/MBq in the kidney of the human patient.

Statement 173: The method of statement 172, wherein the composition comprises a pH of 3.5 to 6.0.

Statement 174: The method of statement 172, wherein the composition comprises ascorbic acid, ethanol, or a combination thereof.

Statement 175: The method of statement 172, wherein the administering is by injection.

Statement 176: The method of statement 172, wherein the composition comprises a 7.4 GBq±0.30 GBq dose of 177Lu-PSMA I&T.

Statement 177: The method of statement 172, wherein the projected cumulative absorbed dose to the kidneys at 6 cycles will be 20.4±10.2 Gy.

Statement 178: A method comprising administering to a human patient in need thereof a radiopharmaceutical composition comprising 177Lu-PSMA I&T, wherein the molar ratio of the PSMA I&T to 177Lu is from 4.4:1.0 to 7.6:1.0, and the radiochemical purity of the composition is ≥95% at the time of administration, and wherein a mean absorbed dose of the radiopharmaceutical is about 1.0 Gy to 7.0 Gy per 7.4 GBq cycle in the kidney of the human patient.

Statement 179: The method of statement 178, wherein the composition comprises a pH of 3.5 to 4.5.

Statement 180: The method of statement 178, wherein the composition comprises ascorbic acid, DTPA, EDTA, ethanol, or a combination thereof.

Statement 181: The method of statement 178, wherein the administering is by injection.

Statement 182: The method of statement 178, wherein the composition comprises a 7.4 GBq±0.30 GBq dose of 177Lu-PSMA I&T.

Statement 183: The method of statement 178, wherein the projected cumulative absorbed dose to the kidneys at 6 cycles will be 20.4±10.2 Gy.

Statement 184: A method comprising administering to a human patient in need thereof a radiopharmaceutical composition comprising 177Lu-PSMA I&T, wherein the molar ratio of the PSMA I&T to 177Lu is from 4.4:1.0 to 7.6:1.0, and the radiochemical purity of the composition is ≥95% at the time of administration, and wherein a mean absorbed dose of the radiopharmaceutical is about 6.0 Gy to 42.0 Gy per cycle for 6 cycles of 7.4 GBq in the kidney of the patient.

Statement 185: A method comprising administering to the human patient in need thereof a radiopharmaceutical composition comprising 177Lu-PSMA I&T at a 7.4 GBq±0.30 GBq dose, wherein the molar ratio of the PSMA I&T to 177Lu is from 4.4:1.0 to 7.6:1.0, and the radiochemical purity of the composition is ≥95% at the time of administration, and wherein the projected cumulative absorbed dose to the kidneys at 6 cycles will be 20.4±10.2 Gy.

Statement 186: The method of statement 185, wherein the composition comprises a pH of 3.5 to 4.5.

Statement 187: The method of statement 185, wherein the composition comprises ascorbic acid, DTPA, EDTA, ethanol, or a combination thereof.

Statement 188: The method of statement 185, wherein the administering is by injection.

Statement 189: A method comprising administering to the human patient in need thereof a radiopharmaceutical composition comprising 177Lu-PSMA I&T at a 7.4 GBq±0.30 GBq dose, wherein the molar ratio of the PSMA I&T to 177Lu is from 4.4:1.0 to 7.6:1.0, and the radiochemical purity of the composition is ≥95% at the time of administration, and wherein the projected cumulative absorbed dose to the kidneys at 6 cycles will be ≤23.0 Gy, ≤22.5 Gy, ≤22.0 Gy, ≤21.5 Gy, ≤21.0 Gy, ≤20.5 Gy, or ≤20.4 Gy.

Statement 190: The method of statement 189, wherein the composition comprises a pH of 3.5 to 4.5.

Statement 191: The method of statement 189, wherein the composition comprises ascorbic acid, ethanol, or a combination thereof.

Statement 192: The method of statement 189, wherein the administering is by injection.

Statement 193: A method comprising administering to the human patient in need thereof a radiopharmaceutical composition comprising 177Lu-PSMA I&T at a 7.4 GBq±0.30 GBq dose, wherein the molar ratio of the PSMA I&T to 177Lu is from 4.4:1.0 to 7.6:1.0, and the radiochemical purity of the composition is ≥95% at the time of administration, and wherein the projected cumulative absorbed dose to the kidneys at 6 cycles will be ≤20.0 Gy, ≤19.5 Gy, or ≤19.0 Gy.

Statement 194: The method of statement 193, wherein the radiopharmaceutical composition has an effective half-life of about 10 hours to 40 hours in the whole body of the patient.

Statement 195: A method comprising administering to a human patient in need thereof a radiopharmaceutical composition comprising 177Lu-PSMA I&T in solution at a 7.4 GBq±0.30 GBq dose, wherein the molar ratio of the PSMA I&T to 177Lu is from 4.4:1.0 to 7.6:1.0, and the radiochemical purity of the composition is ≥95% at the time of administration.

Statement 196: The method of statement 195, wherein a mean whole body effective dose of the radiopharmaceutical composition is about 0.001 mSv/MBq to 0.1 mSv/MBq.

Statement 197: The method of statement 195, wherein the radiopharmaceutical composition comprises ascorbic acid.

Statement 198: The method of statement 195, wherein the radiopharmaceutical composition comprises a pH of 3.5 to 6.0 in solution.

Statement 199: The method of statement 195, wherein the radiopharmaceutical composition is suitable for administration to a human patient in need thereof at least 72 hours after formulation, and wherein the composition has a radiochemical purity of 95% or greater at administration.

Statement 200: The method of statement 195, wherein the mean whole body effective dose of the radiopharmaceutical composition is about 0.095 mSv/MBq, about 0.095 mSv/MBq±0.005 mSv/MBq, about 0.095 mSv/MBq±0.004 mSv/MBq, about 0.095 mSv/MBq±0.004 mSv/MBq, about 0.095 mSv/MBq±0.003 mSv/MBq, about 0.095 mSv/MBq±0.002 mSv/MBq, or about 0.095 mSv/MBq±0.001 mSv/MBq.

Statement 201: The method of statement 195, wherein the composition comprises a pH of 3.5 to 4.5.

Statement 202: The method of statement 195, wherein the composition comprises ascorbic acid, ethanol, or a combination thereof.

Statement 203: The method of statement 195, wherein the administering is by injection.

Statement 204: The method of statement 195, wherein the projected cumulative absorbed dose to the kidneys at 6 cycles is 19.092±10.2 Gy.

Statement 205: The method of statement 195, wherein the projected cumulative absorbed dose to the kidneys at 6 cycles is 19.3±8.0 Gy.

Statement 206: The method of statement 195, wherein a mean absorbed dose of the patient's kidneys is 0.43±0.18 Gy/GBq.

Statement 207: The method of statement 195, wherein the mean absorbed dose of the patient's kidneys is 0.43±0.18 Gy/GBq, 0.43±0.17 Gy/GBq, 0.43±0.16 Gy/GBq, 0.43±0.15 Gy/GBq, 0.43±0.14 Gy/GBq, 0.43±0.13 Gy/GBq, 0.43±0.12 Gy/GBq, 0.43±0.11 Gy/GBq, 0.43±0.10 Gy/GBq, 0.43±0.09 Gy/GBq, 0.43±0.08 Gy/GBq, 0.43±0.07 Gy/GBq, 0.43±0.06 Gy/GBq, 0.43±0.05 Gy/GBq, 0.43±0.04 Gy/GBq, 0.43±0.03 Gy/GBq, 0.43±0.02 Gy/GBq, or 0.43±0.01 Gy/GBq.

Statement 208: The method of statement 195, wherein a mean absorbed dose of the patient's urinary bladder is 0.38±0.03 Gy/GBq.

Statement 209: The method of statement 195, wherein a mean absorbed dose of the patient's urinary bladder is 0.38±0.03 Gy/GBq, 0.38±0.02 Gy/GBq, or 0.38±0.01 Gy/GBq.

Statement 210: The method of statement 195, wherein a mean absorbed dose of the patient's left colon is 0.54±0.42 Gy/GBq.

Statement 211: The method of statement 195, wherein a mean absorbed dose of the patient's left colon is 0.54±0.42 Gy/GBq, 0.54±0.41 Gy/GBq, 0.54±0.40 Gy/GBq, 0.54±0.39 Gy/GBq, 0.54±0.38 Gy/GBq, 0.54±0.37 Gy/GBq, 0.54±0.36 Gy/GBq, 0.54±0.35 Gy/GBq, 0.54±0.34 Gy/GBq, 0.54±0.33 Gy/GBq, 0.54±0.32 Gy/GBq, 0.54±0.31 Gy/GBq, 0.54±0.30 Gy/GBq, 0.54±0.29 Gy/GBq, 0.54±0.28 Gy/GBq, 0.54±0.27 Gy/GBq, 0.54±0.26 Gy/GBq, 0.54±0.25 Gy/GBq, 0.54±0.24 Gy/GBq, 0.54±0.23 Gy/GBq, 0.54±0.22 Gy/GBq, 0.54±0.21 Gy/GBq, 0.54±0.20 Gy/GBq, 0.54±0.19 Gy/GBq, 0.54±0.18 Gy/GBq, 0.54±0.17 Gy/GBq, 0.54±0.16 Gy/GBq, 0.54±0.15 Gy/GBq, 0.54±0.14 Gy/GBq, 0.54±0.13 Gy/GBq, 0.54±0.12 Gy/GBq, 0.54±0.11 Gy/GBq, 0.54±0.10 Gy/GBq, 0.54±0.09 Gy/GBq, 0.54±0.08 Gy/GBq, 0.54±0.07 Gy/GBq, 0.54±0.06 Gy/GBq, 0.54±0.05 Gy/GBq, 0.54±0.04 Gy/GBq, 0.54±0.03 Gy/GBq, 0.54±0.02 Gy/GBq, or 0.54±0.01 Gy/GBq.

Statement 212: The method of statement 195, wherein a mean absorbed dose of patient's right colon is 0.30±0.23 Gy/GBq.

Statement 213: The method of statement 195, wherein a mean absorbed dose of patient's right colon is 0.30±0.23 Gy/GBq, 0.30±0.22 Gy/GBq, 0.30±0.21 Gy/GBq, 0.30±0.20 Gy/GBq, 0.30±0.19 Gy/GBq, 0.30±0.18 Gy/GBq, 0.30±0.17 Gy/GBq, 0.30±0.16 Gy/GBq, 0.30±0.15 Gy/GBq, 0.30±0.14 Gy/GBq, 0.30±0.13 Gy/GBq, 0.30±0.12 Gy/GBq, 0.30±0.11 Gy/GBq, 0.30±0.10 Gy/GBq, 0.30±0.09 Gy/GBq, 0.30±0.08 Gy/GBq, 0.30±0.07 Gy/GBq, 0.30±0.06 Gy/GBq, 0.30±0.05 Gy/GBq, 0.30±0.04 Gy/GBq, 0.30±0.03 Gy/GBq, 0.30±0.02 Gy/GBq, or 0.30±0.01 Gy/GBq.

Statement 214: The method of statement 195, wherein a mean absorbed dose of the patient's rectum is 0.52±0.40 Gy/GBq.

Statement 215: The method of statement 195, wherein a mean absorbed dose of the patient's rectum is 0.52±0.40 Gy/GBq, 0.52±0.39 Gy/GBq, 0.52±0.38 Gy/GBq, 0.52±0.37 Gy/GBq, 0.52±0.36 Gy/GBq, 0.52±0.35 Gy/GBq, 0.52±0.34 Gy/GBq, 0.52±0.33 Gy/GBq, 0.52±0.32 Gy/GBq, 0.52±0.31 Gy/GBq, 0.52±0.30 Gy/GBq, 0.52±0.29 Gy/GBq, 0.52±0.28 Gy/GBq, 0.52±0.27 Gy/GBq, 0.52±0.26 Gy/GBq, 0.52±0.25 Gy/GBq, 0.52±0.24 Gy/GBq, 0.52±0.23 Gy/GBq, 0.52±0.22 Gy/GBq, 0.52±0.21 Gy/GBq, 0.52±0.20 Gy/GBq, 0.52±0.19 Gy/GBq, 0.52±0.18 Gy/GBq, 0.52±0.17 Gy/GBq, 0.52±0.16 Gy/GBq, 0.52±0.15 Gy/GBq, 0.52±0.14 Gy/GBq, 0.52±0.13 Gy/GBq, 0.52±0.12 Gy/GBq, 0.52±0.11 Gy/GBq, 0.52±0.10 Gy/GBq, 0.52±0.09 Gy/GBq, 0.52±0.08 Gy/GBq, 0.52±0.07 Gy/GBq, 0.52±0.06 Gy/GBq, 0.52±0.05 Gy/GBq, 0.52±0.04 Gy/GBq, 0.52±0.03 Gy/GBq, 0.52±0.02 Gy/GBq, or 0.52±0.01 Gy/GBq.

Statement 216: The method of statement 195, wherein a mean absorbed dose of the patient's large intestines is 0.54±0.42 Gy/GBq.

Statement 217: The method of statement 195, wherein a mean absorbed dose of the patient's large intestines is 0.54±0.42 Gy/GBq, 0.54±0.41 Gy/GBq, 0.54±0.40 Gy/GBq, 0.54±0.39 Gy/GBq, 0.54±0.38 Gy/GBq, 0.54±0.37 Gy/GBq, 0.54±0.36 Gy/GBq, 0.54±0.35 Gy/GBq, 0.54±0.34 Gy/GBq, 0.54±0.33 Gy/GBq, 0.54±0.32 Gy/GBq, 0.54±0.31 Gy/GBq, 0.54±0.30 Gy/GBq, 0.54±0.29 Gy/GBq, 0.54±0.28 Gy/GBq, 0.54±0.27 Gy/GBq, 0.54±0.26 Gy/GBq, 0.54±0.25 Gy/GBq, 0.54±0.24 Gy/GBq, 0.54±0.23 Gy/GBq, 0.54±0.22 Gy/GBq, 0.54±0.21 Gy/GBq, 0.54±0.20 Gy/GBq, 0.54±0.19 Gy/GBq, 0.54±0.18 Gy/GBq, 0.54±0.17 Gy/GBq, 0.54±0.16 Gy/GBq, 0.54±0.15 Gy/GBq, 0.54±0.14 Gy/GBq, 0.54±0.13 Gy/GBq, 0.54±0.12 Gy/GBq, 0.54±0.11 Gy/GBq, 0.54±0.10 Gy/GBq, 0.54±0.09 Gy/GBq, 0.54±0.08 Gy/GBq, 0.54±0.07 Gy/GBq, 0.54±0.06 Gy/GBq, 0.54±0.05 Gy/GBq, 0.54±0.04 Gy/GBq, 0.54±0.03 Gy/GBq, 0.54±0.02 Gy/GBq, or 0.54±0.01 Gy/GBq.

Statement 218: The method of statement 195 wherein a mean absorbed dose to the patient's salivary glands is 0.13±0.08 Gy/GBq.

Statement 219: The method of statement 195, wherein a mean absorbed dose to the patient's salivary glands is 0.13±0.08 Gy/GB, 0.13±0.07 Gy/GBq, 0.13±0.06 Gy/GBq, 0.13±0.05 Gy/GBq, 0.13±0.04 Gy/GBq, 0.13±0.03 Gy/GBq, 0.13±0.02 Gy/GBq, or 0.13±0.01 Gy/GBq.

Statement 220: The method of statement 195, wherein a mean absorbed dose to the patient's lacrimal glands is 0.67±0.33 Gy/GBq.

Statement 221: The method of statement 195, wherein the mean absorbed dose to the patient's lacrimal glands is 0.67±0.33 Gy/GBqm, 0.67±0.32 Gy/GBq, 0.67±0.31 Gy/GBq, 0.67±0.30 Gy/GBq, 0.67±0.29 Gy/GBq, 0.67±0.28 Gy/GBq, 0.67±0.27 Gy/GBq, 0.67±0.26 Gy/GBq, 0.67±0.25 Gy/GBq, 0.67±0.24 Gy/GBq, 0.67±0.23 Gy/GBq, 0.67±0.22 Gy/GBq, 0.67±0.21 Gy/GBq, 0.67±0.20 Gy/GBq, 0.67±0.19 Gy/GBq, 0.67±0.18 Gy/GBq, 0.67±0.17 Gy/GBq, 0.67±0.16 Gy/GBq, 0.67±0.15 Gy/GBq, 0.67±0.14 Gy/GBq, 0.67±0.13 Gy/GBq, 0.67±0.12 Gy/GBq, 0.67±0.11 Gy/GBq, 0.67±0.10 Gy/GBq, 0.67±0.09 Gy/GBq, 0.67±0.08 Gy/GBq, 0.67±0.07 Gy/GBq, 0.67±0.06 Gy/GBq, 0.67±0.05 Gy/GBq, 0.67±0.04 Gy/GBq, 0.67±0.03 Gy/GBq, 0.67±0.02 Gy/GBq, or 0.67±0.01 Gy/GBq.

Statement 222: A radiopharmaceutical composition comprising 177Lu-PSMA I&T at a 7.4 GBq±0.30 GBq dose, wherein the molar ratio of the PSMA I&T to 177Lu is from 4.4:1.0 to 7.6:1.0, and the radiochemical purity of the composition is ≥95% at the time of administration, and wherein the composition provides a mean absorbed dose to a human patient's lacrimal glands that is ≤1.10 Gy/GBq.

Statement 223: The radiopharmaceutical composition of statement 222, comprising ascorbic acid.

Statement 224: The radiopharmaceutical composition of statement 222, comprising a pH of 3.5 to 4.5 in solution.

Statement 225: The radiopharmaceutical composition of statement 222, wherein the composition is suitable for administration to a human patient in need thereof at least 72 hours after formulation.

Statement 226: The radiopharmaceutical composition of statement 222, wherein the composition has a radiochemical purity of 95% or greater at administration.

Statement 227: The radiopharmaceutical composition of statement 222, wherein the mean absorbed dose to the patient's lacrimal glands is ≤1.10 Gy/GBq, ≤1.05 Gy/GBq, ≤1.00 Gy/GBq, ≤0.95 Gy/GBq, ≤0.90 Gy/GBq, ≤0.85 Gy/GBq, ≤0.80 Gy/GBq, ≤0.75 Gy/GBq, ≤0.70 Gy/GBq, ≤0.65 Gy/GBq, ≤0.60 Gy/GBq, ≤0.55 Gy/GBq, ≤0.50 Gy/GBq, ≤0.45 Gy/GBq, ≤0.40 Gy/GBq, or ≤0.35 Gy/GBq.

Statement 228: A radiopharmaceutical composition comprising 177Lu-PSMA I&T and ascorbic acid at a pH of 3.5 to 6.0 in solution, a 7.4 GBq±0.30 GBq dose, wherein the molar ratio of the PSMA I&T to 177Lu is from 4.4:1.0 to 7.6:1.0, and the radiochemical purity of the composition is ≥95% at the time of administration, and wherein the composition is suitable for administration to a human patient in need thereof at least 72 hours after formulation, and wherein the composition provides a mean absorbed dose to the patient's lacrimal glands that is 0.67±0.33 Gy/GBqm.

Statement 229: A radiopharmaceutical composition comprising 177Lu-PSMA I&T at a 7.4 GBq±0.30 GBq dose, wherein the molar ratio of the PSMA I&T to 177Lu is from 4.4:1.0 to 7.6:1.0, and the radiochemical purity of the composition is ≥95% at the time of administration, and wherein the composition provides a mean absorbed dose to a human patient's kidneys that is ≤0.70 Gy/GBq.

Statement 230: The radiopharmaceutical composition of statement 229, comprising ascorbic acid.

Statement 231: The radiopharmaceutical composition of statement 229, comprising a pH of 3.5 to 4.5 in solution.

Statement 232: The radiopharmaceutical composition of statement 229, wherein the composition is suitable for administration to a human patient in need thereof at least 72 hours after formulation.

Statement 233: The radiopharmaceutical composition of statement 229, and wherein the composition has a radiochemical purity of 95% or greater at administration.

Statement 234: The radiopharmaceutical composition of statement 229, wherein the mean absorbed dose to the patient's kidneys is ≤0.65 Gy/GBq, ≤0.60 Gy/GBq, ≤0.55 Gy/GBq, ≤0.50 Gy/GBq, ≤0.45 Gy/GBq, ≤0.40 Gy/GBq, ≤0.35 Gy/GBq, ≤0.30 Gy/GBq, or ≤0.25 Gy/GBq.

Statement 235: A radiopharmaceutical composition comprising 177Lu-PSMA I&T at a 7.4 GBq±0.30 GBq dose, wherein the molar ratio of the PSMA I&T to 177Lu is from 4.4:1.0 to 7.6:1.0, and the radiochemical purity of the composition is ≥95% at the time of administration, and wherein the composition provides a mean absorbed dose to a human patient's salivary glands that is ≤0.30 Gy/GBq.

Statement 236: The radiopharmaceutical composition of statement 235, comprising ascorbic acid.

Statement 237: The radiopharmaceutical composition of statement 235, comprising a pH of 3.5 to 6.0 in solution.

Statement 238: The radiopharmaceutical composition of statement 235, wherein the composition is suitable for administration to a human patient in need thereof at least 72 hours after formulation.

Statement 239: The radiopharmaceutical composition of statement 235, wherein the composition has a radiochemical purity of 97.5% or greater at administration.

Statement 240: A radiopharmaceutical composition comprising 177Lu-PSMA I&T at a 6.8 GBq±0.30 GBq dose, wherein the molar ratio of the PSMA I&T to 177Lu is from 4.4:1.0 to 7.6:1.0, and the radiochemical purity of the composition is ≥95% at the time of administration, and wherein the composition provides a mean absorbed dose to a human patient's salivary glands that is ≤0.30 Gy/GBq.

Statement 241: The radiopharmaceutical composition of statement 240, comprising ascorbic acid.

Statement 242: The radiopharmaceutical composition of statement 240, comprising a pH of 3.5 to 6.0 in solution.

Statement 243: The radiopharmaceutical composition of statement 240, wherein the composition is suitable for administration to a human patient in need thereof at least 72 hours after formulation.

Statement 244: The radiopharmaceutical composition of statement 240, wherein the composition has a radiochemical purity of 97.5% or greater at administration.

Statement 245: A radiopharmaceutical composition comprising 177Lu-PSMA I&T at a 6.5 GBq±0.30 GBq dose, wherein the molar ratio of the PSMA I&T to 177Lu is from 4.4:1.0 to 7.6:1.0, and the radiochemical purity of the composition is ≥95% at the time of administration, and wherein the composition provides a mean absorbed dose to a human patient's salivary glands that is ≤0.30 Gy/GBq.

Statement 246: The radiopharmaceutical composition of statement 245, comprising ascorbic acid.

Statement 247: The radiopharmaceutical composition of statement 245, comprising a pH of 3.5 to 4.5 in solution.

Statement 248: The radiopharmaceutical composition of statement 245, wherein the composition is suitable for administration to a human patient in need thereof at least 72 hours after formulation.

Statement 249: The radiopharmaceutical composition of statement 245, wherein the composition has a radiochemical purity of 97.5% or greater at administration.

Statement 250: The radiopharmaceutical composition of statement 245, wherein the mean absorbed dose to the patient's salivary glands is ≤0.30 Gy/GBq, ≤0.25 Gy/GBq, ≤0.25 Gy/GBq, ≤0.20 Gy/GBq, ≤0.15 Gy/GBq, ≤0.10 Gy/GBq, or ≤0.05 Gy/GBq.

Statement 251: The method of statement 195, wherein the mean absorbed dose to the patient's lacrimal glands is 0.67±0.33 Gy/GBq, 0.67±0.32 Gy/GBq, 0.67±0.31 Gy/GBq, 0.67±0.30 Gy/GBq, 0.67±0.29 Gy/GBq, 0.67±0.28 Gy/GBq, 0.67±0.27 Gy/GBq, 0.67±0.26 Gy/GBq, 0.67±0.25 Gy/GBq, 0.67±0.24 Gy/GBq, 0.67±0.23 Gy/GBq, 0.67±0.22 Gy/GBq, 0.67±0.21 Gy/GBq, 0.67±0.20 Gy/GBq, 0.67±0.19 Gy/GBq, 0.67±0.18 Gy/GBq, 0.67±0.17 Gy/GBq, 0.67±0.16 Gy/GBq, 0.67±0.15 Gy/GBq, 0.67±0.14 Gy/GBq, 0.67±0.13 Gy/GBq, 0.67±0.12 Gy/GBq, 0.67±0.11 Gy/GBq, 0.67±0.10 Gy/GBq, 0.67±0.09 Gy/GBq, 0.67±0.08 Gy/GBq, 0.67±0.07 Gy/GBq, 0.67±0.06 Gy/GBq, 0.67±0.05 Gy/GBq, 0.67±0.04 Gy/GBq, 0.67±0.03 Gy/GBq, 0.67±0.02 Gy/GBq, or 0.67±0.01 Gy/GBq.

Statement 252: The method of statement 195, a mean absorbed dose to the patient's salivary glands is 0.13±0.08 Gy/GB, 0.13±0.07 Gy/GBq, 0.13±0.06 Gy/GBq, 0.13±0.05 Gy/GBq, 0.13±0.04 Gy/GBq, 0.13±0.03 Gy/GBq, 0.13±0.02 Gy/GBq, or 0.13±0.01 Gy/GBq.

Statement 253: The method of statement 195, wherein a mean absorbed dose of the patient's large intestines is 0.54±0.42 Gy/GBq, 0.54±0.41 Gy/GBq, 0.54±0.40 Gy/GBq, 0.54±0.39 Gy/GBq, 0.54±0.38 Gy/GBq, 0.54±0.37 Gy/GBq, 0.54±0.36 Gy/GBq, 0.54±0.35 Gy/GBq, 0.54±0.34 Gy/GBq, 0.54±0.33 Gy/GBq, 0.54±0.32 Gy/GBq, 0.54±0.31 Gy/GBq, 0.54±0.30 Gy/GBq, 0.54±0.29 Gy/GBq, 0.54±0.28 Gy/GBq, 0.54±0.27 Gy/GBq, 0.54±0.26 Gy/GBq, 0.54±0.25 Gy/GBq, 0.54±0.24 Gy/GBq, 0.54±0.23 Gy/GBq, 0.54±0.22 Gy/GBq, 0.54±0.21 Gy/GBq, 0.54±0.20 Gy/GBq, 0.54±0.19 Gy/GBq, 0.54±0.18 Gy/GBq, 0.54±0.17 Gy/GBq, 0.54±0.16 Gy/GBq, 0.54±0.15 Gy/GBq, 0.54±0.14 Gy/GBq, 0.54±0.13 Gy/GBq, 0.54±0.12 Gy/GBq, 0.54±0.11 Gy/GBq, 0.54±0.10 Gy/GBq, 0.54±0.09 Gy/GBq, 0.54±0.08 Gy/GBq, 0.54±0.07 Gy/GBq, 0.54±0.06 Gy/GBq, 0.54±0.05 Gy/GBq, 0.54±0.04 Gy/GBq, 0.54±0.03 Gy/GBq, 0.54±0.02 Gy/GBq, or 0.54±0.01 Gy/GBq.

Statement 254: The method of statement 195, wherein a mean absorbed dose of the patient's rectum is 0.52±0.40 Gy/GBq, 0.52±0.39 Gy/GBq, 0.52±0.38 Gy/GBq, 0.52±0.37 Gy/GBq, 0.52±0.36 Gy/GBq, 0.52±0.35 Gy/GBq, 0.52±0.34 Gy/GBq, 0.52±0.33 Gy/GBq, 0.52±0.32 Gy/GBq, 0.52±0.31 Gy/GBq, 0.52±0.30 Gy/GBq, 0.52±0.29 Gy/GBq, 0.52±0.28 Gy/GBq, 0.52±0.27 Gy/GBq, 0.52±0.26 Gy/GBq, 0.52±0.25 Gy/GBq, 0.52±0.24 Gy/GBq, 0.52±0.23 Gy/GBq, 0.52±0.22 Gy/GBq, 0.52±0.21 Gy/GBq, 0.52±0.20 Gy/GBq, 0.52±0.19 Gy/GBq, 0.52±0.18 Gy/GBq, 0.52±0.17 Gy/GBq, 0.52±0.16 Gy/GBq, 0.52±0.15 Gy/GBq, 0.52±0.14 Gy/GBq, 0.52±0.13 Gy/GBq, 0.52±0.12 Gy/GBq, 0.52±0.11 Gy/GBq, 0.52±0.10 Gy/GBq, 0.52±0.09 Gy/GBq, 0.52±0.08 Gy/GBq, 0.52±0.07 Gy/GBq, 0.52±0.06 Gy/GBq, 0.52±0.05 Gy/GBq, 0.52±0.04 Gy/GBq, 0.52±0.03 Gy/GBq, 0.52±0.02 Gy/GBq, or 0.52±0.01 Gy/GBq.

Statement 255: The method of statement 195, wherein a mean absorbed dose of patient's right colon is 0.30±0.23 Gy/GBq, 0.30±0.22 Gy/GBq, 0.30±0.21 Gy/GBq, 0.30±0.20 Gy/GBq, 0.30±0.19 Gy/GBq, 0.30±0.18 Gy/GBq, 0.30±0.17 Gy/GBq, 0.30±0.16 Gy/GBq, 0.30±0.15 Gy/GBq, 0.30±0.14 Gy/GBq, 0.30±0.13 Gy/GBq, 0.30±0.12 Gy/GBq, 0.30±0.11 Gy/GBq, 0.30±0.10 Gy/GBq, 0.30±0.09 Gy/GBq, 0.30±0.08 Gy/GBq, 0.30±0.07 Gy/GBq, 0.30±0.06 Gy/GBq, 0.30±0.05 Gy/GBq, 0.30±0.04 Gy/GBq, 0.30±0.03 Gy/GBq, 0.30±0.02 Gy/GBq, or 0.30±0.01 Gy/GBq.

Statement 256: The method of statement 195, wherein a mean absorbed dose of the patient's left colon is 0.54±0.42 Gy/GBq, 0.54±0.41 Gy/GBq, 0.54±0.40 Gy/GBq, 0.54±0.39 Gy/GBq, 0.54±0.38 Gy/GBq, 0.54±0.37 Gy/GBq, 0.54±0.36 Gy/GBq, 0.54±0.35 Gy/GBq, 0.54±0.34 Gy/GBq, 0.54±0.33 Gy/GBq, 0.54±0.32 Gy/GBq, 0.54±0.31 Gy/GBq, 0.54±0.30 Gy/GBq, 0.54±0.29 Gy/GBq, 0.54±0.28 Gy/GBq, 0.54±0.27 Gy/GBq, 0.54±0.26 Gy/GBq, 0.54±0.25 Gy/GBq, 0.54±0.24 Gy/GBq, 0.54±0.23 Gy/GBq, 0.54±0.22 Gy/GBq, 0.54±0.21 Gy/GBq, 0.54±0.20 Gy/GBq, 0.54±0.19 Gy/GBq, 0.54±0.18 Gy/GBq, 0.54±0.17 Gy/GBq, 0.54±0.16 Gy/GBq, 0.54±0.15 Gy/GBq, 0.54±0.14 Gy/GBq, 0.54±0.13 Gy/GBq, 0.54±0.12 Gy/GBq, 0.54±0.11 Gy/GBq, 0.54±0.10 Gy/GBq, 0.54±0.09 Gy/GBq, 0.54±0.08 Gy/GBq, 0.54±0.07 Gy/GBq, 0.54±0.06 Gy/GBq, 0.54±0.05 Gy/GBq, 0.54±0.04 Gy/GBq, 0.54±0.03 Gy/GBq, 0.54±0.02 Gy/GBq, or 0.54±0.01 Gy/GBq.

Statement 257: The method of statement 195, wherein a mean absorbed dose of the patient's urinary bladder is 0.38±0.03 Gy/GBq, 0.38±0.02 Gy/GBq, or 0.38±0.01 Gy/GBq.

Statement 258: The method of statement 195, the mean absorbed dose of the patient's kidneys is 0.43±0.18 Gy/GBq, 0.43±0.17 Gy/GBq, 0.43±0.16 Gy/GBq, 0.43±0.15 Gy/GBq, 0.43±0.14 Gy/GBq, 0.43±0.13 Gy/GBq, 0.43±0.12 Gy/GBq, 0.43±0.11 Gy/GBq, 0.43±0.10 Gy/GBq, 0.43±0.09 Gy/GBq, 0.43±0.08 Gy/GBq, 0.43±0.07 Gy/GBq, 0.43±0.06 Gy/GBq, 0.43±0.05 Gy/GBq, 0.43±0.04 Gy/GBq, 0.43±0.03 Gy/GBq, 0.43±0.02 Gy/GBq, or 0.43±0.01 Gy/GBq.

Statement 259: The method of statement 195, wherein a mean absorbed dose to the patient's kidneys is ≤0.70 Gy/GBq, a mean absorbed dose to the patient's lacrimal glands is ≤1.10 Gy/GBq, and a mean absorbed dose to the patient's salivary glands is ≤0.30 Gy/GBq.

Statement 260: The method of statement 195, wherein a mean absorbed dose to the patient's kidneys is ≤0.60 Gy/GBq, a mean absorbed dose to the patient's lacrimal glands is ≤1.00 Gy/GBq, and a mean absorbed dose to the patient's salivary glands is ≤0.25 Gy/GBq.

Statement 261: The method of statement 195, wherein a mean absorbed dose to the patient's kidneys is ≤0.50 Gy/GBq, a mean absorbed dose to the patient's lacrimal glands is ≤0.90 Gy/GBq, and a mean absorbed dose to the patient's salivary glands is ≤0.20 Gy/GBq.

Statement 262: The method of statement 195, wherein a mean absorbed dose to the patient's kidneys is ≤0.45 Gy/GBq, a mean absorbed dose to the patient's lacrimal glands is ≤0.80 Gy/GBq, and a mean absorbed dose to the patient's salivary glands is ≤0.15 Gy/GBq.

Statement 263: A method comprising administering to a human patient in need thereof a radiopharmaceutical composition comprising 177Lu-PSMA I&T that provides a mean absorbed dose to the patient's kidneys of 0.43±0.18 Gy/GBq, 0.43±0.17 Gy/GBq, 0.43±0.16 Gy/GBq, 0.43±0.15 Gy/GBq, 0.43±0.14 Gy/GBq, 0.43±0.13 Gy/GBq, 0.43±0.12 Gy/GBq, 0.43±0.11 Gy/GBq, 0.43±0.10 Gy/GBq, 0.43±0.09 Gy/GBq, 0.43±0.08 Gy/GBq, 0.43±0.07 Gy/GBq, 0.43±0.06 Gy/GBq, 0.43±0.05 Gy/GBq, 0.43±0.04 Gy/GBq, 0.43±0.03 Gy/GBq, 0.43±0.02 Gy/GBq, or 0.43±0.01 Gy/GBq.

Statement 264: The method of statement 263, wherein the molar ratio of the PSMA I&T to 177Lu is from 4.4:1.0 to 7.6:1.0, and the radiochemical purity of the composition is ≥95% at the time of administration,

Statement 265: A method comprising administering to a human patient in need thereof a radiopharmaceutical composition comprising 177Lu-PSMA I&T that provides a mean absorbed dose to the patient's kidneys that is ≤0.70 Gy/GBq, ≤0.65 Gy/GBq, ≤0.60 Gy/GBq, ≤0.55 Gy/GBq, ≤0.50 Gy/GBq, ≤0.45 Gy/GBq, ≤0.40 Gy/GBq, ≤0.35 Gy/GBq, ≤0.30 Gy/GBq, or ≤0.25 Gy/GBq.

Statement 266: The method of statement 265, wherein the molar ratio of the PSMA I&T to 177Lu is from 4.4:1.0 to 7.6:1.0, and the radiochemical purity of the composition is ≥95% at the time of administration.

Statement 267: A method comprising administering to a human patient in need thereof a radiopharmaceutical composition comprising 177Lu-PSMA I&T that provides a mean absorbed dose to the patient's kidneys that is ≤0.70 Gy/GBq, ≤0.65 Gy/GBq, ≤0.60 Gy/GBq, ≤0.55 Gy/GBq, ≤0.50 Gy/GBq, ≤0.45 Gy/GBq, ≤0.40 Gy/GBq, ≤0.35 Gy/GBq, ≤0.30 Gy/GBq, or ≤0.25 Gy/GBq and a standard of deviation of 0.25 or less, 0.24 or less, 0.23 or less, 0.22 or less, 0.21 or less, 0.20 or less, 0.19 or less, or 0.18 or less.

Statement 268: The method of statement 267, wherein the molar ratio of the PSMA I&T to 177Lu is from 4.4:1.0 to 7.6:1.0, and the radiochemical purity of the composition is ≥95% at the time of administration,

Statement 269: The method of statement 195, wherein the total cumulative dose to the patient's kidneys over 1, 2, 3, 4, 5, 6, and/or 7 cycles of administration is 23.0 Gy, ≤22.9 Gy, ≤22.8 Gy, ≤22.7 Gy, ≤22.6 Gy, ≤22.5 Gy, ≤22.4 Gy, ≤22.3 Gy, ≤22.2 Gy, 22.1 Gy, ≤22.0 Gy, ≤21.9 Gy, ≤21.8 Gy, ≤21.7 Gy, ≤21.6 Gy, ≤21.5 Gy, ≤21.4 Gy, ≤21.3 Gy, ≤21.2 Gy, ≤21.1 Gy, ≤21.0 Gy, ≤20.9 Gy, ≤20.8 Gy, ≤20.7 Gy, ≤20.6 Gy, ≤20.5 Gy, ≤20.4 Gy, ≤20.3 Gy, ≤20.2 Gy, ≤20.1 Gy, ≤20.0 Gy, ≤19.9 Gy, ≤19.8 Gy, ≤19.7 Gy, ≤19.6 Gy, ≤19.5 Gy, ≤19.4 Gy, ≤19.3 Gy, ≤19.2 Gy, or ≤19.1 Gy.

Statement 270: The method of statement 195, wherein the total cumulative dose to the patient's kidneys over 6 cycles of administration is 23.0 Gy, ≤22.9 Gy, ≤22.8 Gy, ≤22.7 Gy, ≤22.6 Gy, ≤22.5 Gy, ≤22.4 Gy, ≤22.3 Gy, ≤22.2 Gy, ≤22.1 Gy, ≤22.0 Gy, ≤21.9 Gy, ≤21.8 Gy, ≤21.7 Gy, ≤21.6 Gy, ≤21.5 Gy, ≤21.4 Gy, ≤21.3 Gy, ≤21.2 Gy, ≤21.1 Gy, ≤21.0 Gy, ≤20.9 Gy, ≤20.8 Gy, ≤20.7 Gy, ≤20.6 Gy, ≤20.5 Gy, ≤20.4 Gy, ≤20.3 Gy, ≤20.2 Gy, ≤20.1 Gy, ≤20.0 Gy, ≤19.9 Gy, ≤19.8 Gy, ≤19.7 Gy, ≤19.6 Gy, ≤19.5 Gy, ≤19.4 Gy, ≤19.3 Gy, ≤19.2 Gy, or ≤19.1 Gy.

Statement 271: The method of statement 195, wherein the total cumulative dose to the patient's kidneys over 5 cycles of administration is ≤19.2 Gy, ≤19.1 Gy, ≤19.0 Gy, ≤18.9 Gy, ≤18.8 Gy, ≤18.7 Gy, ≤18.6 Gy, ≤18.5 Gy, ≤18.4 Gy, ≤18.3 Gy, ≤18.2 Gy, ≤18.1 Gy, ≤18.0 Gy, ≤17.9 Gy, ≤17.8 Gy, ≤17.7 Gy, ≤17.6 Gy, ≤17.5 Gy, ≤17.4 Gy, ≤17.3 Gy, ≤17.2 Gy, ≤17.1 Gy, ≤17.0 Gy, ≤16.8 Gy, ≤16.7 Gy, ≤16.6 Gy, ≤16.5 Gy, ≤16.4 Gy, ≤16.3 Gy, ≤16.2 Gy, ≤16.1 Gy, ≤16.0 Gy, or ≤15.9 Gy.

Statement 272: The method of statement 195, wherein the total cumulative dose to the patient's kidneys over 4 cycles of administration is ≤15.3 Gy, ≤15.2 Gy, ≤15.1 Gy, ≤15.0 Gy, ≤14.9 Gy, ≤14.8 Gy, ≤14.7 Gy, ≤14.6 Gy, ≤14.3 Gy, ≤14.2 Gy, ≤14.1 Gy, ≤14.0 Gy, ≤13.9 Gy, ≤13.8 Gy, ≤13.7 Gy, ≤13.6 Gy, ≤13.5 Gy, ≤13.4 Gy, ≤13.3 Gy, ≤13.2 Gy, ≤13.1 Gy, ≤13.0 Gy, ≤12.9 Gy, ≤12.8 Gy, or ≤12.7 Gy.

Statement 273: The method of statement 195, wherein the total cumulative dose to the patient's kidneys over 3 cycles of administration is ≤11.5 Gy, ≤11.4 Gy, ≤11.3 Gy, ≤11.2 Gy, ≤11.1 Gy, ≤11.0 Gy, ≤10.9 Gy, ≤10.8 Gy, ≤10.7 Gy, ≤10.6 Gy, ≤10.5 Gy, ≤10.4 Gy, ≤10.3 Gy, ≤10.2 Gy, ≤10.1 Gy, ≤10.0 Gy, ≤9.9 Gy, ≤9.8 Gy, ≤9.7 Gy, ≤9.6 Gy, or ≤9.5 Gy

Statement 274: The method of statement 195, wherein the total cumulative dose to the patient's kidneys over 2 cycles of administration is ≤7.7 Gy, ≤7.6 Gy, ≤7.5 Gy, ≤7.3 Gy, ≤7.2 Gy, ≤7.1 Gy, ≤7.0 Gy, ≤6.9 Gy, ≤6.8 Gy, ≤6.7 Gy, ≤6.6 Gy, ≤6.5 Gy, or ≤6.4 Gy.

Statement 275: The method of statement 195, wherein the total cumulative dose to the patient's kidneys over 1 cycle of administration is ≤3.9 Gy, ≤3.8 Gy, ≤3.7 Gy, ≤3.6 Gy, ≤3.5 Gy, ≤3.4 Gy, ≤3.3 Gy, or 3.2 Gy.

Statement 276: The method of statement 195, wherein the radiopharmaceutical composition comprises radionuclidic identification (HPGe-detector) of the composition showing gamma ray energy peaks at 113±2 keV and 208±4 keV, and no other significant peaks with gamma energy >100 keV are detected.

Statement 277: The method of statement 195, further comprising applying ice packs to the human patient to cool the parotid or submandibular glands of the human patient within the time frame from 30 min prior to up to 4 hours after administration of 177Lu-PSMA I&T to reduce the risk of salivary gland radiation injuries.

Statement 278: A method comprising administering to the human patient in need thereof a radiopharmaceutical composition comprising 177Lu-PSMA I&T at a 7.4 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 8 cycles will be ≤23.0 Gy, ≤22.5 Gy, or ≤22.0 Gy.

Statement 279: A method comprising administering to the human patient in need thereof a radiopharmaceutical composition comprising 177Lu-PSMA I&T at a 7.1 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 8 cycles will be ≤23.0 Gy, ≤22.5 Gy, or ≤22.0 Gy.

Statement 280: A method comprising administering to the human patient in need thereof a radiopharmaceutical composition comprising 177Lu-PSMA I&T at a 6.8 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 8 cycles will be ≤23.0 Gy, ≤22.5 Gy, or ≤22.0 Gy.

Statement 281: A method comprising administering to the human patient in need thereof a radiopharmaceutical composition comprising 177Lu-PSMA I&T at a 6.5 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 8 cycles will be ≤23.0 Gy, ≤22.5 Gy, or ≤22.0 Gy.

Statement 282: A method comprising administering to the human patient in need thereof a radiopharmaceutical composition comprising 177Lu-PSMA I&T at a 6.2 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 8 cycles will be ≤23.0 Gy, ≤22.5 Gy, or ≤22.0 Gy.

Statement 283: A method comprising administering to the human patient in need thereof a radiopharmaceutical composition comprising 177Lu-PSMA I&T at a 5.9 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 8 cycles will be ≤23.0 Gy, ≤22.5 Gy, or ≤22.0 Gy.

Statement 284: A method comprising administering to the human patient in need thereof a radiopharmaceutical composition comprising 177Lu-PSMA I&T at a 7.4 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 8 cycles will be ≤23.0 Gy, ≤22.5 Gy, or ≤22.0 Gy.

Statement 285: A method comprising administering to the human patient in need thereof a radiopharmaceutical composition comprising 177Lu-PSMA I&T at a 7.1 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 7 cycles will be ≤23.0 Gy, ≤22.5 Gy, or ≤22.0 Gy.

Statement 286: A method comprising administering to the human patient in need thereof a radiopharmaceutical composition comprising 177Lu-PSMA I&T at a 6.8 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 7 cycles will be ≤23.0 Gy, ≤22.5 Gy, or ≤22.0 Gy.

Statement 287: A method comprising administering to the human patient in need thereof a radiopharmaceutical composition comprising 177Lu-PSMA I&T at a 6.5 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 7 cycles will be ≤23.0 Gy, ≤22.5 Gy, or ≤22.0 Gy.

Statement 288: A method comprising administering to the human patient in need thereof a radiopharmaceutical composition comprising 177Lu-PSMA I&T at a 6.2 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 7 cycles will be ≤23.0 Gy, ≤22.5 Gy, or ≤22.0 Gy.

Statement 289: A method comprising administering to the human patient in need thereof a radiopharmaceutical composition comprising 177Lu-PSMA I&T at a 5.9 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 7 cycles will be ≤23.0 Gy, ≤22.5 Gy, or ≤22.0 Gy.

Statement 290: A method comprising administering to the human patient in need thereof a radiopharmaceutical composition comprising 177Lu-PSMA I&T at a 7.4 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 6 cycles will be ≤23.0 Gy, ≤22.5 Gy, or ≤22.0 Gy.

Statement 291: A method comprising administering to the human patient in need thereof a radiopharmaceutical composition comprising 177Lu-PSMA I&T at a 7.1 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 6 cycles will be ≤20.0 Gy, ≤19.5 Gy, or ≤19.0 Gy.

Statement 292: A method comprising administering to the human patient in need thereof a radiopharmaceutical composition comprising 177Lu-PSMA I&T at a 6.8 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 6 cycles will be ≤20.0 Gy, ≤19.5 Gy, or ≤19.0 Gy.

Statement 293: A method comprising administering to the human patient in need thereof a radiopharmaceutical composition comprising 177Lu-PSMA I&T at a 6.5 GBq±0.30 GBq dose, wherein the molar ratio of the PSMA I&T to 177Lu is from 4.4:1.0 to 7.6:1.0, the radiochemical purity of the composition is ≥95% at the time of administration, and wherein the projected cumulative absorbed dose to the kidneys at 6 cycles will be ≤20.0 Gy, ≤19.5 Gy, or ≤19.0 Gy.

Statement 294: A method comprising administering to the human patient in need thereof a radiopharmaceutical composition comprising 177Lu-PSMA I&T at a 0.5 GBq to 7.4 GBq dose±0.30 GBq, wherein the molar ratio of the PSMA I&T to 177Lu is from 4.4:1.0 to 7.6:1.0, the radiochemical purity of the composition is ≥95% at the time of administration, and wherein the total cumulative absorbed dose to the patient's kidneys per each administration is ≤3.9 Gy, ≤3.8 Gy, ≤3.7 Gy, ≤3.6 Gy, ≤3.5 Gy, ≤3.4 Gy, ≤3.3 Gy, ≤3.2 Gy, ≤3.1 Gy, ≤3.0 Gy, ≤2.9 Gy, ≤2.8 Gy, ≤2.7 Gy, ≤2.6 Gy, ≤2.5 Gy, or ≤2.4 Gy.

Statement 295: The method of statement 294, wherein the patient is administered a dose one, two, three, four, five, six, seven, eight, or more times.

Statement 296: A method comprising administering to the human patient in need thereof a radiopharmaceutical composition comprising 177Lu-PSMA I&T at a 0.5 GBq to 6.8 GBq dose±0.30 GBq, wherein the molar ratio of the PSMA I&T to 177Lu is from 4.4:1.0 to 7.6:1.0, the radiochemical purity of the composition is ≥95% at the time of administration, and wherein the total cumulative dose to the patient's kidneys per each administration is ≤3.9 Gy, ≤3.8 Gy, ≤3.7 Gy, ≤3.6 Gy, ≤3.5 Gy, ≤3.4 Gy, ≤3.3 Gy, ≤3.2 Gy, ≤3.1 Gy, ≤3.0 Gy, ≤2.9 Gy, ≤2.8 Gy, ≤2.7 Gy, ≤2.6 Gy, ≤2.5 Gy, or ≤2.4 Gy.

Statement 297: The method of statement 296, wherein the patient is administered a dose one, two, three, four, five, six, seven, eight, or more times.

Statement 298: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's kidneys of 0.43 Gy/MBq±0.15 Gy/MBq, ±0.10 Gy/MBq, or ±0.05 Gy/MBq.

Statement 299: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's kidneys of ≤0.43 Gy/MBq for each administration.

Statement 300: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration occurs ≥10 times, and the treatments are administered every 4, 5, 6, 7, or 8 weeks, provided that the total cumulative absorbed dose to the patient's kidneys after all administrations remains below 23 grays (Gy).

Statement 301: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration occurs ≥8 times, ≥10 times, ≥15 times, ≥20 times, ≥25 times, ≥30 times, ≥35 times, ≥40 times, ≥45 times, ≥50 times, ≥55 times, ≥60 times, ≥65 times, ≥70 times, or ≥75 times, provided that the total cumulative absorbed dose to the patient's kidneys after all administrations remains below 23 grays (Gy).

Statement 302: The composition of statement 1, wherein the PSMA I&T to activity ratio at calibration of 0.6 μg/mCi±0.1 μg/mCi.

Statement 303: The composition of statement 1, wherein the composition comprises a concentration from about 0.1 M (20 mg/mL) to about 0.6 M (80 mg/mL).

Statement 304: The composition of statement 1, wherein the composition comprises a concentration from about 0.2 M (40 mg/mL) to about 0.6 M (80 mg/mL).

Statement 305: The composition of statement 1, wherein the composition comprises a radioactive concentration (RAC) of about 90 mCi/mL to about 700 mCi/mL, about 90 mCi/mL to about 650 mCi/mL, about 90 mCi/mL to about 600 mCi/mL, about 90 mCi/mL to about 550 mCi/mL, or about 90 mCi/mL to about 500 mCi/mL.

Statement 306: The composition of statement 1, wherein the composition comprises a drug product to sodium ascorbate content is about 2.8 mg/mL to about 35 mg/mL on the ascorbic acid basis.

Statement 307: The composition of statement 1, wherein the composition comprises a drug product to sodium ascorbate content is about 25 mg/mL to about 40 mg/mL on the ascorbic acid basis.

Statement 308: The composition of statement 1, wherein the composition comprises about 0% to about 7% ethanol (w/w).

Statement 309: The composition of statement 1, wherein the composition comprises about 0% to about 3% ethanol (w/w).

Statement 310: The composition of statement 1, wherein the composition comprises about 0% to about 1% ethanol (w/w).

Statement 311: The composition of statement 1, wherein the composition comprises about 3.5%±3.5% ethanol (w/w).

Statement 312: The composition of statement 1, wherein the composition comprises a DTPA content of about 0 mg/mL to about 0.15 mg/mL.

Statement 313: The composition of statement 1, wherein the composition comprises a DTPA content of about 0.1 mg/mL±0.05 mg/mL.

Statement 314: The composition of statement 1, wherein the composition comprises a concentration of about 7.57 mCi/mL to about 22.6 mCi/mL.

Statement 315: The composition of statement 1, wherein the composition comprises a concentration of about 15.0 mCi/mL to about 22.6 mCi/mL.

Statement 316: The composition of statement 1, wherein the composition comprises a concentration of about 18.2 mCi/mL±2.0 mCi/mL at the time of formulation.

Statement 317: The composition of statement 1, wherein the composition comprises about 5.2 Ci to about 11.5 Ci for at least 96 hours.

Statement 318: The composition of statement 1, wherein the composition comprises about 5.2 Ci to about 11.5 Ci for at least 84 hours.

Statement 319: The composition of statement 1, wherein the composition comprises about 5.2 Ci to about 11.5 Ci for at least 72 hours.

Statement 320: The composition of statement 1, wherein the PSMA I&T content is about 100 μg/dose to about 80 μg/dose, about 105 μg/dose to about 80 μg/dose, about 110 μg/dose to about 80 μg/dose, or about 115 μg/dose to about 80 μg/dose.

Statement 321: The composition of statement 1, wherein the PSMA I&T content is about 40 μg/dose to about 100 μg/dose, about 45 μg/dose to about 100 μg/dose, about 50 μg/dose to about 100 μg/dose, about 55 μg/dose to about 100 μg/dose, about 60 μg/dose to about 100 μg/dose, about 65 μg/dose to about 100 μg/dose, about 70 μg/dose to about 100 μg/dose, about 75 μg/dose to about 100 μg/dose, about 80 μg/dose to about 100 μg/dose, about 85 μg/dose to about 100 μg/dose, about 90 μg/dose to about 100 μg/dose, or about 95 μg/dose to about 100 μg/dose.

Statement 322: The composition of statement 1, wherein the PSMA I&T content is about 45 μg/dose to about 95 μg/dose, 50 μg/dose to about 100 μg/dose, 55 μg/dose to about 95 μg/dose, 60 μg/dose to about 95 μg/dose, 65 μg/dose to about 95 μg/dose, 70 μg/dose to about 95 μg/dose, 75 μg/dose to about 95 μg/dose, 80 μg/dose to about 95 μg/dose, 85 μg/dose to about 95 μg/dose, or about 90 μg/dose to about 95 μg/dose.

Statement 323: The composition of statement 1, wherein the PSMA I&T content is about 40 μg/dose to about 90 μg/dose, about 45 μg/dose to about 90 μg/dose, about 50 μg/dose to about 90 μg/dose, about 55 μg/dose to about 90 μg/dose, about 60 μg/dose to about 90 μg/dose, about 65 μg/dose to about 90 μg/dose, about 70 μg/dose to about 90 μg/dose, about 75 μg/dose to about 90 μg/dose, about 80 μg/dose to about 90 μg/dose, or about 85 μg/dose to about 90 μg/dose.

Statement 324: The composition of statement 1, wherein the PSMA I&T content is about 40 μg/dose to about 85 μg/dose, about 45 μg/dose to about 85 μg/dose, about 50 μg/dose to about 85 μg/dose, about 55 μg/dose to about 85 μg/dose, about 60 μg/dose to about 85 μg/dose, about 65 μg/dose to about 85 μg/dose, about 70 μg/dose to about 85 μg/dose, about 75 μg/dose to about 85 μg/dose, about 80 μg/dose to about 85 μg/dose.

Statement 325: The composition of statement 1, wherein the PSMA I&T content is about 40 μg/dose to about 80 μg/dose, about 45 μg/dose to about 80 μg/dose, about 50 μg/dose to about 80 μg/dose, about 55 μg/dose to about 80 μg/dose, about 60 μg/dose to about 80 μg/dose, about 65 μg/dose to about 80 μg/dose, about 70 μg/dose to about 80 μg/dose, or about 75 μg/dose to about 80 μg/dose.

Statement 326: The composition of statement 1, wherein the PSMA I&T content is about 40 μg/dose to about 75 μg/dose, about 45 μg/dose to about 75 μg/dose, about 50 μg/dose to about 75 μg/dose, about 55 μg/dose to about 75 μg/dose, about 60 μg/dose to about 75 μg/dose, about 65 μg/dose to about 75 μg/dose, about 70 μg/dose to about 75 μg/dose.

Statement 327: The composition of statement 1, wherein the composition comprises a maximum of 120 μg of PSMA I&T, a maximum of 110 μg of PSMA I&T, a maximum of 100 μg of PSMA I&T, a maximum of 90 μg of PSMA I&T, a maximum of 80 μg of PSMA I&T, a maximum of 70 μg of PSMA I&T, a maximum of 60 μg of PSMA I&T, a maximum of 50 μg of PSMA I&T, or a maximum of 40 μg of PSMA I&T.

Statement 328: The composition of statement 1, wherein the composition comprises a maximum of 120 μg of PSMA, a maximum of 110 μg of PSMA, a maximum of 100 μg of PSMA, a maximum of 90 μg of PSMA, a maximum of 80 μg of PSMA, a maximum of 70 μg of PSMA, a maximum of 60 μg of PSMA, a maximum of 50 μg of PSMA, or a maximum of 40 μg of PSMA.

Statement 329: A method comprising administering to a human patient in need thereof a radiopharmaceutical composition comprising 177Lu-PSMA I&T in a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, wherein the molar ratio of the PSMA I&T to 177Lu is from 4.4:1.0 to 7.6:1.0, the radiochemical purity of the composition is ≥95% at the time of administration, and wherein 177Lu-PSMA I&T treatment with 1, 2, 3, 4, 5, 6, 7, or 8 cycles at the dose is possible without the risk of kidney toxicities.

Statement 330: A method comprising administering to a human patient in need thereof a radiopharmaceutical composition comprising 177Lu-PSMA I&T in a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, wherein the molar ratio of the PSMA I&T to 177Lu is from 4.4:1.0 to 7.6:1.0, the radiochemical purity of the composition is ≥95% at the time of administration, and wherein 177Lu-PSMA I&T treatment with 1, 2, 3, 4, 5, 6, 7, 8 or more cycles provides a mean projected dose that is below a cumulative absorbed dose to the kidneys of 23 Gy.

Statement 331: A method comprising administering to a human patient in need thereof a radiopharmaceutical composition comprising 177Lu-PSMA I&T in a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, wherein the molar ratio of the PSMA I&T to 177Lu in the composition is from 5.0:1.0 to 6.0:1.0, 5.1:1.0 to 5.9:1.0, 5.2:1.0 to 5.8:1.0, 5.3:1.0 to 5.7.0:1.0, or 5.4:1.0 to 5.6:1.0, and wherein 177Lu-PSMA I&T treatment with 1, 2, 3, 4, 5, 6, 7, or 8 cycles at the dose is possible without the risk of kidney toxicities, and further wherein the composition has a radiochemical purity (RCP) of 95% or greater, 95.5% or greater, 96% or greater, 96.5% or greater, 97% or greater, 97.5% or greater, 98.0% or greater, 98.5% or greater, 99% or greater, or 99.5% or greater when administered.

Statement 332: A method comprising administering to a human patient in need thereof a radiopharmaceutical composition comprising 177Lu-PSMA I&T in a 7.4 GBq±0.10 GBq dose, 7.4 GBq±0.15 GBq dose, 7.4 GBq±0.20 GBq dose, 7.4 GBq±0.25 GBq dose, or 7.4 GBq±0.30 GBq dose, wherein the molar ratio of the PSMA I&T to 177Lu in the composition is from 5.0:1.0 to 6.0:1.0, 5.1:1.0 to 5.9:1.0, 5.2:1.0 to 5.8:1.0, 5.3:1.0 to 5.7.0:1.0, or 5.4:1.0 to 5.6:1.0, and wherein 177Lu-PSMA I&T treatment with 1, 2, 3, 4, 5, 6, 7, 8 or more cycles provides a mean projected dose that is below a cumulative absorbed dose to the kidneys of 23 Gy, and further wherein the composition has a radiochemical purity (RCP) of 95% or greater when administered.

Statement 333: The method of statement 195, further comprising SPECT imaging the human patient at 1 hour, 4 hours, 24 hours, 48 hours, and/or 168 hours post-administration of 177Lu-PSMA-I&T.

Statement 334: The method of statement 195, further comprising planar imaging the human patient at 1 hour, 4 hours, 24 hours, 48 hours, and/or 168 hours post-administration of 177Lu-PSMA-I&T.

Statement 335: The method of statement 195, further comprising SPECT or planar imaging the human patient at 1 hour, 4 hours, 24 hours, 48 hours, and/or 168 hours post-administration of 177Lu-PSMA-I&T.

Statement 336: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's kidneys of ≤0.43 Gy/MBq and with a standard deviation of less than 0.17, less than 0.16, less than 0.15, less than 0.14, less than 0.13, less than 0.12, less than 0.11, or less than 0.10, as determined by SPECT or planar imaging.

Statement 337: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, wherein the administration results in an absorbed dose to the patient's kidneys of ≤0.42 Gy/MBq and with a standard deviation of less than 0.17, less than 0.16, less than 0.15, less than 0.14, less than 0.13, less than 0.12, less than 0.11, or less than 0.10, as determined by SPECT or planar imaging.

Statement 338: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, wherein the administration results in an absorbed dose to the patient's kidneys of ≤0.41 Gy/MBq and with a standard deviation of less than 0.17, less than 0.16, less than 0.15, less than 0.14, less than 0.13, less than 0.12, less than 0.11, or less than 0.10, as determined by SPECT or planar imaging.

Statement 339: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's kidneys of 0.43 Gy/MBq±0.15 Gy/MBq, ±0.10 Gy/MBq, or ±0.05 Gy/MBq, with a standard deviation of about 0.15, about 0.10, or about 0.05.

Statement 340: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, wherein the administration results in an absorbed dose to the patient's kidneys of 0.42 Gy/MBq±0.15 Gy/MBq, ±0.10 Gy/MBq, or ±0.05 Gy/MBq, with a standard deviation of about 0.15, about 0.10, or about 0.05.

Statement 341: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's kidneys of 0.41 Gy/MBq±0.15 Gy/MBq, ±0.10 Gy/MBq, or ±0.05 Gy/MBq, with a standard deviation of about 0.15, about 0.10, or about 0.05.

Statement 342: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, wherein the administration results in an absorbed dose to the patient's kidneys of 0.41 Gy/MBq±0.15 Gy/MBq, ±0.10 Gy/MBq, or ±0.05 Gy/MBq, with a standard deviation of ≤0.15.

Statement 343: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's lacrimal glands of ≤0.43 Gy/MBq and with a standard deviation of less than 0.39, less than 0.38, less than 0.37, less than 0.36, less than 0.35, less than 0.34, less than 0.33, less than 0.32, less than 0.31, or less than 0.30, as determined by SPECT or planar imaging.

Statement 344: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's lacrimal glands of ≤0.42 Gy/MBq and with a standard deviation of less than 0.39, less than 0.38, less than 0.37, less than 0.36, less than 0.35, less than 0.34, less than 0.33, less than 0.32, less than 0.31, or less than 0.30, as determined by SPECT or planar imaging.

Statement 345: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, wherein the administration results in an absorbed dose to the patient's lacrimal glands of ≤0.41 Gy/MBq and with a standard deviation of less than 0.39, less than 0.38, less than 0.37, less than 0.36, less than 0.35, less than 0.34, less than 0.33, less than 0.32, less than 0.31, or less than 0.30, as determined by SPECT or planar imaging.

Statement 346: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's lacrimal glands of ≤0.40 Gy/MBq and with a standard deviation of less than 0.39, less than 0.38, less than 0.37, less than 0.36, less than 0.35, less than 0.34, less than 0.33, less than 0.32, less than 0.31, or less than 0.30, as determined by SPECT or planar imaging.

Statement 347: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, wherein the administration results in an absorbed dose to the patient's lacrimal glands of 0.40 Gy/MBq±0.15 Gy/MBq, ±0.10 Gy/MBq, or 0.05 Gy/MBq, with a standard deviation of ≤0.37.

Statement 348: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, wherein the administration results in an absorbed dose to the patient's salivary glands of ≤0.30 Gy/MBq and with a standard deviation of less than 0.18, less than 0.17, less than 0.16, less than 0.15, less than 0.14, less than 0.13, less than 0.12, less than 0.11, or less than 0.10, as determined by SPECT or planar imaging.

Statement 349: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's salivary glands of ≤0.25 Gy/MBq and with a standard deviation of less than 0.18, less than 0.17, less than 0.16, less than 0.15, less than 0.14, less than 0.13, less than 0.12, less than 0.11, or less than 0.10, as determined by SPECT or planar imaging.

Statement 350: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's salivary glands of ≤0.23 Gy/MBq and with a standard deviation of less than 0.18, less than 0.17, less than 0.16, less than 0.15, less than 0.14, less than 0.13, less than 0.12, less than 0.11, or less than 0.10, as determined by SPECT or planar imaging.

Statement 351: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's salivary glands of ≤0.20 Gy/MBq and with a standard deviation of less than 0.18, less than 0.17, less than 0.16, less than 0.15, less than 0.14, less than 0.13, less than 0.12, less than 0.11, or less than 0.10, as determined by SPECT or planar imaging.

Statement 352: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's salivary glands of ≤0.18 Gy/MBq and with a standard deviation of less than 0.18, less than 0.17, less than 0.16, less than 0.15, less than 0.14, less than 0.13, less than 0.12, less than 0.11, or less than 0.10, as determined by SPECT or planar imaging.

Statement 353: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's salivary glands of ≤0.30 Gy/MBq, with a standard deviation of ≤0.16.

Statement 354: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's salivary glands of ≤0.28 Gy/MBq, with a standard deviation of ≤0.16.

Statement 355: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's salivary glands of ≤0.26 Gy/MBq, with a standard deviation of ≤0.16.

Statement 356: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's salivary glands of ≤0.24 Gy/MBq, with a standard deviation of ≤0.16.

Statement 357: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's salivary glands of ≤0.22 Gy/MBq, with a standard deviation of ≤0.16.

Statement 358: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's salivary glands of ≤0.20 Gy/MBq, with a standard deviation of ≤0.16.

Statement 359: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, wherein the administration results in an absorbed dose to the patient's salivary glands of ≤0.18 Gy/MBq, with a standard deviation of ≤0.16.

Statement 360: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, wherein the administration results in an absorbed dose to the patient's salivary glands of 0.18 Gy/MBq±0.15 Gy/MBq, ±0.10 Gy/MBq, or ±0.05 Gy/MBq, with a standard deviation of ≤0.16.

Statement 361: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's liver of ≤0.04 Gy/MBq, as determined by SPECT or planar imaging.

Statement 362: A method comprising administering to the human patient in need thereof a radiopharmaceutical composition comprising 177Lu-PSMA I&T, wherein the molar ratio of the PSMA I&T to 177Lu is from 4.4:1.0 to 7.6:1.0, the radiochemical purity of the composition is ≥95% at the time of administration, and wherein the administration results in an absorbed dose to the patient's liver of ≤0.04 Gy/MBq and with a standard deviation of 0.0, as determined by SPECT or planar imaging.

Statement 363: The method of statement 195, wherein administering provides a mean distribution half-life of 1.89 hours±0.25 hours, ±0.15 hours, ±0.10 hours, or ±0.05 hours.

Statement 364: The method of statement 195, wherein administering provides a mean elimination half-life of 14.7 hours±0.25 hours, ±0.15 hours, ±0.10 hours, or ±0.05 hours.

Statement 365: The method of statement 195, wherein administering provides a mean clearance rate of 13.0 L/h±0.5 L/h, ±0.25 L/h, ±0.15 L/h, ±0.10 L/h, or ±0.05 L/h.

A Statement 366: composition comprising 177Lu-PSMA I&T and a PSMA I&T to [177Lu]Lu3+ ratio of ≤0.65 μg:mCi, ≤0.64 μg:mCi, ≤0.63 μg:mCi, ≤0.62 μg:mCi, ≤0.61 μg:mCi≤0.60 μg:mCi, wherein the composition is suitable for administration to a human patient in need thereof for at least 72 hours after formulation.

Statement 367: The composition of statement 366, wherein the composition has a radiochemical purity (RCP) of 95% or greater at administration.

Statement 368: The composition of statement 366, wherein the molar ratio of the PSMA I&T to 177Lu is from 4.4:1.0 to 7.6:1.0.

Statement 369: The composition of statement 366, wherein the composition has a radiochemical purity (RCP) of 98.0% or greater, 98.5% or greater, 99.0% or greater, or 99.5% or greater at administration.

Statement 370: The composition of statement 366, wherein the molar ratio of the PSMA I&T to 177Lu is from 7.0:1.0 to 7.6:1.0, 7.1:1.0 to 7.5:1.0, or 7.2:1.0 to 7.4:1.0.

Statement 371: The composition of statement 366, wherein the molar ratio of the PSMA I&T to 177Lu is from 6.0:1.0 to 7.0:1.0, 6.1:1.0 to 6.9:1.0, 6.2:1.0 to 6.8:1.0, 6.3:1.0 to 6.7:1.0, or 6.4:1.0 to 6.6:1.0.

Statement 372: The composition of statement 366, wherein the molar ratio of the PSMA I&T to 177Lu is from 5.0:1.0 to 6.0:1.0, 5.1:1.0 to 5.9:1.0, 5.2:1.0 to 5.8:1.0, 5.3:1.0 to 5.7.0:1.0, or 5.4:1.0 to 5.6:1.0.

Statement 373: The composition of statement 366, wherein the molar ratio of the PSMA I&T to 177Lu is from 4.4:1.0 to 5.0:1.0, 4.5:1.0 to 4.9:1.0, or 4.6:1.0 to 4.8:1.0.

Statement 374: The composition of statement 366, further comprising a buffering agent or solvent.

Statement 375: The composition of statement 366, wherein the PSMA I&T content is 30 μg/dose to 110 μg/dose, 30 μg/dose to 100 μg/dose, or 30 μg/dose to 90 μg/dose.

Statement 376: The composition of statement 366, wherein the PSMA I&T content is 95 μg/dose±15%, ±10%, or ±5%, 90 μg/dose±15%, ±10%, or ±5%, 85 μg/dose±15%, ±10%, or ±5%, 80 μg/dose±15%, ±10%, or ±5%, 75 μg/dose±15%, ±10%, or ±5%, 70 μg/dose±15%, ±10%, or ±5%, 60 μg/dose±15%, ±10%, or ±5%, 55 μg/dose±15%, ±10%, or ±5%, 50 μg/dose±15%, ±10%, or ±5%, 45 μg/dose±15%, ±10%, or ±5%, or 40 μg/dose±15%, ±10%.

Statement 377: The composition of statement 366, wherein the Fe metal content is ≤0.05 μg/GBq, ≤0.03 μg/GBq, ≤0.01 μg/GBq, or below the detectable limit.

Statement 378: The composition of statement 366, wherein the Cu metal content is ≤0.05 μg/GBq, ≤0.03 μg/GBq, ≤0.01 μg/GBq, or below the detectable limit.

Statement 379: The composition of statement 366, wherein the Zn metal content is ≤0.05 μg/GBq, ≤0.03 μg/GBq, ≤0.01 μg/GBq, or below the detectable limit.

Statement 380: The composition of statement 366, wherein the Pb metal content is ≤0.05 μg/GBq, ≤0.03 μg/GBq, ≤0.01 μg/GBq, or below the detectable limit.

Statement 381: The composition of statement 366, wherein the Cr metal content is ≤0.05 μg/GBq, ≤0.03 μg/GBq, ≤0.01 μg/GBq, or below the detectable limit.

Statement 382: The composition of statement 366, wherein the Co metal content is ≤0.05 μg/GBq, ≤0.03 μg/GBq, ≤0.01 μg/GBq, or below the detectable limit.

Statement 383: The composition of statement 366, wherein the Yb metal content is ≤0.05 μg/GBq, ≤0.03 μg/GBq, ≤0.01 μg/GBq, or below the detectable limit.

Statement 384: The composition of statement 366, wherein the composition is produced as part of a 4 Ci to 10 Ci batch scale.

Statement 385: The composition of statement 366, wherein the composition is produced as part of a 4 Ci to 15 Ci batch scale.

Statement 386: A radiopharmaceutical kit comprising a predetermined amount of the composition of statement 366.

Statement 387: A method of treating cancer in a patient in thereof comprising administering to the human patient the pharmaceutical composition of statement 366.

Statement 388: The method of statement 387, wherein the patient is treatment naïve.

Statement 389: The method of statement 387, wherein the patient is not treatment naïve.

Statement 390: The method of statement 387, wherein the pharmaceutical composition is administered to the cancer patient as a first line therapy or as a regimen.

Statement 391: A composition comprising 177Lu-PSMA I&T and a PSMA I&T to [177Lu]Lu3+ ratio of from 0.20 to 0.60 μg:mCi, wherein the composition is suitable for administration to a human patient in need thereof for at least 72 hours after formulation.

Statement 392: The composition of statement 391, wherein the composition has a radiochemical purity (RCP) of 95% or greater at administration.

Statement 393: The composition of statement 391, wherein the PSMA I&T to [177Lu]Lu3+ ratio is from 0.25 to 0.55 μg:mCi, from 0.30 to 0.50 μg:mCi, or from 0.35 to 0.45 μg:mCi.

Statement 394: The composition of statement 391, wherein the molar ratio of the PSMA I&T to 177Lu is from 4.4:1.0 to 7.6:1.0.

Statement 395: The composition of statement 391, wherein the composition has a radiochemical purity (RCP) of 97.0% or greater, 97.5% or greater, 98.0% or greater, 98.5% or greater, 99.0% or greater, or 99.5% or greater at administration.

Statement 396: The composition of statement 391, wherein the molar ratio of the PSMA I&T to 177Lu is from 7.0:1.0 to 7.6:1.0, 7.1:1.0 to 7.5:1.0, or 7.2:1.0 to 7.4:1.0.

Statement 397: The composition of statement 391, wherein the molar ratio of the PSMA I&T to 177Lu is from 6.0:1.0 to 7.0:1.0, 6.1:1.0 to 6.9:1.0, 6.2:1.0 to 6.8:1.0, 6.3:1.0 to 6.7:1.0, or 6.4:1.0 to 6.6:1.0.

Statement 398: The composition of statement 391, wherein the molar ratio of the PSMA I&T to 177Lu is from 5.0:1.0 to 6.0:1.0, 5.1:1.0 to 5.9:1.0, 5.2:1.0 to 5.8:1.0, 5.3:1.0 to 5.7.0:1.0, or 5.4:1.0 to 5.6:1.0.

Statement 399: The composition of statement 391, wherein the molar ratio of the PSMA I&T to 177Lu is from 4.4:1.0 to 5.0:1.0, 4.5:1.0 to 4.9:1.0, or 4.6:1.0 to 4.8:1.0.

Statement 400: The composition of statement 391, further comprising a buffering agent or solvent.

Statement 401: The composition of statement 391, wherein the PSMA I&T content is 30 μg/dose to 110 μg/dose, 30 μg/dose to 100 μg/dose, or 30 μg/dose to 90 μg/dose.

Statement 402: The composition of statement 391, wherein the PSMA I&T content is 95 μg/dose±15%, ±10%, or ±5%, 90 μg/dose±15%, ±10%, or ±5%, 85 μg/dose±15%, ±10%, or ±5%, 80 μg/dose±15%, ±10%, or ±5%, 75 μg/dose±15%, ±10%, or ±5%, 70 μg/dose±15%, ±10%, or ±5%, 60 μg/dose±15%, ±10%, or ±5%, 55 μg/dose±15%, ±10%, or ±5%, 50 μg/dose±15%, ±10%, or ±5%, 45 μg/dose±15%, ±10%, or ±5%, or 40 μg/dose±15%, ±10%.

Statement 403: The composition of statement 391, wherein the Fe metal content is ≤0.05 μg/GBq, ≤0.03 μg/GBq, ≤0.01 μg/GBq, or below the detectable limit.

Statement 404: The composition of statement 391, wherein the Cu metal content is ≤0.05 μg/GBq, ≤0.03 μg/GBq, ≤0.01 μg/GBq, or below the detectable limit.

Statement 405: The composition of statement 391, wherein the Zn metal content is ≤0.05 μg/GBq, ≤0.03 μg/GBq, ≤0.01 μg/GBq, or below the detectable limit.

Statement 406: The composition of statement 391, wherein the Pb metal content is ≤0.05 μg/GBq, ≤0.03 μg/GBq, ≤0.01 μg/GBq, or below the detectable limit.

Statement 407: The composition of statement 391, wherein the Cr metal content is ≤0.05 μg/GBq, ≤0.03 μg/GBq, ≤0.01 μg/GBq, or below the detectable limit.

Statement 408: The composition of statement 391, wherein the Co metal content is ≤0.05 μg/GBq, ≤0.03 μg/GBq, ≤0.01 μg/GBq, or below the detectable limit.

Statement 409: The composition of statement 391, wherein the Yb metal content is ≤0.05 μg/GBq, ≤0.03 μg/GBq, ≤0.01 μg/GBq, or below the detectable limit.

Statement 410: The composition of statement 391, wherein the composition is produced as part of a 4 Ci to 10 Ci batch scale.

Statement 411: The composition of statement 391, wherein the composition is produced as part of a 4 Ci to 15 Ci batch scale.

Statement 412: The composition of statement 391, wherein the composition is radiolabeled for 5 to 15 minutes at a temperature of about 65 to about 75 degrees C.

Statement 413: A radiopharmaceutical kit comprising a predetermined amount of the composition of statement 391.

Statement 414: A method of treating cancer in a patient in thereof comprising administering to the human patient the pharmaceutical composition of statement 391.

Statement 415: The method of statement 414, wherein the patient is treatment naïve.

Statement 416: The method of statement 414, wherein the patient is not treatment naïve.

Statement 417: The method of statement 414, wherein the pharmaceutical composition is administered to the cancer patient as a first line therapy or as a regimen.

Dosimetry Study Lu-177 PSMA I&T Patient Doses

| | | | Batch specific | | | | |
|---|---|---|---|---|---|---|---|
| | | | | RAC*, | MA*, | Lu- | |
| Subject | DOSE | BATCH | RCP*, | MBq/ | GBq/ | PSMA, | PSMA, |
| ID | ID | NO. | % | mL | μmol | μg/mL | μg/mL |
| 80-018 | 80-018 | LKMI2348A-1 | 99.2 | 559 | 212 | 1.4 | 2.0 |
| 42-027 | 42-027 | LKMI2405A-1 | 98.1 | 608 | 184 | 1.5 | 2.8 |
| 37-015 | 37-015 | LKMI2327A-1 | 98.9 | 596 | 209 | 1.3 | 2.3 |
| 37-016 | 37-016 | LKMI2327A-1 | 98.9 | 596 | 209 | 1.3 | 2.3 |
| 37-023 | 37-023 | LKMI2402A-1 | 99.0 | 592 | 196 | 1.7 | 2.4 |
| 37-022 | 37-022 | LKMI2402A-1 | 99.0 | 592 | 196 | 1.7 | 2.4 |
| 37-017 | 37-017 | LKMI2329G-1 | 99.1 | 565 | 195 | 1.5 | 2.5 |
| 42-030 | 42-030 | LKMI2411A-1 | 98.5 | 586 | 189 | 1.4 | 2.9 |
| 62-032 | 62-032 | LKMI2402A-2 | 98.9 | 570 | 185 | 1.7 | 2.4 |
| 80-024 | 80-024 | LKMI2407A-1 | 98.8 | 604 | 191 | 1.5 | 2.6 |
| 80-023 | 80-023 | LKMI2407A-1 | 98.8 | 604 | 191 | 1.5 | 2.6 |
| 25-008 | 25-008 | LKMI2322A-1 | 98.5 | 616 | 191 | 1.6 | 2.5 |
| 37-019 | 37-019 | LKMI2348G-1 | 99.2 | 590 | 190 | 1.4 | 2.5 |
| 80-019 | 80-019 | LKMI2402A-2 | 98.9 | 570 | 185 | 1.7 | 2.4 |
| 25-009 | 25-009 | LKMI2327G-1 | 98.8 | 580 | 183 | 1.4 | 2.5 |
| 25-007 | 25-007 | LKMI2320G-2 | 99.1 | 604 | 182 | 1.3 | 2.3 |
| 66-024 | 66-024 | LKMI2409G-1 | 98.8 | 556 | 172 | 1.5 | 2.9 |
| 42-028 | 42-028 | LKMI2405G-1 | 98.5 | 572 | 163 | 1.5 | 3.1 |
| 80-022 | 80-022 | LKMI2406G-1 | 98.7 | 548 | 163 | 1.5 | 3.1 |
| 80-020 | 80-020 | LKMI2406G-1 | 98.7 | 548 | 163 | 1.5 | 3.1 |
| 37-020 | 37-020 | LKMI2352A-1 | 99.1 | 594 | 169 | 2.1 | 2.3 |
| 12-004 | 12-004 | LKMI2315G-1 | 99.1 | 563 | 152 | 1.3 | 3.5 |
| 80-007 | 80-007 | LKMI2318B-1 | 98.4 | 586 | 143 | 1.4 | 3.7 |
| 80-001 | 80-001 | LKMI2316G-1 | 98.6 | 588 | 139 | 1.4 | 3.9 |
| 37-013 | 37-013 | LKMI2319A-1 | 98.9 | 576 | 138 | 1.4 | 3.2 |
| 25-005 | 25-005 | LKMI2316A-1 | 98.9 | 585 | 136 | 1.4 | 3.7 |
| 80-004 | 80-004 | LKMI2317G-1 | 98.5 | 585 | 126 | 1.4 | 3.8 |
| | | Theo- | | | Theo- | | |
| | Measured | retical | Lu- | Measured | retical | Dose specific | |
| Other | PSMA | PSMA | PSMA | PSMA | PSMA | Dose | Dose |
| PSMA, | Sum, | Sum, | μg/ | Sum | Sum | Vol, | Act**, |
| μg/mL | μg/mL | μg/mL | dose | μg/dose | μg/dose | mL | MBq |
| 0.7 | 4.1 | 5.9 | 23.5 | 68.9 | 99.8 | 16.80 | 7276 |
| 0.8 | 5.1 | 5.9 | 21.1 | 71.6 | 83.4 | 14.04 | 7380 |
| 0.8 | 4.4 | 5.9 | 21.3 | 72.1 | 97.3 | 16.39 | 7396 |
| 0.8 | 4.4 | 5.9 | 21.3 | 72.1 | 97.3 | 16.39 | 7396 |
| 0.6 | 4.7 | 5.9 | 27.9 | 77.0 | 97.3 | 16.39 | 7469 |
| 0.6 | 4.7 | 5.9 | 27.9 | 77.0 | 97.3 | 16.39 | 7469 |
| 0.5 | 4.5 | 6.4 | 25.9 | 77.7 | 109.7 | 17.26 | 7485 |
| 0.5 | 4.8 | 5.9 | 22.4 | 76.8 | 95.1 | 16.01 | 7401 |
| 0.7 | 4.8 | 5.9 | 28.0 | 79.0 | 97.8 | 16.46 | 7400 |
| 0.8 | 4.9 | 5.9 | 24.3 | 79.5 | 96.3 | 16.22 | 7475 |
| 0.8 | 4.9 | 5.9 | 24.3 | 79.5 | 96.3 | 16.22 | 7475 |
| 0.9 | 5.0 | 5.9 | 25.4 | 79.4 | 94.3 | 15.88 | 7450 |
| 0.9 | 4.8 | 6.4 | 23.5 | 80.4 | 106.5 | 16.75 | 7416 |
| 0.7 | 4.8 | 5.9 | 29.0 | 82.0 | 101.5 | 17.08 | 7462 |
| 1.0 | 4.9 | 6.4 | 23.6 | 82.6 | 107.1 | 16.85 | 7533 |
| 1.5 | 5.1 | 6.4 | 21.0 | 82.2 | 102.5 | 16.12 | 7428 |
| 0.6 | 5.0 | 6.4 | 25.6 | 85.3 | 108.4 | 17.05 | 7445 |
| 0.8 | 5.4 | 6.4 | 24.8 | 89.2 | 104.9 | 16.51 | 7482 |
| 0.6 | 5.2 | 6.4 | 26.7 | 92.6 | 113.2 | 17.81 | 7488 |
| 0.6 | 5.2 | 6.4 | 26.7 | 92.6 | 113.2 | 17.81 | 7488 |
| 1.1 | 5.5 | 5.9 | 34.4 | 90.0 | 97.2 | 16.37 | 7186 |
| 0.9 | 5.7 | 6.4 | 22.6 | 99.2 | 110.6 | 17.40 | 7497 |
| 1.2 | 6.3 | 5.9 | 23.4 | 105.1 | 99.1 | 16.68 | 7549 |
| 1.2 | 6.5 | 6.4 | 23.4 | 108.5 | 106.1 | 16.69 | 7410 |
| 1.8 | 6.4 | 5.9 | 23.7 | 108.5 | 100.7 | 16.95 | 7390 |
| 1.5 | 6.6 | 6.4 | 23.5 | 110.6 | 106.5 | 16.75 | 7458 |
| 1.9 | 7.1 | 6.4 | 23.5 | 119.1 | 106.6 | 16.77 | 7455 |

| | | | | Lacrimal | Salivary | |
|---|---|---|---|---|---|---|
| PSMA | PSMA | Ratio** | Kidneys | Glands | Glands | Liver |
| Content, | Content, | (PSMA: | (Gy/ | (Gy/ | (Gy/ | (Gy/ |
| μg | nmol | Lu-177) | GBq) | GBq) | GBq) | GBq) |
| 68.9 | 44.3 | 4.4 | 0.31 | N/A | 0.11 | 0.04 |
| 71.6 | 46.3 | 4.6 | 0.36 | N/A | 0.16 | 0.02 |
| 72.1 | 46.6 | 4.6 | 0.32 | 0.19 | 0.06 | 0.02 |
| 72.1 | 46.6 | 4.6 | 0.54 | 0.20 | 0.00 | 0.02 |
| 77.0 | 49.4 | 4.8 | 0.28 | 0.07 | 0.19 | 0.06 |
| 77.0 | 49.4 | 4.8 | 0.56 | 0.06 | 0.10 | 0.02 |
| 77.7 | 50.0 | 4.9 | 0.41 | 0.66 | 0.04 | 0.04 |
| 76.8 | 49.7 | 4.9 | 0.23 | 0.08 | 0.11 | 0.03 |
| 79.0 | 50.8 | 5.0 | 0.53 | 0.16 | 0.15 | 0.03 |
| 79.5 | 51.3 | 5.0 | 0.30 | 0.26 | 0.26 | 0.03 |
| 79.5 | 51.3 | 5.0 | 0.38 | 0.47 | 0.19 | 0.07 |
| 79.4 | 51.2 | 5.0 | 0.81 | 0.63 | 0.12 | 0.03 |
| 80.4 | 52.0 | 5.1 | 0.24 | 0.08 | 0.20 | 0.10 |
| 82.0 | 52.7 | 5.1 | 0.17 | 0.07 | 0.32 | 0.05 |
| 82.6 | 53.5 | 5.2 | 0.29 | N/A | 0.16 | 0.02 |
| 82.2 | 53.4 | 5.2 | 0.34 | 0.54 | 0.03 | 0.02 |
| 85.3 | 55.1 | 5.4 | 0.55 | N/A | 0.09 | 0.02 |
| 89.2 | 57.8 | 5.6 | 0.43 | 0.38 | 0.39 | 0.08 |
| 92.6 | 59.9 | 5.8 | 0.05 | 0.05 | 0.13 | 0.09 |
| 92.6 | 59.9 | 5.8 | 0.45 | 0.04 | 0.14 | 0.01 |
| 90.0 | 57.7 | 5.8 | 0.35 | 0.09 | 0.80 | 0.04 |
| 99.2 | 64.6 | 6.3 | 0.57 | N/A | 0.27 | 0.04 |
| 105.1 | 68.5 | 6.6 | 0.48 | 1.19 | 0.18 | 0.06 |
| 108.5 | 70.8 | 6.9 | 0.27 | 0.94 | 0.29 | 0.07 |
| 108.5 | 70.7 | 7.0 | 0.27 | 1.09 | 0.10 | 0.05 |
| 110.6 | 72.1 | 7.0 | 0.64 | 0.68 | 0.03 | 0.03 |
| 119.1 | 77.8 | 7.6 | 0.28 | 0.62 | 0.28 | 0.02 |

RCP = Radiochemical Purity
RAC = Radioactivity Concentration
MA = Molar Activity
*at the time of release
**at the calibration time
Other PSMA means PSMA I&T chelated with non-active metal impurities (mainly Zn, Fe and Cu)

What is claimed is:

1. A composition comprising ¹⁷⁷Lu-PSMA I&T; wherein the molar ratio of the PSMA I&T to ¹⁷⁷Lu is from 3.0:1.0 to 8.0:1.0, and the composition is suitable for administration to a human patient in need thereof for at least 72 hours after formulation.

2. The composition of claim 1; wherein the molar ratio of the PSMA I&T to ¹⁷⁷Lu is from 4.4:1.0 to 7.6:1.0.

3. The composition of claim 2, wherein the composition is suitable for administration to a human patient in need thereof for at least 120 hours after formulation.

4. The composition of claim 1, wherein the composition has a radiochemical purity (RCP) of 95% or greater at administration.

5. The composition of claim 1, wherein the pH of the composition is from 3.5 to 6.0.

6. The composition of claim 1, wherein the pH of the composition is from 3.5 to 5.0.

7. The composition of claim 1, wherein the pH of the composition is from 3.5 to 4.5.

8. The composition of claim 1, wherein the molar ratio of the PSMA I&T to ¹⁷⁷Lu is from 7.0:1.0 to 8.0:1.0, 7.1:1.0 to 7.9:1.0, 7.2:1.0 to 7.8:1.0, 7.3:1.0 to 7.7:1.0, or 7.4:1.0 to 7.6:1.0.

9. The composition of claim 1, wherein the molar ratio of the PSMA I&T to ¹⁷⁷Lu is from 6.0:1.0 to 7.0:1.0, 6.1:1.0 to 6.9:1.0, 6.2:1.0 to 6.8:1.0, 6.3:1.0 to 6.7:1.0, or 6.4:1.0 to 6.6:1.0.

10. The composition of claim 1, wherein the molar ratio of the PSMA I&T to ¹⁷⁷Lu is from 5.0:1.0 to 6.0:1.0, 5.1:1.0 to 5.9:1.0, 5.2:1.0 to 5.8:1.0, 5.3:1.0 to 5.7.0:1.0, or 5.4:1.0 to 5.6:1.0.

11. The composition of claim 1, wherein the molar ratio of the PSMA I&T to ¹⁷⁷Lu is from 4.0:1.0 to 5.0:1.0, 4.1:1.0 to 4.9:1.0, 4.2:1.0 to 4.8:1.0, 4.3:1.0 to 4.7.0:1.0, or 4.4:1.0 to 4.6:1.0.

12. The composition of claim 1, wherein the molar ratio of the PSMA I&T to ¹⁷⁷Lu is from 3.0:1.0 to 4.0:1.0, 3.1:1.0 to 3.9:1.0, 3.2:1.0 to 3.8:1.0, 3.3:1.0 to 3.7.0:1.0, or 3.4:1.0 to 3.6:1.0.

13. The composition of claim 1, wherein the PSMA I&T content is 30 μg/dose to 110 μg/dose, 30 μg/dose to 100 μg/dose, or 30 μg/dose to 90 μg/dose.

14. The composition of claim 1, wherein the PSMA I&T content is 95 μg/dose±15%, ±10%, or ±5%, 90 μg/dose±15%, ±10%, or ±5%, 85 μg/dose±15%, ±10%, or ±5%, 80 μg/dose±15%, ±10%, or ±5%, 75 μg/dose±15%, ±10%, or ±5%, 70 μg/dose±15%, ±10%, or ±5%, 60 μg/dose±15%, ±10%, or ±5%, 55 μg/dose±15%, ±10%, or ±5%, 50 μg/dose±15%, ±10%, or ±5%, 45 μg/dose±15%, ±10%, or ±5%, or 40 μg/dose±15%, ±10%.

15. The composition of claim 1, wherein Fe metal content is ≤0.05 μg/GBq, ≤0.03 μg/GBq, ≤0.01 μg/GBq, or below the detectable limit.

16. The composition of claim 1, wherein Cu metal content is ≤0.05 μg/GBq, ≤0.03 μg/GBq, ≤0.01 μg/GBq, or below the detectable limit.

17. The composition of claim 1, wherein Zn metal content is ≤0.05 μg/GBq, ≤0.03 μg/GBq, ≤0.01 μg/GBq, or below the detectable limit.

18. The composition of claim 1, wherein Pb metal content is ≤0.05 μg/GBq, ≤0.03 μg/GBq, ≤0.01 μg/GBq, or below the detectable limit.

19. The composition of claim 1, wherein the composition has a radiochemical purity (RCP) of 97.5% or greater at 72 hours after production.

20. The composition of claim 1, wherein the composition has a radiochemical purity (RCP) of 98.0% or greater at 72 hours after production.

21. The composition of claim 1, wherein the composition is suitable for human administration for 7 or more cycles of treatment.

22. The composition of claim 1, wherein the solution comprises about 10 mg/ml to about 50 mg/ml ascorbic acid.

23. The composition of claim 1, wherein the solution comprises at least 28 mg/ml ascorbic acid.

24. The composition of claim 1, wherein the composition further comprises DTPA.

25. A radiopharmaceutical kit comprising the composition of claim 1.

26. A method of treating cancer in a human patient in need thereof comprising administering to the human patient the composition of claim 1.

27. A method of treating cancer in a human patient in need thereof comprising administering to the human patient the composition of claim 2.

28. A method of treating or reducing the incidence of a cancer using an radiochemical composition, the method comprising administering a radiochemical composition into a human patient in need thereof, the radiochemical composition comprising ¹⁷⁷Lu-PSMA I&T in a solution having a pH of 3.5 to 6.0, wherein the molar ratio of the PSMA I&T to ¹⁷⁷Lu is from 3.0:1.0 to 8.0:1.0, wherein the solution has a radiochemical purity of more than 95% when administered, and wherein the composition is suitable for administration to a human patient in need thereof for at least 72 hours after formulation.

29. The method of claim 28, wherein the cancer is prostate cancer.

30. The method of claim 28, wherein the cancer is metastatic castration-resistant prostate cancer (mCRPC).
